(19) United States
(12) Patent Application Publication
(10) Pub. No.: US 2024/0174634 A1
(43) Pub. Date: May 30, 2024

(54) Aldehyde Dehydrogenase Inhibitors and Their Therapeutic Use

(71) Applicant: Cancer Research Technology Limited, London (GB)

(72) Inventors: Christopher Stephen KERSHAW, Macclesfield (GB); Mohammed ALJARAH, Macclesfield (GB); Dan NICULESCU-DUVAZ, Macclesfield (GB); Mark Philip DODSWORTH, Glasgow (GB); Cinzia BORDONI, Macclesfield (GB); Caroline SPRINGER, Macclesfield (GB)

(73) Assignee: Cancer Research Technology Limited, London (GB)

(21) Appl. No.: 18/265,063

(22) PCT Filed: Dec. 10, 2021
(86) PCT No.: PCT/EP2021/085260

§ 371 Date: Jun. 2, 2023

(30) Foreign Application Priority Data
Dec. 10, 2020 (GB) 2019475.9

(51) Int. Cl. C07D 401/12; A61P 35/00; C07D 215/227; C07D 401/14; C07D 409/12; C07D 413/12; C07D 417/12; C07D 417/14

(52) U.S. Cl. CPC C07D 401/12; A61P 35/00; C07D 215/227; C07D 401/14; C07D 409/12; C07D 413/12; C07D 417/12; C07D 417/14

(57) ABSTRACT

The present invention pertains generally to the field of therapeutic compounds. More specifically the present invention pertains to certain aldehyde dehydrogenase inhibitor compounds (also referred to herein as “ALDHI compounds”), that, inter alia, inhibit aldehyde dehydrogenase enzyme ALDH1A3. The present invention also pertains to pharmaceutical compositions comprising such compounds, and the use of such compounds and compositions, both in vitro and in vivo, to inhibit ALDH1A3 enzyme; to treat disorder (e.g., diseases) that are ameliorated by the inhibition of ALDHIA3 enzyme; to treat a proliferative disorder, cancer, obesity, diabetes, a cardiovascular disorder, etc.

### RELATED APPLICATION

This application is related to United Kingdom (GB) patent application number 2019475.9 filed 10 Dec. 2020, the contents of which are incorporated herein by reference in their entirety.

### TECHNICAL FIELD

The present invention pertains generally to the field of therapeutic compounds. More specifically the present invention pertains to certain aldehyde dehydrogenase inhibitor compounds (also referred to herein as “ALDHI compounds”), that, inter alia, inhibit aldehyde dehydrogenase enzyme ALDH1A3. The present invention also pertains to pharmaceutical compositions comprising such compounds, and the use of such compounds and compositions, both in vitro and in vivo, to inhibit ALDH1A3 enzyme; to treat disorder (e.g., diseases) that are ameliorated by the inhibition of ALDH1A3 enzyme; to treat a proliferative disorder, cancer, obesity, diabetes, a cardiovascular disorder, etc.

### BACKGROUND

Publications are cited herein in order to more fully describe the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Aldehyde Dehydrogenase Enzymes

The aldehyde dehydrogenase enzymes (ALDH) (EC 1.2.1.3) are a class of evolutionarily conserved NAD(P)-dependent oxido-reductases (19 human isoforms) that catalyse the oxidation of various exogenous and endogenous aldehydes to their corresponding carboxylic acids.

Although the ALDH enzymes are involved in a broad spectrum of biological processes, the biological role of the majority of the isoforms remains to be elucidated. ALDH2, the most studied isoform, plays a key role in alcohol metabolism (oxidising acetaldehyde to acetic acid), in the protection of ischemic hearts and in cancer (see, e.g., Rodriguez-Zavala, 2019). Recent literature highlighted the importance of members of the ALDH1A sub-family and especially ALDH1A3 in different pathologies such as type II diabetes, obesity, cancer, pulmonary arterial hypertension (PAH), and neointimal hyperplasia (NIH). ALDH1A3 is a member of a sub-family of cytosolic and homotetrameric enzymes that includes also ALDH1A1 and ALDH1A2.

Overexpressed ALDH1A3 plays a key role in type II diabetes. ALDH1A3 expression is elevated in rodent models of diabetes and in patients with type II diabetes, and is associated with reduced insulin production by pancreatic islets cells. ALDH1A3 is a marker of dedifferentiated pancreatic β-cells, with impaired insulin secretion and mitochondrial function (see, e.g., Kim-Muller, 2016; Cinti et al., 2016; Burke et al., 2018). Treatment of mice with a pharmacological ALDH1A3 inhibitor restored insulin secretion and improved blood glucose control (see, e.g., Esposito et al., 2021).

ALDH1A3 expression is also elevated in rodent models of obesity leading to type II diabetes (see e.g., Burke et al., 2017) and pancreatic islet cells from obese diabetic mice express high levels of ALDH1A3 (see, e.g., Esposito et al., 2021).

In cancer, an aberrant expression of ALDH1A3 has been associated with progression and poor prognosis of several tumour types and is a hallmark of a subpopulation of cancer cells known as cancer stem cells (CSCs) or tumour-initiating cells (TICs) (see, e.g., Marcato, 2011a; Luo, 2012).

CSCs are a subpopulation of undifferentiated cells within a heterogeneous tumour defined by their ability to self-renew and produce differentiated daughter cells during asymmetric division. They are characterised by an increased tumour-seeding potential, are involved in tumour progression, metastasis and are associated with chemo- and radio-resistance. Current therapies target bulk tumour cells but CSCs escape, resulting in tumour recurrence and treatment failure (see, e.g., Pattabiraman, 2014). As a result, tumours may initially appear eradicated, but later recur because small subpopulations of CSCs have survived. This concept is important clinically because it emphasises the crucial need to target CSCs to achieve durable responses. It follows that therapeutic targeting of the survival mechanisms employed by CSCs will increase the efficacy of anticancer therapies and decrease the risk of relapse and progression.

ALDH activity measured by the Aldefluor assay has been used as a marker of CSCs and to isolate these cells from the bulk tumour (see, e.g., Ginestier, 2007). In many tumour types, ALDH1A3 has been reported as the dominant isoform, and is responsible for Aldefluor activity. ALDH1A3 is a key functional driver for survival, growth, metastasis and resistance of CSCs but also associated with poor prognosis and poor overall survival in patients affected by many types of tumours such as melanoma, breast and glioblastoma (see, e.g., Duan, 2016; Rodriguez-Torres, 2016).

The underlying mechanism of action by which ALDH1A3 contributes to survival and progression of cancer cells and CSCs in particular remains unclear. Two main functions of ALDH1A3 have been suggested to play a key part: the detoxification of cytotoxic aldehydes and the biosynthesis of retinoic acid (RA) (see, e.g., Duan, 2016).

Oxidative stress as a result of chemotherapy or other factors is a recurrent feature in cancer cells and CSCs and leads to an increase of intracellular reactive oxygen species (ROS). The consequences of this increase in ROS are peroxidation of phospholipids and generation of reactive aldehydes such as 4-hydroxynonenal (4-HNE) and malondialdehyde (MDA). An aberrant accumulation of aldehydes can lead to oxidative damage to the cell and apoptosis. These apoptogenic aldehydes need to be metabolised into less toxic carboxylic acids by an overexpressed ALDH1A3 to preserve the homeostasis of the cancer cells and CSCs (see, e.g., Laskar, 2019).

ALDH1A3 along with the other members of the ALDH1A subfamily play also a key role in the biosynthesis of RA from retinal and in the expression of many RA-inducible genes involved in stemness and proliferation through the interaction of RA with nuclear RA receptors, RAR and RXR (see, e.g., Duan, 2016).

In ovarian cancer, ALDH1A3 but also ALDH1A1 are overexpressed in many cell lines according to the Cancer Cell Line Encyclopedia (CCLE) and ALDH1A3 is especially the dominant isoform in several serous adenocarcinoma derived ovarian cancer cell lines (see, e.g., https://portals.broadinstitute.orq/ccle; Chefetz, 2019). Genetic knock-down of ALDH1A3 by siRNA led to the necroptosis of the CD133+ CSC population of these cells in vitro (Chefetz, 2019). Inhibition of ALDH1A3 reverses resistance and synergises with chemotherapy (for example taxol) in a resistant cell line and high grade serous ovarian carcinoma patient derived tumour spheroids (see, e.g., Huddle et al., 2021) and with DNA damage checkpoint inhibitors (ATMi/ATRi) in vitro and in vivo (see, e.g., Grimley et al., 2021).

In melanoma, ALDH1A3 is the most expressed isoform across various cell lines including patient-derived cells (CCLE), while its expression is low in non-malignant human epidermal melanocytes and is regulated by epigenetic mechanisms (see, e.g., Perez-Alea, 2017). ALDH1A3 has been associated with CSCs (see, e.g., Luo, 2012; Kozovska, 2016) and plays a key role in melanomagenesis, progression and metastasis. A role in the elimination of cytotoxic 4-HNE and MDA has been suggested to explain the effect of ALDH1A3 on preserving the cellular homeostasis (see, e.g., Pérez-Alea, 2017) while other studies reported a RA-driven expression of stem cell genes (see, e.g., Luo, 2012). Its inhibition by genetic knock-down (ALDH1A3 shRNA) led to a reduction of number of colonies and spheres formed in vitro and a reduction of tumour growth in vivo with different cell lines (see, e.g., Perez-Alea, 2017) and a sensitization of paclitaxel-resistant ALDH+1205Lu and A375 cells to drug-induced cell death (see, e.g., Luo, 2012). Of note, the genetic knockdown of both ALDH1A1 and ALDH1A3 by shRNA or their inhibition by the pan-ALDH1A inhibitor, DIMATE, led to a superior effect both in vitro and in vivo (see, e.g., Pérez-Alea, 2017).

In breast cancer, ALDH1A3 has been reported as the key contributor to the Aldefluor activity of most cell lines and a marker of breast CSCs, especially of the CD44+CD24-population (see, e.g., Marcato, 2011b). There is evidence that CSCs in breast cancer are responsible for tumour recurrence following targeted therapy (see, e.g., Simões, 2015). Several studies have highlighted ALDH1A3 as a key driver for tumour growth and lung metastasis of several breast cancer cell lines such as MDA-MB-231 and SUM-159 cells (see, e.g., Marcato, 2015; Croker, 2017). A mechanism implicating RA and an upregulation of RA-inducible genes has been suggested to explain the effect of ALDH1A3 on tumour growth (see, e.g., Marcato, 2015). Increased expression of ALDH1A3 in breast cancer patients, especially with the triple-negative subtype (ER⁻/PR⁻/HER2⁻), has been associated with poorer prognosis and worse clinical outcome, tumour stage and grade and is also predictive of metastasis (see, e.g., Opdenaker, 2014; Marcato, 2015).

In glioma patients, aberrant expression of ALDH1A3 has been associated with the most aggressive form, high grade glioblastoma, and is a marker of poor prognosis and poor overall survival (see, e.g., Mao, 2013; Zhang, 2013; Li, 2018; Ni, 2020). On a cellular level, ALDH1A3 is overexpressed in the more aggressive and radiotherapy-resistant mesenchymal subtype of glioma stem cells (Mes-GSC) (see, e.g., Ni, 2020), is a key driver of the transition from the proneural subtype (Pn-GSC) to the mesenchymal one (see, e.g., Li, 2018) and is implicated in processes such as cell proliferation, ECM organisation, cell adhesion and ECM-receptor interaction (see, e.g., Vasilogiannakopoulou, 2018). An association of ALDH1A3 with the transcription factor FOXD1 (see, e.g., Cheng, 2016) or a role of ALDH1A3 in facilitating glucose uptake (see, e.g., Ni, 2020) have been suggested to explain its implication in the maintenance and tumourigenesis of Mes-GSCs.

ALDH1A3 has also been associated with poor prognosis and metastasis in patients with pancreatic cancer (see, e.g., Kong, 2016; Nie, 2020), gallbladder cancer (see, e.g., Yang, 2013), prostate cancer (associated with a high expression of miR-187) (see, e.g., Casanova-Salas, 2015), papillary thyroid cancer (see, e.g., Cai et al., 2021), with aggressive phenotype in neuroblastoma (see, e.g., Flahaut, 2016) and with chemoresistance and metastasis in a colorectal cancer model (see, e.g., Durinikova, 2018). ALDH1A3 has also been associated with testicular germ cells tumours (see, e.g., Schmidtova et al., 2019), gastric cancer (see, e.g., Kawakami et al., 2020), and cholangiocarcinoma (see, e.g., Chen et al., 2016). ALDH1A3 has an essential role for maintaining a population of CSCs in non-small cell lung cancer (see, e.g., Shao, 2014) and is associated with resistance to EGFR inhibitors in NSCLC (see, e.g., Aissa et al., 2021). ALDH1A3 is up-regulated in cisplatin-resistant hepatoblastoma (see, e.g., Marayati et al., 2021) and in sunitinib-resistant renal cells carcinoma (see, e.g., Kamada et al., 2021). The expression of ALDH1A3 is responsible for the survival and activity of malignant pleural mesothelioma (MPM) chemoresistant cell sub-populations (see, e.g., Cioce et al., 2021). In colorectal cancer (CRC) cells, ALDH1A3 knockdown reduces clonogenicity and proliferation and induces apoptosis; ALDH1A3 is suggested as the key protein responsible for the DSF-Cu complex efficacy in CRC xenografts, and a driver of increased glycolysis in CRC tumours (see, e.g., Huang et al., 2021).

ALDH1A3 signaling appears to be important for T-regulatory (Treg) cell induction and function through the production of retinoic acid by multiple cell types (e.g., dendritic cells, macrophages, eosinophils, epithelial cells). ALDH1A3 inhibition could increase the ratio of effector T cells to Treg cells within tumor tissue leading to increased tumor immunity and tumour rejection (see, e.g., Bazewicz et al., 2019). Genetic knockout of ALDH1A3 (RALDH3) in a fibrosarcoma tumour model led to robust T-cell infiltration and impaired tumour growth in immunocompetent mice, and to synergy with immune checkpoint inhibitors, e.g., anti-PD1 antibodies. Therefore, ALDH1A3 inhibitors can enhance anti-tumour response to immunotherapy such as anti-PD1, anti-PDL1, anti-CTLA4, anti-IL3 antibodies (see, e.g., Haldar et al., 2020).

ALDH1A3 display all the attributes of a promising therapeutic target in cancer. In addition, its low expression and minor physiological roles in non-malignant cells limit on-target toxicity of potential ALDH1A3 inhibitors.

Recently, a key role of ALDH1A3 in the proliferation of vascular (see, e.g., Xie et al., 2019) and pulmonary arterial (see, e.g., Li et al., 2021) smooth muscle cells (SMCs) and the resulting formation of a neointima has been reported. This reduction in lumen space leads to pathologies such as neointimal hyperplasia (NIH), a major cause of restenosis, and pulmonary arterial hypertension (PAH). In vitro, genetic inhibition of ALDH1A3 prevents vascular and pulmonary arterial SMCs proliferation. In vivo, perivascular administration of disulfiram reduces NIH in a rat angioplasty model while mice with ALDH1A3 gene knockout do not develop hypoxia-induced PAH.

ALDH1A3 inhibition could lead to intimal hyperplasia mitigation and hence be useful for treatment of restenosis and/or to increase the chance of success of coronary artery angioplasty/stenting or bypass vein grafting, arteriovenous fistula for dialysis access, and allograft transplantation (see, e.g., Xie et al., 2019).

There are currently no ALDH1A3 selective inhibitor approved or in clinical development. Several non-selective, broad-spectrum inhibitors ALDH inhibitors of ALDH1A1, ALDH2 and/or ALDH3A1 have been reported in the literature to also inhibit ALDH1A3. These inhibitors display off-target toxicity, poor pharmacokinetic (PK) properties including short half-life and lack of oral bioavailability and/or lack of in vivo efficacy. Early non-selective small molecules reported to inhibit ALDH1A3 include citral, dimethylthioampal (DIMATE), N,N-diethylaminobenzaldehyde (DEAB) and disulfiram (DSF) (see, e.g., Koppaka, 2012; Pors, 2014; Morgan, 2015; Yasgar, 2017; Dinavahi, 2019). These compounds have shown in vivo reduction of tumour growth and/or metastasis in different models, especially breast and melanoma (see, e.g., Thomas, 2016; Pérez-Alea, 2017; Matsunaga, 2018). All these compounds suffer from poor PK properties, limiting their use to intravenous or intraperitoneal administration. Some of these compounds inhibit multiple other targets and pathways not related to the ALDH1A family, for example DSF, currently in clinical development for patients with advanced lung cancer, although it is not clear which of the possible metabolites generated in vivo and what pharmacological activity is responsible for the anticancer efficacy.

A DEAB analogue, compound 673A (see, e.g., Chefetz, 2019), and a series of thiopyrimidinone (see, e.g., Huddle, 2018; Larsen, 2017) show inhibition of all three ALDH1A isoforms. 673A displayed in vivo reduction of tumour growth in combination with cisplatin in several models of ovarian cancer (see, e.g., Chefetz, 2019). These compounds lack oral bioavailability. Recently reported non-selective, pan-ALDH1A family pyrazolopyrimidinone inhibitors were shown to have efficacy in ovarian cancer cellular models (see, e.g., Huddle et al., 2021).

A series of tetrahydroquinoline derivatives inhibiting selectively ALDH1A3 have been reported recently (see, e.g., Esposito, 2020). Compound MBE-1.5 showed in vivo reduction of tumour growth and metastasis in mice in combination with paclitaxel in paclitaxel-resistant models of breast cancer. Similarly to other compounds reported, these analogues are not administered via the oral route.

Additional heterocyclic inhibitors of ALDH1A3 are described (see, e.g., Esposito et al., 2021) that show anti-metastatic efficacy in combination with paclitaxel, and restored insulin secretion in a diabetes mouse model. The imidazopyridine ALDH1A3 inhibitor NR6 (see, e.g., Gelardi et al., 2021) shows anti-metastatic activity in wound healing and invasion assays in glioblastoma and colorectal cancer cells, but NR6 lacks biochemical potency (IC50˜5 μM). Benzyloxybenzaldehyde ALDH1A3 inhibitors are reported, but without pharmacokinetics data (see, e.g., Ibrahim et al., 2021).

There is a clear need for potent selective ALDH1A3 inhibitors with good pharmacokinetic properties, which are suitable for oral dosing with minimal or no toxicity.

This disclosure provides compounds and compositions that selectively inhibit ALDH1A3 to target cancer and CSC subpopulations, that should lead to the regression of various tumour types, and when combined with conventional or targeted therapy, to tumour elimination. The compounds should also have therapeutic utility in the treatment of other diseases (for example, type II diabetes, etc.), as discussed herein.

### SUMMARY OF THE INVENTION

One aspect of the invention pertains to certain aldehyde dehydrogenase inhibitor compounds (referred to herein as ALDHI compounds), as described herein.

Another aspect of the invention pertains to a composition (e.g., a pharmaceutical composition) comprising an ALDHI compound, as described herein, and a pharmaceutically acceptable carrier or diluent.

Another aspect of the invention pertains to a method of preparing a composition (e.g., a pharmaceutical composition) comprising the step of mixing an ALDHI compound, as described herein, and a pharmaceutically acceptable carrier or diluent.

Another aspect of the present invention pertains to a method of inhibiting ALDH1A3 enzyme (e.g., inhibiting or reducing or blocking the activity or function of ALDH1A3 enzyme), in vitro or in vivo, comprising contacting the ALDH1A3 enzyme with an effective amount of an ALDHI compound, as described herein.

Another aspect of the present invention pertains to a method of inhibiting ALDH1A3 enzyme (e.g., inhibiting or reducing or blocking the activity or function of ALDH1A3 enzyme) in a cell, in vitro or in vivo, comprising contacting the cell with an effective amount of an ALDHI compound, as described herein.

Another aspect of the present invention pertains to an ALDHI compound as described herein for use in a method of treatment of the human or animal body by therapy, for example, for use in a method of treatment of a disorder (e.g., a disease) as described herein.

Another aspect of the present invention pertains to use of an ALDHI compound as described herein in a method of treatment of the human or animal body by therapy, for example, in a method of treatment of a disorder (e.g., a disease) as described herein.

Another aspect of the present invention pertains to use of an ALDHI compound, as described herein, in the manufacture of a medicament, for example, for use in a method of treatment, for example, for use in a method of treatment of a disorder (e.g., a disease) as described herein.

Another aspect of the present invention pertains to a method of treatment, for example, a method of treatment of a disorder (e.g., a disease) as described herein, comprising administering to a subject in need of treatment a therapeutically-effective amount of an ALDHI compound, as described herein, preferably in the form of a pharmaceutical composition.

In one embodiment, the disorder is a disorder that is ameliorated by the inhibition of ALDH1A3 enzyme (e.g., by the inhibition or reduction or blockage of the activity or function of ALDH1A3 enzyme).

In one embodiment, the disorder is, for example, a proliferative condition, cancer, diabetes, a cardiovascular disorder, etc., as described herein.

Another aspect of the present invention pertains to a kit comprising (a) an ALDHI compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

Another aspect of the present invention pertains to an ALDHI compound obtainable by a method of synthesis as described herein, or a method comprising a method of synthesis as described herein.

Another aspect of the present invention pertains to an ALDHI compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION

### Compounds

One aspect of the present invention relates to compounds of the following general formula, wherein -Q-, -J, R¹, R³, and R⁴ are as defined herein (for convenience, collectively referred to herein as “aldehyde dehydrogenase inhibitor compounds”, “ALDH inhibitor compounds” and “ALDHI compounds”):

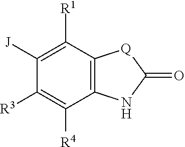

Some embodiments of the compounds include the following:

(1) A compound of the following formula:

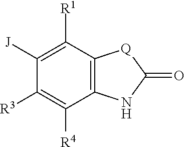

or a pharmaceutically acceptable salt or solvate thereof;
wherein -J is:

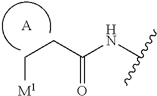

wherein:
Ring A is:
an aromatic monocyclic ring having 5 or 6 ring atoms;
and is optionally substituted with one or more substituents —R^{A};
wherein each —R^{A} is independently —R^{AA}, —R^{AAX}, —OH, —OR^{AA}, —OR^{AAX}, —F, —Cl, —Br, —I, —NH₂, —NHR^{AA}, —NR^{AA}₂, —R^{AAN}, —C(═O)R^{AA}, —C(═O)OH, —C(═O)OR^{AA}, —OC(═O)R^{AA}, —NHC(═O)R^{AA}, —C(═O)NH₂, —C(═O)NHR^{AA}, —C(═O)NR^{AA}₂, —C(═O)R^{AAN}, —S(═O)₂R^{AA}, —S(═O)₂NH₂, —S(═O)₂NHR^{AA}, —S(═O)₂NR^{AA}₂, —S(═O)₂R^{AAN}, —CN, or —NO₂;
wherein:
each —R^{AA} is independently saturated linear or branched C₁₋₄alkyl or saturated C₃₋₆cycloalkyl;
each —R^{AAX} is independently saturated linear or branched C₁₋₄haloalkyl; and
each —R^{AAN} is independently azetidino, pyrrolidino, piperidino, piperazino, morpholino, thiomorpholino, or 1,1-dioxo-thiomorpholino, and is optionally substituted with one or more substituents selected from —R^{AA}, —OH, and —OR^{AA};
either -M¹ is:

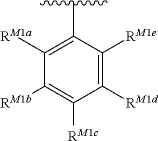

wherein:
each of —R^{M1a} and —R^{M1e} is independently —H or —R^{M1-ortho};
each of —R^{M1b} and —R^{M1d} is independently —H or —R^{M1-meta};
—R^{M1c} is independently —H or —R^{M1-para};
with the proviso that —R^{M1a}, —R^{M1b}, —R^{M1c}, —R^{M1d}, and —R^{M1e} are not all —H;
wherein each —R^{M1-ortho}, each —R^{M1-meta}, —R^{M1-para} is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OH, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —S(═O)₂NH₂, —S(═O)₂NHR^{M11}, —S(═O)₂NR^{M11}₂, —S(═O)₂R^{M11N}, —CN, or —NO₂;
and wherein -M¹ is attached to Ring A by a bond between a ring carbon atom of -M¹ and a ring carbon atom of Ring A;
or -M¹ is an aromatic monocyclic heterocyclic ring having 5 or 6 ring atoms,
and is optionally substituted with one or more substituents —R^{M1};
wherein each —R^{M1} is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OH, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —S(═O)₂NH₂, —S(═O)₂NHR^{M11}, —S(═O)₂NR^{M11}₂, —S(═O)₂R^{M11N}, —CN, or —NO₂;
wherein:
each —R^{M11} is independently saturated linear or branched C₁₋₄alkyl;
each —R^{M11X} is independently saturated linear or branched C₁₋₄haloalkyl; and
each —R^{M11N} is independently azetidino, pyrrolidino, piperidino, piperazino, morpholino, thiomorpholino, or 1,1-dioxo-thiomorpholino, and is optionally substituted with one or more substituents selected from —R^{M11}, —OH, and —OR^{M11};
and wherein -M¹ is attached to Ring A by a bond between a ring carbon atom of -M¹ and a ring carbon atom of Ring A;
   or wherein -J is:

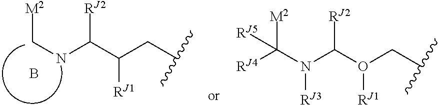

wherein:
each —R^{J1} is independently —H or —R^{JJ};
each —R^{J2} is independently —H or —R^{JJ};
—R^{J} is independently —R^{JJ}, -L^{JJ}-OH, -L^{JJ}-NH₂, -L^{JJ}-NHR^{JJ}, or -L^{JJ}-NR^{JJ}₂;
—R^{J4} is independently —H, —R^{JJ}, -L^{JJ}-OH, -L^{JJ}-NH₂, -L^{JJ}-NHR^{JJ}, or -L^{JJ}-NR^{JJ}₂;
—R^{J5} is independently —H, —R^{JJ}, -L^{JJ}-OH, -L^{JJ}-NH₂, -L^{JJ}-NHR^{JJ}, or -L^{JJ}-NR^{JJ}₂;
or —R^{J4} and —R^{J5} taken together form ═O;
wherein:
each —R^{JJ} is independently saturated linear or branched C₁₋₄alkyl;
each -L^{JJ}- is independently saturated linear or branched C₁₋₄alkylene;
Ring B is independently Ring 1 or Ring B2;
Ring 1 is a non-aromatic monocyclic heterocyclic ring having 4 to 7 ring atoms; and is optionally substituted with one or more substituents —R^{B1};
and/or is optionally substituted with ═O;
wherein each —R^{B1} is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OH, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —S(═O)₂NH₂, —S(═O)₂NHR^{BB}, —S(═O)₂NR^{BB}₂, —S(═O)₂B^{BBN}, —CN, or —NO₂;
Ring B2 is an heteroaromatic monocyclic ring having 5 or 6 ring atoms,
and is optionally substituted with one or more substituents —R^{B2},
wherein each —R^{B2} is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OH, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —S(═O)₂NH₂, —S(═O)₂NHR^{BB}, —S(═O)₂NR^{BB}₂, —S(═O)₂R^{BBN}, —CN, or —NO₂;
wherein:
each —R^{BB} is independently saturated linear or branched C₁₋₄alkyl or saturated C₃₋₆cycloalkyl;
each —R^{BBX} is independently saturated linear or branched C₁₋₄haloalkyl; and
each —R^{BBN} is independently azetidino, pyrrolidino, piperidino, piperazino, morpholino, thiomorpholino, or 1,1-dioxo-thiomorpholino, and is optionally substituted with one or more substituents selected from —R^{BB}, —OH, and —OR^{BB};
-M² is independently:
phenyl,
   and is optionally substituted with one or more substituents —R^{M2}; or an aromatic monocyclic heterocyclic ring having 5 or 6 ring atoms,
   and is optionally substituted with one or more substituents —R^{M2};
wherein each —R^{M2} is independently —R^{M22}, —R^{M22X}, —OH, —OR^{M22}, —OR^{M22X}, —F, —Cl, —Br, —I, —NH₂, —NHR^{M22}, —NR^{M22}₂, —R^{M22N}, —C(═O)R^{M22}, —C(═O)OH, —C(═O)OR^{M22}, —OC(═O)R^{M22}, —NHC(═O)R^{M22}, —C(═O)NH₂, —C(═O)NHR^{M22}, —C(═O)NR^{M22}₂, —C(═O)R^{M22N}, —S(═O)₂R^{M22}, —S(═O)₂NH₂, —S(═O)₂NHR^{M22}, —S(═O)₂NR^{M22}₂, —S(═O)₂R^{M22N}, —CN, or —NO₂;
and wherein:
each —R^{M22} is independently saturated linear or branched C₁₋₄alkyl;
each —R^{M22X} is independently saturated linear or branched C₁₋₄haloalkyl; and
each —R^{M22}N is independently azetidino, pyrrolidino, piperidino, piperazino, morpholino, thiomorpholino, or 1,1-dioxo-thiomorpholino, and is optionally substituted with one or more substituents selected from —R^{M22}, —OH, and —OR^{M22};
   and wherein -Q- is independently:
—CH₂—CR^{Q1}R^{Q2}—;
—O—CR^{Q1}R^{Q2}—;
—S—CR^{Q1}R^{Q2}—;
—CH₂—CH₂—CR^{Q1}R^{Q2}—; or
—CR^{Q3}═CR^{Q4}—;
   wherein:
each —R^{Q1} is independently —H or —R^{QQ};
each —R^{Q2} is independently —H or —R^{QQ};
—R^{Q3} is independently —H or —R^{QQ};
—R^{Q4} is independently —H or —R^{QQ};
wherein:
each —R^{QQ} is independently saturated linear or branched C₁₋₄alkyl;
   and wherein:
—R¹ is independently —H or —R¹¹;
—R³ is independently —H or —R³³;
—R⁴ is independently —H or —R⁴⁴;
each R¹¹ is independently —R, —R^{X}, —OH, —OR, —OR^{X}, —F, —Cl, —Br, —I, —NH₂, —NHR, —NR₂, —R^{N}, —CN, or —NO₂;
each R³³ is independently —R, —RX, —OH, —OR, —OR^{X}, —F, —Cl, —Br, —I, —NH₂, —NHR, —NR₂, —R^{N}, —CN, or —NO₂;
each R⁴⁴ is independently —R, —RX, —OH, —OR, —OR^{X}, —F, —Cl, —Br, —I, —NH₂, —NHR, —NR₂, —R^{N}, —CN, or —NO₂;
   wherein:
each —R is independently saturated linear or branched C₁₋₄alkyl;
each —R^{X} is independently saturated linear or branched C₁₋₄haloalkyl; and
each —R^{N} is independently azetidino, pyrrolidino, piperidino, piperazino, morpholino, thiomorpholino, or 1,1-dioxo-thiomorpholino, and is optionally substituted with one or more substituents selected from —R, —OH, and —OR;
   with the proviso that the compound is not a compound of one of the following formulae, or a pharmaceutically acceptable salt or solvate thereof:

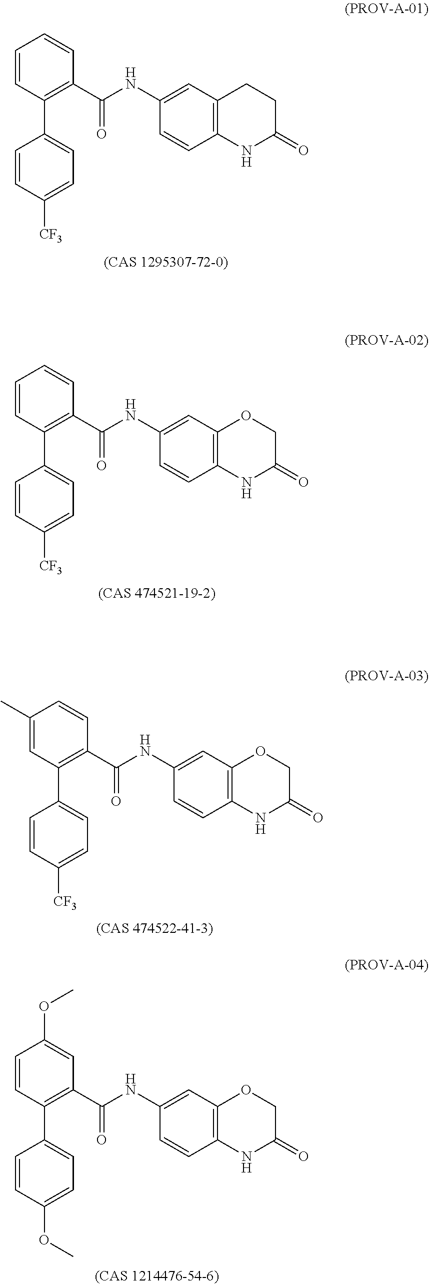 [SMILES: O=C1CCc2cc(NC(=O)c3ccccc3-c3ccc(C(F)(F)F)cc3)ccc2N1 ; O=C1COc2cc(NC(=O)c3ccccc3-c3ccc(C(F)(F)F)cc3)ccc2N1 ; Cc1ccc(C(=O)Nc2ccc3c(c2)OCC(=O)N3)c(-c2ccc(C(F)(F)F)cc2)c1 ; COc1ccc(-c2ccc(OC)cc2C(=O)Nc2ccc3c(c2)OCC(=O)N3)cc1]

and with the proviso that the compound is not a compound of one of the following formulae, or a pharmaceutically acceptable salt or solvate thereof:

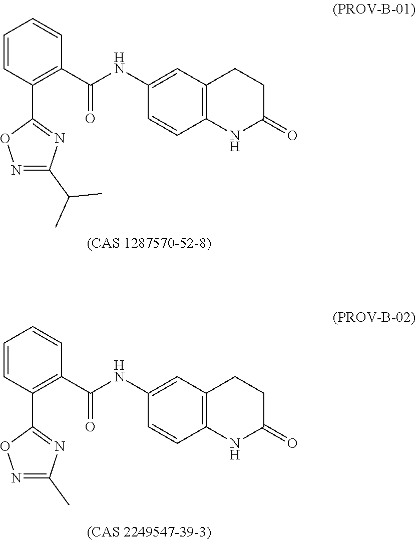 [SMILES: CC(C)c1noc(-c2ccccc2C(=O)Nc2ccc3c(c2)CCC(=O)N3)n1 ; Cc1noc(-c2ccccc2C(=O)Nc2ccc3c(c2)CCC(=O)N3)n1]

and with the proviso that the compound is not a compound of one of the following formulae, or a pharmaceutically acceptable salt or solvate thereof:

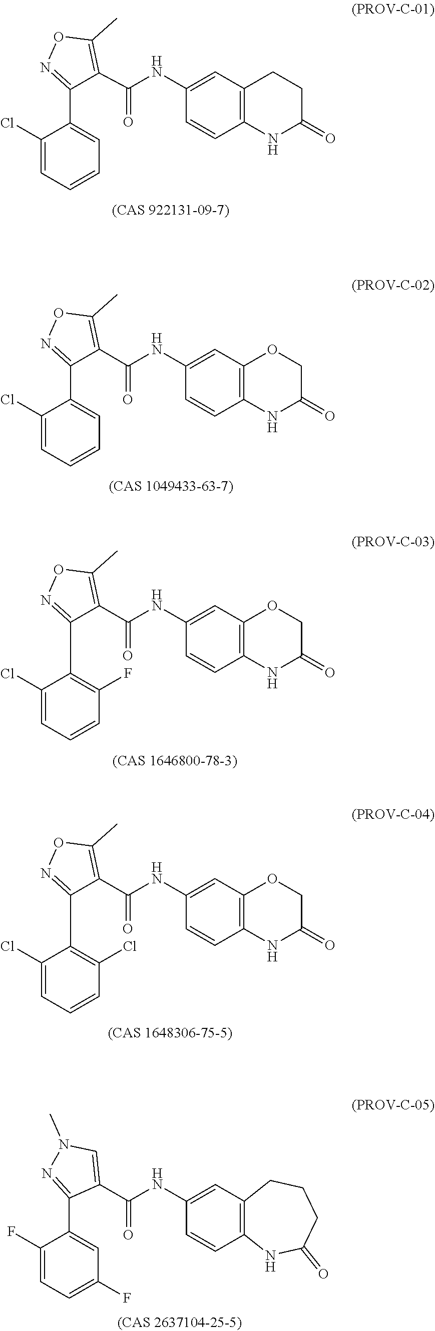 [SMILES: Cc1onc(-c2ccccc2Cl)c1C(=O)Nc1ccc2c(c1)CCC(=O)N2 ; Cc1onc(-c2ccccc2Cl)c1C(=O)Nc1ccc2c(c1)OCC(=O)N2 ; Cc1onc(-c2c(F)cccc2Cl)c1C(=O)Nc1ccc2c(c1)OCC(=O)N2 ; Cc1onc(-c2c(Cl)cccc2Cl)c1C(=O)Nc1ccc2c(c1)OCC(=O)N2 ; Cn1cc(C(=O)Nc2ccc3c(c2)CCCC(=O)N3)c(-c2cc(F)ccc2F)n1]

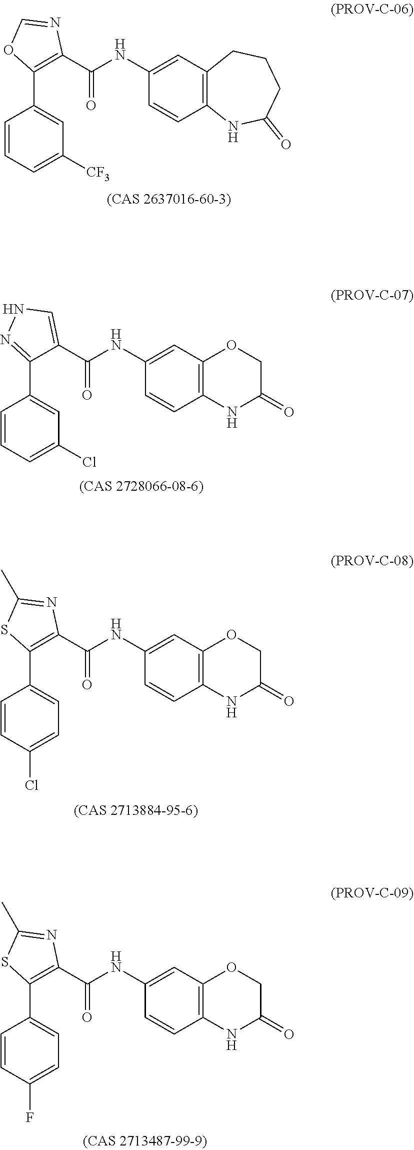 [SMILES: O=C1CCCc2cc(NC(=O)c3ncoc3-c3cccc(C(F)(F)F)c3)ccc2N1 ; O=C1COc2cc(NC(=O)c3c[nH]nc3-c3cccc(Cl)c3)ccc2N1 ; Cc1nc(C(=O)Nc2ccc3c(c2)OCC(=O)N3)c(-c2ccc(Cl)cc2)s1 ; Cc1nc(C(=O)Nc2ccc3c(c2)OCC(=O)N3)c(-c2ccc(F)cc2)s1]

and with the proviso that the compound is not a compound of one of the following formulae, or a pharmaceutically acceptable salt or solvate thereof:

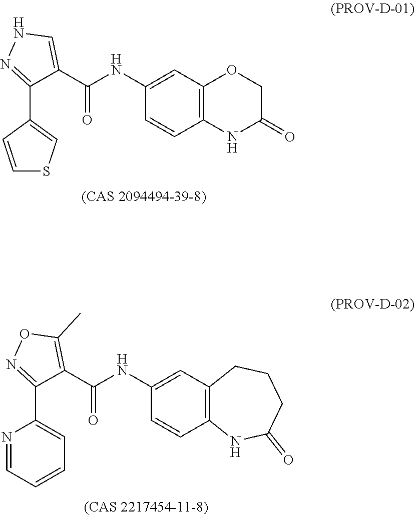 [SMILES: O=C1COc2cc(NC(=O)c3c[nH]nc3-c3ccsc3)ccc2N1 ; Cc1onc(-c2ccccn2)c1C(=O)Nc1ccc2c(c1)CCCC(=O)N2]

and with the proviso that the compound is not a compound of one of the following formulae, or a pharmaceutically acceptable salt or solvate thereof:

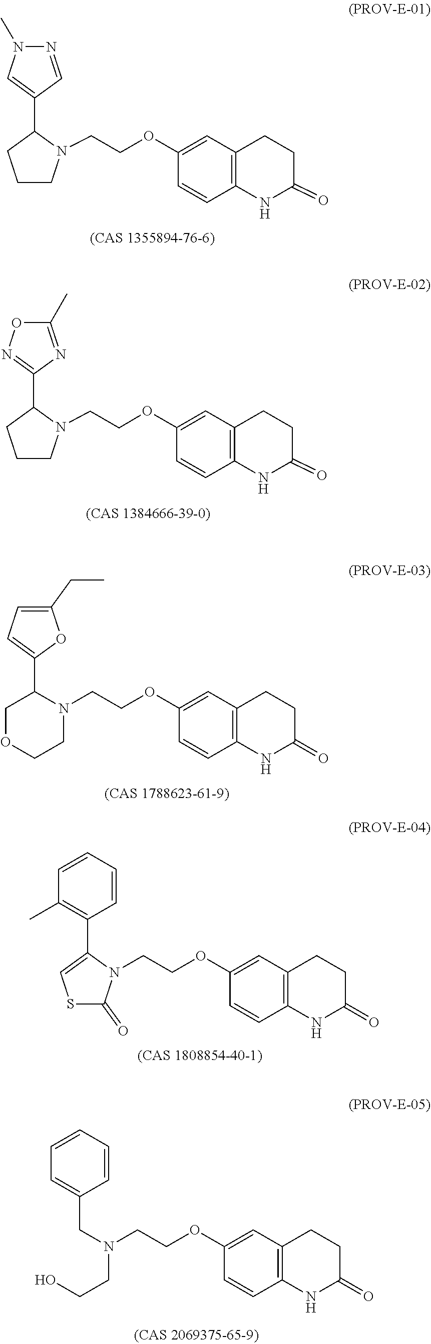 [SMILES: Cn1cc(C2CCCN2CCOc2ccc3c(c2)CCC(=O)N3)cn1 ; Cc1nc(C2CCCN2CCOc2ccc3c(c2)CCC(=O)N3)no1 ; CCc1ccc(C2COCCN2CCOc2ccc3c(c2)CCC(=O)N3)o1 ; Cc1ccccc1-c1csc(=O)n1CCOc1ccc2c(c1)CCC(=O)N2 ; O=C1CCc2cc(OCCN(CCO)Cc3ccccc3)ccc2N1]

For the avoidance of doubt, the group -Q-, as written left-to-right, is attached on the left-side to the benzene ring and on the right-hand side to the carbonyl group. For example, when -Q- is —O—CR^{Q1}R^{Q2}—, the O on the left-side is attached to the benzene ring (marked with an asterisk, below) and the CR^{Q1}R^{Q2} on the right-hand side is attached to the carbonyl group (marked with a hash, below).

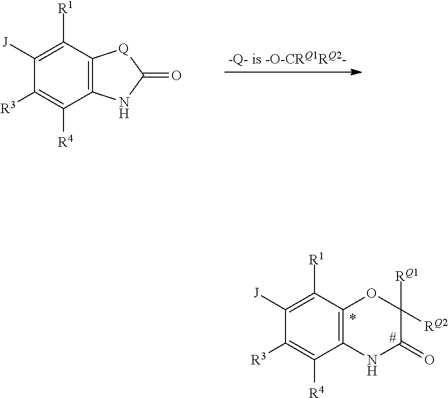

For the avoidance of doubt, it is not intended that the N ring atom in the ring containing -Q- is substituted; instead, it is intended that the N ring atom in the ring containing -Q- is unsubstituted.

For the avoidance of doubt, it is not intended that the groups -Q-, -J, —R¹, —R³, and —R⁴ are linked other than via the ring atoms to which they are attached. For example, it is not intended that -Q- and —R¹ together form a fused ring structure; it is not intended that —R¹ and -J together form a fused ring structure; it is not intended that -J and —R³ together form a fused ring structure; it is not intended that —R³ and —R⁴ together form a fused ring structure; it is not intended that -Q- and -J together form a fused ring structure; it is not intended that —R¹ and —R³ together form a fused ring structure; etc.

Unless otherwise indicated, where a compound is shown or described which has one or more chiral centres, and two or more stereoisomers are possible, all such stereoisomers are disclosed and encompassed, both individually (e.g., as isolated from the other stereoisomer(s)) and as mixtures (e.g., as equimolar or non-equimolar mixtures of two or more stereoisomers). For example, unless otherwise indicated, where a compound has one chiral centre, each of the (R) and (S) enantiomers are disclosed and encompassed, both individually (e.g., as isolated from the other enantiomer) and as a mixture (e.g., as equimolar or non-equimolar mixtures of the two enantiomers).

For example, when -Q- is —O—CR^{Q1}R^{Q2}— and —R^{Q1} and —R^{Q2} are different, then the carbon atom to which —R^{Q1} and —R^{Q2} are attached is a chiral centre, as marked with an asterisk (*) in the following formula. Unless otherwise stated, the carbon atom at this position may be in either (R) or (S) configuration.

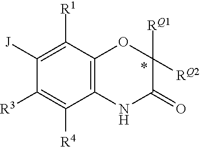

Unless otherwise indicated, where a compound is shown or described which is susceptible to tautomerism, and two tautomers are possible, both tautomers are disclosed and encompassed, both individually (e.g., as isolated from the other tautomer) and as mixtures (e.g., as equimolar or non-equimolar mixtures of two tautomers).

The term “saturated linear or branched C₁₋₄alkyl” means —CH₃ (methyl), —CH₂CH₃ (ethyl), —CH₂CH₂CH₃ (n-propyl), —CH(CH₃)₂ (iso-propyl), —CH₂CH₂CH₂CH₃ (n-butyl), —CH₂CH(CH₃)₂ (iso-butyl), —CH(CH₃)CH₂CH₃ (sec-butyl), and —C(CH₃)₃ (tert-butyl).

The term “saturated linear or branched C₁₋₄haloalkyl” means a saturated linear or branched C₁₋₄alkyl group substituted with one or more halo groups (e.g., —F, —Cl, —Br, —I), and includes, for example, “saturated linear or branched C₁₋₄fluoroalkyl”, e.g., —CF₃, —CHF₂, —CH₂CF₃, —CH₂CH₂F, —CH₂CHF₂, —CH(CH₃)CF₃, —CH₂C(CH₃)₂F, —CH₂CF₂CH₃, —CH₂CH₂CF₂CH₃, —CH₂CH₂CHF₂, and —CH₂CH₂CF₃.

The term “saturated C₃₋₆cycloalkyl” means cyclopropyl, cyclobutyl, cyclopentyl, and cyclohexyl.

The term “linear or branched saturated C₁₋₄alkylene” means a bi-dentate saturated linear or branched C₁₋₄alkyl group, and includes, e.g., —CH₂—, —CH₂CH₂—, —CH₂CH₂CH₂—, —CH₂CH₂CH₂CH₂—, —CH(CH₃)—, and —CH₂CH(CH₃)—.

The term “non-aromatic C₄₋₇heterocyclyl” means a non-aromatic cyclic group having 4 to 7 ring atoms, wherein exactly 1, exactly 2, or exactly 3 of the ring atoms is a ring heteroatom, wherein each ring heteroatom is selected from O, N, and S (wherein a ring S atom may optionally be in an oxidized form, e.g., —S(═O) or S(═O)₂). Such groups may be monocyclic or polycyclic, e.g., bridged or spiro. Examples include, e.g., non-aromatic monocyclic C₄₋₇heterocyclyl, such as oxetanyl, tetrahydrofuranyl, tetrahydropyranyl, oxanyl, dioxanyl, azetidinyl, pyrrolidinyl, piperidinyl, piperazinyl, morpholinyl, thiomorpholinyl, 1,4-thiazinane 1,1-dioxide, azepanyl, oxazepanyl, and diazepanyl; non-aromatic bridged C₇heterocyclyl, such as diazabicyclo[2.2.1]heptane, azabicyclo[3.1.1]heptane, azabicyclo[2.2.1]heptane, and azabicyclo[4.1.0]heptane; and non-aromatic spiro C₇heterocyclyl, such as 6-oxa-3-azaspiro[3.3]heptane.

The term “C₅₋₆heteroaryl” means an aromatic group having 5 to 6 ring atoms, wherein exactly 1, exactly 2, or exactly 3 of the aromatic ring atoms is a ring heteroatom, wherein each ring heteroatom is selected from O, N, and S. Examples include, e.g., “C₅heteroaryl” groups, such as furanyl, thienyl, pyrrolyl, imidazolyl, oxazolyl, isoxazolyl, thiazolyl, isothiazolyl, pyrazolyl, triazolyl, oxadiazolyl, thiadiazolyl, and “C₆heteroaryl” groups, such as pyridyl, pyridazinyl, pyrimidinyl, and pyrazinyl.

### The Group -Q-

(2) A compound according to (1), wherein -Q- is independently —CH₂—CR^{Q1}R^{Q2}—, —O—CR^{Q1}R^{Q2}—, or —S—CR^{Q1}R^{Q2}—.

(3) A compound according to (1), wherein -Q- is —CH₂—CR^{Q1}R^{Q2}—; for example:

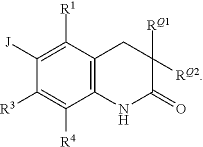

(4) A compound according to (1), wherein -Q- is independently —O—CR^{Q1}R^{Q2}— or —S—CR^{Q1}R^{Q2}—.

(5) A compound according to (1), wherein -Q- is —O—CR^{Q1}R^{Q2}—; for example:

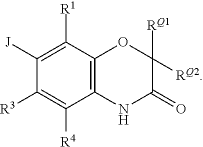

(6) A compound according to (1), wherein -Q- is —S—CR^{Q1}R^{Q2}—; for example:

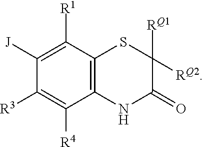

(7) A compound according to (1), wherein -Q- is —CH₂—CH₂—CR^{Q1}R^{Q2}—; for example:

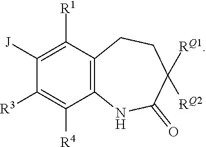

(8) A compound according to (1), wherein -Q- is —CR^{Q3}═CR^{Q4}—; for example:

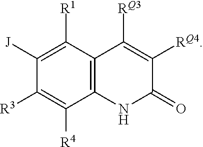

### The Group —R^{Q1}

(9) A compound according to any one of (1) to (8), wherein each —R^{Q1}, if present, is —H.

(10) A compound according to any one of (1) to (8), wherein each —R^{Q1}, if present, is —R^{QQ}.

### The Group —R^{Q2}

(11) A compound according to any one of (1) to (10), wherein each —R^{Q2}, if present, is —H.

(12) A compound according to any one of (1) to (10), wherein each —R^{Q2}, if present, is —R^{QQ}.

### The Group —R^{Q3}

(13) A compound according to any one of (1) to (12), wherein each —R^{Q3}, if present, is —H.

(14) A compound according to any one of (1) to (12), wherein each —R^{Q3}, if present, is —R^{QQ}.

### The Group —R^{Q4}

(15) A compound according to any one of (1) to (14), wherein each —R^{Q4}, if present, is —H.

(16) A compound according to any one of (1) to (14), wherein each —R^{Q4}, if present, is —R^{QQ}.

### The Group —R^{QQ}

(17) A compound according to any one of (1) to (16), wherein —R^{QQ}, if present, is independently saturated linear or branched C₁₋₃alkyl.

(18) A compound according to any one of (1) to (16), wherein —R^{QQ}, if present, is independently -Me or -Et.

(19) A compound according to any one of (1) to (16), wherein —R^{QQ}, if present, is -Me.

### The Group —R¹

(20) A compound according to any one of (1) to (19), wherein —R¹ is —H.

(21) A compound according to any one of (1) to (19), wherein —R¹ is —R¹¹.

### The Group —R³

(22) A compound according to any one of (1) to (21), wherein —R³ is —H.

(23) A compound according to any one of (1) to (21), wherein —R³ is —R³³.

### The Group —R⁴

(24) A compound according to any one of (1) to (23), wherein —R⁴ is —H.

(25) A compound according to any one of (1) to (23), wherein —R⁴ is —R⁴⁴.

### The Group —R¹¹

(26) A compound according to any one of (1) to (25), wherein —R¹¹, if present, is independently —R, —RX, —OH, —OR, —OR^{X}, —F, —Cl, —Br, or —I.

(27) A compound according to any one of (1) to (25), wherein —R¹¹, if present, is independently —R, —RX, —F, or —Cl.

(28) A compound according to any one of (1) to (25), wherein —R¹¹, if present, is independently —F or —Cl.

(29) A compound according to any one of (1) to (25), wherein —R¹¹, if present, is —F.

### The Group —R³³

(30) A compound according to any one of (1) to (29), wherein —R³³, if present, is independently —R, —RX, —OH, —OR, —OR^{X}, —F, —Cl, —Br, or —I.

(31) A compound according to any one of (1) to (29), wherein —R³³, if present, is independently —R, —RX, —F, or —Cl.

(32) A compound according to any one of (1) to (29), wherein —R³³, if present, is independently —R, —F, or —Cl.

(33) A compound according to any one of (1) to (29), wherein —R³³, if present, is independently —F or —Cl.

### The Group —R⁴⁴

(34) A compound according to any one of (1) to (33), wherein —R⁴⁴, if present, is independently —R, —RX, —OH, —OR, —OR^{X}, —F, —Cl, —Br, or —I.

(35) A compound according to any one of (1) to (33), wherein —R⁴⁴, if present, is independently —R, —RX, —F, or —Cl.

(36) A compound according to any one of (1) to (33), wherein —R⁴⁴, if present, is independently —F or —Cl.

(37) A compound according to any one of (1) to (33), wherein —R⁴⁴, if present, is —F.

### The Group —R

(38) A compound according to any one of (1) to (37), wherein each —R, if present, is independently saturated linear or branched C₁₋₃alkyl.

(39) A compound according to any one of (1) to (37), wherein each —R, if present, is independently -Me or -Et.

(40) A compound according to any one of (1) to (37), wherein each —R, if present, is -Me.

### The Group —R^{X}

(41) A compound according to any one of (1) to (40), wherein each —R^{X}, if present, is independently saturated linear or branched C₁₋₃haloalkyl.

(42) A compound according to any one of (1) to (40), wherein each —R^{X}, if present, is —CF₃.

### The Group —R^{N}

(43) A compound according to any one of (1) to (42), wherein each —RN, if present, is independently independently azetidino, pyrrolidino, piperidino, piperazino, or morpholino, and is optionally substituted with one or more substituents selected from —R, —OH, and —OR.

(44) A compound according to any one of (1) to (42), wherein each —RN, if present, is independently independently azetidino, pyrrolidino, piperidino, piperazino, or morpholino.

### The Group -J (Part 1)

(45) A compound according to any one of (1) to (44), wherein -J is:

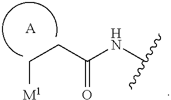

### The Ring A (Heteroaryl)

(46) A compound according to any one of (1) to (45), wherein Ring A, if present, is a C₅₋₆heteroaryl group; and is optionally substituted with one or more substituents —R^{A}.

(47) A compound according to any one of (1) to (45), wherein Ring A, if present, is independently furanyl, thienyl, pyrrolyl, imidazolyl, oxazolyl, isoxazolyl, thiazolyl, isothiazolyl, pyrazolyl, triazolyl, oxadiazolyl, thiadiazolyl, pyridyl, pyridazinyl, pyrimidinyl, or pyrazinyl; and is optionally substituted with one or more substituents —R^{A}.

(48) A compound according to any one of (1) to (45), wherein Ring A, if present, is a C₅heteroaryl group; and is optionally substituted with one or more substituents —R^{A}.

(49) A compound according to any one of (1) to (45), wherein Ring A, if present, is independently furanyl, thienyl, pyrrolyl, imidazolyl, oxazolyl, isoxazolyl, thiazolyl, isothiazolyl, pyrazolyl, triazolyl, oxadiazolyl, or thiadiazolyl; and is optionally substituted with one or more substituents —R^{A}.

(50) A compound according to any one of (1) to (45), wherein Ring A, if present, is independently thienyl, oxazolyl, isoxazolyl, thiazolyl, and pyrazolyl; and is optionally substituted with one or more substituents —R^{A}.

(51) A compound according to any one of (1) to (45), wherein Ring A, if present, is independently thiazolyl or pyrazolyl; and is optionally substituted with one or more substituents —R^{A}.

(52) A compound according to any one of (1) to (45), wherein Ring A, if present, is thiazolyl; and is optionally substituted with a substituent —R^{A}.

(53) A compound according to any one of (1) to (45), wherein Ring A, if present, is a thiazolyl of the following formula, where (*) denotes the point of attachment to -M¹ and (#) denotes the point of attachment to the —C(═O)— of group -J; and is optionally substituted with a substituent —R^{A}:

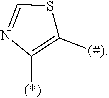

For example, here, -J is:

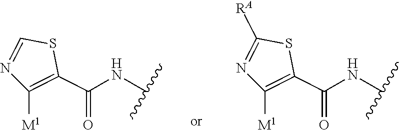

(54) A compound according to any one of (1) to (45), wherein Ring A, if present, is a thiazolyl of the following formula, where (*) denotes the point of attachment to -M¹ and (#) denotes the point of attachment to the —C(═O)— of group -J; and is optionally substituted with a substituent —R^{A}:

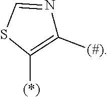

(55) A compound according to any one of (1) to (45), wherein Ring A, if present, is thiazolyl; and is unsubstituted.

(56) A compound according to any one of (1) to (45), wherein Ring A, if present, is an unsubstituted thiazolyl of the following formula, where (*) denotes the point of attachment to -M¹ and (#) denotes the point of attachment to the —C(═O)— of group -J:

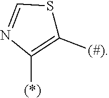

(57) A compound according to any one of (1) to (45), wherein Ring A, if present, is an unsubstituted thiazolyl of the following formula, where (*) denotes the point of attachment to -M¹ and (#) denotes the point of attachment to the —C(═O)— of group -J:

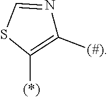

(58) A compound according to any one of (1) to (45), wherein Ring A, if present, is pyrazolyl; and is optionally substituted with one or more substituents —R^{A}.

(59) A compound according to any one of (1) to (45), wherein Ring A, if present, is a pyrazolyl of one of the following formulae, where (*) denotes the point of attachment to -M¹ and (#) denotes the point of attachment to the —C(═O)— of group -J; and is optionally substituted with one or more substituents —R^{A}:

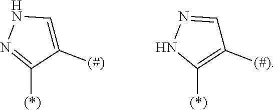

(60) A compound according to any one of (1) to (45), wherein Ring A, if present, is pyrazolyl; and is unsubstituted.

(61) A compound according to any one of (1) to (45), wherein Ring A, if present, is thienyl; and is optionally substituted with one or more substituents —R^{A}.

(62) A compound according to any one of (1) to (45), wherein Ring A, if present, is a thienyl of the following formula, where (*) denotes the point of attachment to -M¹ and (#) denotes the point of attachment to the —C(═O)— of group -J; and is optionally substituted with one or more substituents —R^{A}:

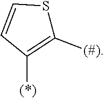

(63) A compound according to any one of (1) to (45), wherein Ring A, if present, is thienyl; and is unsubstituted.

(64) A compound according to any one of (1) to (45), wherein Ring A, if present, is oxazolyl; and is optionally substituted with a substituent —R^{A}.

(65) A compound according to any one of (1) to (45), wherein Ring A, if present, is a oxazolyl of the following formula, where (*) denotes the point of attachment to -M¹ and (#) denotes the point of attachment to the —C(═O)— of group -J; and is optionally substituted with a substituent —R^{A}:

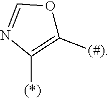

(66) A compound according to any one of (1) to (45), wherein Ring A, if present, is oxazolyl; and is unsubstituted.

(67) A compound according to any one of (1) to (45), wherein Ring A, if present, is isoxazolyl; and is optionally substituted with a substituent —R^{A}.

(68) A compound according to any one of (1) to (45), wherein Ring A, if present, is a isoxazolyl of the following formula, where (*) denotes the point of attachment to -M¹ and (#) denotes the point of attachment to the —C(═O)— of group -J; and is optionally substituted with a substituent —R^{A}:

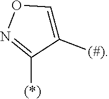

(69) A compound according to any one of (1) to (45), wherein Ring A, if present, is isoxazolyl; and is unsubstituted.

(70) A compound according to any one of (1) to (45), wherein Ring A, if present, is a C₆heteroaryl group; and is optionally substituted with one or more substituents —R^{A}.

(71) A compound according to any one of (1) to (45), wherein Ring A, if present, is independently pyridyl, pyridazinyl, pyrimidinyl, or pyrazinyl; and is optionally substituted with one or more substituents —R^{A}.

(72) A compound according to any one of (1) to (45), wherein Ring A, if present, is independently pyridyl or pyrimidinyl; and is optionally substituted with one or more substituents —R^{A}.

(73) A compound according to any one of (1) to (45), wherein Ring A, if present, is pyridyl; and is optionally substituted with one or more substituents —R^{A}.

(74) A compound according to any one of (1) to (45), wherein Ring A, if present, is a pyridyl of the following formula, where (*) denotes the point of attachment to -M¹ and (#) denotes the point of attachment to the —C(═O)— of group -J; and is optionally substituted with one or more substituents —R^{A}:

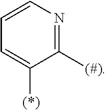

(75) A compound according to any one of (1) to (45), wherein Ring A, if present, is pyridyl; and is unsubstituted.

(76) A compound according to any one of (1) to (45), wherein Ring A, if present, is pyrimidinyl; and is optionally substituted with one or more substituents —R^{A}.

(77) A compound according to any one of (1) to (45), wherein Ring A, if present, is a pyrimidinyl of the following formula, where (*) denotes the point of attachment to -M¹ and (#) denotes the point of attachment to the —C(═O)— of group -J; and is optionally substituted with one or more substituents —R^{A}:

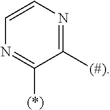

(78) A compound according to any one of (1) to (45), wherein Ring A, if present, is pyrimidinyl; and is unsubstituted.

### The Ring A (Carboaryl)

(79) A compound according to any one of (1) to (45), wherein Ring A, if present, is phenyl; and is optionally substituted with one or more substituents —R^{A}.

### The Group —R^{A}

(80) A compound according to any one of (1) to (79), wherein each —R^{A}, if present, is independently —R^{AA}, —R^{AAX}, —OH, —OR^{AA}, —OR^{AAX}, —F, —Cl, —Br, —I, —NH₂, —NHR^{AA}, —NR^{AA}₂, —R^{AAN}, —C(═O)R^{AA}, —C(═O)OR^{AA}, —OC(═O)R^{AA}, —NHC(═O)R^{AA}, —C(═O)NH₂, —C(═O)NHR^{AA}, —C(═O)NR^{AA}₂, —C(═O)R^{AAN}, —S(═O)₂R^{AA}, —CN, or —NO₂.

(81) A compound according to any one of (1) to (79), wherein each —R^{A}, if present, is independently —R^{AA}, —R^{AAX}, —OH, —OR^{AA}, —OR^{AAX}, —F, —Cl, —Br, —I, —NH₂, —NHR^{AA}, —NR^{AA}₂, —R^{AAN}, —CN, or —NO₂.

(82) A compound according to any one of (1) to (79), wherein each —R^{A}, if present, is independently —NH₂, —NHR^{AA}, —NR^{AA}₂, or —R^{AAN}.

(83) A compound according to any one of (1) to (79), wherein each —R^{A}, if present, is —R^{AA}.

(84) A compound according to any one of (1) to (79), wherein each —R^{A}, if present, is —R^{AAN}.

(85) A compound according to any one of (1) to (79), wherein each —R^{A}, if present, is —NR^{AA}₂.

### The Group —R^{AA}

(86) A compound according to any one of (1) to (85), wherein each —R^{AA}, if present, is independently saturated linear or branched C₁₋₄alkyl.

(87) A compound according to any one of (1) to (85), wherein each —R^{AA}, if present, is independently saturated linear or branched C₁₋₃alkyl.

(88) A compound according to any one of (1) to (85), wherein each —R^{AA}, if present, is independently -Me or -Et.

(89) A compound according to any one of (1) to (85), wherein each —R^{AA}, if present, is -Me.

### The Group —R^{AAX}

(90) A compound according to any one of (1) to (89), wherein each —R^{AAX} if present, is saturated linear or branched C₁₋₄fluoroalkyl.

(91) A compound according to any one of (1) to (89), wherein each —R^{AAX} if present, is —CF₃.

### The Group —R^{AAN}

(92) A compound according to any one of (1) to (91), wherein each —R^{AAN}, if present, is independently azetidino, pyrrolidino, piperidino, piperazino, or morpholino, and is optionally substituted with one or more substituents selected from —R^{AA}, —OH, and —OR^{AA}.

(93) A compound according to any one of (1) to (91), wherein each —R^{AAN}, if present, is independently azetidino, pyrrolidino, piperidino, piperazino, or morpholino.

### The Group -M¹ (Carboaryl)

Again, note that -M¹ is attached to Ring A by a bond between a ring carbon atom of -M¹ and a ring carbon atom of Ring A.

(94) A compound according to any one of (1) to (93), wherein -M¹, if present, is:

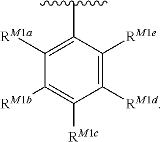

(95) A compound according to any one of (1) to (93), wherein -M¹, if present, is:

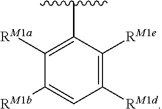

(96) A compound according to any one of (1) to (93), wherein -M¹, if present, is:

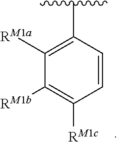

(97) A compound according to any one of (1) to (93), wherein -M¹, if present, is:

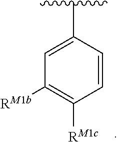

(98) A compound according to any one of (1) to (93), wherein -M¹, if present, is:

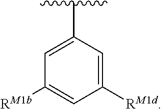

(99) A compound according to any one of (1) to (93), wherein -M¹, if present, is:

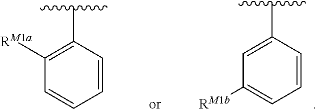

(100) A compound according to any one of (1) to (93), wherein -M¹, if present, is:

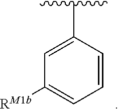

(101) A compound according to any one of (1) to (93), wherein -M¹, if present, is:

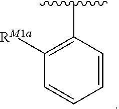

(102) A compound according to any one of (1) to (93), wherein -M¹, if present, is:

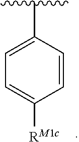

### The Group —R^{M1a}

(103) A compound according to any one of (1) to (102), wherein —R^{M1a}, if present, is —H.

(104) A compound according to any one of (1) to (102), wherein —R^{M1a}, if present, is —R^{M1-ortho}.

### The Group —R^{M1b}

(105) A compound according to any one of (1) to (104), wherein —R^{M1b}, if present, is —H.

(106) A compound according to any one of (1) to (104), wherein —R^{M1b}, if present, is —R^{M1-meta}.

### The Group —R^{M1c}

(107) A compound according to any one of (1) to (106), wherein —R^{M1}c, if present, is —H.

(108) A compound according to any one of (1) to (106), wherein —R^{M1}c, if present, is —R^{M1-para}.

### The Group —R^{M1d}

(109) A compound according to any one of (1) to (108), wherein —R^{M1d}, if present, is —H.

(110) A compound according to any one of (1) to (108), wherein —R^{M1d}, if present, is —R^{M1-meta}.

### The Group —R^{M1e}

(111) A compound according to any one of (1) to (110), wherein —R^{M1e}, if present, is —H.

(112) A compound according to any one of (1) to (110), wherein —R^{M1e}, if present, is —R^{M1-ortho}.

The Group —R^{M1-ortho}

(113) A compound according to any one of (1) to (112), wherein each —R^{M1-ortho}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —CN, or —NO₂.

(114) A compound according to any one of (1) to (112), wherein each —R^{M1-ortho}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —S(═O)₂R^{M11}, —CN, or —NO₂.

(115) A compound according to any one of (1) to (112), wherein each —R^{M1-ortho}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, or —R^{M11N}.

(116) A compound according to any one of (1) to (112), wherein each —R^{M1-ortho}, if present, is independently —R^{M11}, —R^{M11X}, —OH, —OR^{M11}, —OR^{M11X}, —F, —Cl, —Br, or —I.

(117) A compound according to any one of (1) to (112), wherein each —R^{M1-ortho}, if present, is independently —R^{M11}, —OH, —OR^{M11}, —F, —Cl, —Br, or —I.

(118) A compound according to any one of (1) to (112), wherein each —R^{M1-ortho}, if present, is independently —R^{M11}, —OH, —OR^{M11}, or —F.

(119) A compound according to any one of (1) to (112), wherein each —R^{M1-ortho}, if present, is independently —R^{M11}, —F, —Cl, —Br, or —I.

(120) A compound according to any one of (1) to (112), wherein each —R^{M1-ortho}, if present, is independently —R^{M11} or —F

(121) A compound according to any one of (1) to (112), wherein each —R^{M1-ortho}, if present, is independently —F, —Cl, —Br, or —I.

(122) A compound according to any one of (1) to (112), wherein each —R^{M1-ortho}, if present, is —F.

The Group —R^{M1-meta}

(123) A compound according to any one of (1) to (122), wherein each —R^{M1-meta}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —CN, or —NO₂.

(124) A compound according to any one of (1) to (122), wherein each —R^{M1-meta}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —S(═O)₂R^{M11}, —CN, or —NO₂.

(125) A compound according to any one of (1) to (122), wherein each —R^{M1-meta}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, or —R^{M11N}.

(126) A compound according to any one of (1) to (122), wherein each —R^{M1-meta}, if present, is independently —R^{M11}, —R^{M11X}, —OH, —OR^{M11}, —OR^{M11X}, —F, —Cl, —Br, or —I.

(127) A compound according to any one of (1) to (122), wherein each —R^{M1-meta}, if present, is independently —R^{M11}, —OH, —OR^{M11}, —F, —Cl, —Br, or —I.

(128) A compound according to any one of (1) to (122), wherein each —R^{M1-meta}, if present, is independently —R^{M11}, —F, —Cl, —Br, or —I.

(129) A compound according to any one of (1) to (122), wherein each —R^{M1-meta}, if present, is independently —F, —Cl, —Br, or —I.

(130) A compound according to any one of (1) to (122), wherein each —R^{M1-meta}, if present, is independently —F or —Cl.

(131) A compound according to any one of (1) to (122), wherein each —R^{M1-meta}, if present, is —F.

The Group —R^{M1-para}

(132) A compound according to any one of (1) to (131), wherein each —R^{M1-para} if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OR^{M11}, —OC(═O)R^{M11}, —NHC(═O)R^{M11}, —C(═O)NH₂, —C(═O)NHR^{M11}, —C(═O)NR^{M12}, —C(═O)R^{M11N}, —S(═O)₂R^{M11}, —CN, or —NO₂.

(133) A compound according to any one of (1) to (131), wherein each —R^{M1-para} if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —S(═O)₂R^{M11}, —CN, or —NO₂.

(134) A compound according to any one of (1) to (131), wherein each —R^{M1-para} if present, is independently —R^{M11}, —OH, —OR^{M11}, —F, —Cl, —Br, —I, —NH₂, —NHR^{M11}, —NR^{M11}₂, —R^{M11N}, —S(═O)₂R^{M11}, —CN, or —NO₂.

(135) A compound according to any one of (1) to (131), wherein each —R^{M1-para} if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, or —CN.

(136) A compound according to any one of (1) to (131), wherein each —R^{M1-para} if present, is independently —R^{M11}, —OH, —F, —C, —Br, —I, —NH₂, —NHR^{M11}, —NR^{M11}₂, —R^{M11N}, or —CN.

(137) A compound according to any one of (1) to (131), wherein each —R^{M1-para} if present, is independently —R^{M11}, —R^{M11X}, —OH, —OR^{M11}, —OR^{M11X}, —F, —C, —Br, or —I.

(138) A compound according to any one of (1) to (131), wherein each —R^{M1-para} if present, is independently —R^{M11}, —OH, —F, —Cl, —Br, or —I.

(139) A compound according to any one of (1) to (131), wherein each —R^{M1-para} if present, is independently —R^{M11}, —F, —Cl, —Br, or —I.

(140) A compound according to any one of (1) to (131), wherein each —R^{M-para}, if present, is independently —F, —Cl, —Br, or —I.

(141) A compound according to any one of (1) to (131), wherein each —R^{M-para}, if present, is independently —F or —Cl.

(142) A compound according to any one of (1) to (131), wherein each —R^{M-para}, if present, is —F.

The Groups —R^{M1-ortho}, —R^{M1-meta}, and —R^{M1-para}

(143) A compound according to any one of (1) to (112), wherein:
each —R^{M1-ortho}, if present, is —F;
each —R^{M1-meta}, if present, is —F; and
each —R^{M1-para}, if present, is —F.

### A Particular Group -M¹

(144) A compound according to any one of (1) to (93), wherein -M¹, if present, is:

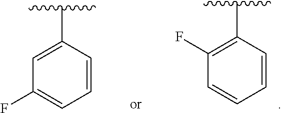 [SMILES: *c1cccc(F)c1 ; "or" ; *c1ccccc1F ; "."]

(145) A compound according to any one of (1) to (93), wherein -M¹, if present, is:

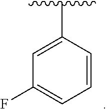 [SMILES: *c1cccc(F)c1 ; "."]

(146) A compound according to any one of (1) to (93), wherein -M¹, if present, is:

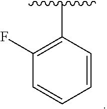 [SMILES: *c1ccccc1F ; "."]

### The Group -M¹ (Heteroaryl)

Again, note that -M¹ is attached to Ring A by a bond between a ring carbon atom of -M¹ and a ring carbon atom of Ring A.

(147) A compound according to any one of (1) to (93), wherein -M¹, if present, is an aromatic monocyclic heterocyclic ring having 5 or 6 ring atoms, and is optionally substituted with one or more substituents —R^{M1}.

(148) A compound according to any one of (1) to (93), wherein -M¹, if present, is a C₅₋₆heteroaryl group; and is optionally substituted with one or more substituents —R^{M1}.

(149) A compound according to any one of (1) to (93), wherein -M¹, if present, is independently furanyl, thienyl, pyrrolyl, imidazolyl, oxazolyl, isoxazolyl, thiazolyl, isothiazolyl, pyrazolyl, triazolyl, oxadiazolyl, thiadiazolyl, pyridyl, pyridazinyl, pyrimidinyl, or pyrazinyl; and is optionally substituted with one or more substituents —R^{M1}.

(150) A compound according to any one of (1) to (93), wherein -M¹, if present, is independently thienyl, oxadiazolyl, or pyridyl; and is optionally substituted with one or more substituents —R^{M1}.

(151) A compound according to any one of (1) to (93), wherein -M¹, if present, is a C₅heteroaryl group; and is optionally substituted with one or more substituents —R^{M1}.

(152) A compound according to any one of (1) to (93), wherein -M¹, if present, is independently furanyl, thienyl, pyrrolyl, imidazolyl, oxazolyl, isoxazolyl, thiazolyl, isothiazolyl, pyrazolyl, triazolyl, oxadiazolyl, or thiadiazolyl; and is optionally substituted with one or more substituents —R^{M1}.

(153) A compound according to any one of (1) to (93), wherein -M¹, if present, is independently thienyl or oxadiazolyl; and is optionally substituted with one or more substituents —R^{M1}.

(154) A compound according to any one of (1) to (93), wherein -M¹, if present, is thienyl; and is optionally substituted with one or more substituents —R^{M1}.

(155) A compound according to any one of (1) to (93), wherein -M¹, if present, is thien-2-yl; and is optionally substituted with one or more substituents —R^{M1}.

(156) A compound according to any one of (1) to (93), wherein -M¹, if present, is oxadiazolyl; and is optionally substituted with one or more substituents —R^{M1}.

(157) A compound according to any one of (1) to (93), wherein -M¹, if present, is 1,2,4-oxadiazol-5-yl; and is optionally substituted with one or more substituents —R^{M1}.

(158) A compound according to any one of (1) to (93), wherein -M¹, if present, is a C₆heteroaryl group; and is optionally substituted with one or more substituents —R^{M1}.

(159) A compound according to any one of (1) to (93), wherein -M¹, if present, is independently pyridyl, pyridazinyl, pyrimidinyl, or pyrazinyl; and is optionally substituted with one or more substituents —R^{M1}.

### The Group —R^{M1}

(160) A compound according to any one of (1) to (159), wherein each —R^{M1}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —CN, or —NO₂.

(161) A compound according to any one of (1) to (159), wherein each —R^{M1}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —S(═O)₂R^{M11}, —CN, or —NO₂.

(162) A compound according to any one of (1) to (159), wherein each —R^{M1}, if present, is independently —R^{M11}, —R^{M11X}, —OH, —OR^{M11}, —OR^{M11X}, —F, —Cl, —Br, —I, —NH₂, —NHR^{M11}, —NR^{M11}₂, or —R^{M11N}.

(163) A compound according to any one of (1) to (159), wherein each —R^{M1}, if present, is independently —R^{M11}, —R^{M11X}, —OH, —OR^{M11}, —OR^{M11X}, —F, —Cl, —Br, or —I.

(164) A compound according to any one of (1) to (159), wherein each —R^{M1}, if present, is independently —R^{M11}, —OH, —OR^{M11}, —F, —Cl, —Br, or —I.

(165) A compound according to any one of (1) to (159), wherein each —R^{M1}, if present, is independently —R^{M11}, —F, —Cl, —Br, or —I.

(166) A compound according to any one of (1) to (159), wherein each —R^{M1}, if present, is —R^{M11}.

(167) A compound according to any one of (1) to (159), wherein each —R^{M1}, if present, is independently —F, —Cl, —Br, or —I.

### The Group —R^{M11}

(168) A compound according to any one of (1) to (167), wherein each —R^{M11}, if present, is independently saturated linear or branched C₁₋₃alkyl.

(169) A compound according to any one of (1) to (167), wherein each —R^{M11}, if present, is independently -Me or -Et.

(170) A compound according to any one of (1) to (167), wherein each —R^{M11}, if present, is -Me.

### The Group —R^{M11X}

(171) A compound according to any one of (1) to (170), wherein each —R^{M11X}, if present, is saturated linear or branched C₁₋₄fluoroalkyl.

(172) A compound according to any one of (1) to (170), wherein each —R^{M11X}, if present, is —CF₃.

### The Group —R^{M11N}

(173) A compound according to any one of (1) to (172), wherein each —R^{M11N}, if present, is independently azetidino, pyrrolidino, piperidino, piperazino, or morpholino, and is optionally substituted with one or more substituents selected from —R^{M11}, —OH, and —OR^{M11}.

(174) A compound according to any one of (1) to (172), wherein each —R^{M11N}, if present, is independently azetidino, pyrrolidino, piperidino, piperazino, or morpholino.

### The Group -J (Part 2)

(175) A compound according to any one of (1) to (44), wherein -J is independently:

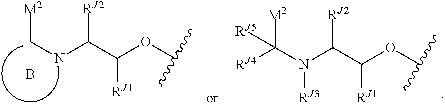

(176) A compound according to any one of (1) to (44), wherein -J is:

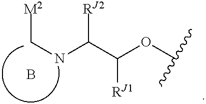

(177) A compound according to any one of (1) to (44), wherein -J is:

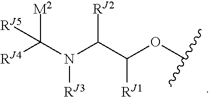

### The Ring B

(178) A compound according to any one of (1) to (44), (175), and (176), wherein Ring B, if present, is Ring E1.

(179) A compound according to any one of (1) to (44), (175), and (176), wherein Ring B, if present, is Ring B2.

### The Ring 1

(180) A compound according to any one of (1) to (44), (175), (176), and (178), wherein Ring E1, if present, is a non-aromatic monocyclic C₄₋₇heterocyclyl group; and is optionally substituted with one or more substituents —R^{B1}; and/or is optionally substituted with ═O.

(181) A compound according to any one of (1) to (44), (175), (176), and (178), wherein Ring 1, if present, is independently azetidinyl, pyrrolidinyl, piperidinyl, piperazinyl, or morpholinyl; and is optionally substituted with one or more substituents —R^{B1}; and/or is optionally substituted with ═O.

(182) A compound according to any one of (1) to (44), (175), (176), and (178), wherein Ring 1, if present, is pyrrolidinyl; and is optionally substituted with one or more substituents —R^{B1}; and/or is optionally substituted with ═O.

### The Ring B2

(183) A compound according to any one of (1) to (44), (175), (176), and (179), wherein Ring B2, if present, is a C₅₋₆heteroaryl group; and is optionally substituted with one or more substituents —R^{B2}.

(184) A compound according to any one of (1) to (44), (175), (176), and (179), wherein Ring B2, if present, is an heteroaromatic monocyclic ring having 5 ring atoms, and is optionally substituted with one or more substituents —R^{B2}.

(185) A compound according to any one of (1) to (44), (175), (176), and (179), wherein Ring B2, if present, is a C₅heteroaryl group; and is optionally substituted with one or more substituents —R^{B2}.

(186) A compound according to any one of (1) to (44), (175), (176), and (179), wherein Ring B2, if present, is independently pyrrolyl, imidazolyl, pyrazolyl, triazolyl, or tetrazolyl; and is optionally substituted with one or more substituents —R^{B2}.

(187) A compound according to any one of (1) to (44), (175), (176), and (179), wherein Ring B2, if present, is independently pyrrolyl, imidazolyl, pyrazolyl, or triazolyl; and is optionally substituted with one or more substituents —R^{B2}.

(188) A compound according to any one of (1) to (44), (175), (176), and (179), wherein Ring B2, if present, is independently pyrrolyl, imidazolyl, or pyrazolyl; and is optionally substituted with one or more substituents —R^{B2}.

(189) A compound according to any one of (1) to (44), (175), (176), and (179), wherein Ring B2, if present, is independently imidazolyl or pyrazolyl; and is optionally substituted with one or more substituents —R^{B2}.

(190) A compound according to any one of (1) to (44), (175), (176), and (179), wherein Ring B2, if present, is imidazolyl; and is optionally substituted with one or more substituents —R^{B2}.

(191) A compound according to any one of (1) to (44), (175), (176), and (179), wherein Ring B2, if present, is pyrazolyl; and is optionally substituted with one or more substituents —R^{B2}.

### The Group —R^{B1}

(192) A compound according to any one of (1) to (44), (175), (176), (178), and (180) to (182), wherein each —R^{B1}, if present, is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —CN, or —NO₂.

(193) A compound according to any one of (1) to (44), (175), (176), (178), and (180) to (182), wherein each —R^{B1}, if present, is independently —R^{BB}, —R^{BBX}, —OH, —OR^{BB}, —OR^{BBX}, —F, —NH₂, —NHR^{BB}, —NR^{BB}₂, —R^{BBN}, —C(═O)R^{BB}, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, or —CN.

(194) A compound according to any one of (1) to (44), (175), (176), (178), and (180) to (182), wherein each —R^{B1}, if present, is independently —R^{BB}, —R^{BBX}, —OH, —OR^{BB}, —OR^{BBX}, —F, —NH₂, —NHR^{BB}, —NR^{BB}₂, —R^{BBN}, or —S(═O)₂R^{BB}.

(195) A compound according to any one of (1) to (44), (175), (176), (178), and (180) to (182), wherein each —R^{B1}, if present, is independently —R^{BB}, —R^{BBX}, —OH, —OR^{BB}, —OR^{BBX}, —F, —Cl, —Br, —I, or —S(═O)₂R^{BB}.

(196) A compound according to any one of (1) to (44), (175), (176), (178), and (180) to (182), wherein each —R^{B1}, if present, is independently —R^{BB}, —R^{BBX}, —OH, —OR^{BB}, —OR^{BBX}, —S(═O)₂R^{BB}, or —F.

(197) A compound according to any one of (1) to (44), (175), (176), (178), and (180) to (182), wherein each —R^{B1}, if present, is independently —R^{BB}, —OH, —OR^{BB}, —S(═O)₂R^{BB}, or —F.

(198) A compound according to any one of (1) to (44), (175), (176), (178), and (180) to (182), wherein each —R^{B1}, if present, is independently —R^{BB}, —OH, —S(═O)₂R^{BB}, or —F.

### The Group —R^{B2}

(199) A compound according to any one of (1) to (44), (175), (176), (179), and (183) to (191), wherein each —R^{B2}, if present, is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —CN, or —NO₂.

(200) A compound according to any one of (1) to (44), (175), (176), (179), and (183) to (191), wherein each —R^{B2}, if present, is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —S(═O)₂R^{BB}, —CN, or —NO₂.

(201) A compound according to any one of (1) to (44), (175), (176), (179), and (183) to (191), wherein each —R^{B2}, if present, is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, or —CN.

(202) A compound according to any one of (1) to (44), (175), (176), (179), and (183) to (191), wherein each —R^{B2}, if present, is independently —R^{BB}, —R^{BBX}, —OH, —OR^{BB}, —OR^{BBX}, —F, —Cl, —Br, or —I.

(203) A compound according to any one of (1) to (44), (175), (176), (179), and (183) to (191), wherein each —R^{B2}, if present, is independently —R^{BB}, —OH, —OR^{BB}, —F, —C, —Br, or —I.

### The Group —R^{BB}

(204) A compound according to any one of (1) to (44) and (175) to (203), wherein each —R^{BB}, if present, is independently saturated linear or branched C₁₋₄alkyl.

(205) A compound according to any one of (1) to (44) and (175) to (203), wherein each —R^{BB}, if present, is independently saturated linear or branched C₁₋₃alkyl.

(206) A compound according to any one of (1) to (44) and (175) to (203), wherein each —R^{BB}, if present, is independently -Me or -Et.

(207) A compound according to any one of (1) to (44) and (175) to (203), wherein each —R^{BB}, if present, is -Me.

### The Group —R^{BBX}

(208) A compound according to any one of (1) to (44) and (175) to (207), wherein each —R^{BBX}, if present, is saturated linear or branched C₁₋₄fluoroalkyl.

(209) A compound according to any one of (1) to (44) and (175) to (207), wherein each —R^{BBX}, if present, is —CF₃.

### The Group —R^{BBN}

(210) A compound according to any one of (1) to (44) and (175) to (209), wherein each —R^{BBN}, if present, is independently azetidino, pyrrolidino, piperidino, piperazino, or morpholino, and is optionally substituted with one or more substituents selected from —R^{BB}, —OH, and —OR^{BB}.

(211) A compound according to any one of (1) to (44) and (175) to (209), wherein each —R^{BBN}, if present, is independently azetidino, pyrrolidino, piperidino, piperazino, or morpholino.

### The Group -M² (Carboaryl)

(212) A compound according to any one of (1) to (44) and (175) to (211), wherein -M², if present, is phenyl; and is optionally substituted with one or more substituents —R^{M2}.

### The Group -M² (Heteroaryl)

(213) A compound according to any one of (1) to (44) and (175) to (211), wherein -M², if present, is a C₅₋₆heteroaryl group; and is optionally substituted with one or more substituents —R^{M2}.

(214) A compound according to any one of (1) to (44) and (175) to (211), wherein -M², if present, is independently furanyl, thienyl, pyrrolyl, imidazolyl, oxazolyl, isoxazolyl, thiazolyl, isothiazolyl, pyrazolyl, triazolyl, oxadiazolyl, thiadiazolyl, pyridyl, pyridazinyl, pyrimidinyl, or pyrazinyl; and is optionally substituted with one or more substituents —R^{M2}.

(215) A compound according to any one of (1) to (44) and (175) to (211), wherein -M², if present, is a C₅heteroaryl group; and is optionally substituted with one or more substituents —R^{M2}.

(216) A compound according to any one of (1) to (44) and (175) to (211), wherein -M², if present, is independently furanyl, thienyl, pyrrolyl, imidazolyl, oxazolyl, isoxazolyl, thiazolyl, isothiazolyl, pyrazolyl, triazolyl, oxadiazolyl, or thiadiazolyl; and is optionally substituted with one or more substituents —R^{M2}.

(217) A compound according to any one of (1) to (44) and (175) to (211), wherein -M², if present, is independently thienyl, pyrrolyl, imidazolyl, oxazolyl, isoxazolyl, thiazolyl, isothiazolyl, triazolyl, or thiadiazolyl; and is optionally substituted with one or more substituents —R^{M2}.

(218) A compound according to any one of (1) to (44) and (175) to (211), wherein -M², if present, is independently thienyl, pyrrolyl, or pyrazolyl; and is optionally substituted with one or more substituents —R^{M2}.

(219) A compound according to any one of (1) to (44) and (175) to (211), wherein -M², if present, is independently thienyl or pyrrolyl; and is optionally substituted with one or more substituents —R^{M2}.

(220) A compound according to any one of (1) to (44) and (175) to (211), wherein -M², if present, is a C₆heteroaryl group; and is optionally substituted with one or more substituents —R^{M2}.

(221) A compound according to any one of (1) to (44) and (175) to (211), wherein -M², if present, is independently pyridyl, pyridazinyl, pyrimidinyl, or pyrazinyl; and is optionally substituted with one or more substituents —R^{M2}.

(222) A compound according to any one of (1) to (44) and (175) to (211), wherein -M², if present, is pyridyl; and is optionally substituted with one or more substituents —R^{M2}.

### The Group —R^{M2}

(223) A compound according to any one of (1) to (44) and (175) to (222), wherein each —R^{M2}, if present, is independently —R^{M22}, —R^{M22X}, —OH, —OR^{M22}, —OR^{M22X}, —F, —Cl, —Br, —I, —NH₂, —NHR^{M22}, —NR^{M22}₂, —R^{M22N}, —C(═O)R^{M22}, —C(═O)OR^{M22}, —OC(═O)R^{M22}, —NHC(═O)R^{M22}, —C(═O)NH₂, —C(═O)NHR^{M22}, —C(═O)NR^{M22}₂, —C(═O)R^{M22N}, —S(═O)₂R^{M22}, —CN, or —NO₂.

(224) A compound according to any one of (1) to (44) and (175) to (222), wherein each —R^{M2}, if present, is independently —R^{M22}, —R^{M22X}, —OH, —OR^{M22}, —OR^{M22X}, —F, —Cl, —Br, —I, —NH₂, —NHR^{M22}, —NR^{M22}₂, —R^{M22N}, —S(═O)₂R^{M22}, —CN, or —NO₂.

(225) A compound according to any one of (1) to (44) and (175) to (222), wherein each —R^{M2}, if present, is independently —R^{M22}, —R^{M22X}, —OH, —OR^{M22}, —OR^{M22X}, —F, —Cl, —Br, —I, —NH₂, —NHR^{M22}, —NR^{M22}₂, or —R^{M22N}.

(226) A compound according to any one of (1) to (44) and (175) to (222), wherein each —R^{M2}, if present, is independently —R^{M22}, —R^{M22X}, —OH, —OR^{M22}, —OR^{M22X}, —F, —Cl, —Br, or —I.

(227) A compound according to any one of (1) to (44) and (175) to (222), wherein each —R^{M2}, if present, is independently —R^{M22}, —OH, —OR^{M22}, —F, —Cl, —Br, or —I.

(228) A compound according to any one of (1) to (44) and (175) to (222), wherein each —R^{M2}, if present, is independently —R^{M22}, —F, —Cl, —Br, or —I.

(229) A compound according to any one of (1) to (44) and (175) to (222), wherein each —R^{M2}, if present, is independently —F, —Cl, —Br, or —I.

### The Group —R^{M22}

(230) A compound according to any one of (1) to (44) and (175) to (229), wherein each —R^{M22}, if present, is independently saturated linear or branched C₁₋₃alkyl.

(231) A compound according to any one of (1) to (44) and (175) to (229), wherein each —R^{M22}, if present, is independently -Me or -Et.

(232) A compound according to any one of (1) to (44) and (175) to (229), wherein each —R^{M22}, if present, is -Me.

### The Group —R^{M22X}

(233) A compound according to any one of (1) to (44) and (175) to (232), wherein each —R^{M22X}, if present, is saturated linear or branched C₁₋₄fluoroalkyl.

(234) A compound according to any one of (1) to (44) and (175) to (232), wherein each —R^{M22X}, if present, is —CF₃.

### The Group —R^{M22N}

(235) A compound according to any one of (1) to (44) and (175) to (234), wherein each —R^{M22}N, if present, is independently azetidino, pyrrolidino, piperidino, piperazino, or morpholino, and is optionally substituted with one or more substituents selected from —R^{M22}, —OH, and —OR^{M22}.

(236) A compound according to any one of (1) to (44) and (175) to (234), wherein each —R^{M22}N, if present, is independently azetidino, pyrrolidino, piperidino, piperazino, or morpholino.

### The Group —R^{J1}

(237) A compound according to any one of (1) to (44) and (175) to (236), wherein each —R^{J1}, if present, is —H.

(238) A compound according to any one of (1) to (44) and (175) to (236), wherein each —R^{J1}, if present, is —R^{JJ}.

### The Group —R^{J2}

(239) A compound according to any one of (1) to (44) and (175) to (238), wherein each —R^{J2}, if present, is —H.

(240) A compound according to any one of (1) to (44) and (175) to (238), wherein each —R^{J2}, if present, is —R^{JJ}.

### The Group —R^{J3}

(241) A compound according to any one of (1) to (44) and (175) to (240), wherein —R^{J3}, if present, is —R^{JJ}.

The Groups —R^{J4} and —R^{J5}

(242) A compound according to any one of (1) to (44) and (175) to (241), wherein:
—R^{J4}, if present, is independently —H, —R^{JJ}, or -L^{JJ}-OH;
—R^{J5}, if present, is independently —H, —R^{JJ}, or -L^{JJ}-OH; or
—R^{J4} and —R^{J5}, if present, taken together form ═O.

(243) A compound according to any one of (1) to (44) and (175) to (241), wherein —R^{J5}, if present, is —H.

(244) A compound according to any one of (1) to (44) and (175) to (241), wherein:
—R^{J4}, if present, is independently —H, —R^{JJ}, or -L^{JJ}-OH;
—R^{J5}, if present, is —H; or
—R^{J4} and —R^{J5}, if present, taken together form ═O.

(245) A compound according to any one of (1) to (44) and (175) to (241), wherein:
—R^{J4}, if present, is —H;
—R^{J5}, if present, is —H.

### The Group —R^{JJ}

(246) A compound according to any one of (1) to (44) and (175) to (245), wherein each —R^{JJ}, if present, is independently saturated linear or branched C₁₋₃alkyl.

(247) A compound according to any one of (1) to (44) and (175) to (245), wherein each —R^{JJ}, if present, is independently -Me or -Et.

(248) A compound according to any one of (1) to (44) and (175) to (245), wherein each —R^{JJ}, if present, is -Me.

### The Group -L^{JJ}-

(249) A compound according to any one of (1) to (44) and (175) to (248), wherein each -L^{JJ}-, if present, is independently saturated linear or branched C₁₋₃alkylene.

(250) A compound according to any one of (1) to (44) and (175) to (248), wherein each -L^{JJ}-, if present, is independently —CH₂—, —CH₂CH₂—, or —CH₂CH₂CH₂—.

(251) A compound according to any one of (1) to (44) and (175) to (248), wherein each -L^{JJ}-, if present, is independently —CH₂— or —CH₂CH₂—.

### Specific Embodiments

(252) A compound according to (1), which is a compound of one of the following formulae, or a pharmaceutically acceptable salt or solvate thereof:

| Code | STRUCTURE |
|---|---|
| ALDHI-1001 | 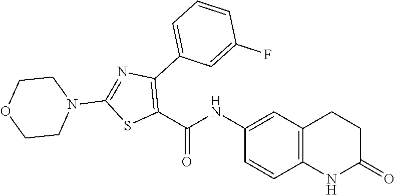 [SMILES: O=C1CCc2cc(NC(=O)c3sc(N4CCOCC4)nc3-c3cccc(F)c3)ccc2N1] |
| ALDHI-1002 | 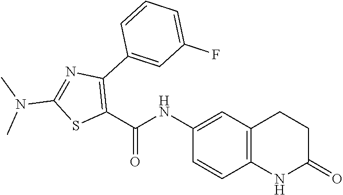 [SMILES: CN(C)c1nc(-c2cccc(F)c2)c(C(=O)Nc2ccc3c(c2)CCC(=O)N3)s1] |
| ALDHI-1003 | 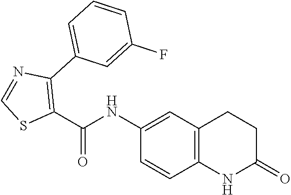 [SMILES: O=C1CCc2cc(NC(=O)c3scnc3-c3cccc(F)c3)ccc2N1] |
| ALDHI-1004 | 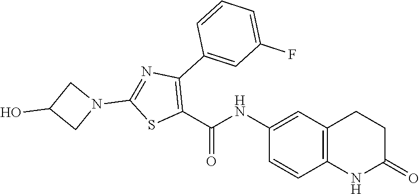 [SMILES: O=C1CCc2cc(NC(=O)c3sc(N4CC(O)C4)nc3-c3cccc(F)c3)ccc2N1] |
| ALDHI-1005 | 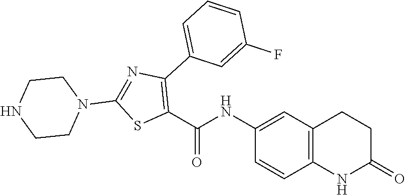 [SMILES: O=C1CCc2cc(NC(=O)c3sc(N4CCNCC4)nc3-c3cccc(F)c3)ccc2N1] |
| ALDHI-1006 | 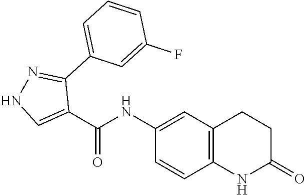 [SMILES: O=C1CCc2cc(NC(=O)c3c[nH]nc3-c3cccc(F)c3)ccc2N1] |
| ALDHI-1007 | 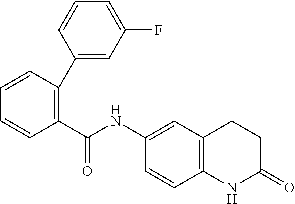 [SMILES: O=C1CCc2cc(NC(=O)c3ccccc3-c3cccc(F)c3)ccc2N1] |
| ALDHI-1008 | 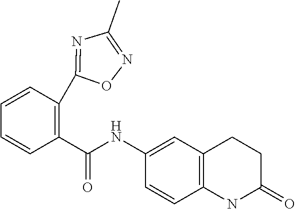 [SMILES: Cc1noc(-c2ccccc2C(=O)Nc2ccc3c(c2)CCC(=O)N3)n1] |
| ALDHI-1009 | 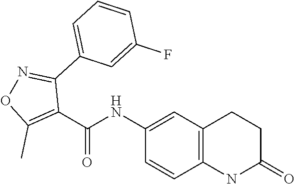 [SMILES: Cc1onc(-c2cccc(F)c2)c1C(=O)Nc1ccc2c(c1)CCC(=O)N2] |
| ALDHI-1010 | 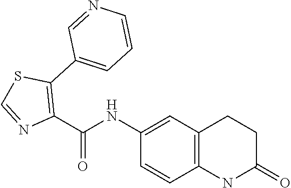 [SMILES: O=C1CCc2cc(NC(=O)c3ncsc3-c3cccnc3)ccc2N1] |
| ALDHI-1011 | 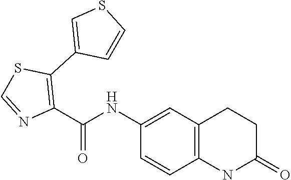 [SMILES: O=C1CCc2cc(NC(=O)c3ncsc3-c3ccsc3)ccc2N1] |
| ALDHI-1012 | 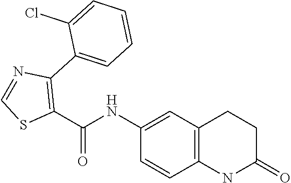 [SMILES: O=C1CCc2cc(NC(=O)c3scnc3-c3ccccc3Cl)ccc2N1] |
| ALDHI-1013 | 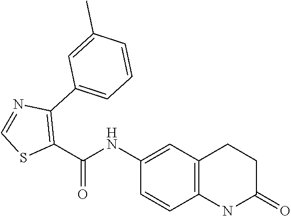 [SMILES: Cc1cccc(-c2ncsc2C(=O)Nc2ccc3c(c2)CCC(=O)N3)c1] |
| ALDHI-1014 | 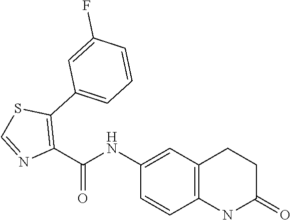 [SMILES: O=C1CCc2cc(NC(=O)c3ncsc3-c3cccc(F)c3)ccc2N1] |
| ALDHI-1015 | 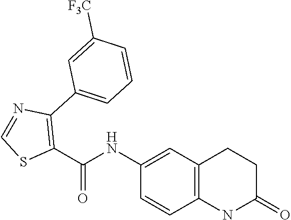 [SMILES: O=C1CCc2cc(NC(=O)c3scnc3-c3cccc(C(F)(F)F)c3)ccc2N1] |
| ALDHI-1016 | 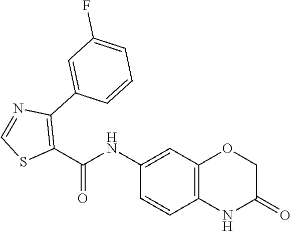 [SMILES: O=C1COc2cc(NC(=O)c3scnc3-c3cccc(F)c3)ccc2N1] |
| ALDHI-1017 | 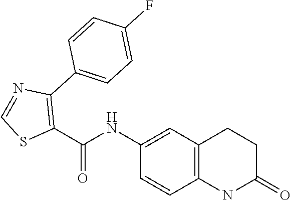 [SMILES: O=C1CCc2cc(NC(=O)c3scnc3-c3ccc(F)cc3)ccc2N1] |
| ALDHI-1018 | 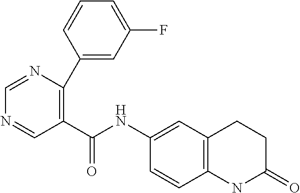 [SMILES: O=C1CCc2cc(NC(=O)c3cncnc3-c3cccc(F)c3)ccc2N1] |
| ALDHI-1019 | 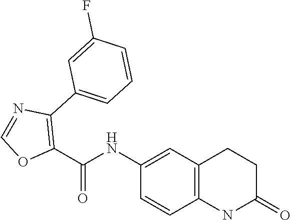 [SMILES: O=C1CCc2cc(NC(=O)c3ocnc3-c3cccc(F)c3)ccc2N1] |
| ALDHI-1020 | 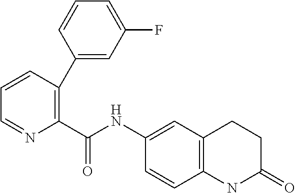 [SMILES: O=C1CCc2cc(NC(=O)c3ncccc3-c3cccc(F)c3)ccc2N1] |
| ALDHI-1021 | 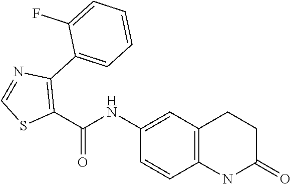 [SMILES: O=C1CCc2cc(NC(=O)c3scnc3-c3ccccc3F)ccc2N1] |
| ALDHI-1022 | 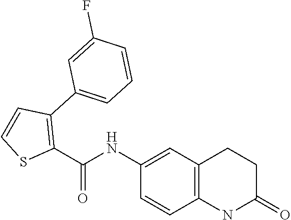 [SMILES: O=C1CCc2cc(NC(=O)c3sccc3-c3cccc(F)c3)ccc2N1] |
| ALDHI-1023 | 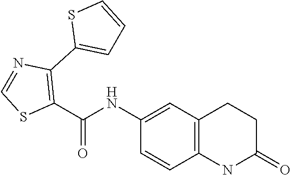 [SMILES: O=C1CCc2cc(NC(=O)c3scnc3-c3cccs3)ccc2N1] |
| ALDHI-1024 | 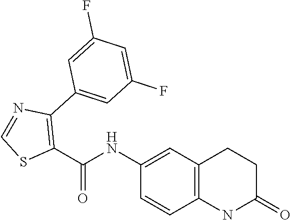 [SMILES: O=C1CCc2cc(NC(=O)c3scnc3-c3cc(F)cc(F)c3)ccc2N1] |
| ALDHI-1025 | 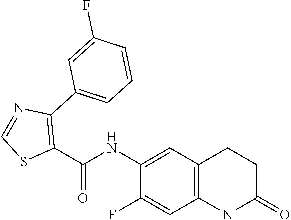 [SMILES: O=C1CCc2cc(NC(=O)c3scnc3-c3cccc(F)c3)c(F)cc2N1] |
| ALDHI-1026 | 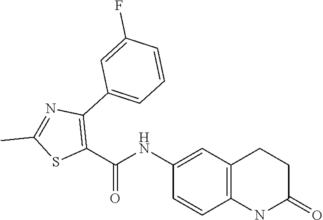 [SMILES: Cc1nc(-c2cccc(F)c2)c(C(=O)Nc2ccc3c(c2)CCC(=O)N3)s1] |

(253) A compound according to (1), which is a compound of one of the following formulae, or a pharmaceutically acceptable salt or solvate thereof:

| Code | STRUCTURE |
|---|---|
| ALDHI-2001 | 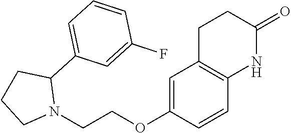 [SMILES: O=C1CCc2cc(OCCN3CCCC3c3cccc(F)c3)ccc2N1] |
| ALDHI-2002 | 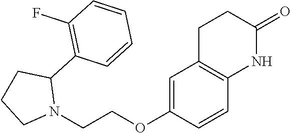 [SMILES: O=C1CCc2cc(OCCN3CCCC3c3ccccc3F)ccc2N1] |
| ALDHI-2003 | 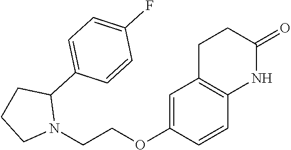 [SMILES: O=C1CCc2cc(OCCN3CCCC3c3ccc(F)cc3)ccc2N1] |
| ALDHI-2004 | 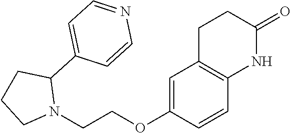 [SMILES: O=C1CCc2cc(OCCN3CCCC3c3ccncc3)ccc2N1] |
| ALDHI-2005 | 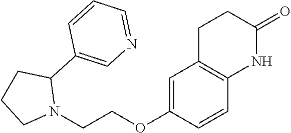 [SMILES: O=C1CCc2cc(OCCN3CCCC3c3cccnc3)ccc2N1] |
| ALDHI-2006 | 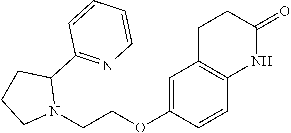 [SMILES: O=C1CCc2cc(OCCN3CCCC3c3ccccn3)ccc2N1] |
| ALDHI-2007 | 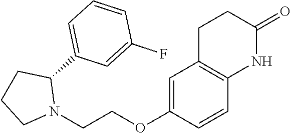 [SMILES: O=C1CCc2cc(OCCN3CCC[C@@H]3c3cccc(F)c3)ccc2N1] |
| ALDHI-2008 | 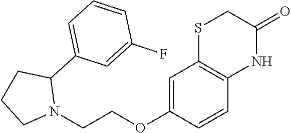 [SMILES: O=C1CSc2cc(OCCN3CCCC3c3cccc(F)c3)ccc2N1] |
| ALDHI-2009 | 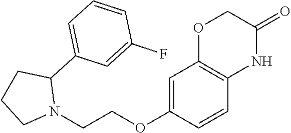 [SMILES: O=C1COc2cc(OCCN3CCCC3c3cccc(F)c3)ccc2N1] |
| ALDHI-2010 | 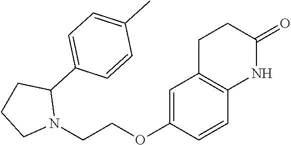 [SMILES: Cc1ccc(C2CCCN2CCOc2ccc3c(c2)CCC(=O)N3)cc1] |
| ALDHI-2011 | 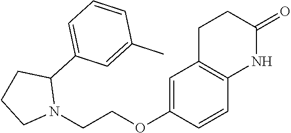 [SMILES: Cc1cccc(C2CCCN2CCOc2ccc3c(c2)CCC(=O)N3)c1] |
| ALDHI-2012 | 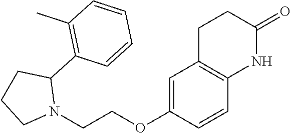 [SMILES: Cc1ccccc1C1CCCN1CCOc1ccc2c(c1)CCC(=O)N2] |
| ALDHI-2013 | 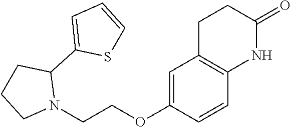 [SMILES: O=C1CCc2cc(OCCN3CCCC3c3cccs3)ccc2N1] |
| ALDHI-2014 | 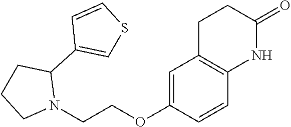 [SMILES: O=C1CCc2cc(OCCN3CCCC3c3ccsc3)ccc2N1] |
| ALDHI-2015 | 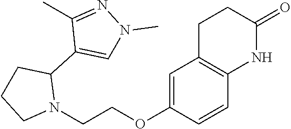 [SMILES: Cc1nn(C)cc1C1CCCN1CCOc1ccc2c(c1)CCC(=O)N2] |
| ALDHI-2016 | 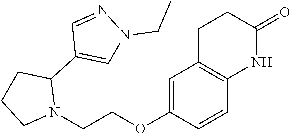 [SMILES: CCn1cc(C2CCCN2CCOc2ccc3c(c2)CCC(=O)N3)cn1] |
| ALDHI-2017 | 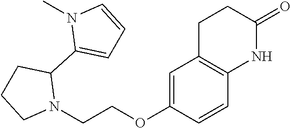 [SMILES: Cn1cccc1C1CCCN1CCOc1ccc2c(c1)CCC(=O)N2] |
| ALDHI-2018 | 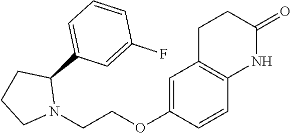 [SMILES: O=C1CCc2cc(OCCN3CCC[C@H]3c3cccc(F)c3)ccc2N1] |
| ALDHI-2019 | 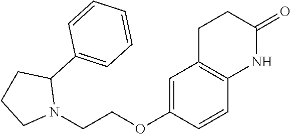 [SMILES: O=C1CCc2cc(OCCN3CCCC3c3ccccc3)ccc2N1] |
| ALDHI-2020 | 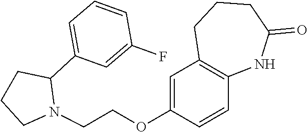 [SMILES: O=C1CCCc2cc(OCCN3CCCC3c3cccc(F)c3)ccc2N1] |
| ALDHI-2021 | 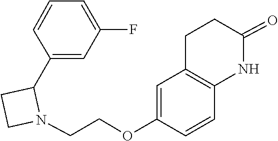 [SMILES: O=C1CCc2cc(OCCN3CCC3c3cccc(F)c3)ccc2N1] |
| ALDHI-2022 | 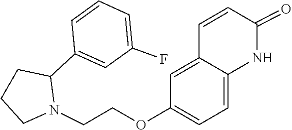 [SMILES: O=c1ccc2cc(OCCN3CCCC3c3cccc(F)c3)ccc2[nH]1] |
| ALDHI-2023 | 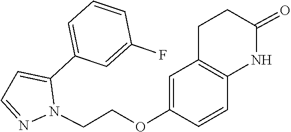 [SMILES: O=C1CCc2cc(OCCn3nccc3-c3cccc(F)c3)ccc2N1] |
| ALDHI-2024 | 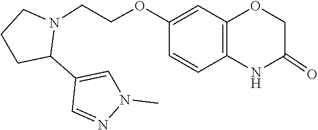 [SMILES: Cn1cc(C2CCCN2CCOc2ccc3c(c2)OCC(=O)N3)cn1] |
| ALDHI-2025 | 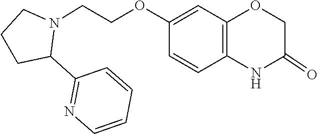 [SMILES: O=C1COc2cc(OCCN3CCCC3c3ccccn3)ccc2N1] |
| ALDHI-2026 | 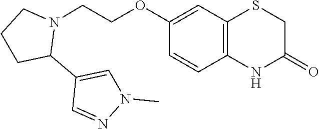 [SMILES: Cn1cc(C2CCCN2CCOc2ccc3c(c2)SCC(=O)N3)cn1] |
| ALDHI-2027 | 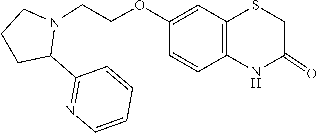 [SMILES: O=C1CSc2cc(OCCN3CCCC3c3ccccn3)ccc2N1] |
| ALDHI-2028 | 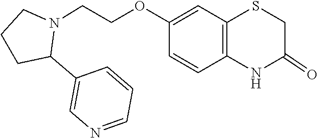 [SMILES: O=C1CSc2cc(OCCN3CCCC3c3cccnc3)ccc2N1] |
| ALDHI-2029 | 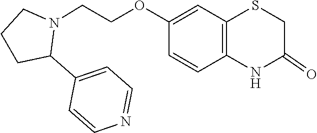 [SMILES: O=C1CSc2cc(OCCN3CCCC3c3ccncc3)ccc2N1] |
| ALDHI-2030 | 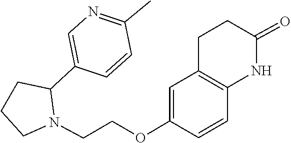 [SMILES: Cc1ccc(C2CCCN2CCOc2ccc3c(c2)CCC(=O)N3)cn1] |
| ALDHI-2031 | 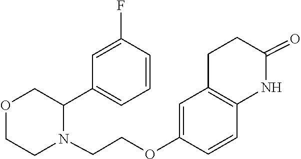 [SMILES: O=C1CCc2cc(OCCN3CCOCC3c3cccc(F)c3)ccc2N1] |
| ALDHI-2032 | 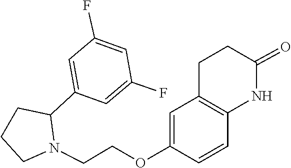 [SMILES: O=C1CCc2cc(OCCN3CCCC3c3cc(F)cc(F)c3)ccc2N1] |
| ALDHI-2033 | 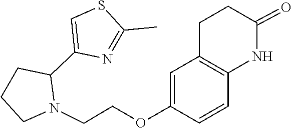 [SMILES: Cc1nc(C2CCCN2CCOc2ccc3c(c2)CCC(=O)N3)cs1] |
| ALDHI-2034 | 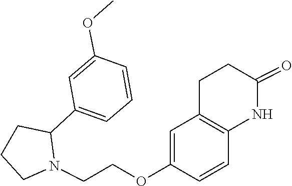 [SMILES: COc1cccc(C2CCCN2CCOc2ccc3c(c2)CCC(=O)N3)c1] |
| ALDHI-2035 | 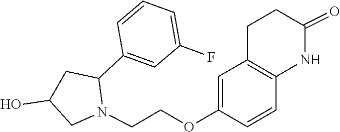 [SMILES: O=C1CCc2cc(OCCN3CC(O)CC3c3cccc(F)c3)ccc2N1] |
| ALDHI-2036 | 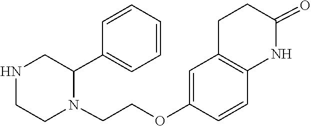 [SMILES: O=C1CCc2cc(OCCN3CCNCC3c3ccccc3)ccc2N1] |
| ALDHI-2037 | 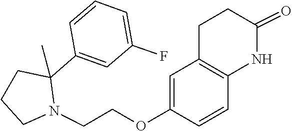 [SMILES: CC1(c2cccc(F)c2)CCCN1CCOc1ccc2c(c1)CCC(=O)N2] |
| ALDHI-2038 | 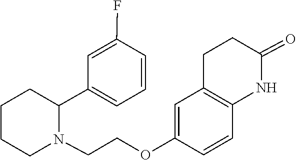 [SMILES: O=C1CCc2cc(OCCN3CCCCC3c3cccc(F)c3)ccc2N1] |
| ALDHI-2039 | 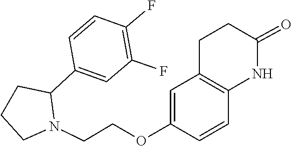 [SMILES: O=C1CCc2cc(OCCN3CCCC3c3ccc(F)c(F)c3)ccc2N1] |
| ALDHI-2040 | 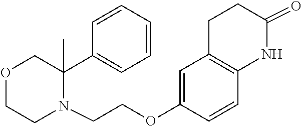 [SMILES: CC1(c2ccccc2)COCCN1CCOc1ccc2c(c1)CCC(=O)N2] |
| ALDHI-2041 | 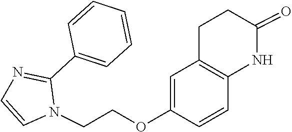 [SMILES: O=C1CCc2cc(OCCn3ccnc3-c3ccccc3)ccc2N1] |
| ALDHI-2042 | 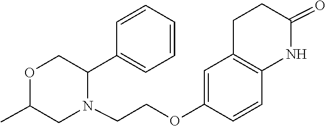 [SMILES: CC1CN(CCOc2ccc3c(c2)CCC(=O)N3)C(c2ccccc2)CO1] |
| ALDHI-2043 | 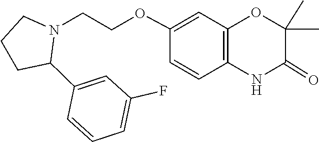 [SMILES: CC1(C)Oc2cc(OCCN3CCCC3c3cccc(F)c3)ccc2NC1=O] |
| ALDHI-2044 | 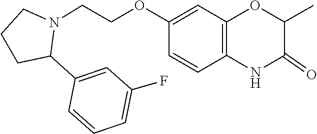 [SMILES: CC1Oc2cc(OCCN3CCCC3c3cccc(F)c3)ccc2NC1=O] |
| ALDHI-2045 | 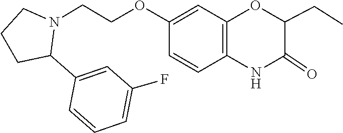 [SMILES: CCC1Oc2cc(OCCN3CCCC3c3cccc(F)c3)ccc2NC1=O] |
| ALDHI-2046 | 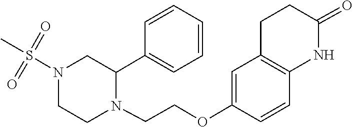 [SMILES: CS(=O)(=O)N1CCN(CCOc2ccc3c(c2)CCC(=O)N3)C(c2ccccc2)C1] |
| ALDHI-2047 | 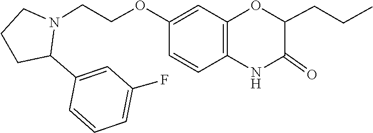 [SMILES: CCCC1Oc2cc(OCCN3CCCC3c3cccc(F)c3)ccc2NC1=O] |
| ALDHI-2048 | 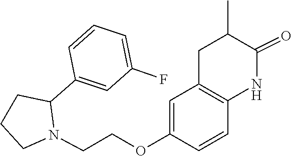 [SMILES: CC1Cc2cc(OCCN3CCCC3c3cccc(F)c3)ccc2NC1=O] |
| ALDHI-2049 | 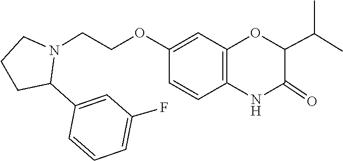 [SMILES: CC(C)C1Oc2cc(OCCN3CCCC3c3cccc(F)c3)ccc2NC1=O] |
| ALDHI-2050 | 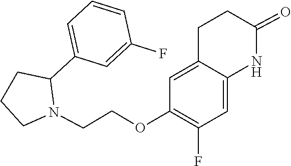 [SMILES: O=C1CCc2cc(OCCN3CCCC3c3cccc(F)c3)c(F)cc2N1] |
| ALDHI-2051 | 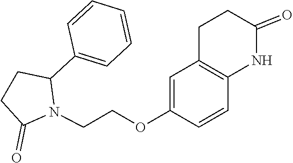 [SMILES: O=C1CCc2cc(OCCN3C(=O)CCC3c3ccccc3)ccc2N1] |
| ALDHI-2052 | 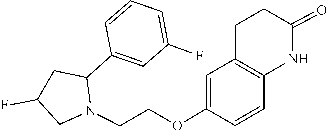 [SMILES: O=C1CCc2cc(OCCN3CC(F)CC3c3cccc(F)c3)ccc2N1] |
| ALDHI-2053 | 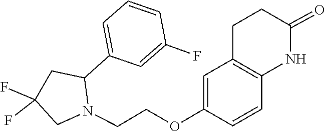 [SMILES: O=C1CCc2cc(OCCN3CC(F)(F)CC3c3cccc(F)c3)ccc2N1] |

(254) A compound according to (1), which is a compound of one of the following formulae, or a pharmaceutically acceptable salt or solvate thereof:

| Code | STRUCTURE |
|---|---|
| ALDHI-3001 | 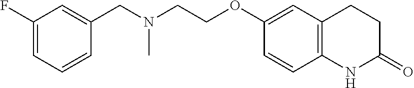 [SMILES: CN(CCOc1ccc2c(c1)CCC(=O)N2)Cc1cccc(F)c1] |
| ALDHI-3002 | 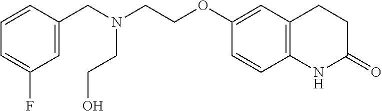 [SMILES: O=C1CCc2cc(OCCN(CCO)Cc3cccc(F)c3)ccc2N1] |
| ALDHI-3003 | 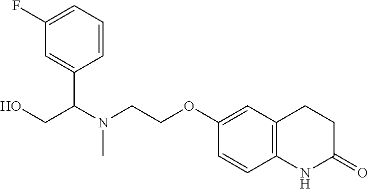 [SMILES: CN(CCOc1ccc2c(c1)CCC(=O)N2)C(CO)c1cccc(F)c1] |
| ALDHI-3004 | 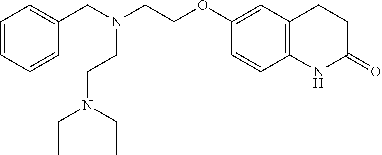 [SMILES: CCN(CC)CCN(CCOc1ccc2c(c1)CCC(=O)N2)Cc1ccccc1] |
| ALDHI-3005 | 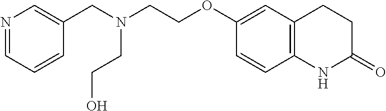 [SMILES: O=C1CCc2cc(OCCN(CCO)Cc3cccnc3)ccc2N1] |
| ALDHI-3006 | 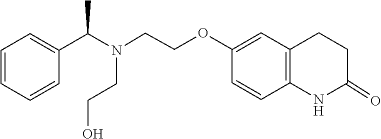 [SMILES: C[C@H](c1ccccc1)N(CCO)CCOc1ccc2c(c1)CCC(=O)N2] |
| ALDHI-3007 | 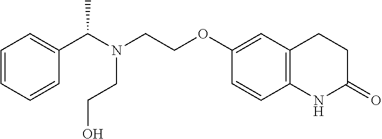 [SMILES: C[C@@H](c1ccccc1)N(CCO)CCOc1ccc2c(c1)CCC(=O)N2] |
| ALDHI-3008 | 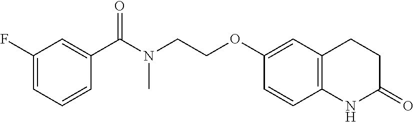 [SMILES: CN(CCOc1ccc2c(c1)CCC(=O)N2)C(=O)c1cccc(F)c1] |
| ALDHI-3009 | 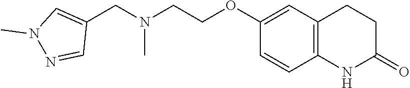 [SMILES: CN(CCOc1ccc2c(c1)CCC(=O)N2)Cc1cnn(C)c1] |
| ALDHI-3010 | 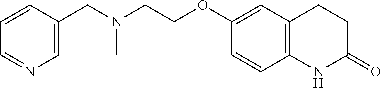 [SMILES: CN(CCOc1ccc2c(c1)CCC(=O)N2)Cc1cccnc1] |
| ALDHI-3011 | 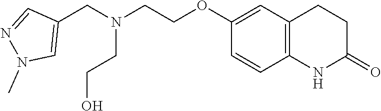 |
| ALDHI-3012 | 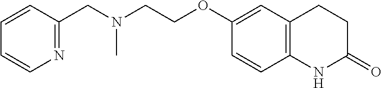 [SMILES: CN(CCOc1ccc2c(c1)CCC(=O)N2)Cc1ccccn1] |
| ALDHI-3013 | 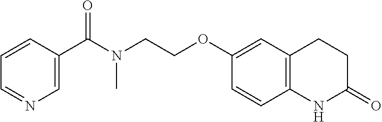 [SMILES: CN(CCOc1ccc2c(c1)CCC(=O)N2)C(=O)c1cccnc1] |
| ALDHI-3014 | 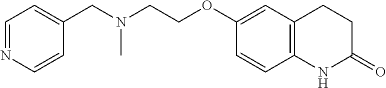 [SMILES: CN(CCOc1ccc2c(c1)CCC(=O)N2)Cc1ccncc1] |

### Combinations

It is appreciated that certain features of the compounds, which are, for clarity, described in the context of separate embodiments, may also be provided in combination in a single embodiment. Conversely, various features of the compounds, which are, for brevity, described in the context of a single embodiment, may also be provided separately or in any suitable sub-combination. All combinations of the embodiments pertaining to the chemical groups represented by the variables (e.g., -Q-, -J, R¹, R³, R⁴, etc.) are specifically embraced by the present disclosure and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present disclosure and are disclosed herein just as if each and every such sub-combination of chemical groups was individually and explicitly disclosed herein.

### Substantially Purified Forms

One aspect of the present invention pertains to ALDHI compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless otherwise specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl). However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference specifically to ortho-chlorophenyl is not to be construed as a reference to its structural isomer, meta-chlorophenyl.

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro. A reference herein to one tautomer is intended to encompass both tautomers.

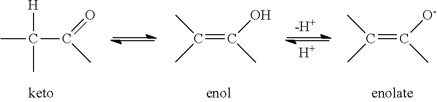

For example, 1H-pyridin-2-one-5-yl and 2-hydroxyl-pyridin-5-yl (shown below) are tautomers of one another. A reference herein to one is intended to encompass both.

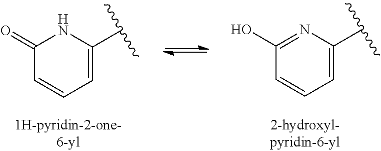 [SMILES: *c1cccc(=O)[nH]1 ; *c1cccc(O)n1]

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group, which may be anionic (e.g., —COOH may be —COO⁻), then a salt may be formed with a suitable cation.

Examples of suitable inorganic cations include, but are not limited to, alkali metal ions such as Na⁺ and K⁺, alkaline earth cations such as Ca²⁺ and Mg²⁺, and other cations such as Al³⁺ as well as the ammonium ion (i.e., NH₄⁺). Examples of suitable organic cations include, but are not limited to substituted ammonium ions (e.g., NH₃R⁺, NH₂R₂⁺, NHR₃⁺, NR₄⁺), for example, where each R is independently linear or branched saturated C₁₋₁₈alkyl, C₃₋₈cycloalkyl, C₃₋₈cycloalkyl-C₁₋₆alkyl, and phenyl-C₁₋₆alkyl, wherein the phenyl group is optionally substituted. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group, which upon protonation may become cationic (e.g., —NH₂ may become —NH₃⁺), then a salt may be formed with a suitable anion.

For example, if a parent structure contains a cationic group (e.g., —NMe₂⁺), or has a functional group, which upon protonation may become cationic (e.g., —NH₂ may become —NH₃⁺), then a salt may be formed with a suitable anion. In the case of a quaternary ammonium compound a counter-anion is generally always present in order to balance the positive charge. If, in addition to a cationic group (e.g., —NMe₂⁺, —NH₃⁺), the compound also contains a group capable of forming an anion (e.g., —COOH), then an inner salt (also referred to as a zwitterion) may be formed.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyloxybenzoic, acetic, trifluoroacetic, ascorbic, aspartic, benzoic, camphorsulfonic, cinnamic, citric, edetic, 1,2-ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, reactive chemical reagents, and the like). In practice, well-known chemical methods are employed to reversibly render unreactive a functional group, which otherwise would be reactive, under specified conditions. In a chemically protected form, one or more reactive functional groups are in the form of a protected or protecting group (alternatively as a masked or masking group or a blocked or blocking group). By protecting a reactive functional group, reactions involving other unprotected reactive functional groups can be performed, without affecting the protected group; the protecting group may be removed or the masking group transformed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an amine group may be protected, for example, as an amide (—NRCO—R), for example: as an acetamide (—NHCO—CH₃); or as a carbamate (—NRCO—OR), for example: as a benzyloxy carbamate (—NHCO—OCH₂C₆H₅, —NH-Cbz), as a t-butoxy carbamate (—NHCO—OC(CH₃)₃, —NH-Boc); as a 2-biphenyl-2-propoxy carbamate (—NHCO—OC(CH₃)₂C₆H₄C₆H₅, —NH-Bpoc), as a 9-fluorenylmethoxy carbamate (—NH—Fmoc), as a 6-nitroveratryloxy carbamate (—NH—Nvoc), as a 2-trimethylsilylethyloxy carbamate (—NH-Teoc), a 2,2,2-trichloroethyloxy carbamate (—NH-Troc), as an allyloxy amide (—NH-Alloc), or as a 2(-phenylsulfonyl)ethyloxy carbamate (—NH—Psec); or, in suitable cases (e.g., cyclic amines), as a nitroxide radical (>N—O·); or, in suitable cases (e.g., heterocyclic nitrogens), as a 2-trimethylsilylethoxymethyl (N-SEM).

### Prodrugs

It may be convenient or desirable to prepare, purify, and/or handle the compound in the form of a prodrug. The term “prodrug,” as used herein, pertains to a compound, which yields the desired active compound in vivo. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

### Compositions

Also described herein is a composition (e.g., a pharmaceutical composition) comprising an ALDHI compound, as described herein, and a pharmaceutically acceptable carrier, diluent, or excipient.

Also described herein is a method of preparing a composition (e.g., a pharmaceutical composition) comprising mixing an ALDHI compound, as described herein, and a pharmaceutically acceptable carrier, diluent, or excipient.

### Uses

The ALDHI compounds, as described herein, inhibit ALDH1A3 enzyme (e.g., inhibit or reduce or block the activity or function of ALDH1A3 enzyme).

Accordingly, the ALDHI compounds, as described herein, are useful, for example, in the treatment of disorders (e.g., diseases) that are ameliorated by the inhibition of ALDH1A3 enzyme (e.g., by the inhibition or reduction or blockage of the activity or function of ALDH1A3 enzyme).

### Use in Methods of Inhibiting ALDH1A3 Enzyme

Also described herein is a method of inhibiting ALDH1A3 enzyme (e.g., inhibiting or reducing or blocking the activity or function of ALDH1A3 enzyme), in vitro or in vivo, comprising contacting the ALDH1A3 enzyme with an effective amount of an ALDHI compound, as described herein.

Also described herein is a method of inhibiting ALDH1A3 enzyme (e.g., inhibiting or reducing or blocking the activity or function of ALDH1A3 enzyme) in a cell, in vitro or in vivo, comprising contacting the cell with an effective amount of an ALDHI compound, as described herein.

In one embodiment, the method is performed in vitro.

In one embodiment, the method is performed in vivo.

In one embodiment, the ALDHI compound is provided in the form of a pharmaceutically acceptable composition.

One of ordinary skill in the art is readily able to determine whether or not a candidate compound inhibits ALDH1A3 enzyme (e.g., inhibits or reduces or blocks the activity or function of ALDH1A3 enzyme). For example, suitable assays are described herein and/or are known in the art.

One of ordinary skill in the art is readily able to determine whether or not a candidate compound inhibits ALDH1A3 enzyme (e.g., inhibits or reduces or blocks or the activity or function of ALDH1A3 enzyme) in a cell. For example, a sample of cells may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a subject (e.g., patient) carrying cells of the same cellular type. As another example of “effect,” the direct interaction of the compound with the target in cells could be measured (e.g., “target engagement assay”) using, e.g., a colorimetric, fluorescent, or luminescent readout.

### Use in Methods of Inhibiting Cell Proliferation, Etc.

The ALDHI compounds described herein may e.g., (a) regulate (e.g., inhibit) cell proliferation; (b) inhibit cell cycle progression; (c) promote apoptosis; (d) reduce clonogenicity; (e) reduce tumoursphere growth or self-renewal; or (f) a combination of one or more of these.

Accordingly, also described herein is a method of regulating (e.g., inhibiting) cell proliferation (e.g., proliferation of a cell), inhibiting cell cycle progression, promoting apoptosis, reducing clonogenicity, reducing tumoursphere growth or self-renewal, or a combination of one or more these, in vitro or in vivo, comprising contacting a cell with an effective amount of an ALDHI compound, as described herein.

In one embodiment, the method is performed in vitro.

In one embodiment, the method is performed in vivo.

In one embodiment, the ALDHI compound is provided in the form of a pharmaceutically acceptable composition.

Any type of cell may be treated or targeted, including for example blood (including, e.g., neutrophils, eosinophils, basophils, lymphocytes, monocytes, erythrocytes, thrombocytes), lung, gastrointestinal (including, e.g., bowel, colon), breast (mammary), ovarian, prostate, liver (hepatic), kidney (renal), bladder, pancreas, brain, and skin cells.

One of ordinary skill in the art is readily able to determine whether or not a candidate compound regulates (e.g., inhibits) cell proliferation, etc. For example, assays which may conveniently be used to assess the activity offered by a particular compound are described herein and/or are known in the art.

The ALDHI compounds described herein may inhibit cell migration and invasion, e.g., inhibit metastasis.

The ALDHI compounds described herein may restore sensitivity to another agent in a resistant cell population.

The ALDHI compounds described herein may prevent emergence of resistance to another agent in a cell population.

### Use in Methods of Therapy

Also described herein is an ALDHI compound, as described herein, for use in a method of treatment of the human or animal body by therapy, for example, for use in a method of treatment of a disorder (e.g., a disease) as described herein.

Also described herein is use of an ALDHI compound, as described herein, in a method of treatment of the human or animal body by therapy, for example, in a method of treatment of a disorder (e.g., a disease) as described herein.

### Use in the Manufacture of Medicaments

Also described herein is use of an ALDHI compound, as described herein, in the manufacture of a medicament, for example, for use in a method of treatment, for example, for use in a method of treatment of a disorder (e.g., a disease) as described herein.

In one embodiment, the medicament comprises the ALDHI compound.

### Methods of Treatment

Also described herein is a method of treatment, for example, a method of treatment of a disorder (e.g., a disease) as described herein, comprising administering to a subject in need of treatment a therapeutically-effective amount of an ALDHI compound, as described herein, preferably in the form of a pharmaceutical composition.

### Disorders Treated—Disorders Ameliorated by the Inhibition of ALDH1A3 Enzyme

In one embodiment (e.g., of compounds for use in methods of therapy, of use in methods of therapy, of use in the manufacture of medicaments, of methods of treatment), the treatment is treatment of a disorder (e.g., a disease) that is ameliorated by the inhibition of ALDH1A3 enzyme (e.g., by the inhibition or reduction or blockage of the activity or function of ALDH1A3 enzyme).

### Disorders Treated

In one embodiment (e.g., of compounds for use in methods of therapy, of use in methods of therapy, of use in the manufacture of medicaments, of methods of treatment), the treatment is treatment of a disorder (e.g., a disease), for example, a proliferative disorder, cancer, diabetes, a cardiovascular disorder, etc., as described herein.

### Proliferative Disorders

In one embodiment, the disorder is: a proliferative disorder.

The term “proliferative disorder,” as used herein, pertains to an unwanted or uncontrolled cellular proliferation of excessive or abnormal cells which is undesired, such as neoplastic or hyperplastic growth.

In one embodiment, the proliferative disorder is characterised by benign, pre-malignant, malignant, pre-metastatic, metastatic, or non-metastatic cellular proliferation, including for example: neoplasms, hyperplasias, tumours (e.g., histocytoma, glioma, astrocyoma, osteoma), cancers, psoriasis, bone diseases, fibroproliferative disorders (e.g., of connective tissues), pulmonary fibrosis, atherosclerosis, and smooth muscle cell proliferation in the blood vessels, such as stenosis or restenosis following angioplasty.

### Cancer

In one embodiment, the disorder is: cancer.

In one embodiment, the cancer is:
a bone or muscle sarcoma, for example: bone cancer; bone sarcoma; chondrosarcoma; Ewing's sarcoma; heart cancer; leiomyosarcoma; malignant fibrous histiocytoma of bone; osteosarcoma; or rhabdomyosarcoma;
a brain and nervous system cancer, for example: astrocytoma; brain cancer; brainstem glioma; cerebellar astrocytoma; cerebral astrocytoma; ependymoma; glioblastoma; glioma; medulloblastoma; neuroblastoma: oligodendroglioma; pilocytic astrocytoma; pineal astrocytoma; pituitary adenoma; primitive neuroectodermal tumor; schwannoma; or visual pathway and hypothalamic glioma;
a breast cancer, for example: breast cancer; invasive cribriform carcinoma; inflammatory breast cancer; invasive lobular carcinoma; medullary carcinoma; male breast cancer; phyllodes tumor; or tubular carcinoma;
an endocrine system cancer, for example: adrenal gland cancer; adrenocortical carcinoma; papillary thyroid cancer; follicular thyroid cancer; islet cell carcinoma; multiple endocrine neoplasia syndrome; parathyroid cancer; pheochromocytoma; thyroid cancer; or thyroid gland cancer;
an eye cancer, for example: retinoblastoma; or uveal melanoma;
a gastrointestinal cancer, for example:
   anal cancer; appendix cancer; biliary tract cancer; bowel cancer; cholangiocarcinoma; colon adenocarcinoma; colon adenoma; colon cancer; exocrine pancreatic carcinoma; extrahepatic bile duct cancer; gallbladder cancer; gastric (stomach) cancer; gastrointestinal cancer; gastrointestinal carcinoid tumor; gastrointestinal carcinoid tumor; gastrointestinal stromal tumor (GIST); hepatocellular cancer; hepatoblastoma; kidney cancer; large bowel cancer; liver cancer; ocolorectal cancer; pancreatic cancer; rectal cancer; or small bowel cancer;
a genitourinary or gynecologic cancer, for example: bladder cancer; cervical cancer; endometrial cancer; extragonadal germ cell tumor; genito-urinary cancer; gestational trophoblastic tumor; gynaecological cancer; ovarian cancer; ovarian epithelial cancer; ovarian germ cell tumor; penile cancer; prostate cancer; renal cell carcinoma; renal pelvis and ureter, transitional cell cancer; seminoma; teratocarcinoma; testicular cancer; transitional cell cancer of the ureter and renal pelvis; urethral cancer; uterine sarcoma; vaginal cancer; vulvar cancer; or Wilms tumor;
a cancer of the head or neck, for example: esophageal cancer; head and neck cancer; head and neck squamous cell carcinoma; hypopharyngeal cancer; nasopharyngeal cancer; nasopharyngeal carcinoma; oral cancer; oropharyngeal cancer; paranasal sinus and nasal cavity cancer; pharyngeal cancer; or salivary gland cancer;
a hematopoietic cancer, for example:
a plasma cell neoplasm, for example, plasmacytoma or multiple myeloma;
a leukemia, for example: acute biphenotypic leukemia; acute eosinophilic leukemia; acute lymphoblastic leukemia; acute myeloid dendritic cell leukemia; acute myeloid leukemia; acute promyelocytic leukemia; B-cell prolymphocytic leukemia; chronic lymphocytic leukemia; chronic myelogenous leukemia; hairy cell leukemia; large granular lymphocytic leukemia; mast cell leukemia; precursor B lymphoblastic leukemia; T-cell prolymphocytic leukemia;
a lymphoma, for example: AIDS-related lymphoma; anaplastic large cell lymphoma; angioimmunoblastic T-cell lymphoma; Burkitt's lymphoma; cutaneous T-cell lymphoma; diffuse large B-cell lymphoma; follicular lymphoma; hepatosplenic T-cell lymphoma; Hodgkin's lymphoma; intravascular large B-cell lymphoma; lymphomatoid granulomatosis; lymphoplasmacytic lymphoma; mantle cell lymphoma; marginal zone B-cell lymphoma; mediastinal large B cell lymphoma; mucosa-associated lymphoid tissue lymphoma; mycosis fungoides; nodal marginal zone B cell lymphoma; non-Hodgkin lymphoma; plasmablastic lymphoma; primary central nervous system lymphoma; primary cutaneous follicular lymphoma; primary cutaneous immunocytoma; primary effusion lymphoma; Sézary syndrome; or splenic marginal zone lymphoma; or
a myelodysplastic syndrome;
a skin cancer, for example: basal cell carcinoma; dermatofibrosarcoma protuberans; fibrosarcoma; keratoacanthoma; malignant melanoma; melanoma; Merkel cell carcinoma; sebaceous carcinoma; or squamous cell carcinoma;
a thoracic and respiratory cancer, for example: adenocarcinoma; bronchial adenoma; bronchial carcinoid; laryngeal cancer; lung cancer; mediastinum cancer; mesothelioma; non-small cell lung cancer; peritoneal cancer; pleuropulmonary blastoma; small cell lung cancer; thymic carcinoma; or thymoma carcinoma;
an HIV/AIDS related cancer, for example, Kaposi sarcoma;
or other cancer, for example, epithelioid hemangioendothelioma; desmoplastic small round cell tumor; or liposarcoma.

In one embodiment, the cancer is:
melanoma;
fibrosarcoma;
breast cancer, e.g., triple-negative subtype breast cancer;
glioma;
glioblastoma;
lung cancer, e.g., non-small cell lung cancer;
mesothelioma;
thyroid cancer;
renal cell carcinoma;
pancreatic cancer;
gastric cancer;
colorectal cancer;
gallbladder cancer;
cholangiocarcinoma;
neuroblastoma;
testicular germ cell cancer;
ovarian cancer; or
prostate cancer.

In one embodiment, the cancer (e.g., as above) is characterised by aberrant expression of ALDH1A3.

In one embodiment, the cancer (e.g., as above) is characterised by overexpression of ALDH1A3.

In one embodiment, the cancer (e.g., as above) is characterised, or further characterised, as chemotherapy-resistant cancer and/or radiotherapy-resistant cancer.

In one embodiment, the cancer (e.g., as above) is characterised, or further characterised, as immunotherapy-resistant cancer.

In one embodiment, the cancer (e.g., as above) is characterised, or further characterised, as immunotherapy-resistant cancer characterised by the presence or elevated presence of T-regulatory cells.

In one embodiment, the cancer (e.g., as above) is characterised, or further characterised, as metastatic cancer.

### Obesity

In one embodiment, the disorder is: obesity or a complication of obesity.

In one embodiment, the disorder is: obesity.

In one embodiment, the disorder is: a complication of obesity, including type II diabetes.

### Diabetes

In one embodiment, the disorder is: diabetes.

In one embodiment, the disorder is: type II diabetes.

### Cardiovascular Disorder

In one embodiment, the disorder is: a cardiovascular disorder.

In one embodiment, the disorder is: restenosis.

In one embodiment, the disorder is: intimal hyperplasia.

In one embodiment, the disorder is: intimal hyperplasia following vascular reconstruction.

In one embodiment, the disorder is: intimal hyperplasia following coronary artery angioplasty/stenting, bypass vein grafting, arteriovenous fistula (e.g., for dialysis access), or allograft transplantation.

In one embodiment, the disorder is: pulmonary arterial hypertension (PAH).

### Treatment

The term “treatment,” as used herein in the context of treating a disorder (e.g., disease), pertains generally to treatment of a human or an animal (e.g., in veterinary applications), in which some desired therapeutic effect is achieved, for example, the inhibition of the progress of the disorder (including, e.g., a reduction in the rate of progress, a halt in the rate of progress), alleviation of symptoms of the disorder, amelioration of the disorder, and cure of the disorder. Treatment as a prophylactic measure (i.e., prophylaxis) is also included. For example, use with subjects (e.g., patients) who have not yet developed the disorder, but who are at risk of developing the disorder, is encompassed by the term “treatment.”

For example, treatment of cancer includes reducing the progress of cancer, alleviating the symptoms of cancer, reducing the incidence of cancer, prophylaxis of cancer, etc.

The term “therapeutically-effective amount,” as used herein, pertains to that amount of a compound, or a material, composition, or dosage form comprising a compound, which is effective for producing some desired therapeutic effect, commensurate with a reasonable benefit/risk ratio, when administered in accordance with a desired treatment regimen.

### Combination Therapies

The term “treatment” as used herein includes combination treatments and therapies, in which two or more treatments or therapies are combined, for example, sequentially or simultaneously. For example, the ALDHI compounds described herein may also be used in combination therapies, e.g., in conjunction with other agents.

Accordingly, also described herein is an ALDHI compound, as described herein, in combination with one or more (e.g., 1, 2, 3, 4, etc.) additional therapeutic agents.

The particular combination would be at the discretion of the physician who would select dosages using their common general knowledge and dosing regimens known to a skilled practitioner.

The agents (e.g., the ALDHI compound as described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (e.g., the ALDHI compound described here, plus one or more other agents) may be formulated together in a single dosage form, or alternatively, the individual agents may be formulated separately, and optionally may be presented together in the form of a kit, optionally with instructions for their use.

In one embodiment, the other agent (e.g., the additional therapeutic agent) is an immunotherapeutic agent, for example, an immune checkpoint inhibitor.

### Other Uses

The ALDHI compounds described herein may also be used as cell culture additives to inhibit ALDH1A3 enzyme (e.g., to inhibit or reduce or block the activity or function of ALDH1A3 enzyme).

The ALDHI compounds described herein may also be used as part of an in vitro assay, for example, in order to determine whether a candidate host is likely to benefit from treatment with the compound in question.

The ALDHI compounds described herein may also be used as a standard, for example, in an assay, in order to identify other active compounds, other ALDH1A3 enzyme inhibitors, etc.

### Kits

Also describes herein is a kit comprising (a) an ALDHI compound, as described herein, preferably provided as a composition (e.g., a pharmaceutical composition) and in a suitable container and/or with suitable packaging; and (b) instructions for use, for example, in a method of treatment of a disorder (e.g., a disease) as described herein, for example, written instructions on how to administer the compound.

The written instructions may also include a list of indications for which the ALDHI compound is a suitable treatment.

### Routes of Administration

The ALDHI compound or pharmaceutical composition comprising the ALDHI compound may be administered to a subject by any convenient route of administration, whether systemically/peripherally or topically (i.e., at the site of desired action).

Routes of administration include, for example: oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject

The subject (e.g., patient) may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

Furthermore, the subject (e.g., patient) may be any of its forms of development, for example, a foetus.

In one preferred embodiment, the subject (e.g., patient) is a human.

### Formulations

While it is possible for an ALDHI compound to be administered alone, it is preferable to present it as a pharmaceutical formulation (e.g., composition, preparation, medicament) comprising at least one ALDHI compound, as described herein, together with one or more other pharmaceutically acceptable ingredients well known to those skilled in the art, including, for example, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, also described herein are pharmaceutical compositions, as defined above, and methods of making a pharmaceutical composition comprising mixing at least one ALDHI compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington: The Science and Practice of Pharmacy, 21st edition, Lippinott Williams and Wilkins, 2005; Remington: The Science and Practice of Pharmacy, 22nd edition, Pharmaceutical Press, 2012; and Handbook of Pharmaceutical Excipients, 7th edition, Pharmaceutical Press, 2012.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or mixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavoured basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichorotetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/mL to about 10 μg/mL, for example from about 10 ng/mL to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the ALDHI compounds, and compositions comprising the ALDHI compounds, can vary from subject to subject (e.g., from patient to patient). Determining the optimal dosage will generally involve balancing the level of therapeutic benefit against any risk or deleterious side effects. The selected dosage level will depend on a variety of factors including, for example: the activity of the particular ALDHI compound; the route of administration; the time of administration; the rate of excretion of the ALDHI compound; the duration of the treatment; other drugs, compounds, and/or materials used in combination; the severity of the disorder; and the species, sex, age, weight, condition, general health, and prior medical history of the subject (e.g., patient). The amount of ALDHI compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the ALDHI compound is in the range of about 0.01 mg to about 5000 mg (more typically about 0.1 mg to about 1000 mg, e.g., about 0.1 mg to about 300 mg) per day.

Where the compound is a salt, a solvate, an ester, an amide, a prodrug, or the like, the amount administered is calculated on the basis of the parent compound and so the actual weight to be used is increased proportionately.

### EXAMPLES

### Chemical Synthesis

### Abbreviations

{1H}: Proton decoupling.
Aq.: Aqueous.
BAST: [bis(2-methoxyethyl)amino]sulphur trifluoride (Deoxo-Fluor®).
t-Boc: tert-butyloxycarbonyl.
DBU: 1,8-diazabicyclo[5.4.0]undec-7-ene.
DCM: Dichloromethane (methylene chloride).
DDQ: 2,3-dichloro-5,6-dicyano-1,4-benzoquinone.
DIAD: diisopropyl azodicarboxylate.
DIPEA: N,N-diisopropylethylamine.
DME: 1,2-dimethoxyethane.
DMP: Dess-Martin periodinane.
DMAP: 4-(dimethylamino) pyridine.
DMSO: Dimethylsulfoxide.
ES: Electrospray ionisation.
Et₂O: Ether (diethyl ether).
EtOAc: Ethyl acetate.
EtOH: Ethanol (ethyl alcohol).
HATU: 2-(7-aza-1H-benzotriazole-1-yl)-1,1,3,3-tetramethyluronium hexafluorophosphate.
MeCN: Acetonitrile.
MeOH: Methanol (methyl alcohol).
NaBH₄: Sodium borohydride.
NaOtBu: Sodium tert-butoxide.
NBS: N-bromosuccinimide.
NaHCO₃: Sodium bicarbonate.
NH₄Cl: Ammonium chloride.
Pd/C: Palladium on carbon.
pTSA: p-toluenesulfonic acid monohydrate.
RT: Room temperature.
Na₂S₂O₃: Sodium thiosulfate.
Sat. aq.: Saturated aqueous.
tBuBrettPhos Pd G3: [(2-di-tert-butylphosphino-3,6-dimethoxy-2′,4′,6′-triisopropyl-1,1′-biphenyl)-2-(2′-amino-1,1′-biphenyl)]palladium(II) methanesulfonate.
TBAF: tetrabutylammonium fluoride.
TBDMS: tert-butyldimethylsilyl.
THF: Tetrahydrofuran.
UPLC: Ultra Performance Liquid Chromatography.

### General Experimental

Flash chromatography was performed using pre-packed silica gel cartridges (RediSep Rf, Isco). Thin layer chromatography was conducted with 5×10 cm plates coated with Merck Type 60 F254 silica gel to a thickness of 0.25 mm. All reagents obtained from commercial sources were used without further purification. Anhydrous solvents were obtained from the Sigma-Aldrich Chemical Company Ltd. or Fisher Chemicals Ltd., and used without further drying. HPLC grade solvents were obtained from Fisher Chemicals Ltd.

All compounds were >90% purity as determined by examination of both the LCMS and ¹H NMR spectra unless otherwise indicated. Where Cl or Br were present, expected isotopic distribution patterns were observed.

### NMR

Proton (¹H) and carbon (¹³C) NMR spectra were recorded on a 300 MHz Bruker spectrometer. Solutions were typically prepared in either deuterated chloroform (CDCl₃), deuterated methanol (Methanol-d4) or deuterated dimethylsulfoxide (DMSO-d6) with chemical shifts referenced to tetramethylsilane (TMS) or deuterated solvent as an internal standard. ¹H NMR data are reported indicating the chemical shift (b), the integration (e.g., ¹H), the multiplicity (s, singlet; d, doublet; t, triplet; q, quartet; m, multiplet; br, broad; dd, doublet of doublets) and the coupling constant (J) in Hz. Deuterated solvents were purchased from the Sigma-Aldrich Chemical Company, Goss or Fluorochem.

### Analytical LC-MS

LCMS analyses were performed on a Waters Acquity UPLC using BEH C₁₈ 1.7 μM columns (2.1×50 mm) with a diode array detector coupled to a SQD mass spectrometer or, a Waters Acquity I-Class UPLC using BEH C₁₈ 1.7 μM columns (2.1×50 mm) with a diode array detector coupled to a QDa mass spectrometer. Analyses were performed with either buffered acidic or basic solvents using gradients as detailed below:

### Low pH:

Solvent A—Water+10 mM ammonium hydrogen carbonate+0.1% formic acid.
Solvent B—MeCN+5% water+0.1% formic acid.

### High pH:

Solvent A—Water+10 mM ammonium hydrogen carbonate+0.1% ammonia solution.
Solvent B—MeCN+5% water+0.1% ammonia solution.

Gradient

| | Flow rate | | |
|---|---|---|---|
| Time | (mL/min) | % Solvent A | % Solvent B |
| 0 | 0.6 | 95 | 5 |
| 1.2 | 0.6 | 5 | 95 |
| 1.7 | 0.6 | 5 | 95 |
| 1.8 | 0.6 | 95 | 5 |

### Preparative HPLC-MS

Some compounds were purified by preparative HPLC on a Waters FractionLynx MS auto-purification system, with a Phenomonex Gemini NX 5 μm C₁₈, 100 mm×21.2 mm i.d. column (for low pH runs) or a Waters XBridge 5 μm C₁₈, 100 mm×19 mm i.d. column (for high pH runs), running at a flow rate of 20 mL/min with UV diode array detection (210-400 nm) and mass-directed collection using both positive and negative mass ion detection.

Purifications were performed using buffered acidic or basic solvent systems as appropriate. Compound retention times on the system were routinely assessed using a 30-50 μL test injection and a standard gradient, then purified using an appropriately chosen focused gradient as detailed below, based upon observed retention time.

### Low pH:

Solvent A—Water+10 mM ammonium formate+0.1% formic acid.
Solvent B—MeCN+5% water+0.1% formic acid.

### High pH:

Solvent A—Water+10 mM ammonium formate+0.1% ammonia solution.
Solvent B—MeCN+5% water+0.1% ammonia solution.

Standard Gradient

| | Flow rate | | |
|---|---|---|---|
| Time | (mL/min) | % Solvent A | % Solvent B |
| 0 | 20 | 90 | 10 |
| 0.3 | 20 | 90 | 10 |
| 8.5 | 20 | 2 | 98 |
| 12 | 20 | 2 | 98 |
| 12.5 | 0 | 2 | 98 |

Focused Gradients

| | | % Solvent B | | | | |
|---|---|---|---|---|---|---|
| | Flow rate | Retention time on standard gradient (min.) | | | | |
| Time | (mL/min) | 0-5.2 | 4.9-6.6 | 6.3-7.5 | 7.3-9.5 | 9.3-12 |
| 0 | 20 | 10 | 10 | 10 | 10 | 10 |
| 0.25 | 20 | 10 | 10 | 10 | 10 | 10 |
| 0.35 | 20 | 10 | 20 | 35 | 45 | 60 |
| 10 | 20 | 45 | 55 | 65 | 75 | 98 |
| 12 | 20 | 98 | 98 | 98 | 98 | 98 |
| 12.5 | 0 | 98 | 98 | 98 | 98 | 98 |

### Synthetic Methods

Several methods for the chemical synthesis of the compounds of the present invention are described herein. These and/or other well-known methods may be modified and/or adapted in known ways in order to facilitate the synthesis of additional compounds within the scope of the present invention.

### Preparation of alkyl-aryl ethers by alkylation

### Scheme 1

### Alkylation of 2-Substituted Cyclic Amines with 6-(2-chloroethoxy)-3,4-dihydro-1H-quinolin-2-one

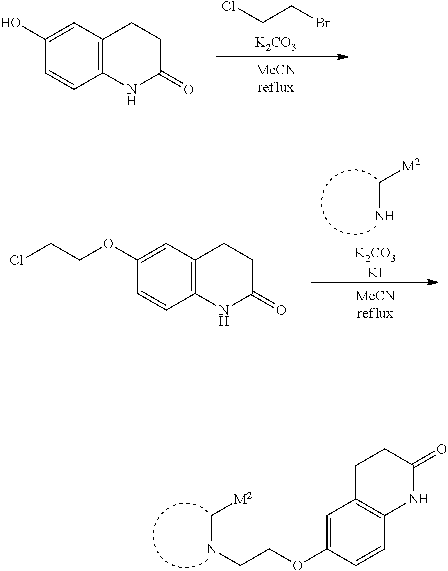

### Synthesis 1

### 6-(2-chloroethoxy)-3,4-dihydro-1H-quinolin-2-one

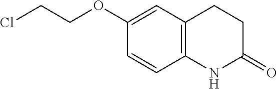 [SMILES: O=C1CCc2cc(OCCCl)ccc2N1]

To a mixture of 6-hydroxy-3,4-dihydro-2(1H)-quinolinone (1.0 g, 6.13 mmol, 1 eq) and K₂CO₃ (1.69 g, 12.3 mmol, 2 eq) in MeCN (30 mL) under nitrogen was added 1-bromo-2-chloroethane (2.6 mL, 30.6 mmol, 5 eq). The mixture was refluxed for 3 days. The mixture was cooled, filtered, washed with MeCN (3×10 mL) and concentrated in vacuo. The resulting solid was stirred in DCM (25 mL) for 0.5 h, filtered, washed with DCM (3×25 mL) and concentrated in vacuo. The solid was chromatographed (C₁₈) using 5-95% MeCN:H₂O as eluent to afford the title compound (766 mg, 3.39 mmol, 55%) as a white powder.

MS (ES+) m/z 226.2/228.2 (M+H), Cl isotope pattern.

¹H NMR (300 MHz, CDCl₃) δ 7.80 (s, 1H), 6.84-6.62 (m, 3H), 4.22 (t, J=5.9 Hz, 2H), 3.82 (t, J=5.9 Hz, 2H), 3.02-2.90 (m, 2H), 2.70-2.58 (m, 2H).

### General Method A

### Alkylation of 2-aryl cyclic amines with 6-(2-chloroethoxy)-3,4-dihydro-1H-quinolin-2-one

A mixture of 6-(2-chloroethoxy)-3,4-dihydro-1H-quinolin-2-one (1 eq), 2-substituted cyclic amine (1.2 to 2.4 eq), K₂CO₃ (2 to 3 eq) and KI (0.2 to 1 eq) in MeCN (0.02 M to 0.11 M) was heated at reflux for 3 to 6 days under nitrogen. When required, additional 2-aryl cyclic amine and KI were added to push the reaction to completion. The mixture was cooled, water followed by DCM or EtOAc were added and the phases separated. The aqueous phase was washed with DCM or EtOAc, the organic extracts combined, filtered through a hydrophobic frit and concentrated in vacuo. The crude material was purified by either normal phase chromatography (SiO₂) using a gradient of MeOH:DCM (optionally containing 1% aqueous NH₃), reverse phase chromatography (C₁₈) using a gradient of MeCN:H₂O and/or by preparative HPLC-MS using a gradient of high or low pH aq. MeCN. Further purification via trituration with Et₂O or petroleum ether as required.

### Synthesis 2

### 6-[2-[2-(3-methoxyphenyl)pyrrolidin-1-yl]ethoxy]-3,4-dihydro-1H-quinolin-2-one (ALDHI-2034)

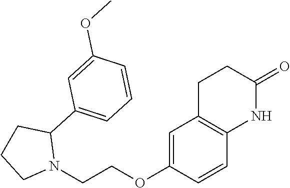 [SMILES: COc1cccc(C2CCCN2CCOc2ccc3c(c2)CCC(=O)N3)c1]

Prepared as described in method A from 6-(2-chloroethoxy)-3,4-dihydro-1H-quinolin-2-one (30 mg, 0.133 mmol, 1 eq), 2-(3-methoxy-phenyl)-pyrrolidine hydrochloride (43 mg, 0.199 mmol, 1.5 eq), K₂CO₃ (55 mg, 0.399 mmol, 3 eq), KI (22 mg, 0.133 mmol, 1 eq) in MeCN (2.5 mL) to return the title compound (27 mg, 0.0723 mmol, 54%) as a white powder after preparative HPLC-MS (high pH).

MS (ES+) m/z 367.3 (M+H).

¹H NMR (300 MHz, Methanol-d₄) δ 7.22 (t, J=7.9 Hz, 1H), 7.04-6.98 (m, 1H), 6.98-6.90 (m, 1H), 6.83-6.64 (m, 4H), 3.97 (t, J=5.8 Hz, 2H), 3.78 (s, 3H), 3.50-3.41 (m, 1H), 3.38-3.29 (m, 1H), 3.01-2.84 (m, 3H), 2.59-2.48 (m, 3H), 2.43 (q, J=9.0 Hz, 1H), 2.28-2.10 (m, 1H), 2.06-1.64 (m, 3H).

The following example compounds were prepared similarly using method A with the appropriate 2-substituted cyclic amine.

### Synthesis 3

### 6-[2-(2-phenylpyrrolidin-1-yl)ethoxy]-3,4-dihydro-1H-quinolin-2-one (ALDHI-2019)

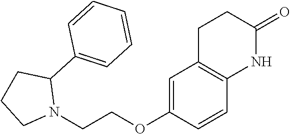 [SMILES: O=C1CCc2cc(OCCN3CCCC3c3ccccc3)ccc2N1]

MS (ES+) m/z 337.3 (M+H).

¹H NMR (300 MHz, DMSO-d₆) δ 9.88 (s, 1H), 7.39-7.19 (m, 5H), 6.75-6.61 (m, 3H), 3.92 (td, J=6.1, 2.3 Hz, 2H), 3.40-3.28 (m, 2H), 2.85-2.71 (m, 3H), 2.45-2.28 (m, 4H), 2.19-2.04 (m, 1H), 1.89-1.70 (m, 2H), 1.62-1.41 (m, 1H).

### Synthesis 4

### 6-[2-[2-(p-tolyl)pyrrolidin-1-yl]ethoxy]-3,4-dihydro-1H-quinolin-2-one (ALDHI-2010)

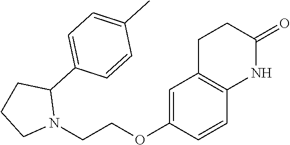 [SMILES: Cc1ccc(C2CCCN2CCOc2ccc3c(c2)CCC(=O)N3)cc1]

MS (ES+) m/z 351.3 (M+H).

¹H NMR (300 MHz, CDCl₃) δ 7.46 (s, 1H), 7.26 (d, J=8.0 Hz, 2H), 7.12 (d, J=7.9 Hz, 2H), 6.66-6.56 (m, 3H), 3.93 (td, J=6.2, 1.5 Hz, 2H), 3.43 (td, J=8.7, 2.6 Hz, 1H), 3.36-3.24 (m, 1H), 2.99-2.84 (m, 3H), 2.63-2.55 (m, 2H), 2.55-2.35 (m, 2H), 2.33 (s, 3H), 2.20-2.07 (m, 1H), 2.02-1.78 (m, 2H), 1.78-1.62 (m, 1H).

### Synthesis 5

### 6-[2-[2-(m-tolyl)pyrrolidin-1-yl]ethoxy]-3,4-dihydro-1H-quinolin-2-one (ALDHI-2011)

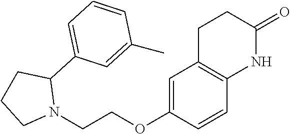 [SMILES: Cc1cccc(C2CCCN2CCOc2ccc3c(c2)CCC(=O)N3)c1]

MS (ES+) m/z 351.5 (M+H).

¹H NMR (300 MHz, CDCl₃) δ 7.38 (s, 1H), 7.24-7.11 (m, 3H), 7.07-7.01 (m, 1H), 6.67-6.55 (m, 3H), 3.94 (t, J=6.2 Hz, 2H), 3.48-3.38 (m, 1H), 3.34-3.25 (m, 1H), 3.00-2.85 (m, 3H), 2.63-2.34 (m, 4H), 2.33 (s, 3H), 2.21-2.07 (m, 1H), 2.05-1.79 (m, 2H), 1.79-1.63 (m, 1H).

### Synthesis 6

### 6-[2-[2-(o-tolyl)pyrrolidin-1-yl]ethoxy]-3,4-dihydro-1H-quinolin-2-one (ALDHI-2012)

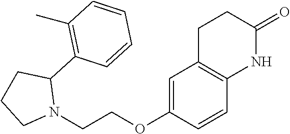 [SMILES: Cc1ccccc1C1CCCN1CCOc1ccc2c(c1)CCC(=O)N2]

MS (ES+) m/z 351.3 (M+H).

¹H NMR (300 MHz, CDCl₃) δ 7.62-7.55 (m, 1H), 7.36 (s, 1H), 7.22-7.14 (m, 1H), 7.13-7.08 (m, 2H), 6.69-6.56 (m, 3H), 3.98 (t, J=6.2 Hz, 2H), 3.60 (t, J=8.2 Hz, 1H), 3.50-3.41 (m, 1H), 3.01-2.86 (m, 3H), 2.63-2.55 (m, 2H), 2.55-2.44 (m, 1H), 2.44-2.34 (m, 1H), 2.33 (s, 3H), 2.29-2.15 (m, 1H), 2.02-1.75 (m, 2H), 1.66-1.45 (m, 1H).

### Synthesis 7

### 6-[2-[2-(4-pyridyl)pyrrolidin-1-yl]ethoxy]-3,4-dihydro-1H-quinolin-2-one (ALDHI-2004)

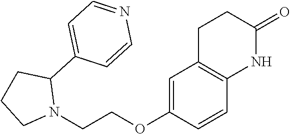 [SMILES: O=C1CCc2cc(OCCN3CCCC3c3ccncc3)ccc2N1]

MS (ES+) m/z 338.3 (M+H).

¹H NMR (300 MHz, CDCl₃) δ 8.56-8.47 (m, 2H), 7.44 (s, 1H), 7.36-7.27 (m, 2H), 6.69-6.55 (m, 3H), 3.95 (t, J=5.9 Hz, 2H), 3.50-3.37 (m, 2H), 2.96-2.84 (m, 3H), 2.66-2.55 (m, 3H), 2.46 (q, J=8.8 Hz, 1H), 2.28-2.13 (m, 1H), 2.04-1.79 (m, 3H), 1.74-1.53 (m, 1H).

### Synthesis 8

### 6-[2-[2-(3-pyridyl)pyrrolidin-1-yl]ethoxy]-3,4-dihydro-1H-quinolin-2-one (ALDHI-2005)

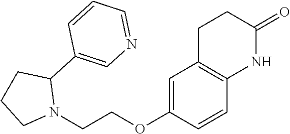 [SMILES: O=C1CCc2cc(OCCN3CCCC3c3cccnc3)ccc2N1]

MS (ES+) m/z 338.3 (M+H).

¹H NMR (300 MHz, CDCl₃) δ 8.61 (d, J=2.2 Hz, 1H), 8.51 (dd, J=4.8, 1.7 Hz, 1H), 7.82-7.72 (m, 1H), 7.47 (s, 1H), 7.33-7.22 (m, 1H), 6.70-6.60 (m, 3H), 3.96 (t, J=5.9 Hz, 2H), 3.54-3.41 (m, 2H), 2.98-2.87 (m, 3H), 2.66-2.55 (m, 3H), 2.48 (q, J=8.8 Hz, 1H), 2.30-2.16 (m, 1H), 2.10-1.83 (m, 3H).

### Synthesis 9

### 6-[2-[2-(2-pyridyl)pyrrolidin-1-yl]ethoxy]-3,4-dihydro-1H-quinolin-2-one (ALDHI-2006)

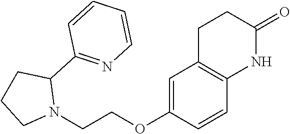 [SMILES: O=C1CCc2cc(OCCN3CCCC3c3ccccn3)ccc2N1]

MS (ES+) m/z 338.3 (M+H).

¹H NMR (300 MHz, CDCl₃) δ 8.53 (ddd, J=4.9, 1.8, 0.9 Hz, 1H), 8.00 (s, 1H), 7.63 (td, J=7.6, 1.8 Hz, 1H), 7.50 (dt, J=7.9, 1.1 Hz, 1H), 7.13 (ddd, J=7.4, 4.9, 1.3 Hz, 1H), 6.80-6.56 (m, 3H), 3.95 (t, J=6.1 Hz, 2H), 3.63 (t, J=8.0 Hz, 1H), 3.51-3.38 (m, 1H), 3.03-2.84 (m, 3H), 2.75-2.64 (m, 1H), 2.62-2.55 (m, 2H), 2.55-2.43 (m, 1H), 2.37-2.19 (m, 1H), 2.08-1.70 (m, 4H).

### Synthesis 10

### 6-[2-[2-(6-methyl-3-pyridyl)pyrrolidin-1-yl]ethoxy]-3,4-dihydro-1H-quinolin-2-one (ALDHI-2030)

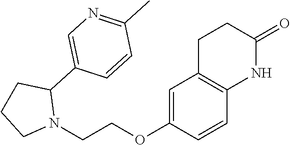 [SMILES: Cc1ccc(C2CCCN2CCOc2ccc3c(c2)CCC(=O)N3)cn1]

MS (ES+) m/z 352.3 (M+H).

¹H NMR (300 MHz, Methanol-d₄) δ 8.42 (d, J=2.2 Hz, 1H), 7.79 (dd, J=8.0, 2.3 Hz, 1H), 7.27 (d, J=8.0 Hz, 1H), 6.79-6.63 (m, 3H), 4.07-3.89 (m, 2H), 3.61-3.43 (m, 2H), 3.00-2.85 (m, 3H), 2.73-2.61 (m, 1H), 2.60-2.48 (m, 6H), 2.36-2.18 (m, 1H), 2.09-1.84 (m, 2H), 1.83-1.64 (m, 1H).

### Synthesis 11

### 6-[2-[(2R)-2-(3-fluorophenyl)pyrrolidin-1-yl]ethoxy]-3,4-dihydro-1H-quinolin-2-one (ALDHI-2007)

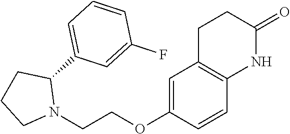 [SMILES: O=C1CCc2cc(OCCN3CCC[C@@H]3c3cccc(F)c3)ccc2N1]

MS (ES+) m/z 335.3 (M+H).

¹H NMR (300 MHz, CDCl₃) δ 7.97 (s, 1H), 7.32-7.18 (m, 2H), 7.20 (s, 2H), 7.18-7.07 (m, 2H), 6.97-6.84 (m, 1H), 6.70-6.61 (m, 3H), 3.95 (t, J=6.1 Hz, 2H), 3.49-3.32 (m, 2H), 3.01-2.85 (m, 3H), 2.65-2.49 (m, 3H), 2.41 (q, J=8.8 Hz, 1H), 2.26-2.08 (m, 1H), 2.06-1.74 (m, 2H), 1.75-1.56 (m, 1H).

¹⁹F {¹H} NMR (282 MHz, CDCl₃) δ −113.48.

### Synthesis 12

### 6-[2-[(2S)-2-(3-fluorophenyl)pyrrolidin-1-yl]ethoxy]-3,4-dihydro-1H-quinolin-2-one (ALDHI-2018)

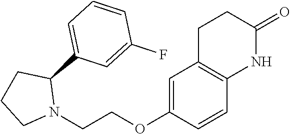 [SMILES: O=C1CCc2cc(OCCN3CCC[C@H]3c3cccc(F)c3)ccc2N1]

MS (ES+) m/z 355.3.

¹H NMR (300 MHz, CDCl₃) δ 7.35 (s, 1H), 7.29-7.19 (m, 1H), 7.18-7.08 (m, 2H), 6.95-6.86 (m, 1H), 6.68-6.56 (m, 3H), 3.95 (t, J=6.1 Hz, 2H), 3.47-3.34 (m, 2H), 2.98-2.85 (m, 3H), 2.64-2.48 (m, 3H), 2.41 (q, J=8.8 Hz, 1H), 2.24-2.10 (m, 1H), 2.01-1.60 (m, 3H).

¹⁹F {¹H} NMR (282 MHz, CDCl₃) δ −113.49.

### Synthesis 13

### 6-[2-[2-(2-fluorophenyl)pyrrolidin-1-yl]ethoxy]-3,4-dihydro-1H-quinolin-2-one (ALDHI-2002)

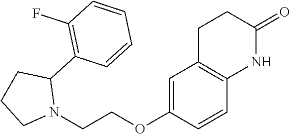 [SMILES: O=C1CCc2cc(OCCN3CCCC3c3ccccc3F)ccc2N1]

MS (ES+) m/z 355.3 (M+H).

¹H NMR (300 MHz, CDCl₃) δ 7.59 (td, J=7.5, 2.0 Hz, 1H), 7.52 (s, 1H), 7.23-7.04 (m, 2H), 6.99 (ddd, J=10.5, 8.0, 1.4 Hz, 1H), 6.71-6.57 (m, 3H), 3.98 (t, J=6.1 Hz, 2H), 3.80 (t, J=8.1 Hz, 1H), 3.50-3.37 (m, 1H), 3.05-2.85 (m, 3H), 2.66-2.51 (m, 3H), 2.43 (q, J=8.8 Hz, 1H), 2.34-2.16 (m, 1H), 1.95-1.80 (m, 2H), 1.73-1.60 (m, 1H).

¹⁹F {¹H} NMR (282 MHz, CDCl₃) δ −120.19.

### Synthesis 14

### 6-[2-[2-(4-fluorophenyl)pyrrolidin-1-yl]ethoxy]-3,4-dihydro-1H-quinolin-2-one (ALDHI-2003)

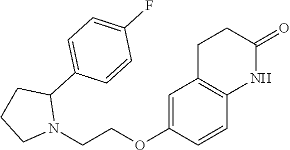 [SMILES: O=C1CCc2cc(OCCN3CCCC3c3ccc(F)cc3)ccc2N1]

MS (ES+) m/z 355.3 (M+H).

¹H NMR (300 MHz, CDCl₃) δ 7.68 (s, 1H), 7.37-7.28 (m, 2H), 7.04-6.93 (m, 2H), 6.67-6.57 (m, 3H), 3.92 (t, J=6.1 Hz, 2H), 3.42 (td, J=8.4, 2.7 Hz, 1H), 3.38-3.27 (m, 1H), 2.97-2.82 (m, 3H), 2.65-2.56 (m, 2H), 2.51 (dt, J=12.4, 6.0 Hz, 1H), 2.40 (q, J=8.8 Hz, 1H), 2.22-2.06 (m, 1H), 2.06-1.75 (m, 2H), 1.73-1.61 (m, 1H).

¹⁹F {¹H} NMR (282 MHz, CDCl₃) δ −116.14.

### Synthesis 15

### 6-[2-[2-(3,4-difluorophenyl)pyrrolidin-1-yl]ethoxy]-3,4-dihydro-1H-quinolin-2-one (ALDHI-2039)

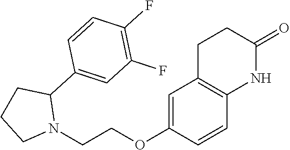 [SMILES: O=C1CCc2cc(OCCN3CCCC3c3ccc(F)c(F)c3)ccc2N1]

MS (ES+) m/z 373.2 (M+H).

¹H NMR (300 MHz, DMSO-d₆) δ 9.88 (s, 1H), 7.46-7.34 (m, 1H), 7.35-7.09 (m, 2H), 6.78-6.61 (m, 3H), 3.96 (t, J=5.8 Hz, 2H), 3.77 (t, J=8.1 Hz, 1H), 3.42-3.32 (m, 1H), 2.90-2.75 (m, 3H), 2.61-2.51 (m, 1H), 2.43-2.33 (m, 3H), 2.29-2.14 (m, 1H), 1.91-1.74 (m, 2H), 1.63-1.44 (m, 1H).

¹⁹F {¹H} NMR (282 MHz, DMSO-d₆) δ −140.05, −145.94.

### Synthesis 16

### 6-[2-[2-(3,5-difluorophenyl)pyrrolidin-1-yl]ethoxy]-3,4-dihydro-1H-quinolin-2-one (ALDHI-2032)

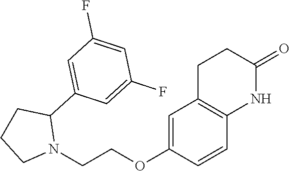 [SMILES: O=C1CCc2cc(OCCN3CCCC3c3cc(F)cc(F)c3)ccc2N1]

MS (ES+) m/z 373.3 (M+H).

¹H NMR (300 MHz, Methanol-d4-d4) δ 7.08-6.98 (m, 2H), 6.81-6.65 (m, 4H), 4.09-3.91 (m, 2H), 3.54-3.39 (m, 2H), 3.00-2.85 (m, 3H), 2.68-2.39 (m, 4H), 2.34-2.15 (m, 1H), 2.03-1.81 (m, 2H), 1.73-1.54 (m, 1H).

¹⁹F {1H} NMR (282 MHz, Methanol-d4) δ −112.19.

### Synthesis 17

### 6-[2-[2-(2-thienyl)pyrrolidin-1-yl]ethoxy]-3,4-dihydro-1H-quinolin-2-one (ALDHI-2013)

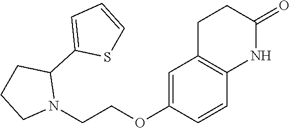 [SMILES: O=C1CCc2cc(OCCN3CCCC3c3cccs3)ccc2N1]

MS (ES+) m/z 343.3 (M+H).

¹H NMR (300 MHz, CDCl₃) δ 7.46 (s, 1H), 7.21 (ddd, J=4.8, 1.5, 0.6 Hz, 1H), 6.96-6.90 (m, 2H), 6.70-6.57 (m, 3H), 3.99 (td, J=6.3, 1.9 Hz, 2H), 3.75 (t, J=7.7 Hz, 1H), 3.41 (td, J=8.6, 2.6 Hz, 1H), 3.12-3.00 (m, 1H), 2.95-2.85 (m, 2H), 2.65-2.53 (m, 3H), 2.44 (q, J=8.6 Hz, 1H), 2.29-2.15 (m, 1H), 2.08-1.74 (m, 3H).

### Synthesis 18

### 6-[2-[2-(3-thienyl)pyrrolidin-1-yl]ethoxy]-3,4-dihydro-1H-quinolin-2-one (ALDHI-2014)

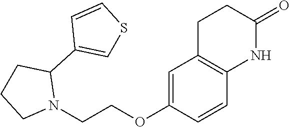 [SMILES: O=C1CCc2cc(OCCN3CCCC3c3ccsc3)ccc2N1]

MS (ES+) m/z 343.3 (M+H).

¹H NMR (300 MHz, CDCl₃) δ 7.67 (s, 1H), 7.29-7.26 (m, 1H), 7.16 (dd, J=2.9, 1.2 Hz, 1H), 7.09 (dd, J=5.0, 1.3 Hz, 1H), 6.67-6.58 (m, 3H), 3.94 (t, J=6.2 Hz, 2H), 3.49 (t, J=8.0 Hz, 1H), 3.39 (td, J=9.0, 2.7 Hz, 1H), 3.04-2.94 (m, 1H), 2.94-2.85 (m, 2H), 2.63-2.47 (m, 3H), 2.38 (q, J=8.7 Hz, 1H), 2.20-2.07 (m, 1H), 2.04-1.69 (m, 3H).

### Synthesis 19

### 6-[2-[2-(2-methylthiazol-4-yl)pyrrolidin-1-yl]ethoxy]-3,4-dihydro-1H-quinolin-2-one (ALDHI-2033)

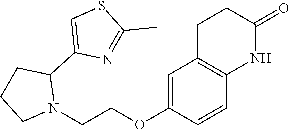 [SMILES: Cc1nc(C2CCCN2CCOc2ccc3c(c2)CCC(=O)N3)cs1]

MS (ES+) m/z 358.3 (M+H).

¹H NMR (300 MHz, Methanol-d₄) δ 7.24 (d, J=0.6 Hz, 1H), 6.83-6.64 (m, 3H), 4.01 (t, J=5.7 Hz, 2H), 3.70 (t, J=7.7 Hz, 1H), 3.46-3.36 (m, 1H), 3.04 (dt, J=13.1, 5.8 Hz, 1H), 2.97-2.86 (m, 2H), 2.77-2.62 (m, 4H), 2.60-2.43 (m, 3H), 2.32-2.15 (m, 1H), 2.06-1.79 (m, 3H).

### Synthesis 20

### 6-[2-[2-(1-ethylpyrazol-4-yl)pyrrolidin-1-yl]ethoxy]-3,4-dihydro-1H-quinolin-2-one (ALDHI-2016)

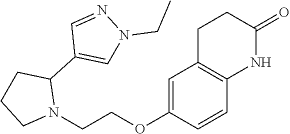 [SMILES: CCn1cc(C2CCCN2CCOc2ccc3c(c2)CCC(=O)N3)cn1]

MS (ES+) m/z 355.4 (M+H).

¹H NMR (300 MHz, CDCl₃) δ 7.48 (s, 1H), 7.46 (d, J=0.8 Hz, 1H), 7.34 (s, 1H), 6.70-6.58 (m, 3H), 4.12 (q, J=7.3 Hz, 2H), 3.96 (t, J=6.1 Hz, 2H), 3.40-3.29 (m, 2H), 3.06 (dt, J=12.5, 6.2 Hz, 1H), 2.95-2.85 (m, 2H), 2.64-2.55 (m, 2H), 2.49 (dt, J=12.5, 6.0 Hz, 1H), 2.35 (q, J=8.6 Hz, 1H), 2.20-2.06 (m, 1H), 2.04-1.68 (m, 3H), 1.46 (t, J=7.3 Hz, 3H).

### Synthesis 21

### 6-[2-[2-(1,3-dimethylpyrazol-4-yl)pyrrolidin-1-yl]ethoxy]-3,4-dihydro-1H-quinolin-2-one (ALDHI-2015)

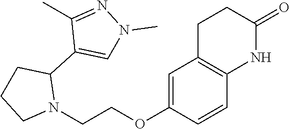 [SMILES: Cc1nn(C)cc1C1CCCN1CCOc1ccc2c(c1)CCC(=O)N2]

MS (ES+) m/z 355.4 (M+H).

¹H NMR (300 MHz, CDCl₃) δ 7.48 (s, 1H), 7.23 (s, 1H), 6.70-6.58 (m, 3H), 3.96 (t, J=6.0 Hz, 2H), 3.78 (s, 3H), 3.42-3.32 (m, 1H), 3.32-3.22 (m, 1H), 3.09-2.98 (m, 1H), 2.94-2.87 (m, 2H), 2.64-2.55 (m, 2H), 2.51-2.40 (m, 1H), 2.30 (q, J=8.9 Hz, 1H), 2.23 (s, 3H), 2.17-2.04 (m, 1H), 1.99-1.62 (m, 3H).

### Synthesis 22

### 6-[2-[2-(1-methylpyrrol-2-yl)pyrrolidin-1-yl]ethoxy]-3,4-dihydro-1H-quinolin-2-one (ALDHI-2017)

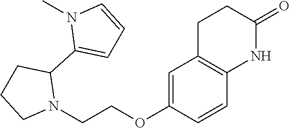 [SMILES: Cn1cccc1C1CCCN1CCOc1ccc2c(c1)CCC(=O)N2]

MS (ES+) m/z 340.3 (M+H).

¹H NMR (300 MHz, CDCl₃) δ 7.40 (s, 1H), 6.68-6.56 (m, 3H), 6.54 (t, J=2.3 Hz, 1H), 6.05 (d, J=2.3 Hz, 2H), 3.91 (t, J=6.1 Hz, 2H), 3.68 (s, 3H), 3.47 (t, J=8.0 Hz, 1H), 3.41-3.31 (m, 1H), 3.01 (dt, J=12.4, 6.1 Hz, 1H), 2.94-2.86 (m, 2H), 2.63-2.46 (m, 3H), 2.39-2.24 (m, 1H), 2.17-2.01 (m, 1H), 2.00-1.75 (m, 3H).

### Synthesis 23

### 6-[2-[2-(3-fluorophenyl)-4-hydroxy-pyrrolidin-1-yl]ethoxy]-3,4-dihydro-1H-quinolin-2-one Mixture of Diastereomers (4:1) (ALDHI-2035)

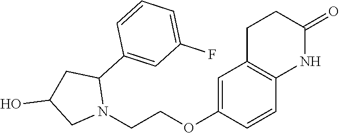 [SMILES: O=C1CCc2cc(OCCN3CC(O)CC3c3cccc(F)c3)ccc2N1]

MS (ES+) m/z 371.3 (M+H).

Major isomer reported: ¹H NMR (300 MHz, DMSO-d₆) δ 9.89 (s, 1H), 7.41-7.14 (m, 3H), 7.09-6.98 (m, 1H), 6.78-6.60 (m, 3H), 4.89 (s, 1H), 4.31-4.23 (m, 1H), 3.91 (t, J=5.8 Hz, 2H), 3.73 (dd, J=9.7, 6.7 Hz, 1H), 3.61 (dd, J=9.9, 6.1 Hz, 1H), 2.86-2.66 (m, 3H), 2.58-2.51 (m, 1H), 2.44-2.27 (m, 3H), 1.96 (ddd, J=12.9, 6.8, 2.5 Hz, 1H), 1.71 (ddd, J=12.8, 9.8, 7.1 Hz, 1H).

Both isomers reported: ¹⁹F {1H} NMR (282 MHz, DMSO-d₆) δ −113.42 (major isomer), −113.54 (minor isomer).

### Synthesis 24

### 6-[2-[2-(3-fluorophenyl)azetidin-1-yl]ethoxy]-3,4-dihydro-1H-quinolin-2-one (ALDHI-2021)

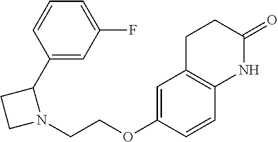 [SMILES: O=C1CCc2cc(OCCN3CCC3c3cccc(F)c3)ccc2N1]

MS (ES+) m/z 341.3 (M+H).

¹H NMR (300 MHz, CDCl₃) δ 7.55 (s, 1H), 7.23-7.10 (m, 3H), 6.89 (tdd, J=8.1, 2.7, 1.2 Hz, 1H), 6.61-6.56 (m, 3H), 4.11 (t, J=8.2 Hz, 1H), 3.87 (t, J=5.7 Hz, 2H), 3.58-3.49 (m, 1H), 3.10-2.98 (m, 1H), 2.93-2.82 (m, 4H), 2.63-2.54 (m, 2H), 2.40-2.27 (m, 1H), 2.17-2.01 (m, 1H).

¹⁹F {¹H} NMR (282 MHz, CDCl₃) δ −113.51.

### Synthesis 25

### 6-[2-[2-(3-fluorophenyl)-1-piperidyl]ethoxy]-3,4-dihydro-1H-quinolin-2-one (ALDHI-2038)

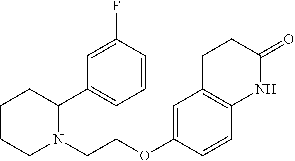 [SMILES: O=C1CCc2cc(OCCN3CCCCC3c3cccc(F)c3)ccc2N1]

MS (ES+) m/z 369.3 (M+H).

¹H NMR (300 MHz, DMSO-d₆) δ 9.87 (s, 1H), 7.42-7.29 (m, 1H), 7.23-7.12 (m, 2H), 7.05 (dddd, J=9.1, 8.2, 2.7, 1.1 Hz, 1H), 6.76-6.57 (m, 3H), 3.95-3.84 (m, 2H), 3.25-3.15 (m, 2H), 2.79 (t, J=7.5 Hz, 2H), 2.75-2.61 (m, 1H), 2.43-2.32 (m, 2H), 2.32-2.13 (m, 2H), 1.80-1.22 (m, 6H).

¹⁹F {¹H} NMR (282 MHz, DMSO-d₆) δ −113.35.

### Synthesis 26

### 6-[2-(4-methylsulfonyl-2-phenyl-piperazin-1-yl)ethoxy]-3,4-dihydro-1H-quinolin-2-one (ALDHI-2046)

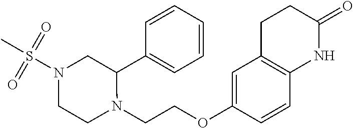 [SMILES: CS(=O)(=O)N1CCN(CCOc2ccc3c(c2)CCC(=O)N3)C(c2ccccc2)C1]

MS (ES+) m/z 430.3 (M+H).

¹H NMR (300 MHz, DMSO-d₆) δ 9.88 (s, 1H), 7.48-7.28 (m, 5H), 6.86-6.55 (m, 3H), 3.93 (t, J=6.0 Hz, 2H), 3.61-3.40 (m, 2H), 3.39-3.25 (m, 2H), 3.02-2.85 (m, 4H), 2.85-2.66 (m, 4H), 2.56-2.50 (m, 1H), 2.44-2.30 (m, 3H).

### Synthesis 27

### 6-[2-[3-(3-fluorophenyl)morpholin-4-yl]ethoxy]-3,4-dihydro-1H-quinolin-2-one (ALDHI-2031)

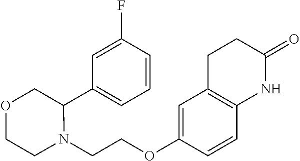 [SMILES: O=C1CCc2cc(OCCN3CCOCC3c3cccc(F)c3)ccc2N1]

MS (ES+) m/z 371.3 (M+H).

¹H NMR (300 MHz, Methanol-d₄) δ 7.43-7.29 (m, 1H), 7.28-7.17 (m, 2H), 7.09-6.96 (m, 1H), 6.82-6.63 (m, 3H), 4.10-3.86 (m, 3H), 3.84-3.64 (m, 2H), 3.52-3.35 (m, 2H), 3.22-3.12 (m, 1H), 2.97-2.80 (m, 3H), 2.63-2.49 (m, 3H), 2.42 (dt, J=13.7, 5.1 Hz, 1H).

¹⁹F {1H} NMR (282 MHz, Methanol-da) 6-114.97.

### Synthesis 28

### 6-[2-(3-methyl-3-phenyl-morpholin-4-yl)ethoxy]-3,4-dihydro-1H-quinolin-2-one (ALDHI-2040)

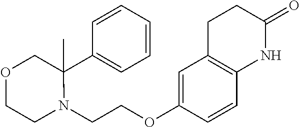 [SMILES: CC1(c2ccccc2)COCCN1CCOc1ccc2c(c1)CCC(=O)N2]

MS (ES+) m/z 367.3 (M+H).

¹H NMR (300 MHz, DMSO-d6) δ 9.87 (s, 1H), 7.69-7.59 (m, 2H), 7.42-7.31 (m, 2H), 7.31-7.21 (m, 1H), 6.75-6.56 (m, 3H), 3.99-3.79 (m, 3H), 3.61 (td, J=11.5, 3.0 Hz, 1H), 3.33-3.24 (m, 2H), 2.97-2.86 (m, 1H), 2.85-2.67 (m, 3H), 2.48-2.44 (m, 1H), 2.42-2.34 (m, 2H), 2.31-2.21 (m, 1H), 1.44 (s, 3H).

### Synthesis 29

### 6-[2-(2-methyl-5-phenyl-morpholin-4-yl)ethoxy]-3,4-dihydro-1H-quinolin-2-one Mixture of Diastereomers (1:4) (ALDHI-2042)

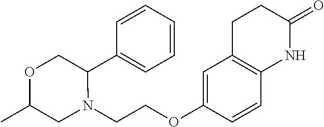 [SMILES: CC1CN(CCOc2ccc3c(c2)CCC(=O)N3)C(c2ccccc2)CO1]

MS (ES+) m/z 367.3 (M+H).

¹H NMR (300 MHz, DMSO-d₆) δ 9.88 (s, 1H), 7.49-7.23 (m, 5H), 6.83-6.57 (m, 3H), 4.00-3.86 (m, 2H), 3.77-3.42 (m, 2H), 3.30-3.23 (m, 2H), 3.14 (dd, J=11.5, 2.2 Hz, 1H), 2.86-2.66 (m, 3H), 2.43-2.22 (m, 3H), 2.16-2.02 (m, 1H), 1.12 (d, J=6.2 Hz, 2H).

### Synthesis 30

### 6-[2-(2-phenylpiperazin-1-yl)ethoxy]-3,4-dihydro-1H-quinolin-2-one (ALDHI-2036)

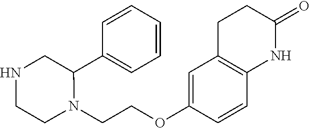 [SMILES: O=C1CCc2cc(OCCN3CCNCC3c3ccccc3)ccc2N1]

A mixture of 6-(2-chloroethoxy)-3,4-dihydro-1H-quinolin-2-one (60 mg, 0.266 mmol, 1 eq), tert-butyl 3-phenylpiperazine-1-carboxylate (105 mg, 0.399 mmol, 1.5 eq), K₂CO₃ (75 mg, 0.532 mmol, 2 eq) and KI (44 mg, 0.266 mmol, 1 eq) in MeCN (5 mL) was heated to reflux for 6 days under nitrogen. The mixture was cooled, water (10 mL) followed by DCM (10 mL) were added and the phases separated. The aqueous phase was washed with DCM (10 mL), the organic extracts combined, filtered through a hydrophobic frit and concentrated in vacuo. The resulting residue was chromatographed (SiO₂) using 0-100% EtOAc:petroleum ether as eluent. The residue was dissolved in DCM (5 mL) and trifluoroacetic acid (2.0 mL, 26.0 mmol, 97.6 eq) added. The mixture was stirred at RT for 1 h. The resulting mixture was diluted with DCM (20 mL), quenched with sat. aq. NaHCO₃ (50 mL), the phases separated and the aqueous extracted with DCM (20 mL). The combined extracts were washed with brine (50 mL), filtered through a hydrophobic frit and concentrated in vacuo. The residue was purified by preparative HPLC-MS (low pH then high pH). The appropriate fractions were combined, sat. aq. NaHCO₃ (10 mL) added and extracted with DCM (2×10 mL). The combined extracts were washed with brine (20 mL), filtered through a hydrophobic frit and concentrated in vacuo. The resulting residue was triturated with Et₂O and dried in vacuo at 50° C. to afford the title compound (15 mg, 0.0435 mmol, 16%) as a white powder.

MS (ES+) m/z 352.3 (M+H).

¹H NMR (300 MHz, DMSO-d₆) δ 9.87 (s, 1H), 7.40-7.19 (m, 5H), 6.74-6.56 (m, 3H), 3.97-3.81 (m, 2H), 3.19 (dd, J=10.2, 3.1 Hz, 1H), 3.11-3.02 (m, 1H), 2.92-2.61 (m, 6H), 2.48-2.19 (m, 5H).

### Synthesis 31

### 6-[2-(2-phenylimidazol-1-yl)ethoxy]-3,4-dihydro-1H-quinolin-2-one (ALDHI-2041)

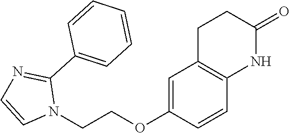 [SMILES: O=C1CCc2cc(OCCn3ccnc3-c3ccccc3)ccc2N1]

Prepared as described in method A from 6-(2-chloroethoxy)-3,4-dihydro-1H-quinolin-2-one (30 mg, 0.133 mmol, 1 eq), 2-phenyl-1H-imidazole (29 mg, 0.199 mmol, 1.5 eq), K₂CO₃ (37 mg, 0.266 mmol, 2 eq) and KI (22 mg, 0.133 mmol, 1.0 eq) in MeCN (2.5 mL) to return the title compound (16.2 mg, 0.0486 mmol, 37%) as a white powder after purification by preparative HPLC-MS (high pH).

MS (ES+) m/z 334.3 (M+H).

¹H NMR (300 MHz, DMSO-d₆) δ 9.91 (s, 1H), 7.70-7.59 (m, 2H), 7.56-7.38 (m, 4H), 7.02 (d, J=1.2 Hz, 1H), 6.86-6.60 (m, 3H), 4.37 (t, J=5.2 Hz, 2H), 4.21 (t, J=5.2 Hz, 2H), 2.80 (dd, J=8.5, 6.5 Hz, 2H), 2.44-2.32 (m, 2H).

### Synthesis 32

### 6-[2-[5-(3-fluorophenyl)pyrazol-1-yl]ethoxy]-3,4-dihydro-1H-quinolin-2-one (ALDHI-2023)

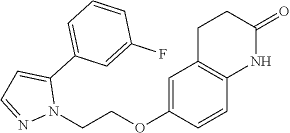 [SMILES: O=C1CCc2cc(OCCn3nccc3-c3cccc(F)c3)ccc2N1]

Prepared as described in method A from 6-(2-chloroethoxy)-3,4-dihydro-1H-quinolin-2-one (100 mg, 0.400 mmol, 1 eq), 3-(3-fluorophenyl)-1H-pyrazole hydrochloride (118.8 mg, 0.600 mmol, 1.5 eq), K₂CO₃ (165.4 mg, 1.2 mmol, 3 eq) and KI (66.2 mg, 0.400 mmol, 1 eq) in MeCN (8 mL) to return the title compound (10 mg, 0.0296 mmol, 7%) as a white powder after purification by preparative HPLC-MS (high pH).

MS (ES+) m/z 352.3 (M+H).

¹H NMR (300 MHz, CDCl₃) δ 7.68 (s, 1H), 7.58 (d, J=1.9 Hz, 1H), 7.50-7.36 (m, 1H), 7.33-7.20 (m, 2H), 7.13 (tdd, J=8.4, 2.6, 1.1 Hz, 1H), 6.61 (s, 3H), 6.31 (d, J=1.8 Hz, 1H), 4.53-4.40 (m, 2H), 4.40-4.32 (m, 2H), 2.95-2.83 (m, 2H), 2.65-2.52 (m, 2H).

¹⁹F {¹H} NMR (282 MHz, CDCl₃) δ −112.20.

### Synthesis 33

### 6-[2-[2-(3-fluorophenyl)-2-methyl-pyrrolidin-1-yl]ethoxy]-3,4-dihydro-1H-quinolin-2-one (ALDHI-2037)

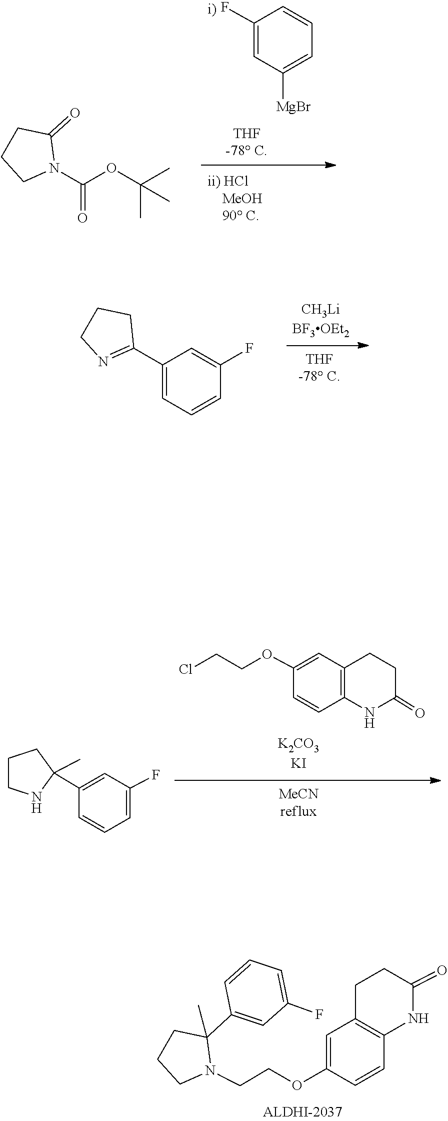

### Step 1: 5-(3-fluorophenyl)-3,4-dihydro-2H-pyrrole

To a mixture of tert-butyl 2-oxopyrrolidine-1-carboxylate (1.8 mL, 10.8 mmol, 1 eq) in THF (40 mL) at −78° C. under nitrogen was added 3-fluorophenylmagnesium bromide (1M in THF, 16 mL, 16.2 mmol, 1.5 eq) dropwise. The mixture was stirred for 18 h during which the temperature was slowly warmed to RT. The mixture was quenched with MeOH (50 mL) and stirred for 1 h. The solvents were removed in vacuo, then EtOAc (100 mL) and brine (100 mL) were added, filtered and the phases separated. The organic phase was washed with brine (3×100 mL), sat. aq. NaHCO₃ (3×100 mL), filtered through a hydrophobic frit and concentrated in vacuo to afford a yellow oil. The oil was dissolved in MeOH (40 mL) and concentrated HCl (3.3 mL, 108 mmol, 10 eq) added. The mixture was stirred at 90° C. for 2 h. The mixture was cooled, carefully neutralized with sat. aq. NaHCO₃ (50 mL), extracted with EtOAc (3×100 mL), the combined extracts filtered through a hydrophobic frit and concentrated in vacuo. The resulting residue was chromatographed (SiO₂) using 0-25% EtOAc:petroleum ether as eluent to afford 5-(3-fluorophenyl)-3,4-dihydro-2H-pyrrole (792 mg, 4.85 mmol, 45%) as a light yellow oil.

MS (ES+) m/z 164.1 (M+H).

¹H NMR (300 MHz, CDCl₃) δ 7.65-7.49 (m, 2H), 7.42-7.32 (m, 1H), 7.12 (tdd, J=8.3, 2.6, 1.0 Hz, 1H), 4.08 (tt, J=7.4, 2.1 Hz, 2H), 2.93 (ddt, J=8.3, 7.3, 2.1 Hz, 2H), 2.14-1.94 (m, 2H).

¹⁹F {1H} NMR (282 MHz, CDCl₃) δ −113.03.

### Step 2: 2-(3-fluorophenyl)-2-methyl-pyrrolidine

To a solution of 5-(3-fluorophenyl)-3,4-dihydro-2H-pyrrole (775 mg, 4.27 mmol, 1 eq) in THF (45 mL) was added at −78° C. boron trifluoride diethyl etherate (1.1 mL, 8.55 mmol, 2 eq) over ca. 5 min. The mixture was stirred for 40 min then methyl lithium (6.7 mL, 10.7 mmol, 2.5 eq) was added dropwise over 10 min. The mixture was stirred for 16 h allowing to warm up slowly to RT. Water (40 mL) followed by aq. HCl (2M, 10 mL) then EtOAc (50 mL) were added and the phases separated. The organic phase was washed with aq. HCl (1M, 25 mL) and the aqueous phases combined. The aqueous phase was basified with aq. NaOH (2M) to pH 12, extracted with EtOAc (2×50 mL), the extracts washed with brine (100 mL), filtered through a hydrophobic frit and concentrated in vacuo to afford 2-(3-fluorophenyl)-2-methyl-pyrrolidine (669 mg, 0.448 mmol, 10%, ca. 12% purity by LCMS) as an orange oil used in the next step without further purification.

MS (ES+) m/z 180.2 (M+H).

### Step 3: 6-[2-[2-(3-fluorophenyl)-2-methyl-pyrrolidin-1-yl]ethoxy]-3,4-dihydro-1H-quinolin-2-one

Prepared as described in method A from 6-(2-chloroethoxy)-3,4-dihydro-1H-quinolin-2-one (30 mg, 0.133 mmol, 1 eq), 2-(3-fluorophenyl)-2-methyl-pyrrolidine (300 mg, 0.201 mmol, 1.5 eq, ca. 12% purity by LCMS), K₂CO₃ (37 mg, 0.266 mmol, 2 eq) and KI (22 mg, 0.133 mmol, 1 eq) in MeCN (5 mL) to return the title compound (10.1 mg, 0.0274 mmol, 21%) as a light beige powder after reverse phase chromatography (C₁₈) using 5-95% MeCN:H₂O as eluent and subsequent purification by preparative HPLC-MS (high pH).

MS (ES+) m/z 369.3 (M+H).

¹H NMR (300 MHz, DMSO-d₆) δ 9.89 (s, 1H), 7.45-7.27 (m, 3H), 7.10-6.93 (m, 1H), 6.80-6.64 (m, 3H), 4.01-3.92 (m, 2H), 2.82 (t, J=8.5, 6.5 Hz, 2H), 2.73-2.56 (m, 2H), 2.44-2.33 (m, 2H), 1.91-1.61 (m, 4H), 1.30 (s, 3H).

¹⁹F {1H} NMR (282 MHz, DMSO-d₆) δ −113.60.

### Synthesis 34

### 6-[2-[4-fluoro-2-(3-fluorophenyl)pyrrolidin-1-yl]ethoxy]-3,4-dihydro-1H-quinolin-2-one (ALDHI-2052)

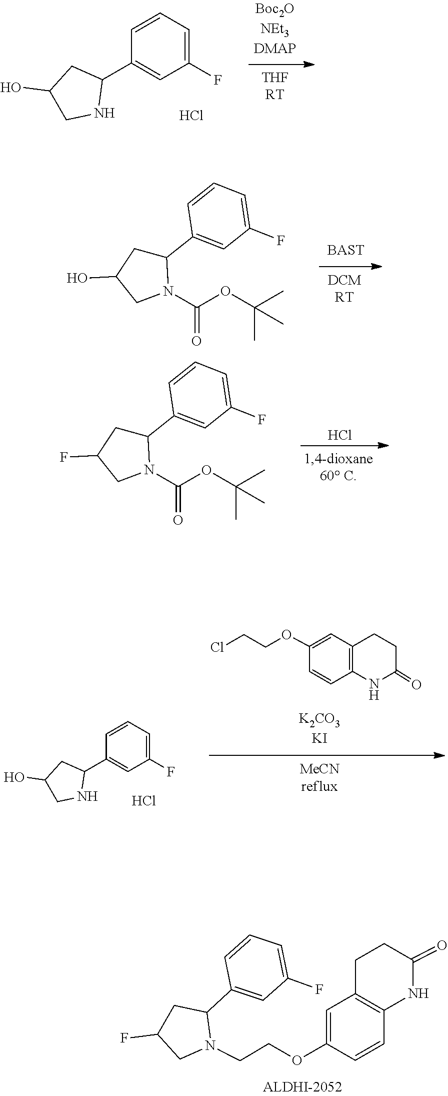

### Step 1: tert-butyl 2-(3-fluorophenyl)-4-hydroxy-pyrrolidine-1-carboxylate

To a suspension of 5-(3-fluorophenyl)pyrrolidin-3-ol hydrochloride, mixture of diastereomers (250 mg, 1.15 mmol, 1 eq) in THF (10 mL) were added triethylamine (0.48 mL, 3.45 mmol, 3 eq), DMAP (7 mg, 0.0574 mmol, 0.05 eq) and di-tert-butyl dicarbonate (276 mg, 1.26 mmol, 1.1 eq). The reaction mixture was stirred at RT 16 h then quenched with water (20 mL) and extracted with DCM (2×25 mL). The extracts were washed with brine (50 mL), filtered through a hydrophobic frit and concentrated in vacuo. The resulting residue was chromatographed (C₁₈) using 5-95% MeCN:H₂O as eluent to afford tert-butyl 2-(3-fluorophenyl)-4-hydroxy-pyrrolidine-1-carboxylate (238 mg, 0.846 mmol, 74%) as an off white powder used in the next step without further purification.

MS (ES+) m/z 262.12 m/z (M+H-tBu).

### Step 2: tert-butyl 4-fluoro-2-(3-fluorophenyl)pyrrolidine-1-carboxylate

To a solution of tert-butyl 2-(3-fluorophenyl)-4-hydroxy-pyrrolidine-1-carboxylate (100 mg, 0.355 mmol, 1 eq) in DCM (10 mL) was added BAST (50% in toluene, 0.27 mL, 0.533 mmol, 1.5 eq) via syringe under nitrogen. The mixture was stirred at RT for 16 h then aq. NaOH (1M, 5 mL) was added and stirred for 0.5 h. The phases were separated and the organic layer was washed with brine (3×10 mL), dried (MgSO₄), filtered and concentrated in vacuo. The residue was chromatographed (SiO₂) using 0-20% EtOAc:petroleum ether as eluent to afford tert-butyl 4-fluoro-2-(3-fluorophenyl)pyrrolidine-1-carboxylate (40 mg, 0.141 mmol, 40%) as a colourless oil which solidified on standing.

MS (ES+) m/z 228.1 (M+H).

¹H NMR (300 MHz, DMSO-d₆) δ 7.35 (td, J=7.9, 6.1 Hz, 1H), 7.10-6.93 (m, 3H), 5.45-5.18 (m, 1H), 5.09-4.85 (m, 1H), 3.83-3.78 (m, 1H), 3.78-3.60 (m, 1H), 2.82-2.54 (m, 1H), 2.20-2.02 (m, 1H), 1.57-1.02 (m, 9H).

¹⁹F {¹H} NMR (282 MHz, DMSO-d₆) δ −114.08, −170.17.

### Step 3: 4-fluoro-2-(3-fluorophenyl)pyrrolidine hydrochloride

To a solution of tert-butyl 4-fluoro-2-(3-fluorophenyl)pyrrolidine-1-carboxylate (33 mg, 0.116 mmol, 1 eq) in 1,4-dioxane (2 mL) was added HCl (4M in 1,4-dioxane, 2.0 mL, 8.00 mmol, 69 eq) and the mixture heated to 60° C. for 1 h. The mixture was then cooled, concentrated in vacuo and the resulting solid triturated with Et₂O and dried in vacuo at 50° C. to afford 4-fluoro-2-(3-fluorophenyl)pyrrolidine hydrochloride (24 mg, 0.109 mmol, 94%) as an off-white powder.

MS (ES+) m/z 184.1 (M+H).

¹H NMR (300 MHz, DMSO-d₆) δ 9.93 (br.s, 2H), 7.60-7.47 (m, 1H), 7.42-7.23 (m, 3H), 5.71-5.45 (m, 1H), 4.79 (t, J=8.8 Hz, 1H), 3.74-3.41 (m, 2H), 3.05-2.82 (m, 1H), 2.46-2.23 (m, 1H).

¹⁹F {¹H} NMR (282 MHz, DMSO-d₆) δ −112.23, −170.12.

### Step 4: 6-[2-[4-fluoro-2-(3-fluorophenyl)pyrrolidin-1-yl]ethoxy]-3,4-dihydro-1H-quinolin-2-one

Prepared as described in method A from 6-(2-chloroethoxy)-3,4-dihydro-1H-quinolin-2-one (22 mg, 0.0956 mmol, 0.9 eq), 4-fluoro-2-(3-fluorophenyl)pyrrolidine hydrochloride (23 mg, 0.105 mmol, 1 eq), K₂CO₃ (44 mg, 0.315 mmol, 3 eq) and KI (17 mg, 0.105 mmol, 1 eq) in MeCN (2.5 mL) to return the title compound (10 mg, 0.0256 mmol, 23%) as a white powder after purification by preparative HPLC-MS (low pH).

MS (ES+) m/z 373.3 (M+H).

¹H NMR (300 MHz, Methanol-d₄) δ 7.39-7.28 (m, 1H), 7.26-7.18 (m, 2H), 7.03-6.94 (m, 1H), 6.82-6.64 (m, 3H), 4.00 (t, J=5.6 Hz, 2H), 3.63 (d, J=11.8 Hz, 1H), 3.48 (t, J=8.4 Hz, 2H), 2.92 (dd, J=9.5, 6.3 Hz, 3H), 2.78-2.49 (m, 5H), 1.94-1.80 (m, 1H).

¹⁹F {¹H} NMR (282 MHz, Methanol-d₄) δ −115.27, −167.41.

### Synthesis 35

### 6-[2-[4,4-difluoro-2-(3-fluorophenyl)pyrrolidin-1-yl]ethoxy]-3,4-dihydro-1H-quinolin-2-one (ALDHI-2053)

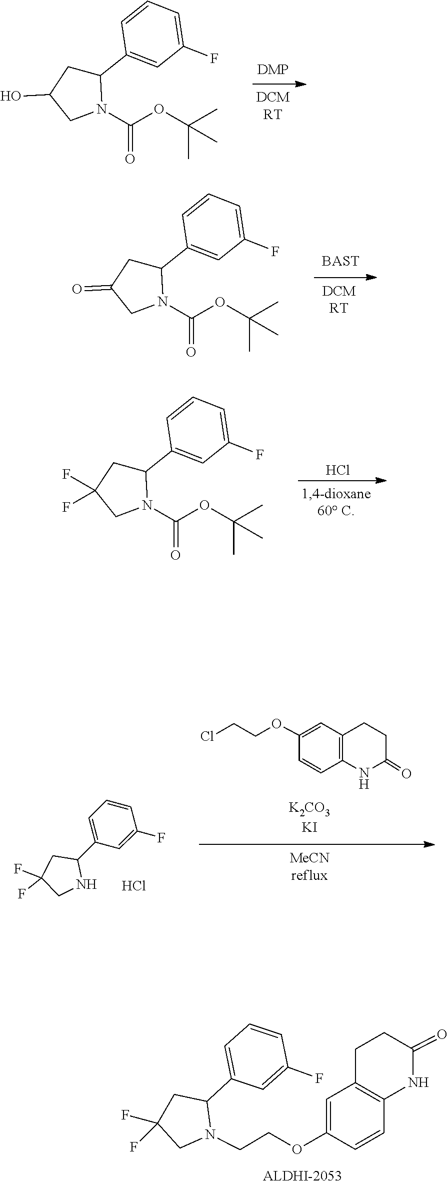

### Step 1: tert-butyl 2-(3-fluorophenyl)-4-oxo-pyrrolidine-1-carboxylate

To a solution of tert-butyl 2-(3-fluorophenyl)-4-hydroxy-pyrrolidine-1-carboxylate (160 mg, 0.569 mmol, 1 eq) in DCM (5 mL) was added DMP (289 mg, 0.682 mmol, 1.2 eq); the reaction mixture was stirred at RT for 40 h then aq. NaOH (1M, 5 mL) was added and the mixture stirred for 1 h. The mixture was diluted with DCM (10 mL), the phases were separated, the aqueous phase extracted with DCM (10 mL) and the extracts combined. The extracts were washed with water (20 mL), brine (25 mL), filtered through a hydrophobic frit and concentrated in vacuo. The resulting solid was chromatographed (SiO₂) using 0-30% EtOAc:petroleum ether as eluent to afford tert-butyl 2-(3-fluorophenyl)-4-oxo-pyrrolidine-1-carboxylate (138 mg, 0.494 mmol, 87%) as a colourless oil used directly in the next step.

MS (ES−) m/z 278.2 (M-H-tBu).

¹H NMR (300 MHz, DMSO-d₆) δ 7.46-7.32 (m, 1H), 7.21-6.95 (m, 3H), 5.26 (br. s, 1H), 3.97 (s, 2H), 3.31-3.20 (m, 1H), 2.40 (dd, J=18.6, 3.4 Hz, 1H), 1.50-1.11 (m, 9H).

¹⁹F NMR {¹H} (282 MHz, DMSO-d₆) δ −111.94-−113.63 (m).

### Step 2: tert-butyl 4,4-difluoro-2-(3-fluorophenyl)pyrrolidine-1-carboxylate

To a solution of tert-butyl 2-(3-fluorophenyl)-4-oxo-pyrrolidine-1-carboxylate (133 mg, 0.476 mmol, 1 eq) in DCM (10 mL) was added BAST (50% in toluene, 0.36 mL, 0.714 mmol, 1.5 eq) via syringe under nitrogen. The mixture was stirred at RT for 18 h. aq. NaOH (1M, 5 mL) was added and stirred for 0.5 h. The phases were separated, the aqueous layer washed with DCM (10 mL) and the extracts combined. The extracts were washed with water (20 mL), brine (20 mL), filtered through a hydrophobic frit and concentrated in vacuo. The resulting residue was chromatographed (SiO₂) using 0-20% EtOAc:petroleum ether to afford tert-butyl 4,4-difluoro-2-(3-fluorophenyl)pyrrolidine-1-carboxylate (113 mg, 0.375 mmol, 79%) as a colourless oil used directly in the next step.

MS (ES+) m/z 246.1 (M+H-tBu).

¹H NMR (300 MHz, DMSO-d₆) δ 7.46-7.32 (m, 1H), 7.25-6.97 (m, 3H), 4.99 (s, 1H), 4.09-3.79 (m, 2H), 3.07-2.85 (m, 1H), 2.46-2.19 (m, 1H), 1.58-0.99 (m, 9H).

¹⁹F {¹H} NMR (282 MHz, DMSO-d₆) δ −98.03-−100.70 (m), −101.00-−103.61 (m), −112.94-−114.40 (m).

### Step 3: 4,4-difluoro-2-(3-fluorophenyl)pyrrolidine hydrochloride

To a solution of tert-butyl 4,4-difluoro-2-(3-fluorophenyl)pyrrolidine-1-carboxylate (110 mg, 0.365 mmol, 1 eq) in 1,4-dioxane (2 mL) was added HCl (4M in 1,4-dioxane, 2.0 mL, 8.00 mmol, 22 eq). The mixture was heated to 60° C. for 1 h then cooled down to RT and concentrated in vacuo. The resulting solid was triturated with Et₂O and dried in vacuo at 50° C. to afford 4,4-difluoro-2-(3-fluorophenyl)pyrrolidine hydrochloride (68 mg, 0.286 mmol, 78%) as a pink powder.

MS (ES+) m/z 202.1 (M+H).

¹H NMR (300 MHz, DMSO-d₆) δ 10.29 (br s, 2H), 7.61-7.26 (m, 4H), 5.01 (dd, J=12.5, 6.6 Hz, 1H), 4.02-3.68 (m, 2H), 3.14-2.95 (m, 1H), 2.95-2.68 (m, 1H).

¹⁹F {1H} NMR (282 MHz, DMSO-d₆) δ −90.76 (d, J=233.0 Hz), −96.40 (d, J=233.0 Hz), −112.16.

### Step 4: 6-[2-[4,4-difluoro-2-(3-fluorophenyl)pyrrolidin-1-yl]ethoxy]-3,4-dihydro-1H-quinolin-2-one

Prepared as described in method A from 6-(2-chloroethoxy)-3,4-dihydro-1H-quinolin-2-one (25 mg, 0.111 mmol, 1 eq), 4,4-difluoro-2-(3-fluorophenyl)pyrrolidine hydrochloride (29 mg, 0.122 mmol, 1.1 eq), K₂CO₃ (47 mg, 0.332 mmol, 3 eq) and KI (18 mg, 0.111 mmol, 1 eq) in MeCN (2.5 mL) to return the title compound (10 mg, 0.0256 mmol, 23%) as a white powder after purification by preparative HPLC-MS (low pH).

MS (ES+) m/z 391.3 (M+H).

¹H NMR (300 MHz, DMSO-d₆) δ 9.89 (s, 1H), 7.47-7.34 (m, 1H), 7.30-7.19 (m, 2H), 7.18-7.04 (m, 1H), 6.81-6.61 (m, 3H), 3.99-3.90 (m, 2H), 3.90-3.80 (m, 1H), 3.80-3.65 (m, 1H), 3.00-2.60 (m, 5H), 2.58-2.53 (m, 1H), 2.45-2.34 (m, 2H), 2.22-1.96 (m, 1H).

¹⁹F {¹H} NMR (282 MHz, DMSO-d₆) δ −89.39 (d, J=226.0 Hz), −93.33 (d, J=226.0 Hz), −112.97.

### Scheme 2

### Alkylation of 2-Substituted Cyclic Amines with 7-(2-chloroethoxy)-4H-1,4-benzoxazin-3-one

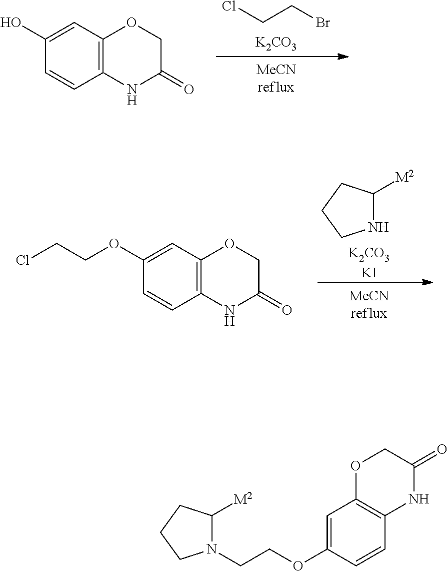

### Synthesis 36

### 7-(2-chloroethoxy)-4H-1,4-benzoxazin-3-one

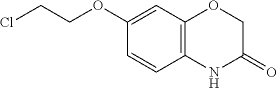 [SMILES: O=C1COc2cc(OCCCl)ccc2N1]

To a mixture of 7-hydroxy-4H-1,4-benzoxazin-3-one (100 mg, 0.606 mmol, 1 eq) (prepared as reported in La et al., Journal of Medicinal Chemistry, 2008, Vol. 51, pp. 1695-1705), and K₂CO₃ (167 mg, 1.21 mmol, 2 eq) in MeCN (5 mL) under nitrogen was added 1-bromo-2-chloroethane (0.25 mL, 3.03 mmol, 5 eq). The mixture was heated to reflux for 3 days then cooled down to RT and water (10 mL) followed by DCM (10 mL) were added. The phases were separated, the aqueous phase washed with DCM (10 mL), the organic extracts combined, filtered through a hydrophobic frit and concentrated in vacuo. The resulting solid was chromatographed (SiO₂) using 0-5% MeOH:DCM (+1% aq. NH3) as eluent to afford the title compound (41 mg, 0.180 mmol, 30%) as an orange powder.

MS (ES−) m/z 226.1/228.1 (M−H), Cl isotope pattern.

¹H NMR (300 MHz, CDCl₃) δ 7.82 (s, 1H), 6.70 (d, J=8.6 Hz, 1H), 6.63-6.49 (m, 2H), 4.60 (s, 2H), 4.18 (t, J=5.9 Hz, 2H), 3.79 (t, J=5.8 Hz, 2H).

### General Method B

### Alkylation of 2-Substituted Cyclic Amines with 7-(2-chloroethoxy)-4H-1,4-benzoxazin-3-one

A mixture of 7-(2-chloroethoxy)-4H-1,4-benzoxazin-3-one (1 eq), 2-substituted cyclic amine (1.5 eq), K₂CO₃ (2 eq) and KI (1 eq) in MeCN (0.04 M to 0.05 M) was heated at reflux for 3 days under nitrogen then cooled down to RT. Water followed by DCM were added and the phases separated. The aqueous phase was washed with DCM, the organic extracts combined, filtered through a hydrophobic frit and concentrated in vacuo. The crude material was purified by preparative HPLC-MS using a gradient of high or low pH aq. MeCN.

### Synthesis 37

### 7-[2-[2-(1-methylpyrazol-4-yl)pyrrolidin-1-yl]ethoxy]-4H-1,4-benzoxazin-3-one (ALDHI-2024)

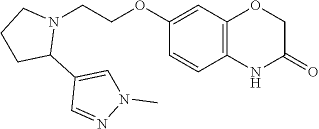 [SMILES: Cn1cc(C2CCCN2CCOc2ccc3c(c2)OCC(=O)N3)cn1]

Prepared as described in method B from 7-(2-chloroethoxy)-4H-1,4-benzoxazin-3-one (20 mg, 0.0879 mmol, 1 eq), 1-methyl-4-(pyrrolidin-2-yl)-1H-pyrazole (20 mg, 0.132 mmol, 1.5 eq), K₂CO₃ (24 mg, 0.176 mmol, 2 eq) and KI (15 mg, 0.0879 mmol, 1 eq) in MeCN (2.5 mL) to return the title compound (17 mg, 0.0497 mmol, 57%) as a colourless gum after purification by preparative HPLC-MS (high pH).

MS (ES+) m/z 343.3 (M+H).

¹H NMR (300 MHz, Methanol-d₄) δ 8.49 (s, 1H), 7.76 (s, 1H), 7.60 (d, J=0.8 Hz, 1H), 6.81 (dd, J=8.3, 0.6 Hz, 1H), 6.61-6.49 (m, 2H), 4.53 (s, 2H), 4.24-4.04 (m, 3H), 3.87 (s, 3H), 3.75-3.61 (m, 1H), 3.42-3.32 (m, 1H), 3.20-3.01 (m, 2H), 2.48-2.29 (m, 1H), 2.25-2.03 (m, 3H).

The following example compounds were prepared similarly using method B with the appropriate 2-substituted cyclic amine.

### Synthesis 38

### 7-[2-[2-(2-pyridyl)pyrrolidin-1-yl]ethoxy]-4H-1,4-benzoxazin-3-one (ALDHI-2025)

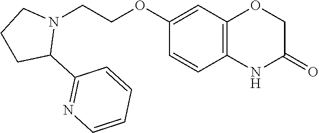 [SMILES: O=C1COc2cc(OCCN3CCCC3c3ccccn3)ccc2N1]

MS (ES+) m/z 340.3 (M+H).

¹H NMR (300 MHz, Methanol-d₄) δ 8.57-8.47 (m, 2H), 7.82 (td, J=7.7, 1.8 Hz, 1H), 7.58 (dt, J=7.9, 1.1 Hz, 1H), 7.32 (ddd, J=7.6, 4.9, 1.2 Hz, 1H), 6.78 (dt, J=9.0, 1.4 Hz, 1H), 6.52-6.42 (m, 2H), 4.54 (s, 2H), 4.26 (t, J=8.1 Hz, 1H), 4.16-3.98 (m, 2H), 3.79-3.65 (m, 1H), 3.31-3.22 (m, 1H), 3.21-3.11 (m, 1H), 3.06-2.92 (m, 1H), 2.57-2.39 (m, 1H), 2.17-2.03 (m, 2H), 2.03-1.83 (m, 1H).

### Scheme 3

### Alkylation of 2-Substituted Cyclic Amines with 7-(2-chloroethoxy)-4H-1,4-benzothiazin-3-one

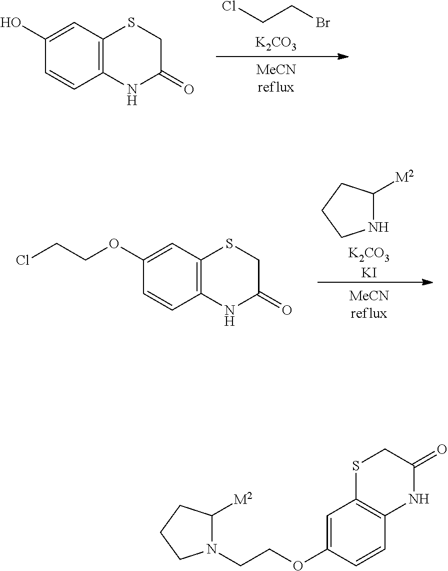

### Synthesis 39

### 7-(2-chloroethoxy)-4H-1,4-benzothiazin-3-one

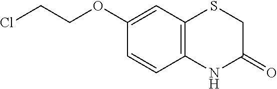 [SMILES: O=C1CSc2cc(OCCCl)ccc2N1]

To a mixture of 7-hydroxy-4H-1,4-benzothiazin-3-one (89 mg, 0.491 mmol, 1 eq) (prepared as reported in Zhang et al., Chemical and Pharmaceutical Bulletin, 2010, Vol. 58, pp. 326-331), and K₂CO₃ (136 mg, 0.982 mmol, 2 eq) in MeCN (5 mL) under nitrogen was added 1-bromo-2-chloroethane (0.20 mL, 2.46 mmol, 5 eq). The mixture was heated to reflux for 3 days then cooled down to RT and water (10 mL) followed by DCM (10 mL) were added. The phases were separated, the aqueous phase washed with DCM (10 mL), the organic extracts combined, filtered through a hydrophobic frit and concentrated in vacuo. The resulting solid was chromatographed (SiO₂) using 0-5% MeOH:DCM (+1% aq. NH₃) as eluent to afford the title compound (83 mg, 0.341 mmol, 69%) as an off-white powder.

MS (ES+) m/z 244.2/246.2 (M+H), Cl isotope pattern.

¹H NMR (300 MHz, CDCl₃) δ 7.77 (s, 1H), 6.93-6.85 (m, 1H), 6.76 (d, J=1.6 Hz, 2H), 4.19 (t, J=5.8 Hz, 2H), 3.79 (t, J=5.8 Hz, 2H), 3.42 (s, 2H).

### General Method C

### Alkylation of 2-Substituted Cyclic Amines with 7-(2-chloroethoxy)-4H-1,4-benzothiazin-3-one

A mixture of 7-(2-chloroethoxy)-4H-1,4-benzothiazin-3-one (1 eq), 2-substituted cyclic amine (1.5 eq), K₂CO₃ (2 eq) and KI (1 eq) in MeCN (0.03-0.05 M) was heated at reflux for 3 days then cooled down to RT. Water followed by DCM were added and the phases separated. The aqueous phase was washed with DCM, the organic extracts combined, filtered through a hydrophobic frit and concentrated in vacuo. The crude material was purified by preparative HPLC-MS using a gradient of high or low pH aq. MeCN.

### Synthesis 40

### 7-[2-[2-(1-methylpyrazol-4-yl)pyrrolidin-1-yl]ethoxy]-4H-1,4-benzothiazin-3-one (ALDHI-2026)

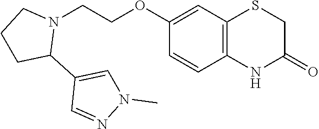 [SMILES: Cn1cc(C2CCCN2CCOc2ccc3c(c2)SCC(=O)N3)cn1]

Prepared as described in method C from 7-(2-chloroethoxy)-4H-1,4-benzothiazin-3-one (20 mg, 0.0821 mmol, 1 eq), 1-methyl-4-(pyrrolidin-2-yl)-1H-pyrazole (19 mg, 0.123 mmol, 1.5 eq), K₂CO₃ (23 mg, 0.164 mmol, 2 eq), KI (14 mg, 0.0821 mmol, 1 eq) in MeCN (1.5 mL) to return the title compound (19 mg, 0.053 mmol, 65%) as a colourless gum after preparative HPLC-MS (high pH) as a formate salt.

MS (ES+) m/z 359.3 (M+H).

¹H NMR (300 MHz, CDCl₃) δ 8.92 (s, 1H), 7.50 (d, J=0.8 Hz, 1H), 7.47 (d, J=0.8 Hz, 1H), 6.83-6.74 (m, 2H), 6.66 (dd, J=8.7, 2.7 Hz, 1H), 4.19-3.98 (m, 2H), 3.88 (s, 3H), 3.79-3.67 (m, 1H), 3.60-3.46 (m, 1H), 3.39 (s, 2H), 3.15 (dt, J=13.2, 5.4 Hz, 1H), 2.81-2.61 (m, 2H), 2.30-2.15 (m, 1H), 2.15-1.86 (m, 3H).

The following example compounds were prepared similarly using method C with the appropriate 2-substituted cyclic amine.

### Synthesis 41

### 7-[2-[2-(2-pyridyl)pyrrolidin-1-yl]ethoxy]-4H-1,4-benzothiazin-3-one (ALDHI-2027)

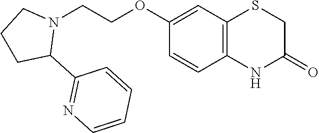 [SMILES: O=C1CSc2cc(OCCN3CCCC3c3ccccn3)ccc2N1]

MS (ES+) m/z 356.3 (M+H).

¹H NMR (300 MHz, CDCl₃) δ 8.59-8.49 (m, 2H), 7.65 (td, J=7.7, 1.8 Hz, 1H), 7.52 (d, J=7.8 Hz, 1H), 7.21-7.10 (m, 1H), 6.79-6.70 (m, 2H), 6.63 (dd, J=8.7, 2.7 Hz, 1H), 3.96 (t, J=5.9 Hz, 2H), 3.71 (t, J=7.9 Hz, 1H), 3.52-3.20 (m, 3H), 2.97 (dt, J=12.3, 6.0 Hz, 1H), 2.74 (dt, J=12.5, 5.8 Hz, 1H), 2.56 (q, J=8.6 Hz, 1H), 2.37-2.21 (m, 1H), 2.08-1.73 (m, 3H).

### Synthesis 42

### 7-[2-[2-(3-pyridyl)pyrrolidin-1-yl]ethoxy]-4H-1,4-benzothiazin-3-one (ALDHI-2028)

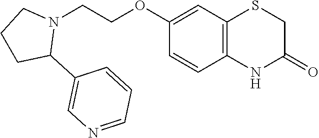 [SMILES: O=C1CSc2cc(OCCN3CCCC3c3cccnc3)ccc2N1]

MS (ES+) m/z 356.2 (M+H).

¹H NMR (300 MHz, Methanol-d₄) δ 8.58-8.52 (m, 1H), 8.44-8.36 (m, 1H), 7.89 (dt, J=7.9, 2.0 Hz, 1H), 7.38 (dd, J=7.9, 4.9 Hz, 1H), 6.89-6.76 (m, 2H), 6.69 (dd, J=8.8, 2.7 Hz, 1H), 4.06-3.88 (m, 2H), 3.57 (t, J=8.2 Hz, 1H), 3.48 (ddd, J=10.1, 7.6, 3.1 Hz, 1H), 3.38 (s, 2H), 2.98-2.84 (m, 1H), 2.72-2.59 (m, 1H), 2.53 (q, J=8.9 Hz, 1H), 2.37-2.19 (m, 1H), 2.07-1.83 (m, 2H), 1.81-1.62 (m, 1H).

### Synthesis 43

### 7-[2-[2-(4-pyridyl)pyrrolidin-1-yl]ethoxy]-4H-1,4-benzothiazin-3-one (ALDHI-2029)

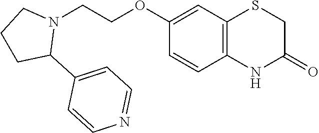 [SMILES: O=C1CSc2cc(OCCN3CCCC3c3ccncc3)ccc2N1]

MS (ES+) m/z 356.2 (M+H).

¹H NMR (300 MHz, Methanol-d₄) δ 8.47-8.39 (m, 2H), 7.53-7.45 (m, 2H), 6.91-6.78 (m, 2H), 6.70 (dd, J=8.8, 2.7 Hz, 1H), 4.09-3.91 (m, 2H), 3.56 (t, J=8.1 Hz, 1H), 3.47 (ddd, J=9.6, 7.2, 3.1 Hz, 1H), 3.40 (s, 2H), 2.92 (ddd, J=13.2, 6.5, 5.0 Hz, 1H), 2.68 (ddd, J=13.2, 5.8, 4.7 Hz, 1H), 2.53 (q, J=8.8 Hz, 1H), 2.30 (dtd, J=12.4, 8.4, 6.0 Hz, 1H), 2.07-1.83 (m, 2H), 1.75-1.57 (m, 1H).

### Alkylation of 2-(3-fluorophenyl)pyrrolidine with 6-(2-chloroethoxy)-1H-quinolin-2-one

### Synthesis 44

### 6-[2-[2-(3-fluorophenyl)pyrrolidin-1-yl]ethoxy]-1H-quinolin-2-one (ALDHI-2022)

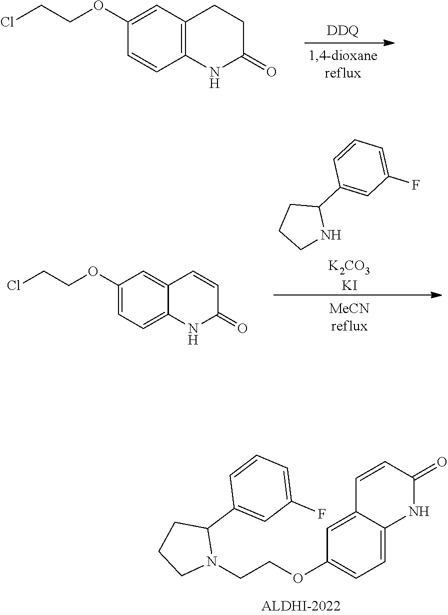

### Step 1: 6-(2-chloroethoxy)-1H-quinolin-2-one

To a solution of 6-(2-chloroethoxy)-3,4-dihydro-1H-quinolin-2-one (150 mg, 0.665 mmol, 1 eq) in 1,4-dioxane (15 mL) was added DDQ (226 mg, 0.997 mmol, 1.5 eq), the reaction mixture was heated to reflux for 16 h, then cooled down to RT, diluted with water (25 mL) and extracted with EtOAc (2×25 mL). The combined organic extracts were washed with brine (50 mL), dried (MgSO₄), filtered and the solvent removed in in vacuo. The residue was chromatographed (SiO₂) using 0-5% MeOH:DCM to afford 6-(2-chloroethoxy)-1H-quinolin-2-one (54 mg, 0.241 mmol, 36%) as a beige powder.

MS (ES+) 224.1/226.1 (M+H), Cl isotope pattern.

¹H NMR (300 MHz, DMSO-d₆) δ 11.65 (s, 1H), 7.84 (d, J=9.6 Hz, 1H), 7.30-7.23 (m, 2H), 7.19 (dd, J=9.0, 2.6 Hz, 1H), 6.50 (d, J=9.5 Hz, 1H), 4.35-4.23 (m, 2H), 4.01-3.90 (m, 2H).

### Step 2: 6-[2-[2-(3-fluorophenyl)pyrrolidin-1-yl]ethoxy]quinolin-2-ol

To a solution of 6-(2-chloroethoxy)-1H-quinolin-2-one (54 mg, 0.241 mmol, 1 eq), 2-(3-fluorophenyl)pyrrolidine (60 mg, 0.362 mmol, 1.5 eq) in MeCN (5 mL), K₂CO₃ (67 mg, 0.483 mmol, 2 eq) and KI (4 mg, 0.0241 mmol, 0.1 eq) were added and the mixture was refluxed for 5 days. The mixture was cooled down to RT and water (20 mL) followed by EtOAc (25 mL) were added. The phases were separated, the aqueous phase washed with EtOAc (2×25 mL), the organic extracts combined, dried (MgSO4), filtered and concentrated in vacuo. The resulting residue was chromatographed (C₁₈) using 5-95% MeCN:H₂O as eluent to afford the title compound (27.8 mg, 0.0789 mmol, 33%) as a yellow powder.

MS (ES+) m/z 353.3 (M+H).

¹H NMR (300 MHz, DMSO-d₆) δ 11.61 (s, 1H), 7.79 (d, J=9.5 Hz, 1H), 7.34 (td, J=8.0, 6.1 Hz, 1H), 7.26-7.07 (m, 5H), 7.06-6.96 (m, 1H), 6.48 (d, J=9.5 Hz, 1H), 4.03 (t, J=5.8 Hz, 2H), 3.50-3.34 (m, 2H), 2.84 (dt, J=12.5, 6.1 Hz, 1H), 2.38 (q, J=8.8 Hz, 1H), 2.24-2.07 (m, 1H), 1.91-1.73 (m, 2H), 1.52 (dtd, J=12.2, 9.8, 6.5 Hz, 1H).

### Scheme 4

### Alkylation of 2-Substituted Alkylamines with 6-(2-chloroethoxy)-3,4-dihydro-1H-quinolin-2-one

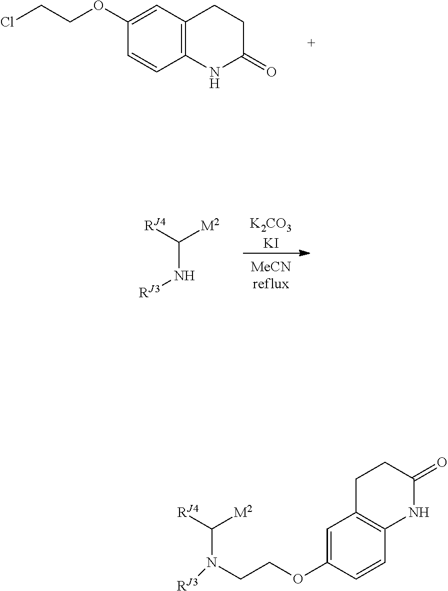

### General Method D

### Alkylation of 2-Substituted Alkylamines with 6-(2-chloroethoxy)-3,4-dihydro-1H-quinolin-2-one

A mixture of 6-(2-chloroethoxy)-3,4-dihydro-1H-quinolin-2-one (1 eq), 2-substituted alkylamine (1.5 eq), K₂CO₃ (2 to 3 eq) and KI (1 eq) in MeCN (0.04 M to 0.05 M) was heated at reflux for 3 to 7 days under nitrogen. When required, additional 2-aryl amine was added to push reaction to completion. The mixture was cooled down to RT, water followed by DCM or EtOAc were added and the phases separated. The aqueous phase was washed with DCM or EtOAc, the organic extracts combined, filtered through a hydrophobic frit and concentrated in vacuo. The crude material was purified by either normal phase chromatography (SiO₂) using a gradient of MeOH:DCM (optionally containing 1% aq. NH₃) or by preparative HPLC-MS using a gradient of high or low pH aq. MeCN. If the resulting solid was isolated as a salt, the solid was dissolved in sat. aq. NaHCO₃, extracted with DCM and concentrated in vacuo to afford the free base. Further purification was preformed via trituration with Et₂O if required.

### Synthesis 45

### 6-[2-[methyl(3-pyridylmethyl)amino]ethoxy]-3,4-dihydro-1H-quinolin-2-one (ALDHI-3010)

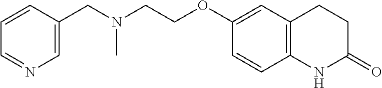 [SMILES: CN(CCOc1ccc2c(c1)CCC(=O)N2)Cc1cccnc1]

Prepared as described in method D from 6-(2-chloroethoxy)-3,4-dihydro-1H-quinolin-2-one (30 mg, 0.133 mmol, 1 eq), N-Methyl-N-(3-pyridylmethyl)amine (24 mg, 0.199 mmol, 1.5 eq), K₂CO₃ (37 mg, 0.266 mmol, 2 eq) and KI (22 mg, 0.133 mmol, 1 eq) in MeCN (2.5 mL) to return the title compound (18 mg, 0.0562 mmol, 42%) as an orange gum after purification by preparative HPLC-MS (high pH).

MS (ES+) m/z 312.2 (M+H).

¹H NMR (300 MHz, CDCl₃) δ 8.56 (dd, J=2.2, 0.8 Hz, 1H), 8.51 (dd, J=4.8, 1.7 Hz, 1H), 8.36 (s, 1H), 7.74-7.63 (m, 1H), 7.31-7.19 (m, 1H), 6.79-6.63 (m, 3H), 4.05 (t, J=5.7 Hz, 2H), 3.63 (s, 2H), 2.92 (dd, J=8.5, 6.4 Hz, 2H), 2.82 (t, J=5.7 Hz, 2H), 2.66-2.54 (m, 2H), 2.33 (s, 3H).

The following example compounds were prepared similarly using method D with the appropriate 2-substituted alkyl amine starting material.

### Synthesis 46

### 6-[2-[methyl(2-pyridylmethyl)amino]ethoxy]-3,4-dihydro-1H-quinolin-2-one (ALDHI-3012)

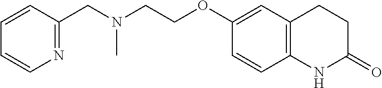 [SMILES: CN(CCOc1ccc2c(c1)CCC(=O)N2)Cc1ccccn1]

MS (ES+) m/z 312.1 (M+H).

¹H NMR (300 MHz, CDCl₃) δ 8.55 (ddd, J=4.9, 1.8, 0.9 Hz, 1H), 7.71-7.56 (m, 2H), 7.44 (dt, J=7.8, 1.1 Hz, 1H), 7.16 (ddd, J=7.5, 4.9, 1.2 Hz, 1H), 6.77-6.59 (m, 3H), 4.08 (t, J=5.9 Hz, 2H), 3.79 (s, 2H), 2.98-2.84 (m, 4H), 2.66-2.54 (m, 2H), 2.40 (s, 3H).

### Synthesis 47

### 6-[2-[methyl(4-pyridylmethyl)amino]ethoxy]-3,4-dihydro-1H-quinolin-2-one (ALDHI-3014)

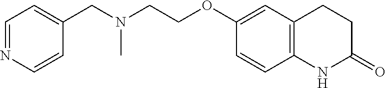 [SMILES: CN(CCOc1ccc2c(c1)CCC(=O)N2)Cc1ccncc1]

MS (ES+) m/z 312.3 (M+H).

¹H NMR (300 MHz, DMSO-d₆) δ 9.90 (s, 1H), 8.54-8.46 (m, 2H), 7.38-7.30 (m, 2H), 6.83-6.67 (m, 3H), 4.05 (t, J=5.8 Hz, 2H), 3.61 (s, 2H), 2.88-2.78 (m, 2H), 2.73 (t, J=5.8 Hz, 2H), 2.45-2.34 (m, 2H), 2.25 (s, 3H).

### Synthesis 48

### 6-[2-[(3-fluorophenyl)methyl-methyl-amino]ethoxy]-3,4-dihydro-1H-quinolin-2-one (ALDHI-3001)

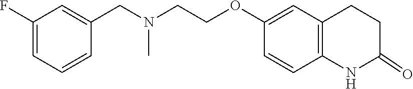 [SMILES: CN(CCOc1ccc2c(c1)CCC(=O)N2)Cc1cccc(F)c1]

MS (ES+) m/z 329.3 (MH).

¹H NMR (300 MHz, CDCl₃) δ 7.74 (s, 1H), 7.31-7.21 (m, 1H), 7.13-7.04 (m, 2H), 6.98-6.88 (m, 1H), 6.75-6.61 (m, 3H), 4.05 (t, J=5.8 Hz, 2H), 3.61 (s, 2H), 2.97-2.87 (m, 2H), 2.81 (t, J=5.8 Hz, 2H), 2.65-2.56 (m, 2H), 2.34 (s, 3H).

¹⁹F {¹H} NMR (282 MHz, CDCl₃) δ −113.73.

### Synthesis 49

### 6-[2-[methyl-[(1-methylpyrazol-4-yl)methyl]amino]ethoxy]-3,4-dihydro-1H-quinolin-2-one (ALDHI-3009)

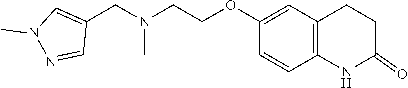 [SMILES: CN(CCOc1ccc2c(c1)CCC(=O)N2)Cc1cnn(C)c1]

MS (ES+) m/z 315.2 (M+H).

¹H NMR (300 MHz, DMSO-d₆) δ 9.90 (s, 1H), 7.56 (s, 1H), 7.33-7.27 (m, 1H), 6.82-6.66 (m, 3H), 3.99 (t, J=6.0 Hz, 2H), 3.79 (s, 3H), 3.45 (s, 2H), 2.88-2.77 (m, 2H), 2.64 (t, J=6.0 Hz, 2H), 2.45-2.34 (m, 2H), 2.20 (s, 3H).

### Synthesis 50

### 6-[2-[(3-fluorophenyl)methyl-(2-hydroxyethyl)amino]ethoxy]-3,4-dihydro-1H-quinolin-2-one (ALDHI-3002)

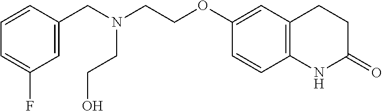 [SMILES: O=C1CCc2cc(OCCN(CCO)Cc3cccc(F)c3)ccc2N1]

MS (ES+) m/z 359.3 (M+H).

¹H NMR (300 MHz, Methanol-d₄) δ 7.39-7.25 (m, 1H), 7.24-7.13 (m, 2H), 7.05-6.91 (m, 1H), 6.87-6.68 (m, 3H), 4.08 (t, J=5.6 Hz, 2H), 3.85 (s, 2H), 3.67 (t, J=6.0 Hz, 2H), 3.04-2.86 (m, 4H), 2.81 (t, J=6.1 Hz, 2H), 2.61-2.49 (m, 2H).

¹⁹F {¹H} NMR (282 MHz, Methanol-d₄) −115.74.

### Synthesis 51

### 6-[2-[2-hydroxyethyl(3-pyridylmethyl)amino]ethoxy]-3,4-dihydro-1H-quinolin-2-one (ALDHI-3005)

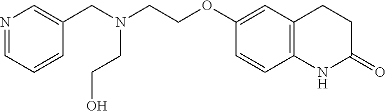 [SMILES: O=C1CCc2cc(OCCN(CCO)Cc3cccnc3)ccc2N1]

MS (ES+) m/z 342.3 (M+H).

¹H NMR (300 MHz, DMSO-d₆) δ 9.89 (s, 1H), 8.57-8.49 (m, 1H), 8.45 (dd, J=4.8, 1.7 Hz, 1H), 7.75 (dt, J=7.9, 2.1 Hz, 1H), 7.34 (ddd, J=7.7, 4.8, 0.9 Hz, 1H), 6.80-6.63 (m, 3H), 4.40 (t, J=5.4 Hz, 1H), 3.99 (t, J=6.0 Hz, 2H), 3.75 (s, 2H), 3.49 (q, J=6.1 Hz, 2H), 2.89-2.76 (m, 4H), 2.61 (t, J=6.4 Hz, 2H), 2.45-2.34 (m, 2H).

### Synthesis 52

### 6-[2-[2-hydroxyethyl-[(1R)-1-phenylethyl]amino]ethoxy]-3,4-dihydro-1H-quinolin-2-one (ALDHI-3006)

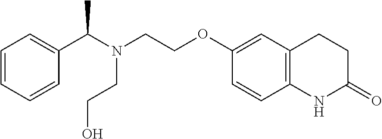 [SMILES: C[C@H](c1ccccc1)N(CCO)CCOc1ccc2c(c1)CCC(=O)N2]

MS (ES+) m/z 355.2 (M+H).

¹H NMR (300 MHz, DMSO-d₆) δ 9.89 (s, 1H), 7.44-7.26 (m, 4H), 7.29-7.17 (m, 1H), 6.78-6.60 (m, 3H), 4.29 (t, J=5.4 Hz, 1H), 4.01-3.84 (m, 3H), 3.42 (q, J=6.3 Hz, 2H), 2.91-2.54 (m, 5H), 2.44-2.34 (m, 2H), 1.32 (d, J=6.7 Hz, 3H).

### Synthesis 53

### 6-[2-[2-hydroxyethyl-[(1S)-1-phenylethyl]amino]ethoxy]-3,4-dihydro-1H-quinolin-2-one (ALDHI-3007)

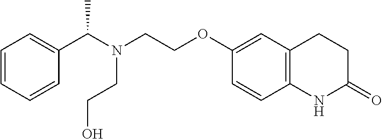 [SMILES: C[C@@H](c1ccccc1)N(CCO)CCOc1ccc2c(c1)CCC(=O)N2]

MS (ES+) m/z 355.3 (M+H).

¹H NMR (300 MHz, DMSO-d₆) δ 9.89 (s, 1H), 7.44-7.26 (m, 4H), 7.29-7.17 (m, 1H), 6.77-6.60 (m, 3H), 4.29 (t, J=5.4 Hz, 1H), 4.01-3.84 (m, 3H), 3.42 (q, J=6.4 Hz, 2H), 2.91-2.55 (m, 5H), 2.44-2.33 (m, 2H), 1.32 (d, J=6.8 Hz, 3H).

### Synthesis 54

### 6-[2-[benzyl-[2-(diethylamino)ethyl]amino]ethoxy]-3,4-dihydro-1H-quinolin-2-one (ALDHI-3004)

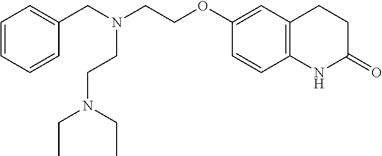 [SMILES: CCN(CC)CCN(CCOc1ccc2c(c1)CCC(=O)N2)Cc1ccccc1]

MS (ES+) m/z 396.4 (M+H).

¹H NMR (300 MHz, Methanol-d₄) δ 7.44-7.20 (m, 5H), 6.85-6.70 (m, 3H), 4.08 (t, J=5.5 Hz, 2H), 3.76 (s, 2H), 2.99-2.87 (m, 4H), 2.81-2.70 (m, 4H), 2.64 (q, J=7.2 Hz, 4H), 2.58-2.49 (m, 2H), 1.04 (t, J=7.2 Hz, 6H).

### Synthesis 55

### 6-[2-[[1-(3-fluorophenyl)-2-hydroxy-ethyl]-methyl-amino]ethoxy]-3,4-dihydro-1H-quinolin-2-one (ALDHI-3003)

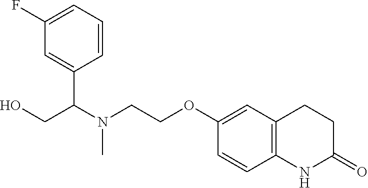 [SMILES: CN(CCOc1ccc2c(c1)CCC(=O)N2)C(CO)c1cccc(F)c1]

MS (ES+) m/z 359.3 (M+H).

¹H NMR (300 MHz, DMSO-d₆) δ 9.89 (s, 1H), 7.43-7.29 (m, 1H), 7.23-7.12 (m, 2H), 7.12-7.00 (m, 1H), 6.80-6.64 (m, 3H), 4.51 (t, J=5.1 Hz, 1H), 3.97 (t, J=6.0 Hz, 2H), 3.85-3.58 (m, 3H), 2.88-2.72 (m, 3H), 2.72-2.58 (m, 1H), 2.45-2.34 (m, 2H), 2.25 (s, 3H).

¹⁹F {¹H} NMR (282 MHz, DMSO-d₆) δ −114.00.

### Synthesis 56

### 6-[2-[2-hydroxyethyl-[(1-methylpyrazol-4-yl)methyl]amino]ethoxy]-3,4-dihydro-1H-quinolin-2-one

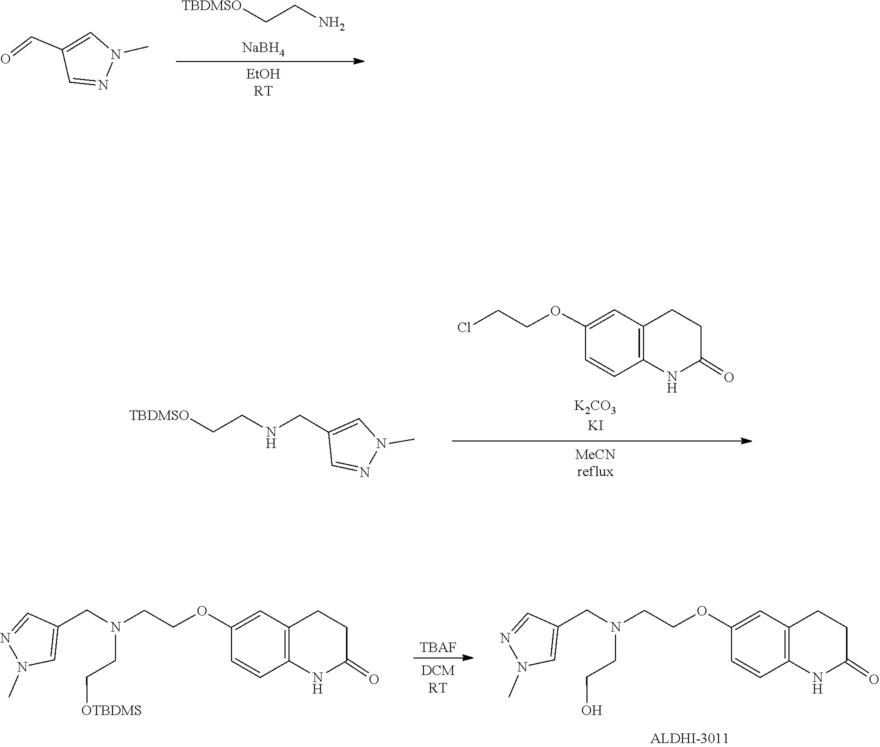

### Step 1: 2-[tert-butyl(dimethyl)silyl]oxy-N-[(1-methylpyrazol-4-yl)methyl]ethanamine

To a solution of 4-formyl-1-methyl-1H-pyrazole (100 mg, 0.908 mmol, 1 eq) in THF (2.5 mL) and ethanol (2.5 mL) was added 2-(tert-butyldimethylsilyloxy)ethanamine (0.20 mL, 1.14 mmol, 1.25 eq). The mixture was stirred at RT for 16 h then NaBH₄ (34 mg, 0.908 mmol, 1 eq) was added and the stirring continued for 3 h. Additional NaBH₄ (34 mg, 0.908 mmol, 1 eq) was added and the mixture was stirred for a further 18 h then quenched with sat. aq. NH₄Cl (5 mL) and extracted with EtOAc (2×10 mL). The combined extracts were washed with brine (10 mL), filtered through a hydrophobic frit and concentrated in vacuo. The resulting residue was chromatographed (SiO₂) using 0-5% MeOH:DCM (+1% aq. NH₃) as eluent to afford 2-[tert-butyl(dimethyl)silyl]oxy-N-[(1-methylpyrazol-4-yl)methyl]ethanamine (192 mg, 0.713 mmol, 78%) as a colourless oil (ca. 10% w/w 2-(tert-butyldimethylsilyloxy)ethanamine). ¹H NMR (300 MHz, Methanol-d₄) δ 7.56 (s, 1H), 7.45 (s, 1H), 3.88 (s, 3H), 3.78 (t, 2H), 3.71 (s, 2H), 2.74 (t, 2H), 0.92 (s, 9H), 0.10 (s, 6H).

### Step 2: 6-[2-[2-[tert-butyl(dimethyl)silyl]oxyethyl-[(1-methylpyrazol-4-yl)methyl]amino]ethoxy]-3,4-dihydro-1H-quinolin-2-one

Prepared as described in method D from 6-(2-chloroethoxy)-3,4-dihydro-1H-quinolin-2-one (50 mg, 0.222 mmol, 1 eq), 2-[tert-butyl(dimethyl)silyl]oxy-N-[(1-methylpyrazol-4-yl)methyl]ethanamine (90 mg, 0.332 mmol, 1.5 eq), K₂CO₃ (62 mg, 0.443 mmol, 2 eq) and KI (37 mg, 0.222 mmol, 1 eq) in MeCN (2.5 mL) to return 6-[2-[2-[tert-butyl(dimethyl)silyl]oxyethyl-[(1-methylpyrazol-4-yl)methyl]amino]ethoxy]-3,4-dihydro-1H-quinolin-2-one (66 mg, 0.144 mmol, 65%) as a colourless residue after normal phase chromatography (SiO₂) using 0-5% MeOH:DCM (+1% aq. NH₃) as eluent. Used directly in the next step. MS (ES+) m/z 459.3 (M+H).

### Step 3: 6-[2-[2-hydroxyethyl-[(1-methylpyrazol-4-yl)methyl]amino]ethoxy]-3,4-dihydro-1H-quinolin-2-on

To a solution of 6-[2-[2-[tert-butyl(dimethyl)silyl]oxyethyl-[(1-methylpyrazol-4-yl)methyl]amino]ethoxy]-3,4-dihydro-1H-quinolin-2-one (65 mg, 0.142 mmol, 1 eq) in THF (5 mL) was added TBAF (1M in THF, 0.15 mL, 0.149 mmol, 1.05 eq). The mixture was stirred at RT for 1 h then quenched with sat. aq. NaHCO₃ (10 mL) and extracted with DCM (2×10 mL). The combined extracts were washed with brine (20 mL), filtered through a hydrophobic frit and the solvent removed under reduced pressure. The resulting residue was chromatographed (SiO₂) using 0-5% MeOH:DCM as eluent then purified by preparative HPLC (high pH) to afford the title compound (7.9 mg, 0.0229 mmol, 16%) as a colourless gum.

MS (ES+) m/z 345.1 (M+H).

¹H NMR (300 MHz, DMSO-d₆) δ 9.90 (s, 1H), 7.57 (d, J=0.8 Hz, 1H), 7.31 (d, J=0.8 Hz, 1H), 6.81-6.65 (m, 3H), 4.32 (t, J=5.4 Hz, 1H), 3.97 (t, J=6.1 Hz, 2H), 3.79 (s, 3H), 3.57 (s, 2H), 3.48 (q, J=6.4, 5.3 Hz, 2H), 2.83 (t, J=7.5 Hz, 2H), 2.74 (t, J=6.1 Hz, 2H), 2.57-2.50 (m, 2H), 2.46-2.34 (m, 2H).

### Scheme 5

### Preparation of alkyl-aryl ethers by Mitsunobu Coupling

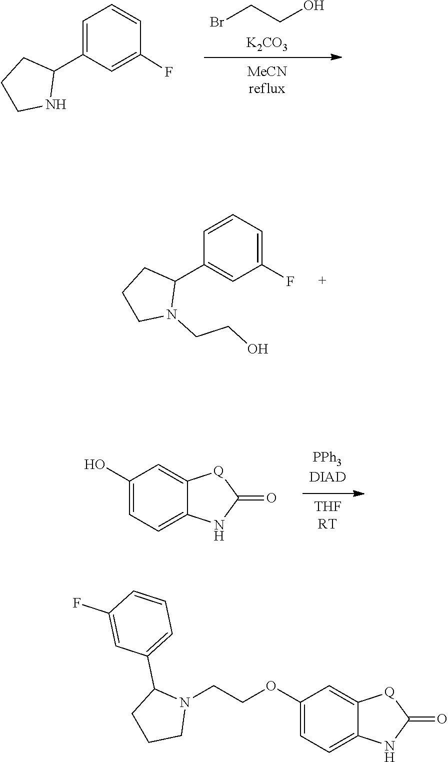

### Synthesis 57

### 2-[2-(3-fluorophenyl)pyrrolidin-1-yl]ethanol

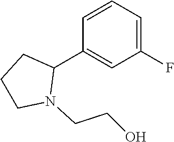 [SMILES: OCCN1CCCC1c1cccc(F)c1]

A mixture of 2-(3-fluorophenyl)pyrrolidine (2.4 mL, 15.7 mmol, 1 eq), 2-bromoethanol (2.2 mL, 31.4 mmol, 2 eq.) and K₂CO₃ (4.34 g, 31.5 mmol, 2 eq) in MeCN (20 mL) was refluxed for 18 h. The mixture was cooled, partitioned between EtOAc (20 mL) and water (20 mL), the phases separated and the aqueous phase washed with EtOAc (20 mL). The combined extracts were washed with brine (50 mL), filtered through a hydrophobic frit and concentrated in vacuo. The residue was chromatographed (SiO₂) using 0-5% MeOH:DCM (+1% aq. NH₃) as eluent to afford 2-[2-(3-fluorophenyl)pyrrolidin-1-yl]ethanol (2.1 g, 10.2 mmol, 65%) as a yellow oil.

MS (ES+) m/z 210.2 (M+H).

¹H NMR (300 MHz, DMSO-d₆) δ 7.41-7.28 (m, 1H), 7.23-7.11 (m, 2H), 7.09-6.97 (m, 1H), 4.43-4.33 (m, 1H), 3.51-3.22 (m, 4H), 2.61-2.51 (m, 1H), 2.24 (q, J=8.8 Hz, 1H), 2.20-2.03 (m, 2H), 1.92-1.66 (m, 2H), 1.57-1.39 (m, 1H).

### General Method E

### Mitsunobu Coupling Between Substituted Phenols and 2-[2-(3-fluorophenyl)pyrrolidin-1-yl]ethanol

To a suspension of 2-[2-(3-fluorophenyl)pyrrolidin-1-yl]ethanol (1 to 1.05 eq) and triphenylphosphine (1.5 eq) in THF (0.05 to 0.1 M) was added DIAD (1.5 eq) then the appropriately substituted phenol (1 to 1.1 eq). The mixture was stirred at RT for 18 h under nitrogen. The mixture was diluted with water, extracted with DCM or EtOAc and the phases separated. The aqueous phase was washed with DCM or EtOAc, the organic extracts combined, washed with brine, filtered through a hydrophobic frit and concentrated in vacuo. The crude material was purified first by normal phase chromatography (SiO₂) using a gradient of MeOH:DCM (optionally containing 1% aq. NH₃) then by reverse phase chromatography (C₁₈) using a gradient of MeCN:H₂O or by preparative HPLC-MS using a gradient of high or low pH aq. MeCN.

### Synthesis 58

### 7-[2-[2-(3-fluorophenyl)pyrrolidin-1-yl]ethoxy]-1,3,4,5-tetrahydro-1-benzazepin-2-one (ALDHI-2020)

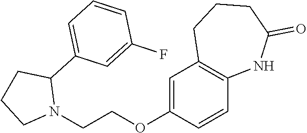 [SMILES: O=C1CCCc2cc(OCCN3CCCC3c3cccc(F)c3)ccc2N1]

Prepared as described in method E using 2-[2-(3-fluorophenyl)pyrrolidin-1-yl]ethanol (100 mg, 0.480 mmol), triphenylphosphine (188 mg, 0.720 mmol), DIAD (0.14 mL, 0.720 mmol) and 7-hydroxy-2,3,4,5-tetrahydro-1H-1-benzazepin-2-one (93.2 mg, 0.530 mmol) to return the title compound (16 mg, 0.0434 mmol, 9%) as a white solid powder after normal phase chromatography (SiO₂) using 0-70% EtOAc:petroleum ether as eluent and subsequent reverse phase chromatography (C₁₈) using 5-95% MeCN:H₂O as eluent.

MS (ES+) m/z 369.3 (M+H).

¹H NMR (300 MHz, DMSO-d₆) δ 9.27 (s, 1H), 7.41-7.28 (m, 1H), 7.25-7.14 (m, 2H), 7.10-6.97 (m, 1H), 6.89-6.68 (m, 3H), 3.98 (t, J=5.9 Hz, 2H), 3.53-3.34 (m, 2H), 2.87-2.75 (m, 1H), 2.67-2.57 (m, 2H), 2.47-2.30 (m, 2H), 2.24-2.01 (m, 5H), 1.91-1.73 (m, 2H), 1.60-1.42 (m, 1H).

The following example compounds were prepared similarly using method E with the appropriate phenol.

### Synthesis 59

### 6-[2-[2-(3-fluorophenyl)pyrrolidin-1-yl]ethoxy]-3,4-dihydro-1H-quinolin-2-one (ALDHI-2001)

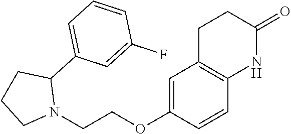 [SMILES: O=C1CCc2cc(OCCN3CCCC3c3cccc(F)c3)ccc2N1]

MS (ES+) m/z 355.3 (M+H).

¹H NMR (300 MHz, CDCl₃) δ 7.59 (s, 1H), 7.30-7.20 (m, 1H), 7.18-7.07 (m, 2H), 6.91 (td, J=8.6, 2.8 Hz, 1H), 6.70-6.56 (m, 3H), 3.96 (t, J=6.0 Hz, 2H), 3.50-3.34 (m, 2H), 3.01-2.85 (m, 3H), 2.65-2.49 (m, 3H), 2.43 (q, J=8.8 Hz, 1H), 2.25-2.09 (m, 1H), 2.05-1.54 (m, 3H).

¹⁹F {¹H} NMR (282 MHz, CDCl₃) δ −113.45.

### Synthesis 60

### 7-[2-[2-(3-fluorophenyl)pyrrolidin-1-yl]ethoxy]-4H-1,4-benzoxazin-3-one (ALDHI-2009)

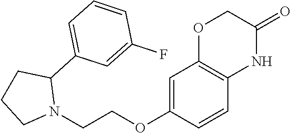 [SMILES: O=C1COc2cc(OCCN3CCCC3c3cccc(F)c3)ccc2N1]

MS (ES+) m/z 357.3 (M+H).

¹H NMR (300 MHz, DMSO-d₆) δ 10.51 (s, 1H), 7.41-7.27 (m, 1H), 7.24-7.14 (m, 2H), 7.04 (dddd, J=9.1, 8.2, 2.4, 1.4 Hz, 1H), 6.82-6.71 (m, 1H), 6.54-6.43 (m, 2H), 4.51 (s, 2H), 3.93 (t, J=5.8 Hz, 2H), 3.43 (t, J=8.1 Hz, 1H), 3.38-3.29 (m, 1H), 2.78 (dt, J=12.6, 6.2 Hz, 1H), 2.50-2.40 (m, 1H), 2.35 (q, J=8.8 Hz, 1H), 2.23-2.06 (m, 1H), 1.90-1.69 (m, 2H), 1.60-1.41 (m, 1H).

### Synthesis 61

### 7-[2-[2-(3-fluorophenyl)pyrrolidin-1-yl]ethoxy]-4H-1,4-benzothiazin-3-one (ALDHI-2008)

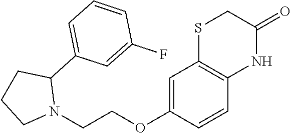 [SMILES: O=C1CSc2cc(OCCN3CCCC3c3cccc(F)c3)ccc2N1]

MS (ES+) m/z 373.3 (M+H).

¹H NMR (300 MHz, DMSO-d₆) δ 10.36 (s, 1H), 7.41-7.28 (m, 1H), 7.24-7.13 (m, 2H), 7.10-6.97 (m, 1H), 6.91-6.80 (m, 2H), 6.71 (dd, J=8.7, 2.8 Hz, 1H), 3.96 (t, J=5.8 Hz, 2H), 3.49-3.38 (m, 3H), 3.38-3.27 (m, 1H), 2.86-2.71 (m, 1H), 2.49-2.41 (m, 1H), 2.35 (q, J=8.8 Hz, 1H), 2.23-2.06 (m, 1H), 1.90-1.72 (m, 2H), 1.60-1.41 (m, 1H).

### Scheme 6

### Preparation of alkyl-aryl ethers by Pd Catalysed Coupling with Aryl Halides Palladium Catalysed Coupling of Aryl Bromides with Primary Alcohols

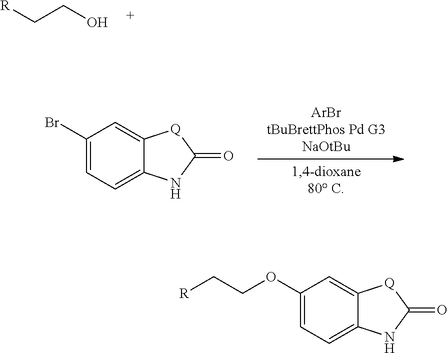

### Synthesis of aryl bromides

### Synthesis 62

### 6-bromo-3-methyl-3,4-dihydro-1H-quinolin-2-one

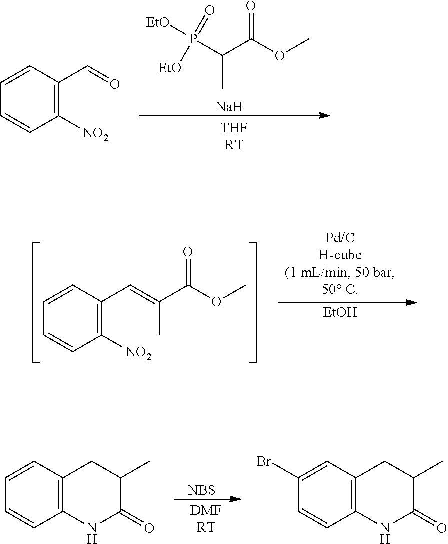

### Step 1: 3-methyl-3,4-dihydro-1H-quinolin-2-one

To a solution of methyl 2-diethoxyphosphorylpropanoate (408 mg, 1.82 mmol, 1.1 eq) in THF (10 mL) was added sodium hydride (60% in mineral oil, 79 mg, 1.99 mmol, 1.2 eq) at 0° C. The mixture was stirred for 0.5 h then a solution of 2-nitrobenzaldehyde (250 mg, 1.65 mmol, 1 eq) in THF (10 mL) was added and stirring continued at RT overnight. Water (5 mL) was added dropwise followed by sat. aq. NaHCO₃ (10 mL). The mixture was extracted with EtOAc (3×25 mL). The organic phase was separated, washed with brine (50 mL), dried (MgSO₄), filtered and concentrated in vacuo. The residue was chromatographed (SiO₂) using 0-20% EtOAc:petroleum ether to afford a yellow oil. The oil was dissolved in EtOH (26 mL) and passed through a 10% Pd/C H-Cube cartridge (1 mL/min, 50° C., 50 bar). The solvent was removed in vacuo to afford 3-methyl-3,4-dihydro-1H-quinolin-2-one (194 mg, 1.20 mmol, 92%) as a colourless oil which solidified on standing to give an off-white powder. Contains ca. 10% un-reduced product by ¹H NMR. Used directly in the next step. MS (ES+) m/z 162.0 (M+H). ¹H NMR (DMSO-d₆) δ: 10.03 (s, 1H), 7.22-7.07 (m, 2H), 6.96-6.67 (m, 2H), 2.93 (dd, J=15.3, 5.7 Hz, 1H), 2.71-2.55 (m, 1H), 1.12 (d, J=6.8 Hz, 3H).

### Step 2: 6-bromo-3-methyl-3,4-dihydro-1H-quinolin-2-one

To a solution of 3-methyl-3,4-dihydro-1H-quinolin-2-one (194 mg, 1.20 mmol, 1 eq) in DMF (5 mL) cooled to 0° C. was added NBS (214 mg, 1.20 mmol, 1 eq) added portion-wise. The mixture was warmed to RT and stirred for 2 h then poured into water and extracted with EtOAc (2×25 mL). The organic phase was washed with aq. Na₂S₂O₃ (25 mL), brine (25 mL), dried (MgSO₄), filtered and concentrated in vacuo. The residue was chromatographed (SiO₂) using 0-50% EtOAc:petroleum ether to afford the title compound (200 mg, 0.833 mmol, 69%) as a white powder.

MS (ES+) m/z 240.0/242.0 (M+H), Br isotope pattern.

¹H NMR (300 MHz, DMSO-d₆) δ 10.16 (s, 1H), 7.37 (d, J=2.3 Hz, 1H), 7.31 (dd, J=8.4, 2.3 Hz, 1H), 6.80 (d, J=8.4 Hz, 1H), 2.95 (dd, J=15.6, 5.8 Hz, 1H), 2.71-2.58 (m, 1H), 2.57-2.42 (m, 1H), 1.11 (d, J=6.8 Hz, 3H).

### General Method F

### Cyclisation of 2-amino-5-bromophenol with Substituted methyl 2-bromoacetates

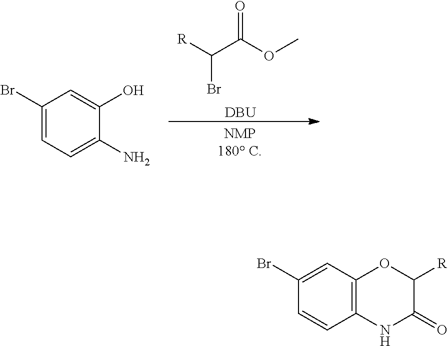

A mixture of 2-amino-5-bromophenol (1.1 to 1.2 eq), DBU (1.1 to 1.2 eq), substituted methyl 2-bromoacetate (1 eq) and NMP (0.22 to 0.51 M) was heated at 180° C. for 3 min in a microwave reactor. The reaction was partitioned between EtOAc (25 mL) and water (25 mL). The organic phase was separated, washed with water (25 mL), brine (3×25 mL), dried (MgSO₄), filtered and concentrated in vacuo. The crude material was purified by normal phase chromatography (SiO₂) using a gradient of EtOAc:petroleum ether. When necessary, the material was triturated with water for further purification.

### Synthesis 63

### 7-bromo-2-ethyl-4H-1,4-benzoxazin-3-one

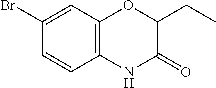

Prepared as described in method F from 2-amino-5-bromophenol (249 mg, 1.33 mmol, 1.2 eq), DBU (0.18 mL, 1.22 mmol, 1.1 eq), methyl 2-bromobutyrate (0.13 mL, 1.10 mmol, 1 eq) in NMP (4 mL) to return the title compound (235 mg, 0.918 mmol, 83%) as an orange solid after normal phase chromatography (SiO₂) using 0-40% EtOAc:petroleum ether as eluent. MS (ES−) m/z 254.0/256.0 (M−H), Br isotope pattern. ¹H NMR (300 MHz, DMSO-d₆) δ 10.77 (s, 1H), 7.19 (d, J=2.1 Hz, 1H), 7.13 (dd, J=8.3, 2.1 Hz, 1H), 6.83 (d, J=8.4 Hz, 1H), 4.56 (dd, J=7.6, 4.7 Hz, 1H), 1.93-1.64 (m, 2H), 0.98 (t, J=7.4 Hz, 3H).

The following intermediate compounds were prepared similarly using method F, using the appropriately substituted methyl 2-bromoacetate.

### Synthesis 63

### 7-bromo-2-propyl-4H-1,4-benzoxazin-3-one

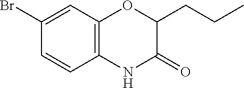

MS (ES+) 270.0/272.0 (M+H), Br isotope pattern.

¹H NMR (300 MHz, DMSO-d₆) δ 10.77 (s, 1H), 7.18 (d, J=2.1 Hz, 1H), 7.13 (dd, J=8.3, 2.1 Hz, 1H), 6.83 (d, J=8.3 Hz, 1H), 4.66-4.56 (m, 1H), 1.85-1.61 (m, 2H), 1.61-1.34 (m, 2H), 0.91 (t, J=7.3 Hz, 3H).

### Synthesis 64

### 7-bromo-2,2-dimethyl-4H-1,4-benzoxazin-3-one

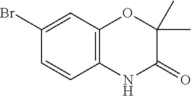

MS (ES+) 254/256 (M−H), Br isotope pattern.

¹H NMR (DMSO-d₆) δ: 10.74 (s, 1H), 7.18-7.10 (m, 2H), 6.88-6.81 (m, 1H), 1.40 (s, 6H).

### Synthesis 65

### 7-bromo-2-isopropyl-4H-1,4-benzoxazin-3-one

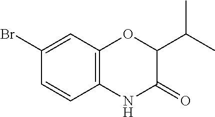

To a solution of 2-amino-5-bromophenol (500 mg, 2.66 mmol, 1 eq) in THF (10 mL) at 0° C. was added NaHCO₃ (670 mg, 7.98 mmol, 3 eq) and the mixture was stirred for 10 min. A solution of 2-bromo-3-methylbutanoyl chloride (530 mg, 2.66 mmol, 1 eq) in THF (1 mL) was added dropwise then stirred for 6 h. The reaction mixture was diluted with water and extracted with ethyl acetate (3×20 mL). The organic extract was washed with brine, dried (MgSO₄), filtered and concentrated in vacuo. The crude amide was dissolved in DMF (5 mL), K₂CO₃ (551 mg, 3.99 mmol, 1.5 eq) added and stirred at RT for 16 h. The reaction mixture was diluted with water (50 mL) and extracted with ethyl acetate (3×20 mL). The organic extract was washed with brine (3×50 mL), dried (MgSO₄), filtered and concentrated in vacuo. The residue was chromatographed (SiO₂) using (0-30% EtOAc:petroleum ether as eluent to afford the title compound (569 mg, 2.11 mmol, 79%) as a yellow powder.

MS (ES+) m/z 268.0/270.0 (M+H), Br isotope pattern.

¹H NMR (300 MHz, DMSO-d₆) δ 10.79 (s, 1H), 7.18 (d, J=2.2 Hz, 1H), 7.11 (dd, J=8.3, 2.1 Hz, 1H), 6.81 (d, J=8.4 Hz, 1H), 4.40 (d, J=5.5 Hz, 1H), 2.15 (pd, J=6.8, 5.5 Hz, 1H), 1.01 (d, J=6.9 Hz, 3H), 0.93 (d, J=6.8 Hz, 3H).

### General Method G

### Preparation of alkyl-aryl ethers by Pd-Catalysed Coupling with aryl halides

A mixture of a primary alcohol (1 to 2 eq), aryl bromide (1 eq), tBuBrettPhos Pd G3 (0.02 to 0.1 eq) and NaOtBu (1 to 2 eq) in 1,4-dioxane (0.1 to 0.2 M) was purged with nitrogen (×3) then heated to 80° C. for 16-36 h. When required, additional tBuBrettPhos Pd G3 (0.05 eq) followed by the primary alcohol (0.2 eq) was added to push reaction to completion. The mixture was cooled, filtered through a pad of celite and washed with EtOAc. The organic phases were washed with water, brine, filtered through a hydrophobic frit or dried over MgSO₄ and concentrated in vacuo. The crude material was purified by either normal phase chromatography (SiO₂) using a gradient of EtOAc:petroleum ether and/or reverse phase chromatography (C₁₈) using a gradient of MeCN:H₂O and/or by preparative HPLC-MS using a gradient of high or low pH aq. MeCN.

### Synthesis 66

### 6-[2-[2-(3-fluorophenyl)pyrrolidin-1-yl]ethoxy]-3-methyl-3,4-dihydro-1H-quinolin-2-one (ALDHI-2048)

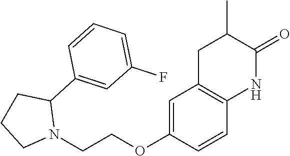 [SMILES: CC1Cc2cc(OCCN3CCCC3c3cccc(F)c3)ccc2NC1=O]

Prepared as described in method G from 2-[2-(3-fluorophenyl)pyrrolidin-1-yl]ethanol (0.25 g, 1.20 mmol, 2 eq), 6-bromo-3-methyl-3,4-dihydro-1H-quinolin-2-one (100 mg, 0.416 mmol, 1 eq), tBuBrettPhos Pd G3 (36 mg, 0.0416 mmol, 0.100 eq) and NaOtBu (48 mg, 0.500 mmol, 1.2 eq) in 1,4-dioxane (2.9 mL) to return the title compound (8 mg, 0.0217 mmol, 5.2%) as a white powder after reverse phase chromatography (C₁₈) using 5-95% MeCN:H₂O as eluent.

MS (ES+) m/z 369.2 (M+H).

¹H NMR (300 MHz, DMSO-d₆) δ 9.86 (s, 1H), 7.41-7.28 (m, 1H), 7.25-7.14 (m, 2H), 7.10-6.97 (m, 1H), 6.77-6.62 (m, 3H), 3.94 (t, J=5.9 Hz, 2H), 3.43 (t, J=8.1 Hz, 1H), 3.35 (ddd, J=9.1, 7.2, 3.3 Hz, 1H), 2.93-2.72 (m, 2H), 2.65-2.53 (m, 1H), 2.52-2.40 (m, 2H), 2.40-2.29 (m, 1H), 2.24-2.06 (m, 1H), 1.90-1.72 (m, 2H), 1.60-1.42 (m, 1H), 1.10 (d, J=6.8 Hz, 3H).

The following example compounds were prepared similarly using method G with the appropriately substituted aryl bromide.

### Synthesis 67

### 7-[2-[2-(3-fluorophenyl)pyrrolidin-1-yl]ethoxy]-2-methyl-4H-1,4-benzoxazin-3-one (ALDHI-2044)

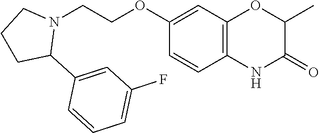 [SMILES: CC1Oc2cc(OCCN3CCCC3c3cccc(F)c3)ccc2NC1=O]

MS (ES+) m/z 371.2 (M+H).

¹H NMR (300 MHz, DMSO-d₆) δ 10.45 (s, 1H), 7.40-7.27 (m, 1H), 7.24-7.14 (m, 2H), 7.10-6.97 (m, 1H), 6.76 (d, J=8.3 Hz, 1H), 6.54-6.43 (m, 2H), 4.60 (q, J=6.8 Hz, 1H), 3.93 (t, J=5.8 Hz, 2H), 3.43 (t, J=8.1 Hz, 1H), 3.39-3.28 (m, 1H), 2.85-2.71 (m, 1H), 2.49-2.40 (m, 1H), 2.35 (q, J=8.8 Hz, 1H), 2.23-2.06 (m, 1H), 1.90-1.72 (m, 2H), 1.60-1.43 (m, 1H), 1.39 (d, J=6.8 Hz, 3H).

### Synthesis 68

### 2-ethyl-7-[2-[2-(3-fluorophenyl)pyrrolidin-1-yl]ethoxy]-4H-1,4-benzoxazin-3-one (ALDHI-2045)

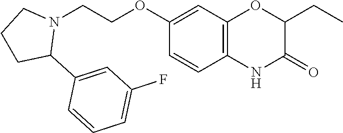 [SMILES: CCC1Oc2cc(OCCN3CCCC3c3cccc(F)c3)ccc2NC1=O]

MS (ES+) m/z 385.2 (M+H).

¹H NMR (300 MHz, DMSO-d₆) δ 10.45 (s, 1H), 7.40-7.27 (m, 1H), 7.24-7.14 (m, 2H), 7.09-6.96 (m, 1H), 6.75 (d, J=8.5 Hz, 1H), 6.55-6.42 (m, 2H), 4.44 (ddd, J=7.8, 4.6, 0.9 Hz, 1H), 3.93 (t, J=5.8 Hz, 2H), 3.43 (t, J=8.1 Hz, 1H), 3.39-3.29 (m, 1H), 2.86-2.71 (m, 1H), 2.49-2.41 (m, 1H), 2.35 (q, J=8.7 Hz, 1H), 2.23-2.06 (m, 1H), 1.90-1.61 (m, 4H), 1.59-1.41 (m, 1H), 0.97 (t, J=7.3 Hz, 3H).

### Synthesis 69

### 7-[2-[2-(3-fluorophenyl)pyrrolidin-1-yl]ethoxy]-2-propyl-4H-1,4-benzoxazin-3-one (ALDHI-2047)

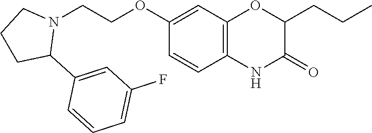 [SMILES: CCCC1Oc2cc(OCCN3CCCC3c3cccc(F)c3)ccc2NC1=O]

MS (ES+) m/z 399.2 (M+H)

¹H NMR (300 MHz, CDCl₃) δ 7.97 (s, 1H), 7.29-7.20 (m, 1H), 7.17-7.09 (m, 2H), 6.91 (tdd, J=8.3, 2.7, 1.1 Hz, 1H), 6.64 (d, J=8.6 Hz, 1H), 6.50 (d, J=2.6 Hz, 1H), 6.43 (ddd, J=8.6, 2.6, 1.0 Hz, 1H), 4.57-4.50 (m, 1H), 3.93 (t, J=6.0 Hz, 2H), 3.49-3.33 (m, 2H), 2.93 (dt, J=12.5, 6.1 Hz, 1H), 2.55 (dt, J=12.4, 5.9 Hz, 1H), 2.41 (q, J=8.8 Hz, 1H), 2.26-2.08 (m, 1H), 2.05-1.75 (m, 4H), 1.72-1.42 (m, 3H), 0.97 (t, J=7.3 Hz, 3H).

### Synthesis 70

### 7-[2-[2-(3-fluorophenyl)pyrrolidin-1-yl]ethoxy]-2,2-dimethyl-4H-1,4-benzoxazin-3-one (ALDHI-2043)

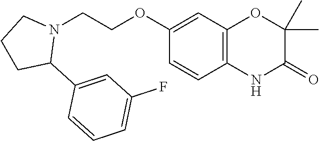 [SMILES: CC1(C)Oc2cc(OCCN3CCCC3c3cccc(F)c3)ccc2NC1=O]

MS (ES+) m/z 385.2 (M+H).

¹H NMR (300 MHz, DMSO-d₆) δ 10.41 (s, 1H), 7.40-7.27 (m, 1H), 7.24-7.14 (m, 2H), 7.09-6.96 (m, 1H), 6.80-6.70 (m, 1H), 6.52-6.42 (m, 2H), 3.93 (t, J=5.8 Hz, 2H), 3.43 (t, J=8.1 Hz, 1H), 3.39-3.31 (m, 1H), 2.86-2.73 (m, 1H), 2.48-2.40 (m, 1H), 2.35 (q, J=8.8 Hz, 1H), 2.23-2.06 (m, 1H), 1.90-1.72 (m, 2H), 1.59-1.41 (m, 1H), 1.37 (s, 6H).

### Synthesis 71

### 7-[2-[2-(3-fluorophenyl)pyrrolidin-1-yl]ethoxy]-2-isopropyl-4H-1,4-benzoxazin-3-one (ALDHI-2049)

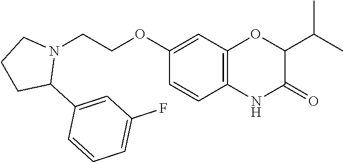 [SMILES: CC(C)C1Oc2cc(OCCN3CCCC3c3cccc(F)c3)ccc2NC1=O]

MS (ES+) m/z 399.2 (M+H).

¹H NMR (300 MHz, DMSO-d₆) δ 10.47 (s, 1H), 7.40-7.27 (m, 1H), 7.25-7.14 (m, 2H), 7.09-6.96 (m, 1H), 6.73 (d, J=8.5 Hz, 1H), 6.54-6.39 (m, 2H), 4.27 (dd, J=5.7, 1.7 Hz, 1H), 3.93 (t, J=5.8 Hz, 2H), 3.43 (t, J=8.1 Hz, 1H), 3.35 (ddd, J=9.1, 7.2, 3.4 Hz, 1H), 2.86-2.71 (m, 1H), 2.49-2.40 (m, 1H), 2.35 (q, J=8.8 Hz, 1H), 2.22-2.04 (m, 2H), 1.90-1.72 (m, 2H), 1.59-1.41 (m, 1H), 1.01 (d, J=6.9 Hz, 3H), 0.92 (dd, J=6.7, 1.7 Hz, 3H).

### Synthesis 72

### 7-fluoro-6-[2-[2-(3-fluorophenyl)pyrrolidin-1-yl]ethoxy]-3,4-dihydro-1H-quinolin-2-one (ALDHI-2050)

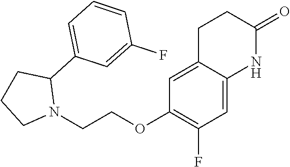 [SMILES: O=C1CCc2cc(OCCN3CCCC3c3cccc(F)c3)c(F)cc2N1]

MS (ES+) m/z 373.2 (M+H)

¹H NMR (300 MHz, DMSO-d₆) δ 9.93 (s, 1H), 7.40-7.27 (m, 1H), 7.24-7.13 (m, 2H), 7.10-6.98 (m, 1H), 6.95 (d, J=9.0 Hz, 1H), 6.67 (d, J=12.2 Hz, 1H), 4.10-3.93 (m, 2H), 3.44 (t, J=8.1 Hz, 1H), 3.41-3.27 (m, 1H), 2.89-2.74 (m, 3H), 2.49-2.30 (m, 4H), 2.24-2.06 (m, 1H), 1.93-1.69 (m, 2H), 1.60-1.41 (m, 1H).

### Synthesis 73

### 6-[2-(2-oxo-5-phenyl-pyrrolidin-1-yl)ethoxy]-3,4-dihydro-1H-quinolin-2-one (ALDHI-2051)

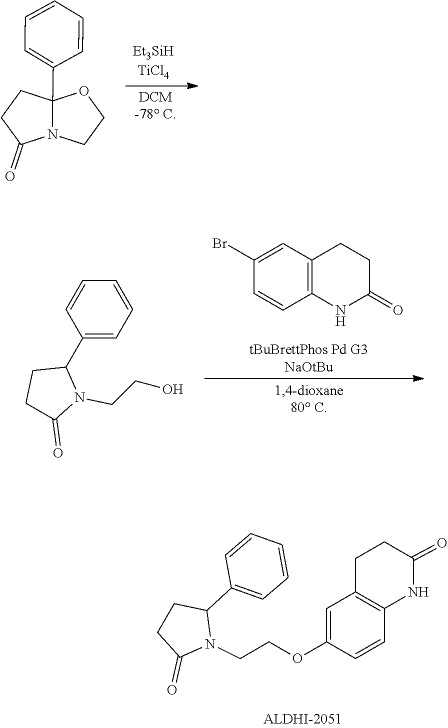 [SMILES: O=C1CCC2(c3ccccc3)OCCN12 ; O=C1CCC(c2ccccc2)N1CCO ; O=C1CCc2cc(Br)ccc2N1 ; O=C1CCc2cc(OCCN3C(=O)CCC3c3ccccc3)ccc2N1]

### Step 1: 1-(2-hydroxyethyl)-5-phenyl-pyrrolidin-2-one

To a solution of 7a-phenyl-2,3,6,7-tetrahydropyrrolo[2,1-b]oxazol-5-one (758 mg, 3.73 mmol, 1 eq) (prepared as reported in Trapani et al., Journal of Pharmacy and Pharmacology, 1996, Vol., 48, pp. 834-840), and triethylsilane (1.8 mL, 11.2 mmol, 3 eq) in DCM (40 mL) under nitrogen at −78° C. was added titanium (IV) tetrachloride (1M in toluene, 7.5 mL, 7.46 mmol, 2 eq). The mixture was allowed to warm to RT overnight, then was cooled to 0° C. Sat. aq. NH₄Cl (25 mL) and water (25 mL) were added and the phases separated. The organic phase was washed with brine (50 mL), filtered through a hydrophobic frit and concentrated in vacuo. The resulting solid was chromatographed (SiO₂) using 0-10% MeOH:DCM as eluent to afford 1-(2-hydroxyethyl)-5-phenyl-pyrrolidin-2-one (578 mg, 2.82 mmol, 76%) as an oil which solidified to a white solid.

MS (ES+) m/z 206.1 (M+H).

¹H NMR (300 MHz, DMSO-d₆) δ 7.44-7.28 (m, 3H), 7.28-7.20 (m, 2H), 4.82-4.72 (m, 1H), 4.65 (t, J=5.6 Hz, 1H), 3.59-3.46 (m, 1H), 3.46-3.23 (m, 2H), 2.59-2.23 (m, 4H), 1.83-1.67 (m, 1H).

### Step 2: 6-[2-(2-oxo-5-phenyl-pyrrolidin-1-yl)ethoxy]-3,4-dihydro-1H-quinolin-2-one

Prepared as described in method G from 1-(2-hydroxyethyl)-5-phenyl-pyrrolidin-2-one (200 mg, 0.974 mmol, 1 eq), 6-bromo-1,2,3,4-tetrahydro-2-quinolinone (220 mg, 0.974 mmol, 1 eq), tBuBrettPhos Pd G3 (83 mg, 0.0974 mmol, 0.1 eq) and NaOtBu (187 mg, 1.95 mmol, 2 eq) in 1,4-dioxane (10 mL) to return the title compound (40 mg, 0.114 mmol, 12%) as a white powder after reverse phase chromatography (C₁₈) using 5-95% MeCN:H₂O as eluent and subsequent purification by preparative HPLC-MS (high pH). Additional tBuBrettPhos Pd G3 (42 mg, 0.0487 mmol, 0.05 eq) followed by 1-(2-hydroxyethyl)-5-phenyl-pyrrolidin-2-one (40 mg, 0.195 mmol, 0.2 eq) were added after 16 h and stirred at 80° C. for a further 16 h to push to completion.

MS (ES+) mz 351.3 (M+H).

¹H NMR (300 MHz, DMSO-d₆) δ 9.90 (s, 1H), 7.46-7.24 (m, 5H), 6.78-6.61 (m, 3H), 4.85-4.75 (m, 1H), 3.99-3.80 (m, 2H), 3.83-3.69 (m, 1H), 2.87-2.74 (m, 3H), 2.47-2.25 (m, 5H), 1.89-1.68 (m, 1H).

### Synthesis 74

### 3-fluoro-N-methyl-N-[2-[(2-oxo-3,4-dihydro-1H-quinolin-6-yl)oxy]ethyl]benzamide (ALDHI-3008)

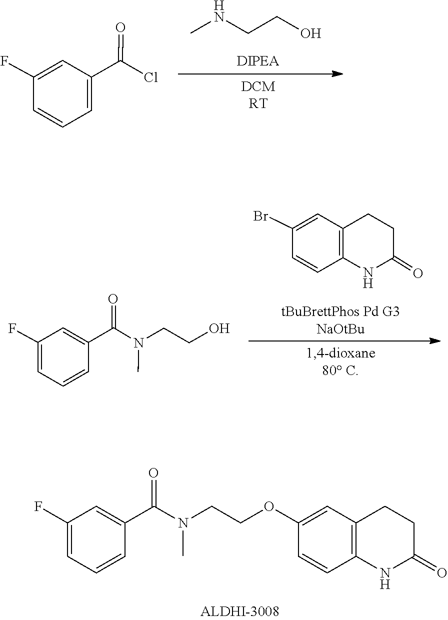

### Step 1: 3-fluoro-N-(2-hydroxyethyl)-N-methyl-benzamide

To a solution of 3-fluorobenzoyl chloride (0.77 mL, 6.31 mmol, 1 eq) in DCM (10 mL) at 0° C. was added DIPEA (1.6 mL, 9.46 mmol, 1.5 eq) followed by 2-(methylamino)ethanol (0.76 mL, 9.46 mmol, 1.5 eq) under nitrogen. The mixture was stirred at RT for 19 h then quenched with water (10 mL) and the phases separated. The organic phase was washed with aq. HCl (1M, 10 mL), brine (10 mL), filtered through a hydrophobic frit and concentrated in vacuo to afford 3-fluoro-N-(2-hydroxyethyl)-N-methyl-benzamide (1.04 g, 5.27 mmol, 84%) as a colourless oil. Used directly in the next step.

MS (ES+) m/z 198.1 (M+H).

¹H NMR (300 MHz, CDCl₃) δ 7.39 (q, J=7.4 Hz, 1H), 7.25-7.01 (m, 3H), 3.98-2.90 (m, 8H).

### Step 2: 3-fluoro-N-methyl-N-[2-[(2-oxo-3,4-dihydro-1H-quinolin-6-yl)oxy]ethyl]benzamide

Prepared as described in method G from 3-fluoro-N-(2-hydroxyethyl)-N-methyl-benzamide (1.01 g, 5.12 mmol, 1 eq), 6-bromo-1,2,3,4-tetrahydro-2-quinolinone (1.16 g, 5.12 mmol, 1 eq), NaOtBu (591 mg, 6.15 mmol, 1.2 eq) and tBuBrettPhos Pd G3 (438 mg, 0.512 mmol, 0.1 eq) in 1,4-dioxane (25 mL) to return the title compound (288 mg, 0.842 mmol, 16%) as a white powder after normal phase chromatography (SiO₂) using 0-100% EtOAc:petroleum ether as eluent and subsequent reverse phase chromatography (C₁₈) using 5-95% MeCN:H₂O as eluent.

MS (ES+) m/z 343.0 (M+H).

¹H NMR (300 MHz, DMSO-d₆) δ 9.91 (s, 1H), 7.56-7.40 (m, 1H), 7.35-7.15 (m, 3H), 6.90-6.59 (m, 3H), 4.17 (br.s, 1H), 4.02 (br.s, 1H), 3.79 (br.s, 1H), 3.56 (br.s, 1H), 3.08-2.91 (m, 3H), 2.88-2.74 (m, 2H), 2.40 (t, J=7.5 Hz, 2H).

¹⁹F NMR {¹H} (282 MHz, DMSO-d₆) δ −112.30-−112.64 (m).

### Synthesis 75

### N-methyl-N-[2-[(2-oxo-3,4-dihydro-1H-quinolin-6-yl)oxy]ethyl]pyridine-3-carboxamide (ALDHI-3013)

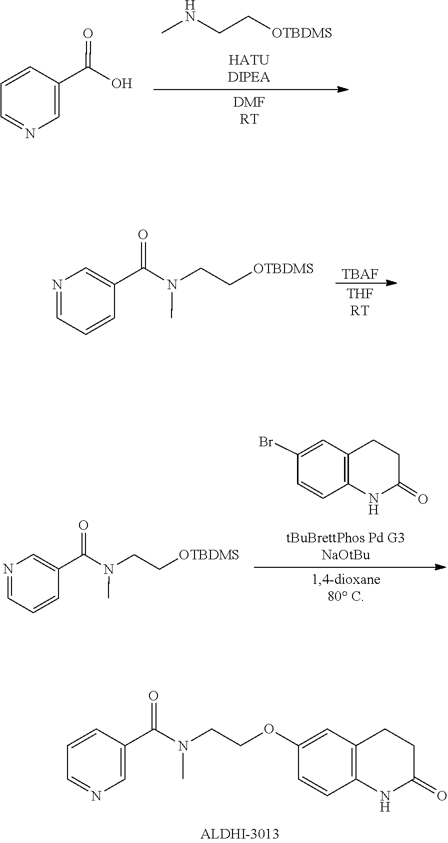

### Step 1: N-[2-[tert-butyl(dimethyl)silyl]oxyethyl]-N-methyl-pyridine-3-carboxamide

To a mixture of nicotinic acid (290 mg, 2.36 mmol, 1 eq) and HATU (1.75 g, 2.83 mmol, 1.2 eq) in DMF (5 mL) (anhydrous) was added DIPEA (0.82 mL, 4.71 mmol, 2 eq). The mixture was stirred for 0.25 h then N-[2-(tert-butyldimethylsilyloxy)ethyl]methylamine (491 mg, 2.59 mmol, 1.1 eq) added and stirring continued at RT for 24 h. Water (20 mL) was added and the mixture extracted with EtOAc (2×10 mL). The combined extracts were washed with brine (25 mL), dried (MgSO₄), filtered and concentrated in vacuo. The resulting residue was chromatographed (C₁₈) using 5-95% MeCN:H₂O as eluent and the fractions containing product combined and extracted with DCM (2×100 mL). The organic extracts were filtered through a hydrophobic frit and concentrated in vacuo to afford N-[2-[tert-butyl(dimethyl)silyl]oxyethyl]-N-methyl-pyridine-3-carboxamide (533 mg, 1.81 mmol, 77%) as an orange oil.

MS (ES+) m/z 295.2 (M+H).

¹H NMR (300 MHz, CDCl₃) δ 8.74-8.60 (m, 2H), 7.85-7.71 (m, 1H), 7.38-7.29 (m, 1H), 3.99-3.83 (m, 1H), 3.78-3.58 (m, 2H), 3.49-3.33 (m, 1H), 3.19-3.03 (m, 3H), 0.99-0.80 (m, 9H), 0.07 (dd, J=12.4, 5.3 Hz, 6H).

### Step 2: N-(2-hydroxyethyl)-N-methyl-pyridine-3-carboxamide

To a solution of N-[2-[tert-butyl(dimethyl)silyl]oxyethyl]-N-methyl-pyridine-3-carboxamide (532 mg, 1.81 mmol, 1 eq) in THF (5 mL) under nitrogen was added TBAF (1M in THF, 1.9 mL, 1.90 mmol, 1.05 eq). The mixture was stirred at RT for 1 h then was quenched with sat. aq. NaHCO₃ (10 mL) and extracted with DCM (2×20 mL). The combined extracts were washed with brine (20 mL), filtered through a hydrophobic frit and concentrated in vacuo. The residue was chromatographed (C₁₈) using 5-95% MeCN:H₂O as eluent to afford N-(2-hydroxyethyl)-N-methyl-pyridine-3-carboxamide (138 mg, 0.766 mmol, 42%) as a colourless gum.

MS (ES+) m/z 181.3 (M+H).

¹H NMR (300 MHz, DMSO-d₆) δ 8.66-8.57 (m, 2H), 7.89-7.79 (m, 1H), 7.51-7.40 (m, 1H), 4.90-4.76 (m, 1H), 3.68-3.58 (m, 1H), 3.58-3.44 (m, 2H), 3.32-3.22 (m, 1H), 3.03-2.93 (m, 3H).

### Step 3: N-methyl-N-[2-[(2-oxo-3,4-dihydro-1H-quinolin-6-yl)oxy]ethyl]pyridine-3-carboxamide

Prepared as described in method G from N-(2-hydroxyethyl)-N-methyl-pyridine-3-carboxamide (136 mg, 0.755 mmol, 1 eq), 6-bromo-1,2,3,4-tetrahydro-2-quinolinone (171 mg, 0.755 mmol, 1 eq), tBuBrettPhos Pd G3 (64 mg, 0.0755 mmol, 0.1 eq) and NaOtBu (87 mg, 0.906 mmol, 1.2 eq) in 1,4-dioxane (5 mL) to return the title compound (28 mg, 0.0861 mmol, 11%) as a yellow powder after reverse phase chromatography (C₁₈) using 5-95% MeCN:H₂O as eluent and subsequent purification by preparative HPLC-MS (low pH). MS (ES+) m/z 326.1 (M+H). ¹H NMR (300 MHz, DMSO-d₆) δ 9.93 (s, 1H), 8.71-8.52 (m, 2H), 7.83 (d, J=7.8 Hz, 1H), 7.51-7.40 (m, 1H), 6.89-6.58 (m, 3H), 4.26-4.11 (m, 1H), 4.07-3.97 (m, 1H), 3.87-3.76 (m, 1H), 3.63-3.53 (m, 1H), 3.07-2.97 (m, 3H), 2.89-2.75 (m, 2H), 2.40 (t, J=7.5 Hz, 2H).

### Scheme 7

### Preparation of Amides from Amines and Carboxylic Acids

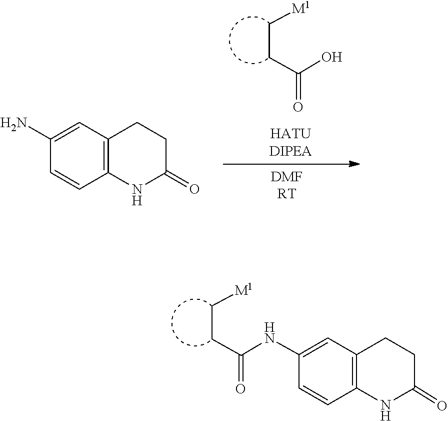

### Preparation of Alkyl Carboxylates

### Synthesis 76

### Ethyl 2-bromo-4-(3-fluorophenyl)thiazole-5-carboxylate

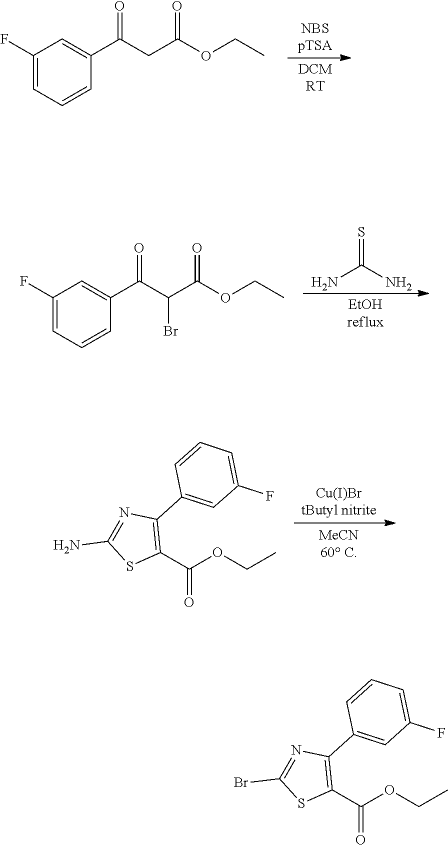

### Step 1: ethyl 2-bromo-3-(3-fluorophenyl)-3-oxo-propanoate

To a solution of 3-(3-fluoro-phenyl)-3-oxo-propionic acid ethylester (1.4 mL, 7.66 mmol, 1 eq) in DCM (60 mL) was added pTSA (291 mg, 1.53 mmol, 0.2 eq) followed by NBS (1.70 g, 9.57 mmol, 1.25 eq). The mixture was stirred at RT for 20 h. The solvent was removed in vacuo, the resulting residue dissolved in Et₂O (25 mL) and the precipitate filtered and washed with Et₂O (2×25 mL). The filtrate was washed with sat. aq. NaHCO₃ (25 mL) and water (25 mL), filtered through a hydrophobic frit and concentrated in vacuo. The resulting residue was chromatographed (SiO₂) using 0-5% EtOAc:petroleum ether as eluent to afford ethyl 2-bromo-3-(3-fluorophenyl)-3-oxo-propanoate (1.91 g, 6.61 mmol, 86%) as a light yellow oil used directly in the next step. MS (ES−) m/z 287.0/289.0 (M−H), Br isotope pattern. ¹H NMR (300 MHz, CDCl₃) δ 7.82-7.73 (m, 1H), 7.74-7.64 (m, 1H), 7.54-7.43 (m, 1H), 7.33 (tdd, J=8.2, 2.6, 1.0 Hz, 1H), 5.59 (s, 1H), 4.30 (q, J=7.1 Hz, 2H), 1.26 (t, J=7.1 Hz, 3H). ¹⁹F NMR {¹H} (282 MHz, CDCl₃) δ −110.71.

### Step 2: ethyl 2-amino-4-(3-fluorophenyl)thiazole-5-carboxylate

To a solution of ethyl 2-bromo-3-(3-fluorophenyl)-3-oxo-propanoate (1.90 g, 6.57 mmol, 1 eq) in EtOH (50 mL) was added thiourea (600 mg, 7.89 mmol, 1.2 eq). The mixture was refluxed for 2 h. The solution was cooled, concentrated in vacuo and the resulting residue was dissolved in DCM (20 mL) and washed with sat. aq. NaHCO₃ (20 mL). The aqueous layer was extracted with DCM (20 mL), the organic extracts combined and concentrated in vacuo. The solid was stirred in a minimum amount of Et₂O, filtered, washed with Et₂O (2×20 mL) and petroleum ether (2×20 mL) to afford ethyl 2-amino-4-(3-fluorophenyl)thiazole-5-carboxylate (1.43 g, 5.37 mmol, 82%) as a light yellow powder.

MS (ES+) m/z 267.0 (M+H). ¹H NMR (300 MHz, DMSO-d₆) δ 7.89 (s, 2H), 7.54-7.38 (m, 3H), 7.29-7.16 (m, 1H), 4.11 (q, J=7.1 Hz, 2H), 1.16 (t, J=7.1 Hz, 3H). ¹⁹F {¹H} NMR (282 MHz, DMSO-d₆) δ −114.52.

### Step 3: ethyl 2-bromo-4-(3-fluorophenyl)thiazole-5-carboxylate

To a mixture of ethyl 2-amino-4-(3-fluorophenyl)thiazole-5-carboxylate (1.40 g, 5.26 mmol, 1 eq) and copper (1) bromide (905 mg, 6.31 mmol, 1.2 eq) in MeCN (50 mL) was added t-butyl nitrite (0.94 mL, 7.89 mmol, 1.5 eq) under nitrogen. The mixture was heated to 60° C. for 1 h then cooled down, filtered through celite, washed with MeCN (4×25 mL) and concentrated in vacuo. The residue was partitioned between DCM (50 mL) and sat. aq. NaHCO₃ (50 mL) and the phases separated. The aqueous layer was washed with DCM (2×50 mL), the combined organic extracts washed with brine (100 mL), filtered through a hydrophobic frit and concentrated in vacuo. The resulting solid was chromatographed (SiO₂) using 0-10% EtOAc:petroleum ether as eluent to afford ethyl 2-bromo-4-(3-fluorophenyl)thiazole-5-carboxylate (1.22 g, 3.70 mmol, 70%) as a white powder. MS (ES+) m/z 329.9/331.9 (M+H), Br isotope pattern. ¹H NMR (300 MHz, DMSO-d₆) δ 7.63-7.44 (m, 3H), 7.40-7.27 (m, 1H), 4.24 (q, J=7.1 Hz, 2H), 1.21 (t, J=7.1 Hz, 3H). ¹⁹F NMR {¹H} (282 MHz, DMSO-d₆) δ −113.74.

### Synthesis 77

### Ethyl 4-(3-fluorophenyl)thiazole-5-carboxylate

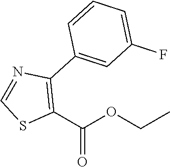 [SMILES: CCOC(=O)c1scnc1-c1cccc(F)c1]

A 0.05 M solution of ethyl 2-bromo-4-(3-fluorophenyl)thiazole-5-carboxylate (225 mg, 0.681 mmol, 1 eq) in EtOH (14 mL) was passed through a 10% Pd/C H-Cube cartridge (1 mL/min., 80° C., 40 bar). The solvent was removed in vacuo and the resulting residue was chromatographed (SiO₂) using 0-10% EtOAc:petroleum ether as eluent to afford ethyl 4-(3-fluorophenyl)thiazole-5-carboxylate (133 mg, 0.529 mmol, 78%) as a white powder.

MS (ES+) m/z 252.0 (M+H).

¹H NMR (300 MHz, DMSO-d₆) δ 9.36 (s, 1H), 7.63-7.55 (m, 2H), 7.55-7.46 (m, 1H), 7.35-7.26 (m, 1H), 4.25 (q, J=7.1 Hz, 2H), 1.23 (t, J=7.1 Hz, 3H).

¹⁹F NMR {¹H} (282 MHz, DMSO-d₆) δ −113.99.

### General Method H

### Preparation of Carboxylic Acids

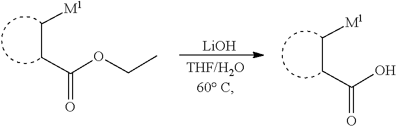

A mixture of ethyl carboxylate (1 eq) and lithium hydroxide monohydrate (2 eq) or sodium hydroxide (1 eq) in water and THF (1:5) was heated to 60° C. for 3.5 to 4 h. The mixture was cooled, concentrated in vacuo and the remaining aqueous layer acidified with aq. HCl (2M), diluted with water, the precipitate filtered, washed with water (2×25 mL) and dried in vacuo at 50° C. to afford the desired compound.

### Synthesis 78

### 2-bromo-4-(3-fluorophenyl)thiazole-5-carboxylic acid

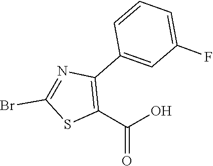 [SMILES: O=C(O)c1sc(Br)nc1-c1cccc(F)c1]

Prepared as described in method H from ethyl 2-bromo-4-(3-fluorophenyl)thiazole-5-carboxylate (1.18 g, 3.57 mmol, 1 eq) and lithium hydroxide monohydrate (307 mg, 7.15 mmol, 2 eq) in water (4 mL) and THF (20 mL) to return the title compound (1.05 g, 3.48 mmol, 97%) as a white powder.

MS (ES+) m/z 301.8/303.8 (M+H), Br isotope pattern.

¹H NMR (300 MHz, DMSO-d₆) δ 13.91 (s, 1H), 7.64-7.53 (m, 2H), 7.56-7.43 (m, 1H), 7.37-7.24 (m, 1H).

¹⁹F {¹H} NMR (282 MHz, DMSO-d₆) δ −113.76.

The following intermediate compounds were prepared similarly using method H with the appropriate ester.

### Synthesis 79

### 4-(3-fluorophenyl)thiazole-5-carboxylic acid

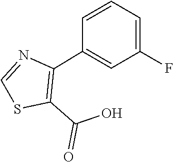 [SMILES: O=C(O)c1scnc1-c1cccc(F)c1]

MS (ES+) m/z 223.9 (M+H).

¹H NMR (300 MHz, DMSO-d₆) δ 13.54 (s, 1H), 9.30 (s, 1H), 7.65-7.55 (m, 2H), 7.55-7.43 (m, 1H), 7.28 (dddd, J=9.2, 8.3, 2.6, 1.1 Hz, 1H).

¹⁹F {¹H} NMR (282 MHz, DMSO-d₆) δ −114.02.

### General Method I

### Preparation of Amides

To a mixture of carboxylic acid (1 eq), 6-amino-3,4-dihydroquinolin-2(1H)-one (1 eq) and HATU (1.1 eq) in DMF (0.2 M) was added DIPEA (2 eq). The mixture was stirred at RT for 2 h then was quenched with brine and stirred for 0.25 h. The resulting precipitate was filtered, washed with water and dried in vacuo at 50° C. The crude material was purified by normal phase chromatography (SiO₂) using a gradient of EtOAc:petroleum ether or by reverse phase chromatography (C₁₈) using a gradient of MeCN:H₂O or by preparative HPLC-MS (high pH).

### Synthesis 80

### 2-bromo-4-(3-fluorophenyl)-N-(2-oxo-3,4-dihydro-1H-quinolin-6-yl)thiazole-5-carboxamide

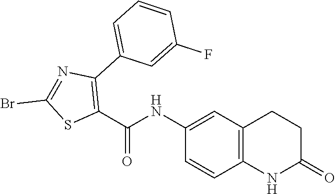 [SMILES: O=C1CCc2cc(NC(=O)c3sc(Br)nc3-c3cccc(F)c3)ccc2N1]

Prepared as described in method I from 2-bromo-4-(3-fluorophenyl)thiazole-5-carboxylic acid (1.04 g, 3.44 mmol, 1 eq), 6-amino-3,4-dihydroquinolin-2(1H)-one (558 mg, 3.44 mmol, 1 eq), HATU (1.44 g, 3.79 mmol, 1.1 eq) and DIPEA (1.2 mL, 6.88 mmol, 2 eq) in DMF (20 mL) to return the title compound (730 mg, 1.64 mmol, 48%) as a light orange powder after normal phase chromatography (SiO₂) using 0-100% EtOAc:petroleum ether as eluent.

MS (ES+) m/z 445.8/447.8 (M+H), Br isotope pattern.

¹H NMR (300 MHz, DMSO-d₆) δ 10.56 (s, 1H), 10.08 (s, 1H), 7.59-7.44 (m, 3H), 7.44-7.40 (m, 1H), 7.34-7.21 (m, 2H), 6.81 (d, J=8.5 Hz, 1H), 2.85 (t, J=7.5 Hz, 2H), 2.49-2.38 (m, 2H).

¹⁹F {¹H} NMR (282 MHz, DMSO-d₆) δ −112.68.

The following example compounds were prepared similarly using method I with the appropriate carboxylic acid.

### Synthesis 81

### 4-(3-fluorophenyl)-N-(2-oxo-3,4-dihydro-1H-quinolin-6-yl)thiazole-5-carboxamide (ALDHI-1003)

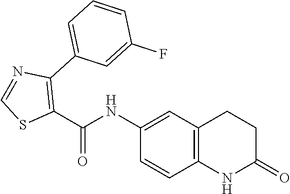 [SMILES: O=C1CCc2cc(NC(=O)c3scnc3-c3cccc(F)c3)ccc2N1]

MS (ES+) m/z 367.9 (M+H).

¹H NMR (300 MHz, DMSO-d₆) δ 10.53 (s, 1H), 10.07 (s, 1H), 9.28 (s, 1H), 7.65-7.42 (m, 4H), 7.36-7.27 (m, 1H), 7.27-7.18 (m, 1H), 6.82 (d, J=8.5 Hz, 1H), 2.86 (t, J=7.5 Hz, 2H), 2.48-2.39 (m, 2H).

¹⁹F {¹H} NMR (282 MHz, DMSO-d₆) δ −112.95.

### Synthesis 82

### 3-(3-fluorophenyl)-N-(2-oxo-1,2,3,4-tetrahydroquinolin-6-yl)-1H-pyrazole-4-carboxamide (ALDHI-1006)

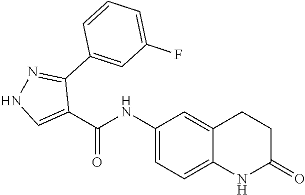 [SMILES: O=C1CCc2cc(NC(=O)c3c[nH]nc3-c3cccc(F)c3)ccc2N1]

MS (ES+) m/z 349.0 (M−H).

¹H NMR (300 MHz, CDCl₃) δ 8.21 (s, 1H), 7.57-7.33 (m, 4H), 7.04 (d, J=10.7 Hz, 1H), 6.66 (d, J=8.4 Hz, 2H), 3.03-2.93 (m, 2H), 2.66-2.54 (m, 2H).

### Synthesis 83

### 3′-fluoro-N-(2-oxo-1,2,3,4-tetrahydroquinolin-6-yl)-[1,1′-biphenyl]-2-carboxamide (ALDHI-1007)

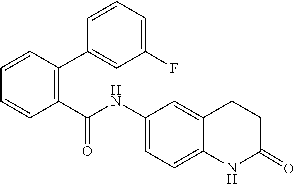 [SMILES: O=C1CCc2cc(NC(=O)c3ccccc3-c3cccc(F)c3)ccc2N1]

MS (ES+) m/z 361.1 (M+H).

¹H NMR (300 MHz, DMSO-d₆) δ 10.15 (s, 1H), 10.00 (s, 1H), 7.62-7.35 (m, 6H), 7.29-7.11 (m, 4H), 6.75 (d, J=8.5 Hz, 1H), 2.81 (t, J=7.5 Hz, 2H), 2.46-2.37 (m, 2H)

### General Method J

### Amination of 2-bromo-4-aryl-N-(2-oxo-3,4-dihydro-1H-quinolin-6-yl)thiazole-5-carboxamide

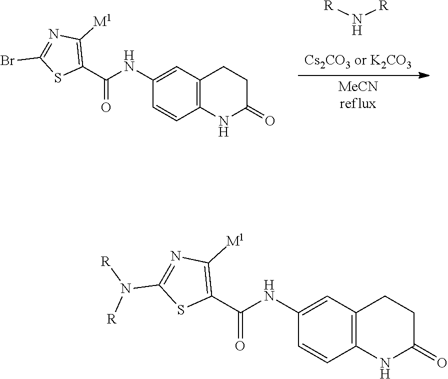

A mixture of 2-bromo-4-(3-fluorophenyl)-N-(2-oxo-3,4-dihydro-1H-quinolin-6-yl)thiazole-5-carboxamide (1 eq), substituted amine (2 eq) and Cs₂CO₃ (3 eq) or K₂CO₃ (3 eq) in MeCN (0.02 to 0.04 M) was heated to reflux for 1 to 3 days. When required, additional substituted amine and base were added to push reaction to completion. The mixture was cooled then water followed by DCM were added. At this stage, if precipitation occurred the solid was stirred for 0.5 h, filtered, washed with DCM, water and dried in vacuo at 50° C. If no precipitate formed, phases were separated and the aqueous phase was washed with DCM; the organic extracts were combined, washed with brine, filtered through a hydrophobic frit and concentrated in vacuo. The crude material was purified by normal phase chromatography (SiO₂) using a gradient of MeOH:DCM (optionally containing 1% aq. NH₃), or by preparative HPLC-MS using a gradient of high or low pH aq. MeCN or via trituration with Et₂O or petroleum ether as required.

### Synthesis 84

### 4-(3-fluorophenyl)-2-(3-hydroxyazetidin-1-yl)-N-(2-oxo-3,4-dihydro-1H-quinolin-6-yl)thiazole-5-carboxamide (ALDHI-1004)

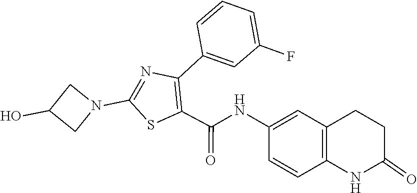 [SMILES: O=C1CCc2cc(NC(=O)c3sc(N4CC(O)C4)nc3-c3cccc(F)c3)ccc2N1]

Prepared as described in method J from 2-bromo-4-(3-fluorophenyl)-N-(2-oxo-3,4-dihydro-1H-quinolin-6-yl)thiazole-5-carboxamide (40 mg, 0.0896 mmol, 1 eq), 3-hydroxyazetidine hydrochloride (20 mg, 0.179 mmol, 2 eq) and Cs₂CO₃ (88 mg, 0.269 mmol, 3 eq) in MeCN (2.5 mL) to return the title compound (16 mg, 0.0358 mmol, 40%) as a light yellow powder after preparative HPLC-MS (high pH).

MS (ES+) m/z 439.0 (M+H).

¹H NMR (300 MHz, DMSO-d₆) δ 10.02 (s, 1H), 9.80 (s, 1H), 7.51-7.33 (m, 4H), 7.27-7.14 (m, 2H), 6.76 (d, J=8.5 Hz, 1H), 5.92 (d, J=6.6 Hz, 1H), 4.73-4.60 (m, 1H), 4.37-4.25 (m, 2H), 3.86 (dd, J=9.0, 4.5 Hz, 2H), 2.82 (t, J=7.5 Hz, 2H), 2.46-2.37 (m, 2H).

¹⁹F NMR {¹H} (282 MHz, DMSO-d₆) δ −113.61.

The following example compounds were prepared similarly using method J with the appropriate amine.

### Synthesis 85

### 2-(dimethylamino)-4-(3-fluorophenyl)-N-(2-oxo-3,4-dihydro-1H-quinolin-6-yl)thiazole-5-carboxamide (ALDHI-1002)

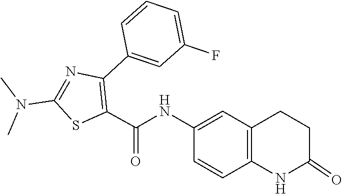 [SMILES: CN(C)c1nc(-c2cccc(F)c2)c(C(=O)Nc2ccc3c(c2)CCC(=O)N3)s1]

MS (ES+) m/z 411.4 (M+H).

¹H NMR (300 MHz, DMSO-d₆) δ 10.02 (s, 1H), 9.71 (s, 1H), 7.53-7.32 (m, 4H), 7.26-7.14 (m, 2H), 6.76 (d, J=8.5 Hz, 1H), 3.13 (s, 6H), 2.82 (t, J=7.5 Hz, 2H), 2.46-2.37 (m, 2H).

¹⁹F {¹H} NMR (282 MHz, DMSO-d₆) δ −113.68.

### Synthesis 86

### 4-(3-fluorophenyl)-2-morpholino-N-(2-oxo-3,4-dihydro-1H-quinolin-6-yl)thiazole-5-carboxamide (ALDHI-1001)

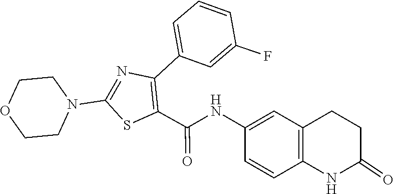 [SMILES: O=C1CCc2cc(NC(=O)c3sc(N4CCOCC4)nc3-c3cccc(F)c3)ccc2N1]

MS (ES+) m/z 453.4 (M+H).

¹H NMR (300 MHz, DMSO-d₆) δ 10.03 (s, 1H), 9.87 (s, 1H), 7.53-7.34 (m, 4H), 7.27-7.14 (m, 2H), 6.77 (d, J=8.5 Hz, 1H), 3.79-3.70 (m, 4H), 3.55-3.46 (m, 4H), 2.83 (t, J=7.5 Hz, 2H), 2.48-2.37 (m, 2H).

¹⁹F {¹H} NMR (282 MHz, DMSO-d₆) δ −113.55.

### Synthesis 87

### 4-(3-fluorophenyl)-N-(2-oxo-3,4-dihydro-1H-quinolin-6-yl)-2-piperazin-1-yl-thiazole-5-carboxamide (ALDHI-1005)

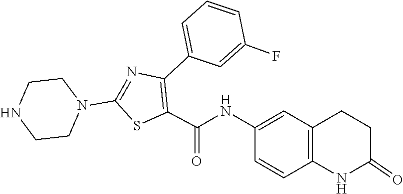 [SMILES: O=C1CCc2cc(NC(=O)c3sc(N4CCNCC4)nc3-c3cccc(F)c3)ccc2N1]

A mixture of 2-bromo-4-(3-fluorophenyl)-N-(2-oxo-3,4-dihydro-1H-quinolin-6-yl)thiazole-5-carboxamide (49 mg, 0.110 mmol, 1 eq), Cs₂CO₃ (108 mg, 0.329 mmol, 3 eq) and 1-t-Boc-piperazine (41 mg, 0.220 mmol, 2 eq) in MeCN (2.5 mL) was reflux for 5 days. The mixture was cooled, water (10 mL) followed by DCM (10 mL) was added and the resulting mixture stirred for 0.5 h. The phases were separated, the aqueous phase washed with DCM (10 mL), the organic extracts combined, filtered through a hydrophobic frit and concentrated in vacuo. The resulting solid was chromatographed (SiO₂) using 0-5% MeOH:DCM (+1% aq. NH₃) as eluent to afford the t-Boc protected intermediate (31 mg) as a brown powder. MS (ES+) m/z 552.3 (M+H). This residue was dissolved in DCM (2 mL) and trifluoroacetic acid (0.50 mL, 6.49 mmol, 59.1 eq) added. The mixture was stirred for 1.5 h, concentrated in vacuo and the resulting solid triturated with Et₂O, then purified by preparative HPLC-MS (low pH) to afford the title compound (8 mg, 0.0177 mmol, 16%) as a yellow powder.

MS (ES+) m/z 452.0 (M+H).

¹H NMR (300 MHz, DMSO-d₆) δ 10.03 (s, 1H), 9.81 (s, 1H), 7.54-7.33 (m, 4H), 7.26-7.13 (m, 2H), 6.77 (d, J=8.5 Hz, 1H), 3.49-3.40 (m, 4H), 2.91-2.76 (m, 6H), 2.47-2.35 (m, 2H).

¹⁹F NMR {¹H} (282 MHz, DMSO-d₆) δ −113.60.

### Synthesis 88

### methyl 3-(3-fluorophenyl)-5-methylisoxazole-4-carboxylate

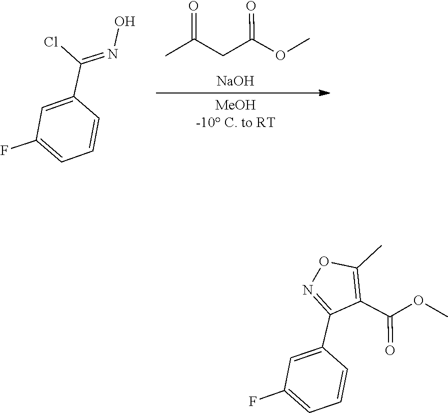

To a solution of (1Z)-3-fluoro-N-hydroxy-benzimidoyl chloride (90 mg, 0.519 mmol, 1.00 eq) and methyl 3-oxobutanoate (120 mg, 1.04 mmol, 2.00 eq) in methanol (6.6724 mL) was added at −10° C. sodium methoxide (25% in methanol, 0.36 mL, 1.56 mmol, 3.00 eq). The reaction mixture was allowed to warm up to RT over 2 h then evaporated to dryness. The resulting residue was dissolved in ethyl acetate and this organic layer was washed with water and brine, dried over magnesium sulfate, filtered and evaporated to dryness. The crude mixture was purified by silica (SiO₂) gel column chromatography using a 0-10% gradient of ethyl acetate in petroleum ether to give methyl 3-(3-fluorophenyl)-5-methyl-isoxazole-4-carboxylate (30 mg, 0.128 mmol, 25%) as a colourless oil.

MS (ES+) m/z 236.2 (M+H).

¹H NMR (300, CDCl₃) δ 7.49-7.35 (m, 3H), 7.25-7.15 (m, 1H), 3.82 (s, 3H), 2.76 (s, 3H) ppm.

### Synthesis 89

### ethyl 4-(3-fluorophenyl)-2-methylthiazole-5-carboxylate

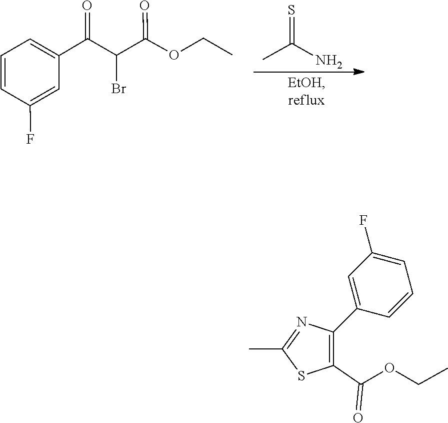

To a solution of ethyl 2-bromo-3-(3-fluorophenyl)-3-oxo-propanoate (1.23 g, 3.87 mmol, 1 eq) in EtOH (20 mL) was added thioacetamide (349 mg, 4.65 mmol, 1.2 eq). The mixture was refluxed for 3 h, then cooled down and concentrated in vacuo. The resulting residue was dissolved in DCM (30 mL) and washed with sat. aq. NaHCO₃ (30 mL). The organic layer was filtered through a hydrophobic frit and concentrated in vacuo. The crude mixture was purified by flash chromatography (SiO₂) using a 0-100% gradient of EtOAc in petroleum ether to afford a residue that was used in the next step without further purification.

MS (ES+) m/z 266.2 (M+H).

### General Method K

### Synthesis of Thiazoles from β-ketoesters

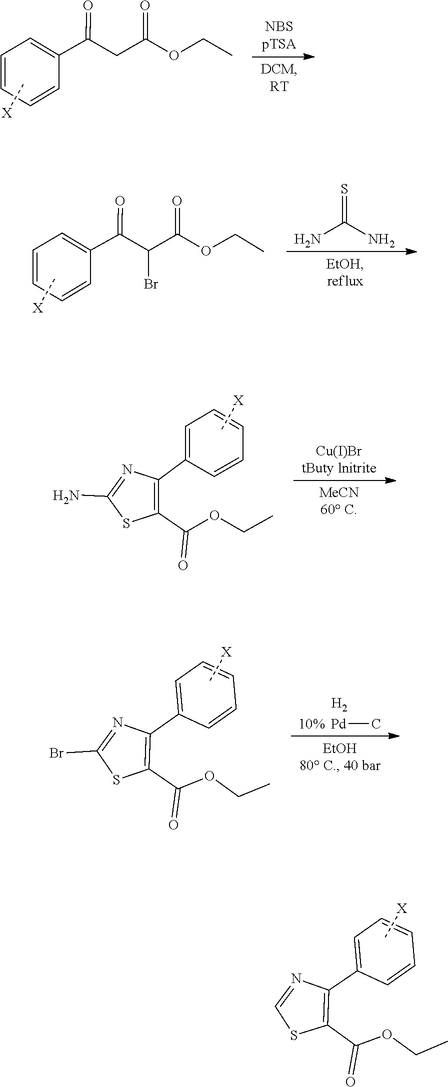

### Step 1: Bromination of a Beta-Keto Ester

To a solution of the appropriately substituted phenyl 3-oxo-propionic acid ethylester (1 eq) in DCM (0.13 M) were added pTSA (0.2 eq) and NBS (1.25 eq). The mixture was stirred at RT for 20 h, then evaporated to dryness. The resulting residue was dissolved in Et₂O and the precipitate filtered and washed with Et₂O. The filtrate was washed with sat. aq. NaHCO₃ and water, filtered through a hydrophobic frit and concentrated in vacuo. The resulting residue was chromatographed (SiO₂) using a gradient of EtOAc in petroleum ether as eluent to afford the appropriately substituted phenyl ethyl 2-bromo-3-oxo-propanoate.

### Step 2: Formation of the Thiazole Ring

To a solution of the appropriately substituted phenyl ethyl 2-bromo-3-oxo-propanoate (1 eq) in EtOH (0.13 M) was added thiourea (1.2 eq). The mixture was refluxed for 2 h, then cooled down and concentrated in vacuo. The resulting residue was dissolved in DCM and washed with sat. aq. NaHCO₃. The aqueous layer was extracted with DCM, the organic extracts combined and concentrated in vacuo. The resulting solid was stirred in a minimum amount of Et₂O, filtered, washed with Et₂O and petroleum ether to afford the appropriately substituted phenyl ethyl 2-amino-thiazole-5-carboxylate.

### Step 3: Conversion of an Amine to a Bromine Substituent

To a mixture of the appropriately substituted phenyl ethyl 2-amino-thiazole-5-carboxylate (1 eq) and copper (1) bromide (1.2 eq) in MeCN (0.1 M) was added t-butyl nitrite (1.5 eq) under nitrogen. The mixture was heated to 60° C. for 1-6 hours then cooled down, filtered through celite, washed with MeCN and concentrated in vacuo. The residue was partitioned between DCM and sat. aq. NaHCO₃ and the phases separated. The aqueous layer was washed with DCM, the combined organic extracts washed with brine, filtered through a hydrophobic frit and concentrated in vacuo. The resulting solid was either used directly in the next step without further purification or chromatographed (SiO₂) using a gradient of EtOAc in petroleum ether as eluent to afford the appropriately substituted ethyl 2-bromo-thiazole-5-carboxylate.

### Step 4: Conversion of the Bromine Hydrogen Atom

A solution of the appropriately substituted ethyl 2-bromo-thiazole-5-carboxylate (225 mg, 1 eq) in EtOH (0.05 M) was passed through a 10% Pd/C H-Cube cartridge (1 mL/min., 80° C., 40 bar). The solvent was removed in vacuo and the resulting residue was chromatographed (SiO₂) using a gradient of EtOAc in petroleum ether as eluent to afford the desired intermediate.

The following intermediate compounds were prepared in 4 steps as described in method K.

### Synthesis 90

### ethyl 4-(2-chlorophenyl)thiazole-5-carboxylate

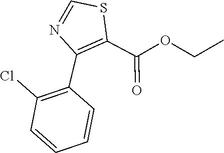 [SMILES: CCOC(=O)c1scnc1-c1ccccc1Cl]

MS (ES+) m/z 268.1 (M+H).

¹H NMR (300 MHz, DMSO-d6) δ 9.39 (s, 1H), 7.61-7.54 (m, 1H), 7.54-7.41 (m, 3H), 4.15 (q, J=7.1 Hz, 2H), 1.09 (t, J=7.1 Hz, 3H).

### Synthesis 91

### ethyl 4-(m-tolyl)thiazole-5-carboxylate

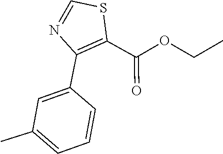 [SMILES: CCOC(=O)c1scnc1-c1cccc(C)c1]

MS (ES+) m/z 249.1 (M+H).

¹H NMR (300 MHz, DMSO-d₆) δ 9.32 (s, 1H), 7.59-7.49 (m, 2H), 7.40-7.23 (m, 2H), 4.23 (q, J=7.1 Hz, 2H), 2.36 (d, J=0.8 Hz, 3H), 1.21 (t, J=7.1 Hz, 3H).

### General Method L

### Suzuki Coupling on Brominated Heteroaromatic Esters

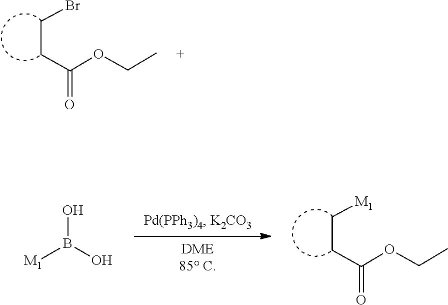

To a solution of brominated ethyl carboxylate (1.00 eq) and boronic acid (1.00 eq) in DME (0.1 M) were added Pd(PPh₃)₄ (0.05 eq) and K₂CO₃ (0.4 M in water, 2.00 eq). The resulting mixture was degassed for 15 mins, stirred at 85° C. for 2 hours, cooled down to RT then diluted with water, filtered and extracted with ethyl acetate. The combined organic layers were dried over anhydrous MgSO₄, filtered and evaporated to dryness. The crude material was purified by flash chromatography (SiO₂) using a gradient of EtOAc in petroleum ether to afford the desired compound.

### Synthesis 92

### ethyl 4-(3-fluorophenyl)oxazole-5-carboxylate

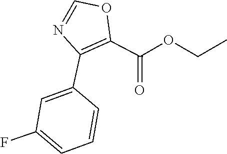 [SMILES: CCOC(=O)c1ocnc1-c1cccc(F)c1]

Prepared as described in method L from ethyl 4-bromo-oxazole-5-carboxylate (200 mg, 0.909 mmol, 1.00 eq), 3-fluorobenzeneboronic acid (127 mg, 0.909 mmol, 1.00 eq), Pd(PPh₃)₄ (53 mg, 0.0455 mmol, 0.0500 eq) and K₂CO₃ (0.4M in water, 4.5 mL, 1.82 mmol, 2.00 eq) in DME (8 mL) to return the title compound ethyl 4-(3-fluorophenyl)oxazole-5-carboxylate (48 mg, 0.196 mmol, 22%) as a yellow oil.

MS (ES+) m/z 236.2 (M+H).

¹H NMR (DMSO-d₆) δ 8.74 (s, 1H), 7.94-7.83 (m, 2H), 7.55 (td, J=8.2, 6.3 Hz, 1H), 7.33 (tdd, J=8.6, 2.5 Hz, 1H), 4.35 (q, J=7.1 Hz, 2H), 1.31 (t, J=7.1 Hz, 3H) ppm.

The following intermediate compounds were prepared similarly using method L with the appropriate brominated ester and boronic acid.

### Synthesis 93

### ethyl 3-(3-fluorophenyl)pyridine-2-carboxylate

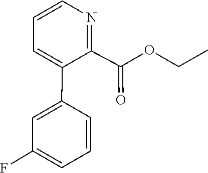 [SMILES: CCOC(=O)c1ncccc1-c1cccc(F)c1]

MS (ES+) m/z 246.2 (M+H).

¹H NMR (DMSO-d₆) δ 8.67 (dt, J=4.8, 1.5 Hz, 1H), 7.99 (dt, J=7.9, 1.5 Hz, 1H), 7.67 (ddd, J=7.9, 4.7, 1.3 Hz, 1H), 7.58-7.48 (m, 1H), 7.37-7.12 (m, 3H), 4.15 (q, J=7.1 Hz, 2H), 1.05 (t, J=7.1 Hz, 3H) ppm.

### Synthesis 94

### ethyl 4-(2-fluorophenyl)thiazole-5-carboxylate

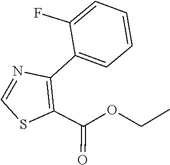 [SMILES: CCOC(=O)c1scnc1-c1ccccc1F]

MS (ES+) m/z 252.2 (M+H).

¹H NMR (300 MHz, DMSO-d₆) δ 9.40 (s, 1H), 7.62-7.48 (m, 2H), 7.36-7.26 (m, 2H), 4.20 (q, J=7.1 Hz, 2H), 1.16 (t, J=7.1 Hz, 3H) ppm.

¹⁹F {1H} NMR (282 MHz, DMSO-d₆) δ −114.52 ppm.

### Synthesis 95

### ethyl 3-(3-fluorophenyl)thiophene-2-carboxylate

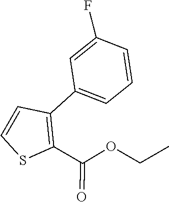 [SMILES: CCOC(=O)c1sccc1-c1cccc(F)c1]

MS (ES+) m/z 251.2 (M+H).

¹H NMR (300 MHz, DMSO-d₆) δ 7.96 (d, J=5.1 Hz, 1H), 7.45 (dddd, J=8.2, 7.5, 6.2, 0.6 Hz, 1H), 7.37-7.18 (m, 4H), 4.18 (q, J=7.1 Hz, 2H), 1.17 (t, J=7.1 Hz, 3H) ppm.

¹⁹F {1H} NMR (282 MHz, DMSO-d₆) δ −114.18 ppm.

### Synthesis 96

### ethyl 4-(thiophen-2-yl)thiazole-5-carboxylate

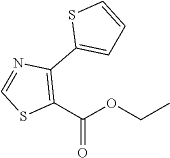

MS (ES+) m/z 240.1 (M+H).

¹H NMR (300 MHz, DMSO-d₆) δ 9.29 (s, 1H), 8.31 (dd, J=3.8, 1.2 Hz, 1H), 7.75 (dd, J=5.1, 1.2 Hz, 1H), 7.20 (dd, J=5.1, 3.8 Hz, 1H), 4.34 (q, J=7.1 Hz, 2H), 1.32 (t, J=7.1 Hz, 3H) ppm.

### Synthesis 97

### ethyl 4-(3,5-difluorophenyl)thiazole-5-carboxylate

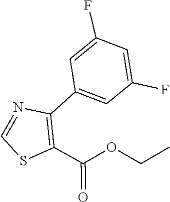 [SMILES: CCOC(=O)c1scnc1-c1cc(F)cc(F)c1]

MS (ES+) m/z 287.2 (M+NH₄).

¹H NMR (300 MHz, DMSO-d₆) δ 9.37 (s, 1H), 7.55-7.45 (m, 2H), 7.43-7.33 (m, 1H), 4.26 (q, J=7.1 Hz, 2H), 1.23 (t, J=7.1 Hz, 3H) ppm.

¹⁹F {1H} NMR (282 MHz, DMSO-d₆) δ −110.53 ppm.

### General Method M

### One-Pot Suzuki Coupling and Ester Deprotection to Carboxylic Acids

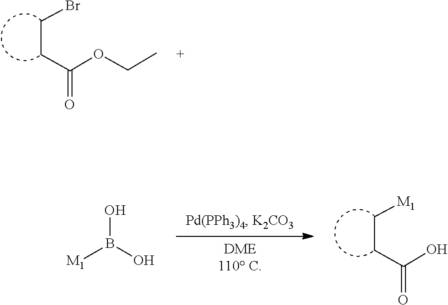

To a solution of brominated ethyl carboxylate (1.00 eq) and boronic acid (1.00 eq) in DME (0.1 M) were added Pd(PPh₃)₄ (0.05 eq) and K₂CO₃ (0.4 M in water, 2.00 eq). The resulting mixture was degassed for 20 mins, stirred at 110° C. for 24 hours, cooled down to RT then filtered through a celite pad and evaporated to dryness. The resulting residue was diluted with aqueous HCl 2M and extracted with diethyl ether. The combined organic layers were dried over anhydrous MgSO₄, filtered and evaporated to dryness. This residue was used in the next step without further purification.

### Synthesis 98

### 5-(pyridin-3-yl)thiazole-4-carboxylic acid

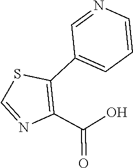 [SMILES: O=C(O)c1ncsc1-c1cccnc1]

Prepared as described in method M from ethyl 5-bromothiazole-4-carboxylate (250 mg, 1.06 mmol, 1.00 eq), pyridine-3-boronic acid (130 mg, 1.06 mmol, 1.00 eq), Pd(PPh₃)₄ (61 mg, 0.05 mmol, 0.05 eq) and potassium carbonate (0.4M in water, 5.4 mL, 2.14 mmol, 2.00 eq) in DME (9.5 mL) to return a residue that was used in the next step without further purification.

MS (ES+) m/z 207.1 (M+H).

The following intermediate compounds were prepared similarly using method M with the appropriate brominated ester and boronic acid.

### Synthesis 99

### 5-(thiophen-3-yl)thiazole-4-carboxylic acid

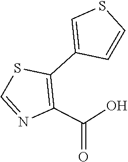 [SMILES: O=C(O)c1ncsc1-c1ccsc1]

MS (ES+) m/z 212.1 (M+H).

### Synthesis 100

### 5-(3-fluorophenyl)thiazole-4-carboxylic acid

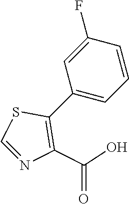 [SMILES: O=C(O)c1ncsc1-c1cccc(F)c1]

MS (ES+) m/z 224.2 (M+H).

### Synthesis 101

### 4-(3-(trifluoromethyl)phenyl)thiazole-5-carboxylic acid

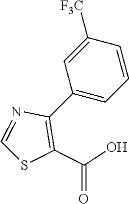 [SMILES: O=C(O)c1scnc1-c1cccc(C(F)(F)F)c1]

MS (ES+) m/z 274.1 (M+H).

### Synthesis 102

### 4-(4-fluorophenyl)thiazole-5-carboxylic acid

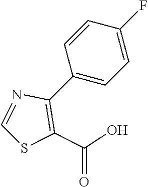 [SMILES: O=C(O)c1scnc1-c1ccc(F)cc1]

MS (ES+) m/z 224.1 (M+H).

### Synthesis 103

### 4-(3-fluorophenyl)pyrimidine-5-carboxylic acid

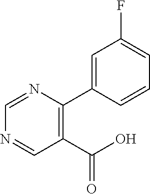 [SMILES: O=C(O)c1cncnc1-c1cccc(F)c1]

MS (ES−) m/z 217.0 (M−H).

### Synthesis 104

### 3-(3-fluorophenyl)-5-methyl-isoxazole-4-carboxylic acid

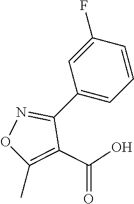 [SMILES: Cc1onc(-c2cccc(F)c2)c1C(=O)O]

Prepared as described in method H from ethyl 3-(3-fluorophenyl)-5-methyl-isoxazole-4-carboxylate (30 mg, 0.12 mmol, 1.00 eq) and sodium hydroxide (4.9 mg, 0.12 mmol, 1.00 eq) in water (1.5 mL) and THF (3.7 mL) to afford the title compound 3-(3-fluorophenyl)-5-methyl-isoxazole-4-carboxylic acid (22 mg, 0.099 mmol, 83%) as a colourless oil.

MS (ES+) m/z 222.0 (M+H).

¹H NMR (300 MHz, CDCl₃) δ 7.57-7.35 (m, 3H), 7.27-7.10 (m, 1H), 2.77 (s, 3H) ppm.

¹⁹F {1H} NMR (282 MHz, CDCl₃) 5-113.07 ppm.

The following intermediate compounds were prepared similarly using method H with the appropriate ester.

### Synthesis 105

### 4-(2-chlorophenyl)thiazole-5-carboxylic acid

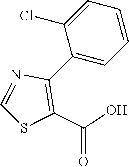 [SMILES: O=C(O)c1scnc1-c1ccccc1Cl]

MS (ES+) m/z 240.1 (M+H).

¹H NMR (300 MHz, DMSO-d₆) δ 9.33 (s, 1H), 7.58-7.51 (m, 1H), 7.50-7.37 (m, 3H) ppm.

### Synthesis 106

### 4-(m-tolyl)thiazole-5-carboxylic acid

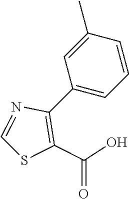

MS (ES+) m/z 220.0 (M+H); used in the next step without further purification.

### Synthesis 107

### 4-(3-fluorophenyl)oxazole-5-carboxylic acid

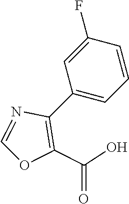 [SMILES: O=C(O)c1ocnc1-c1cccc(F)c1]

MS (ES−) m/z 206.0 (M−H).

¹H NMR (300 MHz, DMSO-d₆) δ 8.68 (s, 1H), 7.98-7.90 (m, 2H), 7.54 (td, J=8.2, 6.2 Hz, 1H), 7.31 (tdd, J=8.5, 2.6, 1.1 Hz, 1H) ppm.

¹⁹F {1H} NMR (282 MHz, DMSO-d₆) δ −113.24 ppm.

### Synthesis 108

### 3-(3-fluorophenyl)pyridine-2-carboxylic acid

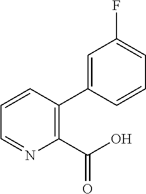 [SMILES: O=C(O)c1ncccc1-c1cccc(F)c1]

MS (ES+) m/z 218.2 (M+H).

¹H NMR (300 MHz, DMSO-d₆) δ 13.39 (s, 1H), 8.64 (dd, J=4.7, 1.6 Hz, 1H), 7.95 (dd, J=7.9, 1.6 Hz, 1H), 7.62 (dd, J=7.9, 4.7 Hz, 1H), 7.57-7.47 (m, 1H), 7.32-7.23 (m, 3H) ppm.

¹⁹F {1H} NMR (282 MHz, DMSO-d₆) δ −113.11 ppm.

### Synthesis 109

### 4-(2-fluorophenyl)thiazole-5-carboxylic acid

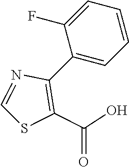 [SMILES: O=C(O)c1scnc1-c1ccccc1F]

MS (ES+) m/z 224.1 (M+H).

¹H NMR (300 MHz, DMSO-d₆) δ 13.43 (s, 1H), 9.34 (s, 1H), 7.60-7.46 (m, 2H), 7.34-7.24 (m, 2H) ppm.

¹⁹F {1H} NMR (282 MHz, DMSO-d₆) δ −114.21 ppm.

### Synthesis 110

### 3-(3-fluorophenyl)thiophene-2-carboxylic acid

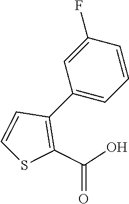 [SMILES: O=C(O)c1sccc1-c1cccc(F)c1]

MS (ES−) m/z 220.9 (M−H).

¹H NMR (300 MHz, DMSO-d₆) δ 12.98 (s, 1H), 7.89 (d, J=5.1 Hz, 1H), 7.49-7.39 (m, 1H), 7.35-7.27 (m, 2H), 7.25-7.16 (m, 2H) ppm.

¹⁹F {1H} NMR (282 MHz, DMSO-d₆) δ −114.22 ppm.

### Synthesis 111

### 4-(2-thienyl)thiazole-5-carboxylic acid

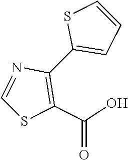 [SMILES: O=C(O)c1scnc1-c1cccs1]

MS (ES+) m/z 212.1 (M+H).

¹H NMR (300 MHz, DMSO-d₆) δ 13.63 (s, 1H), 9.23 (s, 1H), 8.34 (dd, J=3.8, 1.2 Hz, 1H), 7.72 (dd, J=5.1, 1.2 Hz, 1H), 7.18 (dd, J=5.1, 3.8 Hz, 1H) ppm.

### Synthesis 112

### 4-(3,5-difluorophenyl)thiazole-5-carboxylic acid

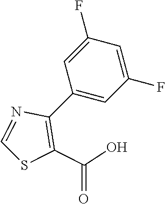 [SMILES: O=C(O)c1scnc1-c1cc(F)cc(F)c1]

MS (ES−) m/z 239.9 (M−H).

¹H NMR (300 MHz, DMSO-d₆) δ 13.67 (s, 1H), 9.32 (s, 1H), 7.58-7.46 (m, 2H), 7.36 (tt, J=9.4, 2.4 Hz, 1H) ppm.

¹⁹F {1H} NMR (282 MHz, DMSO-d₆) δ −110.58 ppm.

### Synthesis 113

### 4-(3-fluorophenyl)-2-methyl-thiazole-5-carboxylic acid

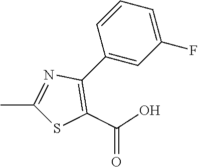 [SMILES: Cc1nc(-c2cccc(F)c2)c(C(=O)O)s1]

MS (ES+) m/z 238.9 (M+H).

¹H NMR (300 MHz, DMSO-d₆) δ 13.39 (s, 1H), 7.61-7.52 (m, 2H), 7.46 (td, J=8.0, 6.0 Hz, 1H), 7.32-7.21 (m, 1H), 2.70 (s, 3H) ppm.

¹⁹F {1H} NMR (282 MHz, DMSO-d₆) δ −114.06 ppm.

### Synthesis 114

### 2-(3-methyl-1,2,4-oxadiazol-5-yl)-N-(2-oxo-1,2,3,4-tetrahydroquinolin-6-yl)benzamide (ALDHI-1008)

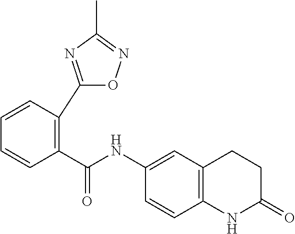 [SMILES: Cc1noc(-c2ccccc2C(=O)Nc2ccc3c(c2)CCC(=O)N3)n1]

Prepared as described in method I from 2-(3-methyl-1,2,4-oxadiazol-5-yl)benzoic acid (50 mg, 0.245 mmol, 1.00 eq; commercially available intermediate), 6-Amino-3,4-dihydroquinolin-2(1H)-one (40 mg, 0.245 mmol, 1.00 eq), N,N-diisopropylethylamine (0.085 mL, 0.490 mmol, 2.00 eq) and 2-(7-aza-1H-benzotriazole-1-yl)-1,1,3,3-tetramethyluronium hexafluorophosphate (HATU) (102 mg, 0.269 mmol, 1.10 eq) in DMF (1 mL) to return the title compound 2-(3-methyl-1,2,4-oxadiazol-5-yl)-N-(2-oxo-3,4-dihydro-1H-quinolin-6-yl)benzamide (29 mg, 0.0821 mmol, 34%) as a yellow powder after purification by preparative HPLC-MS (high pH).

MS (ES+) m/z 349.1 (M+H).

¹H NMR (300 MHz, DMSO-d₆) δ 10.40 (s, 1H), 10.05 (s, 1H), 8.07-7.99 (m, 1H), 7.83-7.67 (m, 3H), 7.51 (d, J=2.3 Hz, 1H), 7.35 (dd, J=8.5, 2.3 Hz, 1H), 6.82 (d, J=8.5 Hz, 1H), 2.87 (t, J=7.5 Hz, 2H), 2.49-2.41 (m, 2H), 2.38 (s, 3H) ppm.

The following example compounds were prepared similarly using method I with the appropriate carboxylic acid.

### Synthesis 115

### 3-(3-fluorophenyl)-5-methyl-N-(2-oxo-3,4-dihydro-1H-quinolin-6-yl)isoxazole-4-carboxamide (ALDHI-1009)

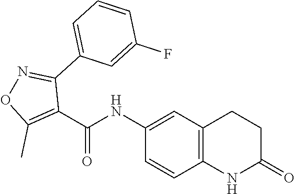 [SMILES: Cc1onc(-c2cccc(F)c2)c1C(=O)Nc1ccc2c(c1)CCC(=O)N2]

MS (ES+) m/z 366.3 (M+H).

¹H NMR (300 MHz, CDCl₃) δ 7.64-7.51 (m, 1H), 7.51-7.39 (m, 3H), 7.34 (d, J=6.0 Hz, 2H), 6.94 (d, J=26.9 Hz, 2H), 6.65 (d, J=8.4 Hz, 1H), 2.96 (t, J=7.5 Hz, 2H), 2.81 (s, 3H), 2.63 (dd, J=8.6, 6.5 Hz, 2H) ppm.

¹⁹F {1H} NMR (282 MHz, CDCl₃) 5-110.09 ppm.

### Synthesis 116

### N-(2-oxo-3,4-dihydro-1H-quinolin-6-yl)-5-(3-pyridyl)thiazole-4-carboxamide (ALDHI-1010)

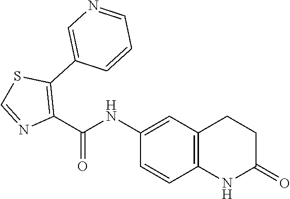 [SMILES: O=C1CCc2cc(NC(=O)c3ncsc3-c3cccnc3)ccc2N1]

¹H NMR (300 MHz, DMSO-d₆) δ 10.29 (s, 1H), 10.03 (s, 1H), 9.30 (s, 1H), 8.76 (dd, J=2.4, 0.9 Hz, 1H), 8.61 (dd, J=4.8, 1.6 Hz, 1H), 8.02 (ddd, J=7.9, 2.4, 1.7 Hz, 1H), 7.63 (d, J=2.3 Hz, 1H), 7.59-7.40 (m, 2H), 6.79 (d, J=8.5 Hz, 1H), 2.84 (t, J=7.6 Hz, 2H), 2.43 (dd, J=8.5, 6.4 Hz, 2H) ppm.

### Synthesis 117

### N-(2-oxo-3,4-dihydro-1H-quinolin-6-yl)-5-(3-thienyl)thiazole-4-carboxamide (ALDHI-1011)

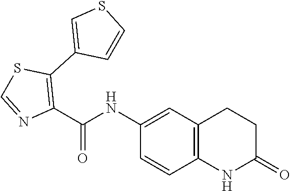 [SMILES: O=C1CCc2cc(NC(=O)c3ncsc3-c3ccsc3)ccc2N1]

MS (ES+) m/z 356.0 (M+H).

¹H NMR (300 MHz, DMSO-d₆) δ 10.24 (s, 1H), 10.04 (s, 1H), 9.14 (s, 1H), 8.04 (dd, J=3.0, 1.3 Hz, 1H), 7.68-7.62 (m, 2H), 7.49 (dd, J=8.5, 2.4 Hz, 1H), 7.44 (dd, J=5.0, 1.4 Hz, 1H), 6.81 (d, J=8.5 Hz, 1H), 2.86 (t, J=7.5 Hz, 2H), 2.44 (dd, J=8.5, 6.5 Hz, 2H) ppm.

### Synthesis 118

### 4-(2-chlorophenyl)-N-(2-oxo-3,4-dihydro-1H-quinolin-6-yl)thiazole-5-carboxamide (ALDHI-1012)

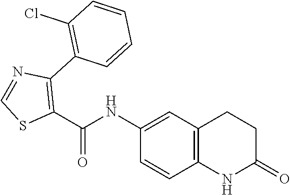 [SMILES: O=C1CCc2cc(NC(=O)c3scnc3-c3ccccc3Cl)ccc2N1]

MS (ES+) m/z 384.0 (M+H).

¹H NMR (300 MHz, DMSO-d₆) δ 10.04 (d, J=4.3 Hz, 2H), 9.29 (s, 1H), 7.59-7.49 (m, 2H), 7.47-7.42 (m, 2H), 7.38 (s, 1H), 7.23 (d, J=8.9 Hz, 1H), 6.76 (d, J=8.5 Hz, 1H), 2.82 (t, J=7.5 Hz, 2H), 2.45-2.38 (m, 2H) ppm.

### Synthesis 119

### 4-(m-tolyl)-N-(2-oxo-3,4-dihydro-1H-quinolin-6-yl)thiazole-5-carboxamide (ALDHI-1013)

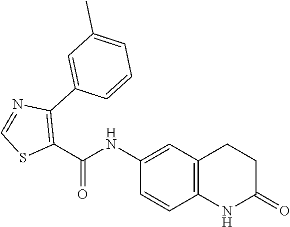 [SMILES: Cc1cccc(-c2ncsc2C(=O)Nc2ccc3c(c2)CCC(=O)N3)c1]

MS (ES+) m/z 364.3 (M+H).

¹H NMR (300 MHz, DMSO-d₆) δ 10.40 (s, 1H), 10.06 (s, 1H), 9.25 (s, 1H), 7.66-7.19 (m, 6H), 6.81 (d, J=8.5 Hz, 1H), 2.85 (t, J=7.5 Hz, 2H), 2.44 (dd, J=8.5, 6.5 Hz, 2H), 2.32 (s, 3H) ppm.

### Synthesis 120

### 5-(3-fluorophenyl)-N-(2-oxo-3,4-dihydro-1H-quinolin-6-yl)thiazole-4-carboxamide (ALDHI-1014)

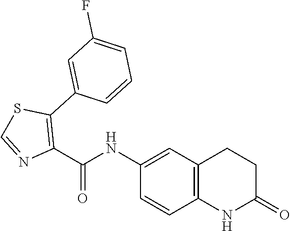 [SMILES: O=C1CCc2cc(NC(=O)c3ncsc3-c3cccc(F)c3)ccc2N1]

¹H NMR (300 MHz, DMSO-d₆) δ 10.27 (s, 1H), 10.03 (s, 1H), 9.25 (s, 1H), 7.67-7.52 (m, 2H), 7.52-7.39 (m, 4H), 7.29 (dddd, J=9.1, 7.8, 2.8, 1.2 Hz, 1H), 6.80 (d, J=8.5 Hz, 1H), 2.85 (t, J=7.5 Hz, 2H), 2.47-2.38 (m, 2H) ppm.

¹⁹F {1H} NMR (282 MHz, DMSO-d₆) δ −113.34 ppm.

### Synthesis 121

### N-(2-oxo-3,4-dihydro-1H-quinolin-6-yl)-4-[3-(trifluoromethyl)phenyl]thiazole-5-carboxamide (ALDHI-1015)

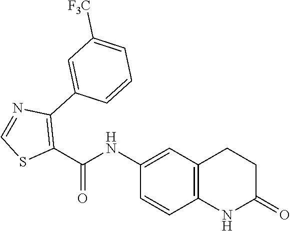 [SMILES: O=C1CCc2cc(NC(=O)c3scnc3-c3cccc(C(F)(F)F)c3)ccc2N1]

MS (ES+) m/z 417.9 (M+H).

¹H NMR (300 MHz, DMSO-d₆) δ 10.53 (s, 1H), 10.07 (s, 1H), 9.32 (s, 1H), 8.09 (s, 1H), 8.04 (d, J=7.8 Hz, 1H), 7.78 (d, J=7.9 Hz, 1H), 7.68 (d, J=7.7 Hz, 1H), 7.41 (s, 1H), 7.29 (d, J=8.3 Hz, 1H), 6.81 (d, J=8.5 Hz, 1H), 2.85 (t, J=7.5 Hz, 2H), 2.46-2.41 (m, 2H) ppm.

¹⁹F {1H} NMR (282 MHz, DMSO-d₆) δ −61.32 ppm.

### Synthesis 122

### 4-(3-fluorophenyl)-N-(3-oxo-4H-1,4-benzoxazin-7-yl)thiazole-5-carboxamide (ALDHI-1016)

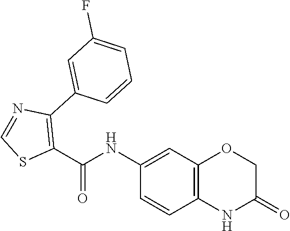 [SMILES: O=C1COc2cc(NC(=O)c3scnc3-c3cccc(F)c3)ccc2N1]

MS (ES+) m/z 370.2 (M+H).

¹H NMR (300 MHz, DMSO-d₆) δ 10.69 (s, 1H), 10.59 (s, 1H), 9.29 (s, 1H), 7.60-7.45 (m, 3H), 7.28 (d, J=1.5 Hz, 1H), 7.26-7.23 (m, 1H), 7.15 (d, J=8.5 Hz, 1H), 6.85 (d, J=8.5 Hz, 1H), 4.57 (s, 2H) ppm.

¹⁹F {1H} NMR (282 MHz, DMSO-d₆) δ −112.95 ppm.

### Synthesis 123

### 4-(4-fluorophenyl)-N-(2-oxo-1,2,3,4-tetrahydroquinolin-6-yl)thiazole-5-carboxamide (ALDHI-1017)

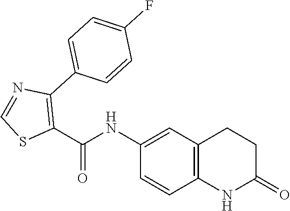 [SMILES: O=C1CCc2cc(NC(=O)c3scnc3-c3ccc(F)cc3)ccc2N1]

MS (ES+) m/z 367.9 (M+H).

¹H NMR (300 MHz, CD₃OD) δ 9.14 (s, 1H), 7.81 (dd, J=8.7, 5.5 Hz, 2H), 7.40 (s, 1H), 7.31 (dd, J=8.5, 2.4 Hz, 1H), 7.25-7.14 (m, 2H), 6.85 (d, J=8.5 Hz, 1H), 2.95 (t, J=7.6 Hz, 2H), 2.58 (dd, J=8.5, 6.7 Hz, 2H) ppm.

¹⁹F {1H} NMR (282 MHz, CD₃OD) 5-114.36 (s) ppm.

### Synthesis 124

### 4-(3-fluorophenyl)-N-(2-oxo-3,4-dihydro-1H-quinolin-6-yl)pyrimidine-5-carboxamide (ALDHI-1018)

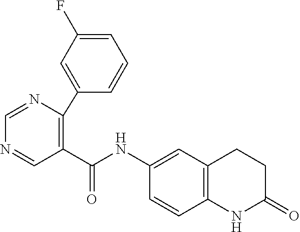 [SMILES: O=C1CCc2cc(NC(=O)c3cncnc3-c3cccc(F)c3)ccc2N1]

MS (ES+) m/z 363.3 (M+H).

¹H NMR (300 MHz, DMSO-d₆) δ 10.59 (s, 1H), 10.07 (s, 1H), 9.38 (s, 1H), 9.04 (s, 1H), 7.64-7.51 (m, 3H), 7.45-7.33 (m, 2H), 7.30 (dd, J=8.5, 2.4 Hz, 1H), 6.81 (d, J=8.5 Hz, 1H), 2.85 (dd, J=8.6, 6.5 Hz, 2H), 2.47-2.39 (m, 2H) ppm.

¹⁹F {1H} NMR (282 MHz, DMSO-d₆) δ −112.61 ppm.

### Synthesis 125

### 4-(3-fluorophenyl)-N-(2-oxo-3,4-dihydro-1H-quinolin-6-yl)oxazole-5-carboxamide (ALDHI-1019)

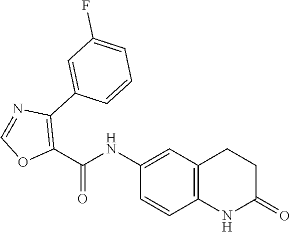 [SMILES: O=C1CCc2cc(NC(=O)c3ocnc3-c3cccc(F)c3)ccc2N1]

MS (ES+) m/z 352.3 (M+H).

¹H NMR (300 MHz, DMSO-d₆) δ 10.44 (s, 1H), 10.09 (s, 1H), 8.76 (s, 1H), 8.09 (dt, J=11.0, 2.1 Hz, 1H), 8.03 (dt, J=7.9, 1.1 Hz, 1H), 7.65-7.60 (m, 1H), 7.58-7.45 (m, 2H), 7.29 (tdd, J=8.4, 2.7, 1.0 Hz, 1H), 6.84 (d, J=8.5 Hz, 1H), 2.89 (t, J=7.5 Hz, 2H), 2.48-2.42 (m, 2H) ppm.

¹⁹F {1H} NMR (282 MHz, DMSO-d₆) δ −113.16 ppm.

### Synthesis 126

### 3-(3-fluorophenyl)-N-(2-oxo-3,4-dihydro-1H-quinolin-6-yl)pyridine-2-carboxamide (ALDHI-1020)

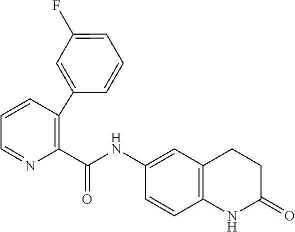 [SMILES: O=C1CCc2cc(NC(=O)c3ncccc3-c3cccc(F)c3)ccc2N1]

MS (ES+) m/z 362.3 (M+H).

¹H NMR (300 MHz, DMSO-d₆) δ 10.47 (s, 1H), 10.03 (s, 1H), 8.68 (dd, J=4.7, 1.6 Hz, 1H), 7.95 (dd, J=7.9, 1.6 Hz, 1H), 7.65 (dd, J=7.9, 4.8 Hz, 1H), 7.52-7.42 (m, 2H), 7.36 (dd, J=8.5, 2.4 Hz, 1H), 7.31-7.17 (m, 3H), 6.79 (d, J=8.5 Hz, 1H), 2.83 (t, J=7.5 Hz, 2H), 2.47-2.37 (m, 2H) ppm.

¹⁹F {1H} NMR (282 MHz, DMSO-d₆) δ −113.43 ppm.

### Synthesis 127

### 4-(2-fluorophenyl)-N-(2-oxo-3,4-dihydro-1H-quinolin-6-yl)thiazole-5-carboxamide (ALDHI-1021)

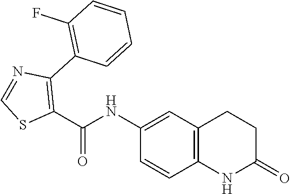 [SMILES: O=C1CCc2cc(NC(=O)c3scnc3-c3ccccc3F)ccc2N1]

MS (ES+) m/z 368.3 (M+H).

¹H NMR (300 MHz, DMSO-d₆) δ 10.24 (s, 1H), 10.07 (s, 1H), 9.31 (s, 1H), 7.77-7.66 (m, 1H), 7.51-7.38 (m, 2H), 7.36-7.20 (m, 3H), 6.78 (d, J=8.5 Hz, 1H), 2.83 (t, J=7.5 Hz, 2H), 2.43 (dd, J=8.6, 6.5 Hz, 2H) ppm.

¹⁹F {1H} NMR (282 MHz, DMSO-d₆) δ −114.47 ppm.

### Synthesis 128

### 3-(3-fluorophenyl)-N-(2-oxo-3,4-dihydro-1H-quinolin-6-yl)thiophene-2-carboxamide (ALDHI-1022)

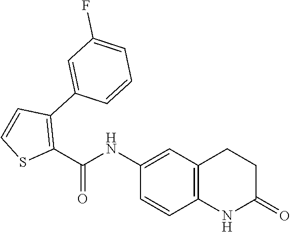 [SMILES: O=C1CCc2cc(NC(=O)c3sccc3-c3cccc(F)c3)ccc2N1]

MS (ES+) m/z 367.3 (M+H).

¹H NMR (300 MHz, DMSO-d₆) δ 10.11 (s, 1H), 10.06 (s, 1H), 7.81 (d, J=5.1 Hz, 1H), 7.49-7.13 (m, 7H), 6.77 (d, J=8.5 Hz, 1H), 2.83 (t, J=7.5 Hz, 2H), 2.46-2.37 (m, 2H) ppm.

¹⁹F {1H} NMR (282 MHz, DMSO-d₆) δ −113.27 ppm.

### Synthesis 129

### N-(2-oxo-3,4-dihydro-1H-quinolin-6-yl)-4-(2-thienyl)thiazole-5-carboxamide (ALDHI-1023)

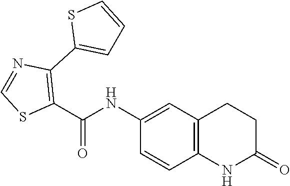 [SMILES: O=C1CCc2cc(NC(=O)c3scnc3-c3cccs3)ccc2N1]

MS (ES+) m/z 356.2 (M+H).

¹H NMR (300 MHz, DMSO-d₆) δ 10.67 (s, 1H), 10.11 (s, 1H), 9.21 (s, 1H), 7.63 (dd, J=5.1, 1.1 Hz, 1H), 7.58 (dd, J=3.7, 1.2 Hz, 1H), 7.54 (s, 1H), 7.48-7.32 (m, 1H), 7.12 (dd, J=5.1, 3.7 Hz, 1H), 6.84 (d, J=8.5 Hz, 1H), 2.88 (t, J=7.5 Hz, 2H), 2.48-2.42 (m, 2H) ppm.

### Synthesis 130

### 4-(3,5-difluorophenyl)-N-(2-oxo-3,4-dihydro-1H-quinolin-6-yl)thiazole-5-carboxamide (ALDHI-1024)

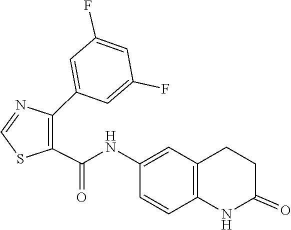 [SMILES: O=C1CCc2cc(NC(=O)c3scnc3-c3cc(F)cc(F)c3)ccc2N1]

MS (ES+) m/z 386.3 (M+H).

¹H NMR (300 MHz, DMSO-d₆) δ 10.62 (s, 1H), 10.11 (s, 1H), 9.30 (s, 1H), 7.50-7.39 (m, 3H), 7.38-7.28 (m, 2H), 6.83 (d, J=8.5 Hz, 1H), 2.86 (t, J=7.5 Hz, 2H), 2.44 (dd, J=8.6, 6.5 Hz, 2H) ppm.

¹⁹F {1H} NMR (282 MHz, DMSO-d₆) δ −109.39 (s) ppm.

### Synthesis 131

### N-(7-fluoro-2-oxo-3,4-dihydro-1H-quinolin-6-yl)-4-(3-fluorophenyl)thiazole-5-carboxamide (ALDHI-1025)

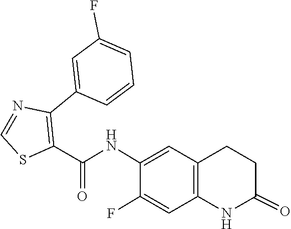 [SMILES: O=C1CCc2cc(NC(=O)c3scnc3-c3cccc(F)c3)c(F)cc2N1]

MS (ES+) m/z 386.3 (M+H).

¹H NMR (300 MHz, DMSO-d₆) δ 10.30 (s, 1H), 10.20 (s, 1H), 9.29 (s, 1H), 7.70-7.45 (m, 3H), 7.36 (d, J=8.0 Hz, 1H), 7.27 (tdd, J=8.2, 2.7, 1.0 Hz, 1H), 6.73 (d, J=11.3 Hz, 1H), 2.86 (t, J=7.6 Hz, 2H), 2.53-2.42 (m, 2H) ppm.

¹⁹F {1H} NMR (282 MHz, DMSO-d₆) δ −113.16 (s), −123.59 (s) ppm.

### Synthesis 132

### 4-(3-fluorophenyl)-2-methyl-N-(2-oxo-3,4-dihydro-1H-quinolin-6-yl)thiazole-5-carboxamide (ALDHI-1026)

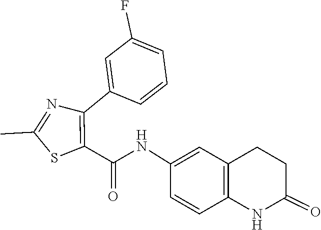 [SMILES: Cc1nc(-c2cccc(F)c2)c(C(=O)Nc2ccc3c(c2)CCC(=O)N3)s1]

MS (ES+) m/z 382.3 (M+H).

¹H NMR (300 MHz, DMSO-d₆) δ 10.45 (s, 1H), 10.09 (s, 1H), 7.60-7.40 (m, 4H), 7.34-7.18 (m, 2H), 6.80 (d, J=8.5 Hz, 1H), 2.85 (t, J=7.5 Hz, 2H), 2.75 (s, 3H), 2.43 (dd, J=8.6, 6.5 Hz, 2H) ppm.

¹⁹F {1H} NMR (282 MHz, DMSO-d₆) δ −113.05 (s) ppm.

### Biological Methods

### Recombinant Protein Production: ALDH1A3, ALDH1A1, ALDH1A2

Cloning: Open reading frames corresponding to the protein coding sequences of full length human ALDH1A1 (Uniprot ID P00352), ALDH1A2 (094788) and ALDH1A3 (P47895) were polymerase chain reaction (PCR) amplified from human cDNA and inserted into the multi-cloning sites of the commercially available (Novagen) E. coli expression vectors pET47b (ALDH1A1, EcoRI restriction site; ALDH1A2, BamHI and EcoRI sites) or pET28a (ALDH1A3, NdeI and BamHI sites), using standard molecular genetic techniques. Recombinant DNA products were sequence verified prior to use in expression studies.

Expression: Expression constructs were transformed into the E. coli strain Rosetta (DE3) (Merck) and individual colonies used to inoculate 10 mL lysogen broth (LB) medium supplemented with kanamycin and chloramphenicol at 50 μg/mL and 34 μg/mL, respectively. Cultures were grown to saturation overnight at 37° C., prior to being used to sub-inoculate fresh 1 L LB medium cultures (plus antibiotics, as before). 1 L cultures were grown at 37° C., 200 rpm, throughout the day until the optical density at 600 nm (OD600) reached ˜0.8, at which point growth temperature was reduced to 18° C. and 1 mM isopropyl β-d-1-thiogalacto pyranoside (IPTG) added to induce protein expression.

Cultures were left overnight (at least 16 h) under these conditions and the following day harvested by centrifugation (4000 G, 10 minutes). Culture supernatant was discarded and cell pellets stored at −80° C. until further processing.

Purification: Cell pellets were thawed on ice, re-suspended in Ni Affinity Buffer A (20 mM HEPES pH 7.5, 300 mM NaCl, 20 mM imidazole, 1 mM tris(2-carboxyethyl)phosphine (TCEP)) with protease inhibitors (cOmplete Ultra protease inhibitor, Roche), and lysed by sonication. Lysates were clarified by centrifugation at 18,000 G, 4° C., for 40 minutes, and loaded directly onto a pre-equilibrated (Buffer A) 5 mL HisTrap Fast Flow column (GE Healthcare) by way of an AKTA Fast Protein Liquid Chromatography (FPLC) system. The column was sequentially treated with 6 column volumes (CV) 100% Buffer A, 8 CV of 90% Buffer A:10% Buffer B (20 mM 4-(2-hydroxyethyl)-1-piperazineethanesulfonic acid (HEPES) pH 7.5, 300 mM NaCl, 500 mM imidazole, 1 mM TCEP) and finally a linear gradient from 10-70% Buffer B over 8 CV to elute recombinant His-tagged protein. Relevant fractions (as measured by 280 nm absorbance) collected during elution were pooled and subjected to further purification by size exclusion chromatography (Superdex 200 26/600, GE Healthcare, running buffer 20 mM 2-(carbamoylmethylamino)ethanesulfonic acid (ACES) pH 7.5, 1 mM TCEP). Relevant fractions from this step were pooled, aliquoted, and stored at −80° C. until required for compound profiling assays.

### Biochemical Assays (Primary and Selectivity Screen)

Materials: ALDH2 was purchased from Abnova. Tris(hydroxymethyl)aminomethane (Tris) was purchased from MP Biomedicals. TCEP, ethylenediaminetetraacetic acid (EDTA), propionaldehyde, DMSO and Tween-20 were purchased from Sigma. Nicotinamide adenine dinucleotide (NAD) was obtained from Abcam. The NAD(P)H-Glo™ Detection System was purchased from Promega. White, non-binding surface, 384 well plates were supplied by Corning (#3574).

Method: Inhibition of ALDH1A3 was assessed using the NAD(P)H-Glo™ Detection System (Cat. No. G9062) from Promega. This assay detects the production of NADH from NAD by ALDH1A3 via the coupled enzymatic conversion of a proluciferin reductase substrate to generate a luminescent signal. Inhibition of ALDH1A3 by small molecule inhibitors will result in reduction or abrogation of luminescence signal increase above background. Inhibition of ALDH1A1, ALDH1A2 and ALDH2 (selectivity assays) were assessed using the same assay.

Compounds were dosed into Corning 384 well assay plates using an Echo acoustic dispenser to give 10 point curves in a 3-fold dilution series. Compounds were dispensed from a 20 mM DMSO stock and initially screened at a top concentration of 20 μM (ALDH1A3) or 250 μM (ALDH1A1, ALDH1A2 & ALDH2). Enzyme was added to the plate in a 5 μL volume, according to the final concentrations in table 1 below, and pre-incubated with compound for 30 minutes at room temperature before the addition of 5 μL of the substrate (propionaldehyde) and cofactor (NAD), to effect final concentrations equal to the Km as shown in Table 1. The assay buffer used consisted of 40 mM Tris for 1A1 (pH 7.6), 1A2 (pH 7.6) and 1A3 (pH 9.0) and 10 mM Tris for ALDH2 (pH 8.0) supplemented with 1 mM TCEP, 0.01% Tween-20, 0.1 mM EDTA. The reaction mix was incubated at 26° C. on a shaking incubator for 30 minutes before being stopped by the addition of 10 μL of the Promega detection reagent and 100×IC₅₀ of inhibitors of the respective isoforms. After stopping the reaction the plate was incubated for 60 minutes at room temperature and the luminescence signal read on a PheraStar FS microplate reader (BMG Labtech) and resulting IC₅₀ values were calculated using Dotmatics software.

TABLE 1

| Reagent | ALDH1A1 | ALDH1A2 | ALDH1A3 | ALDH2 |
|---|---|---|---|---|
| Enzyme (nM) | 16 | 4 | 5 | 6 |
| Propionaldehyde | 1.4 | 100 | 300 | 6 |
| (μM) | | | | |
| NAD (μM) | 13 | 2.2 | 12 | 54 |

Enzyme, substrate and cofactor concentrations were selected to ensure the reaction ran under initial velocity conditions.

### ALDH1A3 Cellular Assay

Materials: WM266.4 cells were procured from ATCC. (WM266-4 is a metastatic human melanoma cell line.) The ALDEFLUOR™ Assay kit (cat. no. 01700) and Aldefluor buffer (cat. no. 01702) were procured from StemCell Technologies. Dulbecco's Modified Eagle Medium (DMEM), FluoroBrite DMEM, Glutamax and Hoechst 33342 were purchased from ThermoFisher. Verapamil was obtained from Fluorochem. FBS was obtained from Sigma. Echo Qualified 384-well low-dead-volume microplate and Echo Qualified 384-well polypropylene microplate were purchased from Labcyte, and the Microclear black sterile polystyrene microplate from Greiner Bio-One.

Method: Intracellular inhibition of ALDH1A3 was assessed using the ALDEFLUOR™ Assay kit. The activated ALDEFLUOR™ reagent, BODIPY-aminoacetaldehyde (BAAA), is a fluorescent non-toxic substrate for ALDH enzymes, which freely diffuses into intact and viable cells. In the presence of ALDH enzyme activity, BAAA is converted into BODIPY-aminoacetate (BAA), which is retained inside the cells. The amount of fluorescent reaction product is proportional to the ALDH activity in the cells and is measured using an imaging cytometer. Active efflux of the reaction product is inhibited by an efflux inhibitor (Verapamil) in the ALDEFLUOR™ Assay Buffer.

The WM266.4 melanoma cell line was characterised and shown to predominately express the ALDH1A3 isoform and hence assessment of inhibitors in this cell line constitutes the assay for cellular ALDH1A3 activity. A frozen vial of cells was recovered into a T225 flask containing DMEM, high glucose, 10% FBS, 1% glutamax, 1% HEPES. The following day, media was changed and cells were ready for assay from 72 hours after recovery. Cells were transferred into FluoroBrite DMEM (plus 10% FBS, 1% glutamax and 1% HEPES) and diluted to 2.5×10⁵ cells/mL. 30 μL of cells were added to each well to give 7500 cells/well and then incubated overnight at 37° C., 5% CO₂. After overnight incubation, the media was aspirated and replaced by 20 μL substrate/Hoechst buffer solution. Each plate requires 9 mL of solution comprising, 9 mL ALDEFLUOR assay buffer, 18 μL BAAA substrate (final concentration 500 nM), 4.5 μL Hoechst 33342 (final concentration 5 μg/mL) and 4.5 μL Verapamil (final concentration 50 μM). Compounds were prepared for dosing using the Echo acoustic dispenser by aliquoting into a source plate and dosed directly onto the cell assay plate to give a 10 point dose response per compound ranging from 10 μM to 0.0005 μM final concentration in 3-fold dilution steps. Plates were then incubated for 60 minutes at 37° C., 5% CO₂. The buffer was then aspirated and cells washed twice in ice-cold PBS. Plates were kept on ice and 30 μL of cold Aldefluor buffer with 50 μM verapamil dispensed per well. The plates were then imaged using the CellInsight fluorescent microscope. Hoechst fluorescence in the 405 nm channel indicated the nuclear area and this was used to define nuclei and then the surrounding cellular area. Fluorescence in the 488 nm channel of the defined cellular area indicates ALDH1A3 activity. The percentage of cells positive for ALDH activity was plotted versus compound concentration to generate EC50 values using curve fitting parameters defined in Dotmatics software.

### Biological Data

The data for the primary biochemical assay (ALDH1A3 IC₅₀), Aldefluor cellular assay (ALDH1A3 Aldefluor IC₅₀), and selectivity assays (ALDH1A1 IC₅₀, ALDH1A2 IC₅₀, ALDH2 IC₅₀) are summarised in the following table.

TABLE 2

| | | ALDH1A3 | | | |
|---|---|---|---|---|---|
| Code | ALDH1A3 | Aldefluor | ALDH1A1 | ALDH1A2 | ALDH2 |
| ALDHI-1001 | A | A | E | E | E |
| ALDHI-1002 | A | A | E | C | E |
| ALDHI-1003 | A | A | E | C | E |
| ALDHI-1004 | A | A | E | E | E |
| ALDHI-1005 | A | A | E | D | E |
| ALDHI-1006 | B | | E | | D |
| ALDHI-1007 | C | | E | C | E |
| ALDHI-1008 | B | B | E | C | E |
| ALDHI-1009 | C | | E | | |
| ALDHI-1010 | C | | E | C | E |
| ALDHI-1011 | B | C | E | C | E |
| ALDHI-1012 | C | | D | | E |
| ALDHI-1013 | C | | E | C | E |
| ALDHI-1014 | B | C | E | D | E |
| ALDHI-1015 | C | | E | | |
| ALDHI-1016 | B | A | E | C | E |
| ALDHI-1017 | A | B | E | C | E |
| ALDHI-1018 | B | B | E | | E |
| ALDHI-1019 | B | C | E | E | E |
| ALDHI-1020 | C | C | | | |
| ALDHI-1021 | A | A | E | C | E |
| ALDHI-1022 | B | | D | C | E |
| ALDHI-1023 | A | A | E | B | E |
| ALDHI-1024 | A | | E | | |
| ALDHI-1025 | A | | E | | |
| ALDHI-1026 | A | | E | | |
| ALDHI-2001 | A | A | E | B | E |
| ALDHI-2002 | A | C | E | D | E |
| ALDHI-2003 | A | C | E | C | D |
| ALDHI-2004 | B | C | E | D | E |
| ALDHI-2005 | A | B | E | D | E |
| ALDHI-2006 | B | C | E | C | E |
| ALDHI-2007 | A | A | E | B | E |
| ALDHI-2008 | A | A | E | D | E |
| ALDHI-2009 | B | B | E | C | E |
| ALDHI-2010 | A | B | E | E | E |
| ALDHI-2011 | B | C | E | D | E |
| ALDHI-2012 | B | C | E | E | E |
| ALDHI-2013 | A | B | E | C | E |
| ALDHI-2014 | A | B | E | E | E |
| ALDHI-2015 | C | | E | | |
| ALDHI-2016 | B | C | E | E | E |
| ALDHI-2017 | C | | E | | |
| ALDHI-2018 | A | A | E | B | E |
| ALDHI-2019 | B | B | E | B | E |
| ALDHI-2020 | A | B | E | C | E |
| ALDHI-2021 | B | C | E | C | E |
| ALDHI-2022 | A | B | E | C | E |
| ALDHI-2023 | A | A | E | A | E |
| ALDHI-2024 | C | | E | | |
| ALDHI-2025 | C | | E | | |
| ALDHI-2026 | B | C | E | E | E |
| ALDHI-2027 | B | C | E | D | E |
| ALDHI-2028 | A | B | E | E | E |
| ALDHI-2029 | B | B | E | D | E |
| ALDHI-2030 | B | B | E | D | E |
| ALDHI-2031 | A | A | E | B | E |
| ALDHI-2032 | A | A | D | B | E |
| ALDHI-2033 | B | C | E | E | E |
| ALDHI-2034 | B | C | E | D | E |
| ALDHI-2035 | A | A | D | B | E |
| ALDHI-2036 | C | | E | | |
| ALDHI-2037 | A | A | E | B | E |
| ALDHI-2038 | A | C | D | C | E |
| ALDHI-2039 | A | B | C | C | E |
| ALDHI-2040 | B | B | E | B | E |
| ALDHI-2041 | B | C | E | C | E |
| ALDHI-2042 | B | B | D | B | E |
| ALDHI-2043 | B | B | E | C | E |
| ALDHI-2044 | A | B | E | D | E |
| ALDHI-2045 | A | A | E | C | E |
| ALDHI-2046 | C | | D | | |
| ALDHI-2047 | C | | E | | |
| ALDHI-2048 | A | A | D | B | E |
| ALDHI-2049 | C | | E | | |
| ALDHI-2050 | A | B | E | C | E |
| ALDHI-2051 | A | A | E | C | E |
| ALDHI-2052 | A | A | E | B | E |
| ALDHI-2053 | A | A | E | C | E |
| ALDHI-3001 | B | B | E | C | E |
| ALDHI-3002 | A | A | E | B | E |
| ALDHI-3003 | B | B | E | C | E |
| ALDHI-3004 | B | A | E | C | E |
| ALDHI-3005 | A | B | E | D | E |
| ALDHI-3006 | B | B | D | C | E |
| ALDHI-3007 | B | B | D | C | E |
| ALDHI-3008 | B | B | E | C | E |
| ALDHI-3009 | C | | D | | |
| ALDHI-3010 | B | B | D | D | E |
| ALDHI-3011 | C | | D | | |
| ALDHI-3012 | C | | E | | |
| ALDHI-3013 | B | C | E | E | E |
| ALDHI-3014 | C | | E | | |

Legend:
A: <0.1 μM
B: ≥0.1 μM and <1 μM
C: ≥1 μM and <10 μM
D: ≥10 μM and <100 μM
E: ≥100 μM

### In Vivo Pharmacokinetics (PK)

All procedures involving animals were performed in accordance with national Home Office regulations under the Animals (Scientific Procedures) Act 1986 and within guidelines set out by the Institute's Animal Ethics Committee and the United Kingdom Coordinating Committee for Cancer Research's ad hoc Committee on the Welfare of Animals in Experimental Neoplasia. Female BALB/c mice (Charles River Laboratories) at 6 weeks of age were used for the PK analyses. The mice were dosed orally by gavage (5 mg/kg in DMSO:water 1:19 v:v; n=6) or intravenously in the tail vein (1 mg/kg in DMSO:Tween20:saline 10:1:89 v:v:v; n=6). Blood samples (˜20 μL) were taken from the tail vein from each group of mice (2 groups, 3 mice/group) alternated at 5, 15 and 30 minutes, 1, 2, 4 and 6 and 8 hours after dosing. For clarity, group 1 was bled at 5 minutes, 30 minutes, 2 hours and 6 hours; group 2 was bled at 15 minutes, 1 hour, 4 hours and 8 hours. Plasma samples were snap frozen in liquid nitrogen and then stored at −80° C. prior to analysis.

Test compound solutions (1 mg/mL in DMSO) were used to make stock Standard Curve (SC) and Quality Control (QC) solutions at appropriate concentrations. Blank plasma was spiked with stock solutions to produce a 9-point standard curve ranging from 1.5-10,000 ug/mL; with 2 QC concentrations within this range and where the DMSO concentration was 10% of the plasma volume. Plasma PK samples, Standards and QC's were added to individual Eppendorf tubes and DMSO (10% of plasma volume) was added to the plasma PK samples. SC, QC and plasma samples were extracted with methanol (100 μL) containing internal standard. Following protein precipitation, the samples were centrifuged for 10 minutes in a refrigerated centrifuge (4° C.) at 14000 rpm. The supernatants were removed to a 96-well plate and centrifuged for a further 10 minutes in a refrigerated centrifuge (4° C.) at 3700 rpm. Samples were analysed by Liquid Chromatography Mass Spectrometry (LC-MS/MS) for the compound plasma concentrations. Non-compartmental analysis was performed on plasma concentration data using the Excel macro PK Solver 2.0.

The pharmacokinetics data are summarised in the following table.

TABLE 3
Pharmacokinetics Data

| | Cₘₐₓ po | AUC po | Cl iv | F |
|---|---|---|---|---|
| Code | (μM) | (μM · hr) | (mL/min/kg) | (%) |
| ALDHI-3008 | 1.8 | 1.5 | — | — |
| ALDHI-1003 | 6.1 | 13.3 | 22.1 | 100 |

The foregoing has described the principles, preferred embodiments, and modes of operation of the present invention. However, the invention should not be construed as limited to the particular embodiments discussed. Instead, the above-described embodiments should be regarded as illustrative rather than restrictive. It should be appreciated that variations may be made in those embodiments by workers skilled in the art without departing from the scope of the present invention.

### REFERENCES

Publications are cited herein in order to more fully describe the state of the art to which the invention pertains. Full citations for these references are provided below.

Each of these publications is incorporated herein by reference in its entirety into the present disclosure, to the same extent as if each individual reference was specifically and individually indicated to be incorporated by reference.
Aissa et al., 2021, “Single-cell transcriptional changes associated with drug tolerance and response to combination therapies in cancer”, Nature Communications, Vol. 12, Article 1628.
Bazewicz et al., 2019, “Aldehyde dehydrogenase in regulatory T-cell development, immunity and cancer”, Immunology, Vol. 156, No. 1, pp. 47-55.
Burke et al., 2017, “db/db Mice exhibit features of human type 2 diabetes that are not present in weight-matched C57BL/6J mice fed a Western diet”, Journal of Diabetes Research, Article ID 8503754.
Burke et al., 2018, “Pancreatic deletion of the interleukin-1 receptor disrupts whole body glucose homeostasis and promotes islet β-cell de-differentiation”, Molecular Metabolism, Vol. 14, pp. 95-107.
Cai et al., 2021, “TNRC6C Functions as a Tumor Suppressor and Is Frequently Downregulated in Papillary Thyroid Cancer”, International Journal of Endocrinology, Article ID 6686998.
Casanova-Salas et al., 2015, “MiR-187 targets the androgen-regulated gene ALDH1A3 in prostate cancer”, PLOS One, Vol. 10, No. 5, e0125576.
Chefetz et al., 2019, “A Pan-ALDH1A Inhibitor Induces Necroptosis in Ovarian Cancer Stem-like Cells”, Cell Rep., Vol. 26, No. 11, pp. 3061-3075.e6.
Chen et al., 2016, “ALDH1A3, the Major Aldehyde Dehydrogenase Isoform in Human Cholangiocarcinoma Cells, Affects Prognosis and Gemcitabine Resistance in Cholangiocarcinoma Patients”, Clin Cancer Res, Vol. 22, No. 16. pp. 4225-4235.
Cheng et al., 2016, “FOXD1-ALDH1A3 Signaling Is a Determinant for the Self-Renewal and Tumorigenicity of Mesenchymal Glioma Stem Cells”, Cancer Res., Vol. 76, No. 24, pp. 7219-7230.
Cinti et al., 2016, “Evidence of β-cell dedifferentiation in human type 2 diabetes”, Journal of Clinical Endocrinology and Metabolism, Vol. 101, No. 3, pp. 1044-54.
Cioce et al., 2021, “Insights into Intra-Tumoral Heterogeneity: Transcriptional Profiling of Chemoresistant MPM Cell Subpopulations Reveals Involvement of NFkB and DNA Repair Pathways and Contributes a Prognostic Signature”, Int. J. Mol. Sci., Vol. 22, No. 21, 12071.
Croker et al., 2017, “Differential Functional Roles of ALDH1A1 and ALDH1A3 in Mediating Metastatic Behavior and Therapy Resistance of Human Breast Cancer Cells”, Int. J. Mol. Sci., Vol. 18, No. 10, 2039.
Dinavahi et al., 2019, “Aldehyde Dehydrogenase Inhibitors for Cancer Therapeutics”, Trends Pharmacol. Sci., Vol. 40, No. 10, pp. 774-789.
Duan et al., 2016, “ALDH1A3, a metabolic target for cancer diagnosis and therapy”, Int. J. Cancer, Vol. 139, No. 5, pp. 965-975.
Durinikova et al., 2018, “ALDH1A3 upregulation and spontaneous metastasis formation is associated with acquired chemoresistance in colorectal cancer cells”, BMC Cancer, Vol. 18, No. 1, 848.
Esposito et al., “Heterocyclic Compounds and Uses Thereof”, international (PCT) patent publication number WO 2021/151062 A1 published 29 Jul. 2021.
Esposito et al., “Tetrahydroquinolino derivatives for the treatment of metastatic and chemoresistant cancers”, international (PCT) patent publication number WO 2020/028461 A1 published 6 Feb. 2020.
Flahaut et al., 2016, “Aldehyde dehydrogenase activity plays a key role in the aggressive phenotype of neuroblastoma”, BMC Cancer, Vol. 16, No. 1, 781.
Gelardi et al., 2021, “A Selective Competitive Inhibitor of Aldehyde Dehydrogenase 1A3 Hinders Cancer Cell Growth, Invasiveness and Stemness In Vitro”, Cancers (Basel), Vol. 13, No. 2, 356.
Gerner Seitzberg et al., 2016, “Compounds active towards bromodomains”, international (PCT) patent publication number WO 2016/016316 A1 published 4 Feb. 2016.
Ginestier et al., 2007, “ALDH1 is a marker of normal and malignant human mammary stem cells and a predictor of poor clinical outcome”, Cell Stem Cell, Vol. 1, No. 5, pp. 555-567.
Grimley et al., 2021, “Aldehyde dehydrogenase inhibitors promote DNA damage in ovarian cancer and synergize with ATM/ATR inhibitors”, Theranostics, Vol. 11, No. 8, pp. 3540-3551.
Haldar et al., “Methods And Compositions For Targeting Retinoic Acid For Solid Tumor Immunotherapy”, US patent publication number US 2020/0352958 A1 published 12 Nov. 2020.
Huang et al., 2021, “Diethyldithiocarbamate-copper complex (CuET) inhibits colorectal cancer progression via miR-16-5p and 15b-5p/ALDH1A3/PKM2 axis-mediated aerobic glycolysis pathway”, Oncogenesis, Vol. 10, Article No. 4.
Huddle et al., 2018, “Structure-Based Optimization of a Novel Class of Aldehyde Dehydrogenase 1A (ALDH1A) Subfamily-Selective Inhibitors as Potential Adjuncts to Ovarian Cancer Chemotherapy”, J. Med. Chem., Vol. 61, No. 19, pp. 8754-8773.
Huddle et al., 2021, “Development of 2,5-dihydro-4H-pyrazolo[3,4-d]pyrimidin-4-one inhibitors of aldehyde dehydrogenase 1A (ALDH1A) as potential adjuncts to ovarian cancer chemotherapy”, Eur. J. Med. Chem., Vol. 211, 113060.
Ibrahim et al., 2021, “Design, Synthesis, Biological Evaluation and In Silico Study of Benzyloxybenzaldehyde Derivatives as Selective ALDH1A3 Inhibitors”, Molecules, Vol. 26, No. 19, 5770.
Kamada et al., 2021, “Functional inhibition of cancer stemness-related protein DPP4 rescues tyrosine kinase inhibitor resistance in renal cell carcinoma”, Oncogene, Vol. 40, No. 22, pp. 3899-3913.
Kawakami et al., 2020, “ALDH1A3-mTOR axis as a therapeutic target for anticancer drug-tolerant persister cells in gastric cancer”, Cancer Science, Vol. 111, No. 3, pp. 962-973.
Kim-Muller et al., 2016, “Aldehyde dehydrogenase 1a3 defines a subset of failing pancreatic β cells in diabetic mice”, Nat. Commun., Vol. 7, 12631.
Koga et al., 1997, “Carbostyril derivatives as antithrombic agents”, international (PCT) patent publication number WO 97/12869 A1 published 10 Apr. 1997.
Kong et al., 2016, “A subset of metastatic pancreatic ductal adenocarcinomas depends quantitatively on oncogenic Kras/Mek/Erk-induced hyperactive mTOR signalling”, Gut, Vol. 65, No. 4, pp. 647-657.
Koppaka et al., 2012, “Aldehyde dehydrogenase inhibitors: a comprehensive review of the pharmacology, mechanism of action, substrate specificity, and clinical application”, Pharmacol. Rev., Vol. 64, No. 3, pp. 520-539.
Kozovska et al., 2016, “Malignant melanoma: diagnosis, treatment and cancer stem cells”, Neoplasma, Vol. 63, No. 4, pp. 510-517.
Larsen et al., “Small molecule inhibitors of ALDH and uses thereof”, international (PCT) patent publication number WO 2017/223086 A1 published 28 Dec. 2017.
Laskar and Younus, 2019, “Aldehyde toxicity and metabolism: the role of aldehyde dehydrogenases in detoxification, drug resistance and carcinogenesis”, Drug Metab. Rev., Vol. 51, No. 1, pp. 42-64.
Li et al., 2018, “ALDH1A3 induces mesenchymal differentiation and serves as a predictor for survival in glioblastoma”, Cell Death Dis., Vol. 9, No. 12, 1190.
Li et al., 2021, “ALDH1A3 coordinates metabolism with gene regulation in pulmonary arterial hypertension”, Circulation, Vol. 143, pp. 2074-2090.
Luo et al., 2012, “ALDH1A isozymes are markers of human melanoma stem cells and potential therapeutic targets”, Stem Cells, Vol. 30, No. 10, pp. 2100-2013.
Mao et al., 2013, “Mesenchymal glioma stem cells are maintained by activated glycolytic metabolism involving aldehyde dehydrogenase 1A3”, Proc. Natl. Acad. Sci. USA, Vol. 110, No. 21, pp. 8644-8649.
Marayati et al., 2021, “PIM kinases mediate resistance to cisplatin chemotherapy in hepatoblastoma”, Scientific Reports, Vol. 11, No. 1, 5984.
Marcato et al., 2011a, “Aldehyde dehydrogenase: its role as a cancer stem cell marker comes down to the specific isoform”, Cell Cycle, Vol. 10, No. 9, pp. 1378-1384.
Marcato et al., 2011b, “Aldehyde dehydrogenase activity of breast cancer stem cells is primarily due to isoform ALDH1A3 and its expression is predictive of metastasis”, Stem Cells, Vol. 29, No. 1, pp. 32-45.
Marcato et al., 2015, “Aldehyde dehydrogenase 1A3 influences breast cancer progression via differential retinoic acid signaling”, Mol. Oncol., Vol. 9, No. 1, pp. 17-31.
Masui et al., 2008, “Nirogenous heterocyclic derivatives substituted with cyclic groups”, European patent publication number EP 1988077 A1 published 5 Nov. 2008.
Matsunaga et al., 2018, “Optimized Dosing Schedule Based on Circadian Dynamics of Mouse Breast Cancer Stem Cells Improves the Antitumor Effects of Aldehyde Dehydrogenase Inhibitor”, Cancer Res., Vol. 78, No. 13, pp. 3698-3708.
Morgan et al., 2015, “N,N-diethylaminobenzaldehyde (DEAB) as a substrate and mechanism-based inhibitor for human ALDH isoenzymes”, Chem. Biol. Interact., Vol. 234, pp. 18-28.
Ni et al., 2020, “High expression of ALDH1A3 might independently influence poor progression-free and overall survival in patients with glioma via maintaining glucose uptake and lactate production”, Cell Biol. Int., Vol. 44, No. 2, pp. 569-582.
Nie et al., 2020, “ALDH1A3 accelerates pancreatic cancer metastasis by promoting glucose metabolism”, Front. Oncol., Vol. 10, 915.
Nishi et al., 1987, “Carbostyryl derivatives, process for preparing them, pharmaceutical compositions, and use”, European patent publication number EP 0240015 A2 published 7 Oct. 1987.
Opdenaker et al., 2014, “Immunohistochemical analysis of aldehyde dehydrogenase isoforms and their association with estrogen-receptor status and disease progression in breast cancer”, Breast Cancer (Dove Med Press), Vol. 6, pp. 205-209.
Pattabiraman et al., 2014, “Tackling the cancer stem cells—what challenges do they pose?”, Nat. Rev. Drug Discov., Vol. 13, No. 7, pp. 497-512.
Pérez-Alea et al., 2017, “ALDH1A3 is epigenetically regulated during melanocyte transformation and is a target for melanoma treatment”, Oncogene, Vol. 36, No. 41, pp. 5695-5708.
Pors et al., 2014, “Aldehyde dehydrogenases in cancer: an opportunity for biomarker and drug development?”, Drug Discov. Today, Vol. 19, No. 12, pp. 1953-1963.
Rodriguez-Torres and Allan, 2016, “Aldehyde dehydrogenase as a marker and functional mediator of metastasis in solid tumors”, Clin. Exp. Metastasis, Vol. 33, No. 1, pp. 97-113.
Rodriguez-Zavala et al., 2019, “Role of Aldehyde Dehydrogenases in Physiopathological Processes”, Chem. Res. Toxicol., Vol. 32, No. 3, pp. 405-420.
Schmidtova et al., 2019, “Disulfiram Overcomes Cisplatin Resistance in Human Embryonal Carcinoma Cells”, Cancers, Vol. 11, No. 9, 1224.
Shao et al., 2014, “Essential role of aldehyde dehydrogenase 1A3 for the maintenance of non-small cell lung cancer stem cells is associated with the STAT3 pathway”, Clin. Cancer Res., Vol. 20, No. 15, pp. 4154-4166.
Simões et al., 2015, “Anti-estrogen Resistance in Human Breast Tumors Is Driven by JAG1-NOTCH4-Dependent Cancer Stem Cell Activity”, Cell Rep., Vol. 12, No. 12, pp. 1968-1977.
Takasugi et al., 2002, “Amide Compounds”, international (PCT) patent publication number WO 02/090347 A1 published 14 Nov. 2002.
Thomas et al., 2016, “Citral reduces breast tumor growth by inhibiting the cancer stem cell marker ALDH1A3”, Mol. Oncol., Vol. 10, No. 9, pp. 1485-1496.
Tominaga et al., 1982, “Cardiotonic agent”, Japanese patent publication number JP S5780322 A published 19 May 1982.
Vasilogiannakopoulou et al., 2018, “Impact of Aldehyde Dehydrogenase Activity on Gliomas”, Trends Pharmacol. Sci., Vol. 39, No. 7, pp. 605-609.
Xie et al., 2019, “ALDH1A3 Regulations of Matricellular Proteins Promote Vascular Smooth Muscle Cell Proliferation”, iScience, Vol. 27, pp. 872-882.
Yang et al., 2013, “Positive ALDH1A3 and negative GPX3 expressions are biomarkers for poor prognosis of gallbladder cancer”, Dis. Markers, Vol. 35, No. 3, pp. 163-172.
Yasgar et al., 2017, “A High-Content Assay Enables the Automated Screening and Identification of Small Molecules with Specific ALDH1A1-Inhibitory Activity”, PLOS One, Vol. 12, No. 1, e0170937.
Zhang et al., 2013, “Genome-wide DNA methylation profiling identifies ALDH1A3 promoter methylation as a prognostic predictor in G-CIMP-primary glioblastoma”, Cancer Lett., Vol. 328, No. 1, pp. 120-125.

1. A compound of the following formula: 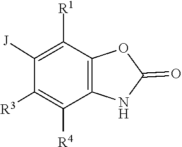
or a pharmaceutically acceptable salt or solvate thereof;
wherein -J is: 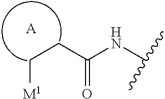
wherein:
Ring A is:
an aromatic monocyclic ring having 5 or 6 ring atoms;
and is optionally substituted with one or more substituents —R^{A};
wherein each —R^{A} is independently —R^{AA}, —R^{AAX}, —OH, —OR^{AA}, —OR^{AAX}, —F, —Cl, —Br, —I, —NH₂, —NHR^{AA}, —NR^{AA}₂, —R^{AAN}, —C(═O)R^{AA}, —C(═O)OH, —C(═O)OR^{AA}, —OC(═O)R^{AA}, —NHC(═O)R^{AA}, —C(═O)NH₂, —C(═O)NHR^{AA}, —C(═O)NR^{AA}₂, —C(═O)R^{AAN}, —S(═O)₂R^{AA}, —S(═O)₂NH₂, —S(═O)₂NHR^{AA}, —S(═O)₂NR^{AA}₂, —S(═O)₂R^{AAN}, —CN, or —NO₂;
wherein:
each —R^{AA} is independently saturated linear or branched C₁₋₄alkyl or saturated C₃₋₆cycloalkyl;
each —R^{AAX} is independently saturated linear or branched C₁₋₄haloalkyl; and
each —R^{AAN} is independently azetidino, pyrrolidino, piperidino, piperazino, morpholino, thiomorpholino, or 1,1-dioxo-thiomorpholino, and is optionally substituted with one or more substituents selected from —R^{AA}, —OH, and —OR^{AA};
either -M¹ is: 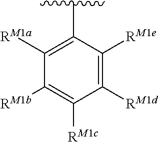
wherein:
each of —R^{M1a} and —R^{M1e} is independently —H or —R^{M1-ortho};
each of —R^{M1b} and —R^{M1d} is independently —H or —R^{M1-meta};
—R^{M1c} is independently —H or —R^{M1-para};
with the proviso that —R^{M1a}, —R^{M1b}, —R^{M1c}, —R^{M1d}, and —R^{M1e} are not all —H;
wherein each —R^{M1-ortho}, each —R^{M1-meta}, —R^{M1-para} is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OH, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —S(═O)₂NH₂, —S(═O)₂NHR^{M11}, —S(═O)₂NR^{M11}₂, —S(═O)₂R^{M11N}, —CN, or —NO₂;
and wherein -M¹ is attached to Ring A by a bond between a ring carbon atom of -M¹ and a ring carbon atom of Ring A;
or -M¹ is an aromatic monocyclic heterocyclic ring having 5 or 6 ring atoms, and is optionally substituted with one or more substituents —R^{M1};
wherein each —R^{M1} is independently —R^{M11}, —R^{M11X}, —OH, —OR^{M11}, —OR^{M11X}, —F, —Cl, —Br, —I, —NH₂, —NHR^{M11}, —NR^{M11}₂, —R^{M11N}, —C(═O)R^{M11}, —C(═O)OH, —C(═O)OR^{M11}, —OC(═O)R^{M11}, —NHC(═O)R^{M11}, —C(═O)NH₂, —C(═O)NHR^{M11}, —C(═O)NR^{M11}₂, —C(═O)R^{M11N}, —S(═O)₂R^{M11}, —S(═O)₂NH₂, —S(═O)₂NHR^{M11}, —S(═O)₂NR^{M11}₂, —S(═O)₂R^{M11N}, —CN, or —NO₂;
wherein:
each —R^{M11} is independently saturated linear or branched C₁₋₄alkyl;
each —R^{M11X} is independently saturated linear or branched C₁₋₄haloalkyl; and
each —R^{M11N} is independently azetidino, pyrrolidino, piperidino, piperazino, morpholino, thiomorpholino, or 1,1-dioxo-thiomorpholino, and is optionally substituted with one or more substituents selected from —R^{M11}, —OH, and —OR^{M11};
and wherein -M¹ is attached to Ring A by a bond between a ring carbon atom of -M¹ and a ring carbon atom of Ring A;
or wherein -J is: 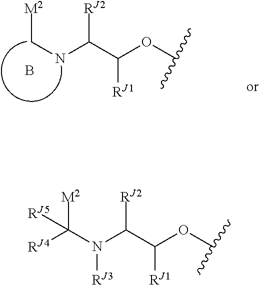
wherein:
each —R^{J1} is independently —H or —R^{JJ};
each —R^{J2} is independently —H or —R^{JJ};
—R^{J3} is independently —R^{JJ}, -L^{JJ}-OH, -L^{JJ}-NH₂, -L^{JJ}-NHR^{JJ}, or -L^{JJ}-NR^{JJ}₂;
—R^{J4} is independently —H, —R^{JJ}, -L^{JJ}-OH, -L^{JJ}-NH₂, -L^{JJ}-NHR^{JJ}, or -L^{JJ}-NR^{JJ}₂;
—R^{J5} is independently —H, —R^{JJ}, -L^{JJ}-OH, -L^{JJ}-NH₂, -L^{JJ}-NHR^{JJ}, or -L^{JJ}-NR^{JJ}₂;
or —R^{J4} and —R^{J5} taken together form ═O;
wherein:
each —R^{JJ} is independently saturated linear or branched C₁₋₄alkyl;
each -L^{JJ}- is independently saturated linear or branched C₁₋₄alkylene;
Ring B is independently Ring 1 or Ring B2;
Ring B1 is a non-aromatic monocyclic heterocyclic ring having 4 to 7 ring atoms;
and is optionally substituted with one or more substituents —R^{B1};
and/or is optionally be substituted with ═O;
wherein each —R^{B1} is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OH, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —S(═O)₂NH₂, —S(═O)₂NHR^{BB}, —S(═O)₂NR^{BB}₂, —S(═O)₂R^{BBN}, —CN, or —NO₂;
Ring B2 is an heteroaromatic monocyclic ring having 5 or 6 ring atoms, and is optionally substituted with one or more substituents —R^{B2},
wherein each —R^{B2} is independently —R^{BB}, —R^{BBX}, —OH, —OR^{BB}, —OR^{BBX}, —F, —Cl, —Br, —I, —NH₂, —NHR^{BB}, —NR^{BB}₂, —R^{BBN}, —C(═O)R^{BB}, —C(═O)OH, —C(═O)OR^{BB}, —OC(═O)R^{BB}, —NHC(═O)R^{BB}, —C(═O)NH₂, —C(═O)NHR^{BB}, —C(═O)NR^{BB}₂, —C(═O)R^{BBN}, —S(═O)₂R^{BB}, —S(═O)₂NH₂, —S(═O)₂NHR^{BB}, —S(═O)₂NR^{BB}₂, —S(═O)₂R^{BBN}, —CN, or —NO₂;
wherein:
each —R^{BB} is independently saturated linear or branched C₁₋₄alkyl or saturated C₃₋₆cycloalkyl;
each —R^{BBX} is independently saturated linear or branched C₁₋₄haloalkyl; and
each —R^{BBN} is independently azetidino, pyrrolidino, piperidino, piperazino, morpholino, thiomorpholino, or 1,1-dioxo-thiomorpholino, and is optionally substituted with one or more substituents selected from —R^{BB}, —OH, and —OR^{BB};
-M² is independently:
phenyl,
and is optionally substituted with one or more substituents —R^{M2}; or
an aromatic monocyclic heterocyclic ring having 5 or 6 ring atoms,
and is optionally substituted with one or more substituents —R^{M2};
wherein each —R^{M2} is independently —R^{M22}, —R^{M22X}, —OH, —OR^{M22}, —OR^{M22X}, —F, —Cl, —Br, —I, —NH₂, —NHR^{M22}, —NR^{M22}₂, —R^{M22N}, —C(═O)R^{M22}, —C(═O)OH, —C(═O)OR^{M22}, —OC(═O)R^{M22}, —NHC(═O)R^{M22}, —C(═O)NH₂, —C(═O)NHR^{M22}, —C(═O)NR^{M22}₂, —C(═O)R^{M22N}, —S(═O)₂R^{M22}, —S(═O)₂NH₂, —S(═O)₂NHR^{M22}, —S(═O)₂NR^{M22}₂, —S(═O)₂R^{M22N}, —CN, or —NO₂;
and wherein:
each —R^{M22} is independently saturated linear or branched C₁₋₄alkyl;
each —R^{M22X} is independently saturated linear or branched C₁₋₄haloalkyl; and
each —R^{M22}N is independently azetidino, pyrrolidino, piperidino, piperazino, morpholino, thiomorpholino, or 1,1-dioxo-thiomorpholino, and is optionally substituted with one or more substituents selected from —R^{M22}, —OH, and —OR^{M22};
and wherein -Q- is independently:
—CH₂—CR^{Q1}R^{Q2}—;
—O—CR^{Q1}R^{Q2}—;
—S—CR^{Q1}R^{Q2}—;
—CH₂—CH₂—CR^{Q1}R^{Q2}—; or
—CR^{Q3}═CR^{Q4}—;
wherein:
each —R^{Q1} is independently —H or —R^{QQ};
each —R^{Q2} is independently —H or —R^{QQ};
—R^{Q3} is independently —H or —R^{QQ};
—R^{Q4} is independently —H or —R^{QQ};
wherein:
each —R^{QQ} is independently saturated linear or branched C₁₋₄alkyl;
and wherein:
—R¹ is independently —H or —R¹¹;
—R³ is independently —H or —R³³;
—R⁴ is independently —H or —R⁴⁴;
each R¹¹ is independently —R, —R^{X}, —OH, —OR, —OR^{X}, —F, —Cl, —Br, —I, —NH₂, —NHR, —NR₂, —R^{N}, —CN, or —NO₂;
each R³³ is independently —R, —R^{X}, —OH, —OR, —OR^{X}, —F, —Cl, —Br, —I, —NH₂, —NHR, —NR₂, —R^{N}, —CN, or —NO₂;
each R⁴⁴ is independently —R, —R^{X}, —OH, —OR, —OR^{X}, —F, —Cl, —Br, —I, —NH₂, —NHR, —NR₂, —R^{N}, —CN, or —NO₂;
wherein:
each —R is independently saturated linear or branched C₁₋₄alkyl;
each —R^{X} is independently saturated linear or branched C₁₋₄haloalkyl; and
each —R^{N} is independently azetidino, pyrrolidino, piperidino, piperazino, morpholino, thiomorpholino, or 1,1-dioxo-thiomorpholino, and is optionally substituted with one or more substituents selected from —R, —OH, and —OR;
with the proviso that the compound is not a compound of one of the following formulae, or a pharmaceutically acceptable salt or solvate thereof: 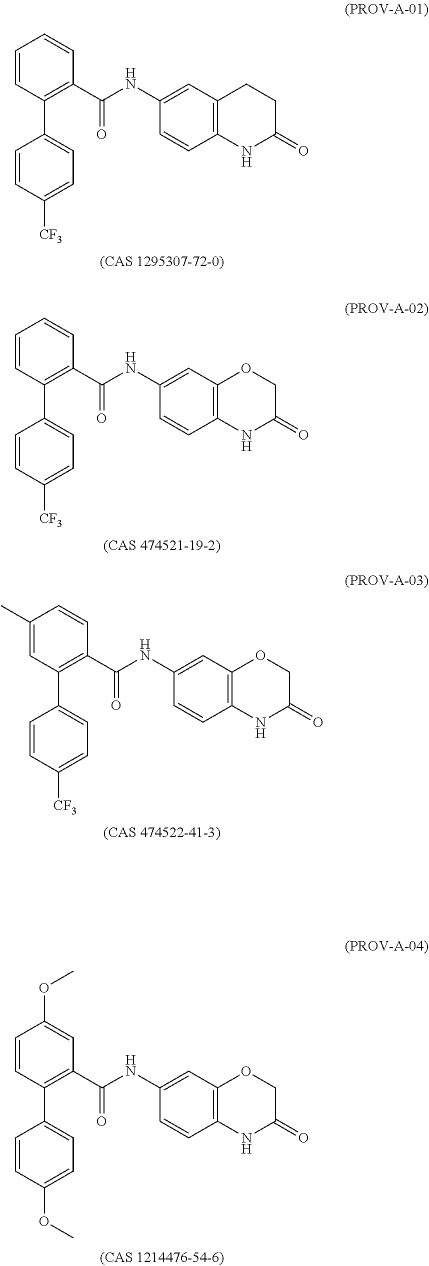 [SMILES: O=C1CCc2cc(NC(=O)c3ccccc3-c3ccc(C(F)(F)F)cc3)ccc2N1 ; O=C1COc2cc(NC(=O)c3ccccc3-c3ccc(C(F)(F)F)cc3)ccc2N1 ; Cc1ccc(C(=O)Nc2ccc3c(c2)OCC(=O)N3)c(-c2ccc(C(F)(F)F)cc2)c1 ; COc1ccc(-c2ccc(OC)cc2C(=O)Nc2ccc3c(c2)OCC(=O)N3)cc1]
and with the proviso that the compound is not a compound of one of the following formulae, or a pharmaceutically acceptable salt or solvate thereof: 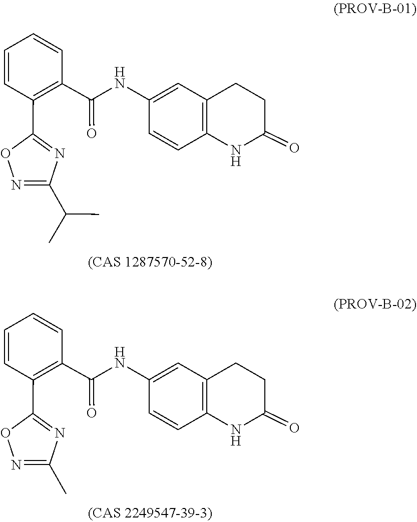 [SMILES: CC(C)c1noc(-c2ccccc2C(=O)Nc2ccc3c(c2)CCC(=O)N3)n1 ; Cc1noc(-c2ccccc2C(=O)Nc2ccc3c(c2)CCC(=O)N3)n1]
and with the proviso that the compound is not a compound of one of the following formulae, or a pharmaceutically acceptable salt or solvate thereof: 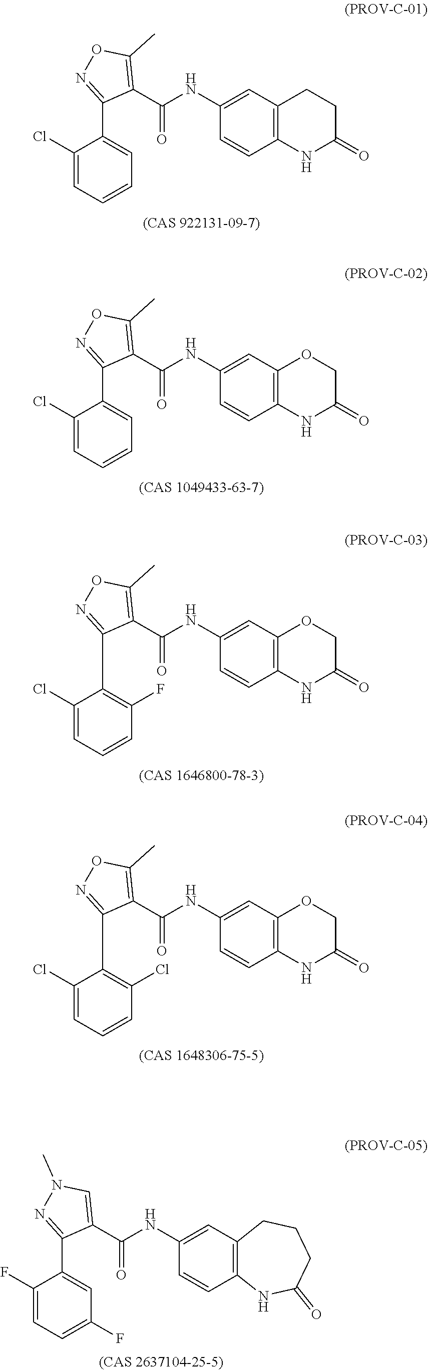 [SMILES: Cc1onc(-c2ccccc2Cl)c1C(=O)Nc1ccc2c(c1)CCC(=O)N2 ; Cc1onc(-c2ccccc2Cl)c1C(=O)Nc1ccc2c(c1)OCC(=O)N2 ; Cc1onc(-c2c(F)cccc2Cl)c1C(=O)Nc1ccc2c(c1)OCC(=O)N2 ; Cc1onc(-c2c(Cl)cccc2Cl)c1C(=O)Nc1ccc2c(c1)OCC(=O)N2 ; Cn1cc(C(=O)Nc2ccc3c(c2)CCCC(=O)N3)c(-c2cc(F)ccc2F)n1] 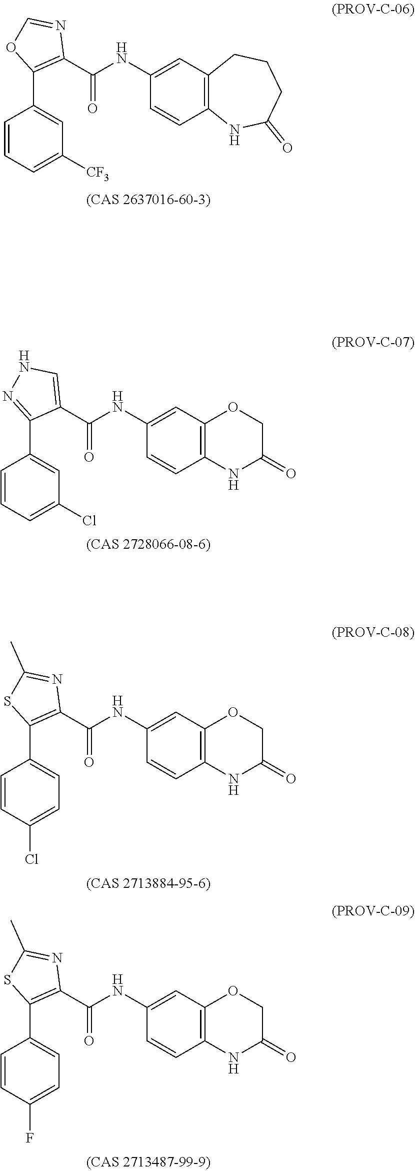 [SMILES: O=C1CCCc2cc(NC(=O)c3ncoc3-c3cccc(C(F)(F)F)c3)ccc2N1 ; O=C1COc2cc(NC(=O)c3c[nH]nc3-c3cccc(Cl)c3)ccc2N1 ; Cc1nc(C(=O)Nc2ccc3c(c2)OCC(=O)N3)c(-c2ccc(Cl)cc2)s1 ; Cc1nc(C(=O)Nc2ccc3c(c2)OCC(=O)N3)c(-c2ccc(F)cc2)s1]
and with the proviso that the compound is not a compound of one of the following formulae, or a pharmaceutically acceptable salt or solvate thereof: 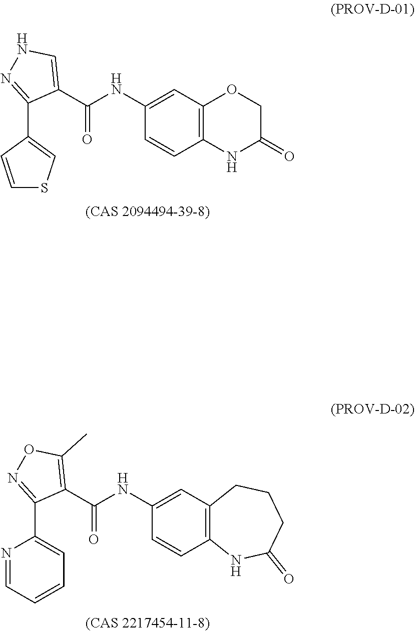 [SMILES: O=C1COc2cc(NC(=O)c3c[nH]nc3-c3ccsc3)ccc2N1 ; Cc1onc(-c2ccccn2)c1C(=O)Nc1ccc2c(c1)CCCC(=O)N2]
and with the proviso that the compound is not a compound of one of the following formulae, or a pharmaceutically acceptable salt or solvate thereof:

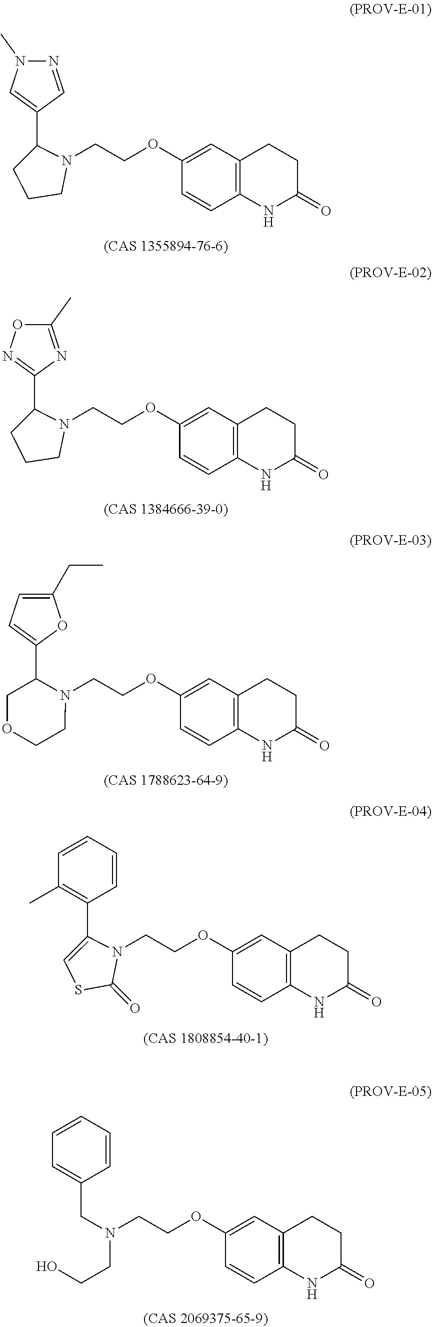 [SMILES: Cn1cc(C2CCCN2CCOc2ccc3c(c2)CCC(=O)N3)cn1 ; Cc1nc(C2CCCN2CCOc2ccc3c(c2)CCC(=O)N3)no1 ; CCc1ccc(C2COCCN2CCOc2ccc3c(c2)CCC(=O)N3)o1 ; Cc1ccccc1-c1csc(=O)n1CCOc1ccc2c(c1)CCC(=O)N2 ; O=C1CCc2cc(OCCN(CCO)Cc3ccccc3)ccc2N1]

2. A compound according to claim 1, wherein -Q- is —CH₂—CR^{Q1}R^{Q2}—.

3. A compound according to claim 1 or 2, wherein each —R^{Q1}, if present, is —H.

4. A compound according to any one of claims 1 to 3, wherein each —R^{Q2}, if present, is —H.

5. A compound according to any one of claims 1 to 4, wherein —R¹ is —H.

6. A compound according to any one of claims 1 to 5, wherein —R³ is —H.

7. A compound according to any one of claims 1 to 5, wherein —R³ is —R³³, and —R³³ is independently —R, —F, or —Cl.

8. A compound according to any one of claims 1 to 7, wherein —R⁴ is —H.

9. A compound according to any one of claims 1 to 8, wherein -J is:

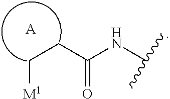

10. A compound according to any one of claims 1 to 9, wherein Ring A, if present, is a C₅heteroaryl group; and is optionally substituted with one or more substituents —R^{A}.

11. A compound according to any one of claims 1 to 9, wherein Ring A, if present, is thiazolyl; and is optionally substituted with a substituent —R^{A}.

12. A compound according to any one of claims 1 to 9, wherein Ring A, if present, is a thiazolyl of the following formula, where (*) denotes the point of attachment to -M¹ and (#) denotes the point of attachment to the —C(═O)— of group -J; and is optionally substituted with a substituent —R^{A}:

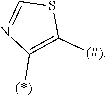

13. A compound according to any one of claims 1 to 12, wherein each —R^{A}, if present, is independently —R^{AA}, —R^{AAX}, —OH, —OR^{AA}, —OR^{AAX}, —F, —Cl, —Br, —I, —NH₂, —NHR^{AA}, —NR^{AA}₂, —R^{AAN}, —CN, or —NO₂.

14. A compound according to any one of claims 1 to 13, wherein each —R^{AA}, if present, is independently -Me or -Et.

15. A compound according to any one of claims 1 to 14, wherein each —R^{AAN}, if present, is independently azetidino, pyrrolidino, piperidino, piperazino, or morpholino, and is optionally substituted with one or more substituents selected from —R^{AA}, —OH, and —OR^{AA}.

16. A compound according to any one of claims 1 to 15, wherein -M¹, if present, is:

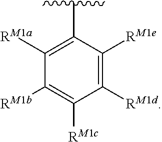

17. A compound according to any one of claims 1 to 15, wherein -M¹, if present, is:

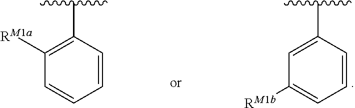

18. A compound according to any one of claims 1 to 17, wherein —R^{M1b}, if present, is —R^{M1-meta}.

19. A compound according to any one of claims 1 to 18, wherein:
each —R^{M1-ortho}, if present, is —F;
each —R^{M1-meta}, if present, is —F; and
each —R^{M1-para}, if present, is —F.

20. A compound according to any one of claims 1 to 15, wherein -M¹, if present, is:

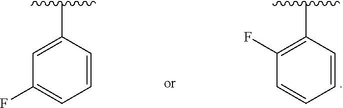 [SMILES: *c1cccc(F)c1 ; *c1ccccc1F]

21. A compound according to any one of claims 1 to 15, wherein -M¹, if present, is a C₅heteroaryl group; and is optionally substituted with one or more substituents —R^{M1}.

22. A compound according to any one of claims 1 to 15, wherein -M¹, if present, is thienyl; and is optionally substituted with one or more substituents —R^{M1}.

23. A compound according to any one of claims 1 to 22, wherein each —R^{M1}, if present, is independently —R^{M11}, —F, —Cl, —Br, or —I.

24. A compound according to any one of claims 1 to 23, wherein each —R^{M11}, if present, is independently -Me or -Et.

25. A compound according to any one of claims 1 to 8, wherein -J is:

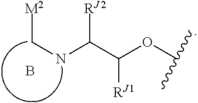

26. A compound according to any one of claims 1 to 8, wherein -J is:

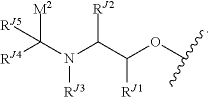

27. A compound according to any one of claims 1 to 8 and 25, wherein Ring 1, if present, is independently azetidinyl, pyrrolidinyl, piperidinyl, piperazinyl, or morpholinyl; and is optionally substituted with one or more substituents —R^{B1} and/or is optionally substituted with ═O.

28. A compound according to any one of claims 1 to 8 and 25, wherein Ring B2, if present, is a C₅heteroaryl group; and is optionally substituted with one or more substituents —R^{B2}.

29. A compound according to any one of claims 1 to 8 and 25, wherein Ring B2, if present, is independently imidazolyl or pyrazolyl; and is optionally substituted with one or more substituents —R^{B2}.

30. A compound according to any one of claims 1 to 8 and 25 to 29, wherein -M², if present, is phenyl; and is optionally substituted with one or more substituents —R^{M2}.

31. A compound according to any one of claims 1 to 8 and 25 to 29, wherein -M², if present, is independently furanyl, thienyl, pyrrolyl, imidazolyl, oxazolyl, isoxazolyl, thiazolyl, isothiazolyl, pyrazolyl, triazolyl, oxadiazolyl, thiadiazolyl, pyridyl, pyridazinyl, pyrimidinyl, or pyrazinyl; and is optionally substituted with one or more substituents —R^{M2}.

32. A compound according to any one of claims 1 to 8 and 25 to 31, wherein each —R^{M2}, if present, is independently —R^{M22}, —F, —Cl, —Br, or —I.

33. A compound according to any one of claims 1 to 8 and 25 to 32, wherein each —R^{M22}, if present, is independently saturated linear or branched C₁₋₃alkyl.

34. A compound according to any one of claims 1 to 8 and 25 to 33, wherein each —R^{J1}, if present, is —H.

35. A compound according to any one of claims 1 to 8 and 25 to 34, wherein each —R^{J2}, if present, is —H.

36. A compound according to any one of claims 1 to 8 and 25 to 35, wherein —R^{J3}, if present, is —R^{JJ}.

37. A compound according to any one of claims 1 to 8 and 25 to 36, wherein:
—R^{J4}, if present, is independently —H, —R^{JJ}, or -L^{JJ}-OH;
—R^{J5}, if present, is independently —H, —R^{JJ}, or -L^{JJ}-OH; or
—R^{J4} and —R^{J5}, if present, taken together form ═O.

38. A compound according to any one of claims 1 to 8 and 25 to 37, wherein each —R^{JJ}, if present, is independently saturated linear or branched C₁₋₃alkyl.

39. A compound according to claim 1, which is a compound of one of the following formulae, or a pharmaceutically acceptable salt or solvate thereof:
ALDHI-1001 through ALDHI-1026;
ALDHI-2001 through ALDHI-2053; and
ALDHI-3001 through ALDHI-3014.

40. A pharmaceutical composition comprising a compound according to any one of claims 1 to 39, and a pharmaceutically acceptable carrier or diluent.

41. A method of preparing a pharmaceutical composition comprising the step of mixing a compound according to any one of claims 1 to 39, and a pharmaceutically acceptable carrier or diluent.

42. A method of inhibiting aldehyde dehydrogenase enzyme ALDH1A3, in vitro or in vivo, comprising contacting the enzyme with an effective amount of a compound according to any one of claims 1 to 39.

43. A method of inhibiting aldehyde dehydrogenase enzyme ALDH1A3 function in a cell, in vitro or in vivo, comprising contacting the cell with an effective amount of a compound according to any one of claims 1 to 39.

44. A compound according to any one of claims 1 to 39, for use in a method of treatment of the human or animal body by therapy.

45. A compound according to any one of claims 1 to 39, for use in a method of treatment of a disorder of the human or animal body that is ameliorated by the inhibition of aldehyde dehydrogenase enzyme ALDH1A3.

46. Use of a compound according to any one of claims 1 to 39 in the manufacture of a medicament for the treatment of a disorder of the human or animal body that is ameliorated by the inhibition of aldehyde dehydrogenase enzyme ALDH1A3.

47. A method of treatment of a disorder of the human or animal body that is ameliorated by the inhibition of aldehyde dehydrogenase enzyme ALDH1A3, comprising administering to a subject in need of treatment a therapeutically-effective amount of a compound according to any one of claims 1 to 39.

48. A compound according to any one of claims 1 to 39, for use in a method of treatment of a proliferative disorder.

49. Use of a compound according to any one of claims 1 to 39 in the manufacture of a medicament for the treatment of a proliferative disorder.

50. A method of treatment of a proliferative disorder of the human or animal body, comprising administering to a subject in need of treatment a therapeutically-effective amount of a compound according to any one of claims 1 to 39.

51. A compound for use according to claim 48, use according to claim 49, or a method according to claim 50, wherein the proliferative disorder is cancer.

52. A compound for use according to claim 48, use according to claim 49, or a method according to claim 50, wherein the proliferative disorder is: melanoma; fibrosarcoma; breast cancer; glioma; glioblastoma; lung cancer; mesothelioma; thyroid cancer; renal cell carcinoma; pancreatic cancer; gastric cancer; colorectal cancer; gallbladder cancer; cholangiocarcinoma; neuroblastoma; testicular germ cell cancer; ovarian cancer; or prostate cancer.

53. A compound for use according to claim 51 or 52, use according to claim 51 or 52, or a method according to claim 51 or 52, wherein the cancer is characterised by aberrant expression of ALDH1A3.

54. A compound for use according to claim 51 or 52, use according to claim 51 or 52, or a method according to claim 51 or 52, wherein the cancer characterised by overexpression of ALDH1A3.

55. A compound for use according to any one of claims 51 to 54, use according to any one of claims 51 to 54, or a method according to any one of claims 51 to 54, wherein the cancer is characterised, or further characterised, as chemotherapy-resistant cancer and/or radiotherapy-resistant cancer.

56. A compound for use according to any one of claims 51 to 55, use according to any one of claims 51 to 55, or a method according to any one of claims 51 to 55, wherein the cancer is characterised, or further characterised, as immunotherapy-resistant cancer.

57. A compound for use according to any one of claims 51 to 55, use according to any one of claims 51 to 55, or a method according to any one of claims 51 to 55, wherein the cancer is characterised, or further characterised, as immunotherapy-resistant cancer characterised by the presence or elevated presence of T-regulatory cells.

58. A compound according to any one of claims 1 to 39, for use in a method of treatment of obesity or a complication of obesity, including type II diabetes.

59. Use of a compound according to any one of claims 1 to 39 in the manufacture of a medicament for the treatment of obesity or a complication of obesity, including type II diabetes.

60. A method of treatment of obesity or a complication of obesity, including type II diabetes, of the human or animal body, comprising administering to a subject in need of treatment a therapeutically-effective amount of a compound according to any one of claims 1 to 39.

61. A compound according to any one of claims 1 to 39, for use in a method of treatment of diabetes, including type II diabetes.

62. Use of a compound according to any one of claims 1 to 39 in the manufacture of a medicament for the treatment of diabetes, including type II diabetes.

63. A method of treatment of diabetes, including type II diabetes, of the human or animal body, comprising administering to a subject in need of treatment a therapeutically-effective amount of a compound according to any one of claims 1 to 39.

64. A compound according to any one of claims 1 to 39, for use in a method of treatment of a cardiovascular disorder, including restenosis, intimal hyperplasia, intimal hyperplasia following vascular reconstruction, intimal hyperplasia following coronary artery angioplasty/stenting, intimal hyperplasia following bypass vein grafting, intimal hyperplasia following arteriovenous fistula, intimal hyperplasia following allograft transplantation, and pulmonary arterial hypertension.

65. Use of a compound according to any one of claims 1 to 39 in the manufacture of a medicament for the treatment of a cardiovascular disorder, including restenosis, intimal hyperplasia, intimal hyperplasia following vascular reconstruction, intimal hyperplasia following coronary artery angioplasty/stenting, intimal hyperplasia following bypass vein grafting, intimal hyperplasia following arteriovenous fistula, intimal hyperplasia following allograft transplantation, and pulmonary arterial hypertension.

66. A method of treatment a cardiovascular disorder, including restenosis, intimal hyperplasia, intimal hyperplasia following vascular reconstruction, intimal hyperplasia following coronary artery angioplasty/stenting, intimal hyperplasia following bypass vein grafting, intimal hyperplasia following arteriovenous fistula, intimal hyperplasia following allograft transplantation, and pulmonary arterial hypertension, of the human or animal body, comprising administering to a subject in need of treatment a therapeutically-effective amount of a compound according to any one of claims 1 to 39.
